(19) United States
(12) Patent Application Publication
(10) Pub. No.: US 2024/0165239 A1
(43) Pub. Date: May 23, 2024

(54) Covalent Binding Compounds for the Treatment of Disease

(71) Applicant: Hyku Biosciences Inc., Lexington, MA (US)

(72) Inventors: Guillaume Barbe, Milton, MA (US); Mark George Sauliner, Higganum, CT (US); William John Greenlee, Teaneck, NJ (US); Soumya Ray, Quincy, MA (US)

(73) Assignee: Hyku Biosciences Inc., Lexington, MA (US)

(21) Appl. No.: 18/242,470

(22) Filed: Sep. 5, 2023

Related U.S. Application Data
(63) Continuation of application No. PCT/US2022/019000, filed on Mar. 4, 2022.
(60) Provisional application No. 63/157,412, filed on Mar. 5, 2021.

(51) Int. Cl. A61K 47/54

(52) U.S. Cl. CPC A61K 47/545; A61K 47/548

(57) ABSTRACT

Compounds and compositions that have a Protein Recognition Moiety bound to an electrophile for the selective covalent modification of targeted proteins to treat disorders mediated by the targeted protein are described.

### CROSS-REFERENCE TO RELATED APPLICATIONS

This application is a continuation of International Patent Application No. PCT/US2022/019000, filed in the U.S. Receiving Office on Mar. 4, 2022, which claims the benefit of U.S. Provisional Application No. 63/157,412, filed on Mar. 5, 2021. The entirety of each of these applications is hereby incorporated by reference for all purposes.

### INCORPORATION BY REFERENCE

The contents of the xml file named “20122-004WO1US1_ST26_2023-11-17” which was created on Nov. 17, 2023, and is 11.5 KB in size, are hereby incorporated by reference in their entirety.

### FIELD OF THE INVENTION

This invention provides heteroaryl sulfonyl compounds and compositions that have a Protein Recognition Moiety bound to a heteroaryl sulfonyl compound for the selective covalent modification of a selected Target Protein to treat a disorder mediated by the Target Protein.

### BACKGROUND OF THE INVENTION

Most cells in the body are terminally differentiated with protective mechanisms to prevent cellular proliferation. A small subset of cells undergo cellular proliferation mainly to replenish tissue or blood components, such as hematopoietic cells and their progeny. The body maintains a careful balance between terminally differentiation and cellular proliferation through a complex network of cellular signaling. Disease states can be caused by dysfunction of the carefully balanced cell proliferation natural pathways or through dysfunction in signaling cascades or dysfunctional gene expression. One way to modify disease states is to disable the function of a protein mediating the disease by covalently modifying it.

The Scripps Research Institute filed three PCT Applications, WO2015/188120, WO2018/102433, and WO2019/139979 describing fluorosulfur(VI) compounds and uses thereof via reactions with phenols. This chemistry, known as SuFEx (Sulfur-Fluoride Exchange), has also been studied by the Sharpless lab at the Scripps Research Institute, which has published a number of papers on the topic (Dong et al. Angew. Chem. Int. Ed Engl. 53(36), 9466-9470 (2014); Qin et al. Angew. Chem. Int. Ed 55(45), 14155-14158 (2016); Gao et al. Angew. Chem. Int. Ed 57(7), 1939-1943 (2018); Guo et al. Angew. Chem. Int. Ed 57(10), 2605-2610 (2017); Gahtory et al. Chemistry 24(41), 10550-10556, (2018); Smedley et al. Angew. Chem. Int. Ed 58(14), 4552-4556 (2019); Liu et al. Angew. Chem. Int. Ed 58(24), 8029-8033 (2019); Dong et al. Angew. Chem. Int. Ed 53(36), 9430-9448 (2014); Li et al. Angew. Chem. Int. Ed 56(11), 2903-2908 (2017); Zheng et al. PNAS 116(38) 18808-18814 (2019); Wang et al. Angew. Chem. Int. Ed 56(37), 11203-11208 (2017); Liu et al. J. Am. Chem. Soc. 140, 2919-2925 (2018); and Chen et al. J. Am. Chem. Soc. 138, 7353-7364 (2016)). Similar sulfonyl fluoride chemistries have been developed for the purpose of biorthogonal protein labelling (Narayanan et al. Chem. Sci. 6(5): 2650-2659 (2015) and Gu et al. J Chem. Biol. 20(4), 541-548 (2013)) These strategies employ electrophilic sulfonyl compounds with a fluoride leaving group to react with a variety of nucleophiles.

Ku-Lung Hsu, et al., at University of Virginia have described sulfonyl-containing heteroaryl compound which have been named SuTEx compounds (Sulfur-Triazole Exchange)(Hahm et al. Nat. Chem. Bio. 16, 150-159 (2020); Brulet et al. J. Am. Chem. Soc. 142(18), 8270-8280 (2020); Borne et al. Development and biological applications of sulfur-triazole exchange (SuTEx) chemistry RSC Chem. Biol. (2021); and Huang et al. Chemoproteomic profiling of kinases in live cells using electrophilic sulfonyl triazole probes Chem. Sci. (2021)). See also WO 2020/214336 (Sulfur-heterocycle exchange chemistry) and WO 2021/016263 (Cysteine Binding Compositions and Methods of Use Thereof), filed by University of Virginia as assignee, and Hsu, et al. as inventors. Additional publications on the use of SuTEx molecules include Grams et al. Reactive chemistry for covalent probe and therapeutic development Trends in Pharmacological Sciences (2022) and Toroitich et al. Discovery off a cell-active SuTEx ligand of prostaglandin reductase 2 ChemBioChem (2021).

Despite many years of medical research there are still disorders for which there are no cure or an insufficient cure. It is an object of the present invention to provide new compounds, compositions, treatments and manufactures thereof for medical disorders.

### SUMMARY OF THE INVENTION

Heteroaryl sulfonyl compounds and their uses and manufacture are provided that covalently modify a Target Protein to treat a disease mediated by the Target Protein in a host, typically a human. The heteroaryl sulfonyl compound is first typically selectively non-covalently bound to the Target Protein by association of the Target Protein with a Protein Recognition Moiety in the heteroaryl sulfonyl compound. In a typical second step, a reactive tyrosine residue on the Target Protein attacks the sulfonyl moiety in the heteroaryl sulfonyl compound of the present invention to form a covalent bond between the tyrosine and the compound and force the elimination of a Leaving Group from the compound. In another aspect a reactive lysine residue on the Target Protein attacks the heteroaryl sulfonyl compound of the present invention to form a covalent bond between the lysine and the compound and force the elimination of a Leaving Group from the compound.

The heteroaryl sulfonyl compounds of the present invention are uniquely designed for specificity to their respective Target Protein to maximize therapeutic effect and minimize off-target toxicity, by inclusion of a specific Protein Recognition Moiety as described further herein that selectively binds the selected Targeted Protein for further covalent linkage. In this way, the heteroaryl sulfonyl compound of the present invention exerts precise control over the targeted silencing, destruction or inactivation of the Target Protein while limiting unacceptable off-target effects.

The Protein Recognition Moiety is a molecule that has a functional group linking it to the heteroaryl sulfonyl compound of the present invention, and is, for example, a synthetic or naturally occurring small molecule that binds to the Target Protein as an inhibitor or alternatively with no apparent biological effect on the Target Protein. In non-limiting embodiments, the Protein Recognition Moiety is a protein binding domain of a drug or pharmaceutically active compound which modulates the Targeted Protein (or the full drug or pharmaceutically active compound). In alternative embodiments, the Protein Recognition Moiety may be a peptide, RNA, DNA, oligonucleotide, or another biologic compound or fragment thereof which can be suitably stabilized, as necessary.

The heteroaryl sulfonyl compounds described herein can take advantage of the variable electrophilic properties of heteroaryl sulfonyl compounds to covalently modify Targeted Proteins, resulting in a decrease or termination of its biological activity.

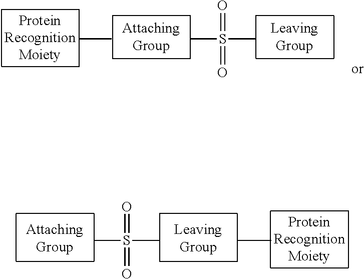

The covalent-binding heteroaryl sulfonyl compounds of the present invention include a Protein Recognition Moiety, a Leaving Group, and an Attaching Group. The heteroaryl sulfonyl compounds are oriented such that the Leaving Group is on one side of the S(O)₂ electrophile and the Attaching Group is on the other. The Protein Recognition Moiety is located either on the Leaving Group or the Attaching Group in a manner that allows it to associate with the Target Protein as described herein.

In some embodiments, the Leaving Group is a monocyclic or bicyclic heteroaryl group bound to the sulfur atom through a S—N bond. For example, as used herein, R¹ and R⁴ are typically Leaving Groups. The Leaving Group is eliminated when the heteroaryl sulfonyl compound undergoes nucleophilic attack by an amino acid, for example a tyrosine or lysine, of the Target Protein. The Attaching Group and the sulfonyl to which it is attached remains on the Target Protein after covalent modification. For example, as used herein, R², R⁵, and R¹³ are Attaching Groups.

A non-limiting example of the covalent modification of a Target Protein via a tyrosine that reacts with the heteroaryl sulfonyl compound of the present invention is provided below:

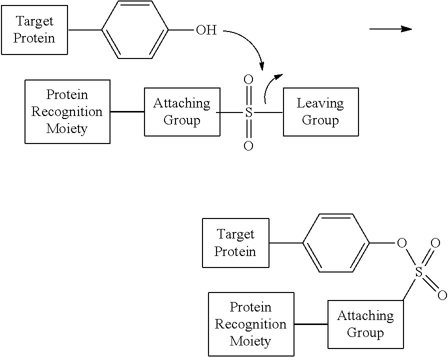

The Protein Recognition Moiety brings the activated heteroaryl sulfonyl compound of the present invention into close proximity with a reactive amino acid of the Target Protein resulting in covalent modification of the Target Protein and resultant amelioration or elimination of a disease or Target Protein-mediated disorder.

The heteroaryl sulfonyl compound of the present invention is used to modulate a Target Protein's biological activity by covalently modifying the protein, for example, by covalently modifying a tyrosine in or near the active site, or alternatively, a lysine moiety.

In one aspect a heteroaryl sulfonyl compound of Formula I, Formula II, Formula III, Formula IV, Formula V, or Formula VI, is provided:

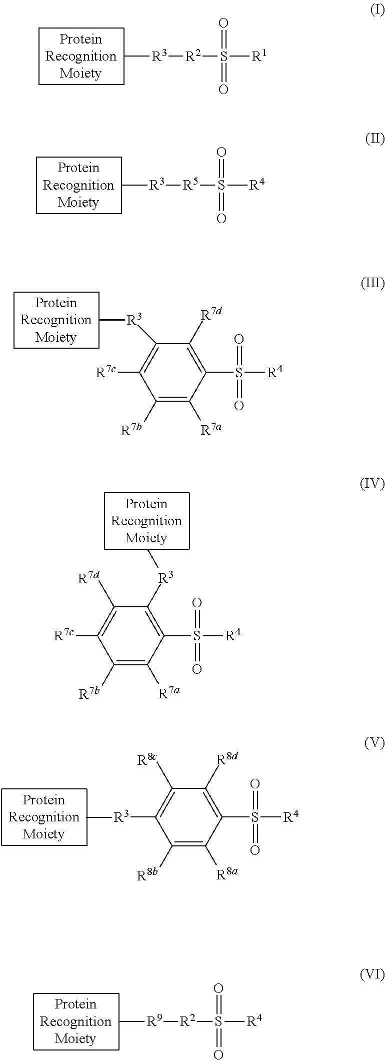

or a pharmaceutically acceptable salt, N-oxide, isotopic derivative, or prodrug thereof, optionally in a pharmaceutically acceptable carrier to form a composition;
wherein:
R¹ is selected from:

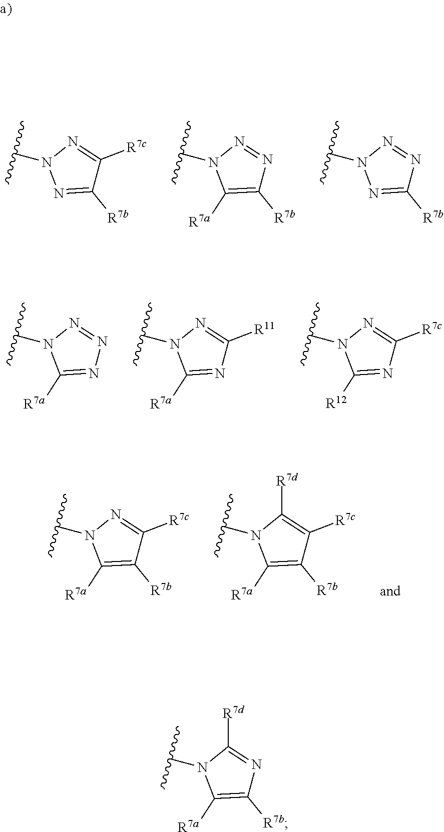

or
b) a fused bicyclic heteroaryl optionally substituted as allowed by valence with 1, 2, 3, or 4 substituents selected from R⁷;
R² is a bivalent moiety independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which except bond is optionally substituted as allowed by valence with 1, 2, 3, or 4 substituents selected from R⁷;
in principal embodiments R² is selected from alkyl, alkenyl, haloalkyl, cycloalkyl, aryl, bicycle, tricycle, and heteroaryl, each of which is optionally substituted as allowed by valence with 1, 2, 3, or 4 substituents selected from R⁷;
R³ is a bivalent moiety independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which except bond is optionally substituted as allowed by valence with 1, 2, 3, or 4 substituents selected from R⁷;
or R³ is a bivalent moiety independently selected at each instance from bond, alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents selected from R⁷;
and wherein R² and R³ are selected such that a suitably stable and suitably nontoxic compound for in vivo administration in a host is achieved, and in certain embodiments, in a way that avoids undesired repetition of atoms or moieties, such as in nonlimiting examples, S, 0, or a combination thereof (i.e., that would otherwise form a disulfide or peroxide bond), as well known to skilled artisans;
p is independently selected from 1, 2, 3, 4, 5, and 6;
R⁴ is a heteroaryl group, where the bond to the sulfur atom is through one of the nitrogen atoms present in the cycle, and each heteroaryl is optionally substituted as allowed by valence with 1, 2, 3, or 4 substituents selected from R⁷;
R⁵ is a bivalent moiety selected from alkyl, alkenyl, haloalkyl, cycloalkyl, naphthyl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl-C(O)—NR⁶—, bicycle, tricycle, and heteroaryl, each of which is optionally substituted as allowed by valence with 1, 2, 3, or 4 substituents selected from R⁷;
in principal embodiments R⁵ is selected from alkyl, alkenyl, haloalkyl, cycloalkyl, naphthyl, heterocycle, bicycle, tricycle, and heteroaryl, each of which is optionally substituted as allowed by valence with 1, 2, 3, or 4 substituents selected from R⁷;
R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;
in certain embodiments R⁶ is not substituted;
R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, —S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;
in certain embodiments R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;
R¹⁷ is independently selected in each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶;
R¹⁸ is independently selected in each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, ═O, and —SR¹⁹;
R¹⁹ is independently selected at each instance from hydrogen, alkyl, haloalkyl, cycloalkyl, aryl, heterocycle, and heteroaryl;
R^{8a}, R^{8b}, R^{8c}, and R^{8d} are independently selected at each instance from R⁷ and R¹² wherein at least one of R^{8a}, R^{8b}, R^{8c}, and R^{8d} is R¹²;
R⁹ is a bivalent moiety selected from, alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, and heteroaryl, each of which is optionally substituted as allowed by valence with 1, 2, 3, or 4 substituents selected from R⁷;
R¹¹ is selected from hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, and —SR⁶, wherein each alkyl, haloalkyl, alkenyl, cycloalkyl, heterocycle, aryl, and heteroaryl optionally substituted as allowed by valence with 1, 2, 3, or 4 substituents selected from R¹⁷;
R¹² is independently selected from halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷; and
Protein Recognition Moiety is a molecule, for example a small molecule, peptide, protein, oligonucleotide, nucleotide, RNA, DNA, SiRNA, a biologic, an antibody or a fragment thereof, which can bind to or otherwise interact with a Target Protein; and
Target Protein is a mediator of disease.

In certain embodiments the Target Protein has a reactive tyrosine which covalently binds to the heteroaryl sulfonyl compounds of the present invention. In certain embodiments the reactive tyrosine is in an active site. In certain embodiments the reactive tyrosine is not in an active site.

In certain embodiments the Target Protein has a reactive lysine which covalently binds to the heteroaryl sulfonyl compounds of the present invention. In certain embodiments the reactive lysine is in an active site. In certain embodiments the reactive lysine is not in an active site.

In certain embodiments the Target Protein has a reactive cysteine which covalently binds to the heteroaryl sulfonyl compounds of the present invention. In certain embodiments the reactive cysteine is in an active site. In certain embodiments the reactive cysteine is not in an active site.

Assays and/or spectroscopic techniques to confirm covalent binding are described in the paper by Brulet et. al. titled “Liganding Functional Tyrosine Sites on Proteins Using Sulfur-Triazole Exchange Chemistry” JACS 2020, 142, 8270-8280 or the paper by Hahm et. al. titled “Global targeting of functional tyrosines using sulfur triazole exchange chemistry” Nature Chem. Biol. 2020, 16(2), 150-159.

In certain embodiments the heteroaryl sulfonyl compound of the present invention primarily covalently modifies a specific tyrosine or lysine in the Target Protein. In other embodiments, a selected heteroaryl sulfonyl compound of the present invention reacts with two or more different tyrosines and/or lysines in the Target Protein. In certain embodiments the heteroaryl sulfonyl compound of the present invention is more than about 5-, 10-, 15-, 20-, 25-, 50-, 75-, or 100-fold more selective for one specific amino acid, for example a specific tyrosine, than other amino acids of the Target Protein.

In certain embodiments one or more amino acids other than tyrosine or lysine is covalently modified by a heteroaryl sulfonyl compound of the present invention. For example, the amino acid that is covalently modified is cysteine, arginine, histidine, serine, threonine, or tryptophan.

In one aspect a heteroaryl sulfonyl compound of Formula VII is provided:

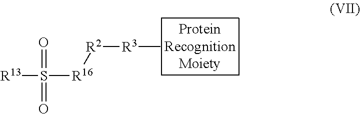

or a pharmaceutically acceptable salt, N-oxide, isotopic derivative, or prodrug thereof, optionally in a pharmaceutically acceptable carrier to form a composition;
wherein:
R¹³ is selected from hydrogen, alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, and —NR⁶C(O)N(R⁶)₂, each of which except hydrogen is optionally substituted as allowed by valence with 1, 2, 3, or 4 substituents selected from R⁷;
in certain embodiments R¹³ is selected from hydrogen, alkyl, haloalkyl, cycloalkyl, heterocycle, heteroaryl, and aryl, each of which except hydrogen is optionally substituted as allowed by valence with 1, 2, 3, or 4 substituents selected from R⁷;
R¹⁶ is a heteroaryl group, where the bond to the sulfur atom is through one of the a nitrogen present in the cycle, and R¹⁶ is optionally substituted as allowed by valence with 1, 2, 3, or 4 substituents selected from R⁷;
   and all other variables are as defined herein.

In one aspect a heteroaryl sulfonyl compound of Formula VIII is provided:

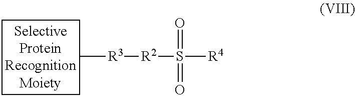

or a pharmaceutically acceptable salt, N-oxide, isotopic derivative, or prodrug thereof, optionally in a pharmaceutically acceptable carrier to form a composition;
wherein Selective Protein Recognition Moiety is a Protein Recognition Moiety as defined herein wherein at least one of the following is satisfied:
i. there are fewer than 80, 70, 60, 50, 40, 30, 20, 15, 10, or 5 endogenous protein kinases to which the Selective Protein Recognition Moiety binds with an K_{d}50 of 2 μM or less;
ii. the Selective Protein Recognition Moiety has an K_{d}50 greater than 1 μM against aurora B kinase, c-Src kinase domain, human serine/threonine-protein kinase MST4, activin receptor type-IIA (ACVR2A), human calcium calmodulin dependent protein kinase II delta isoform 1 (CAMKD), and/or human ste20-like kinase;
   and wherein all other variables are as defined herein.

In a typical embodiment, R² is selected in each instance from bond, alkyl, alkenyl, haloalkyl, cycloalkyl, aryl, heterocycle, —(CH₂)ₚ—C(O)—, —(OCH₂CH₂)ₚ—, —C(O)—, —NR⁶C(O)—, and heteroaryl, each of which except bond is optionally substituted as allowed by valence with 1, 2, 3, or 4 substituents selected from R⁷, wherein if R² is bond, R³ is R³*; wherein
R^{3*} is selected in each instance from bond, alkyl, alkenyl, haloalkyl, cycloalkyl, aryl, heterocycle, —(CH₂)ₚ—C(O)—, —(OCH₂CH₂)ₚ—, —C(O)—, —NR⁶C(O)—, bicycle, and heteroaryl, each of which except bond is optionally substituted as allowed by valence with 1, 2, 3, or 4 substituents selected from R⁷.

In a typical embodiment R⁵ is selected from alkyl, alkenyl, haloalkyl, cycloalkyl, naphthyl, heterocycle, —(OCH₂CH₂)ₚ—, —C(O)—, —NR⁶C(O)—, bicycle, and heteroaryl, each of which is optionally substituted as allowed by valence with 1, 2, 3, or 4 substituents selected from R⁷.

In certain embodiments of the invention, the Protein Recognition Moiety is a small organic molecule (i.e., a non-biologic) that adequately binds to the Target Protein in a manner that it is covalently modified. In an embodiment of the invention, the Protein Recognition Moiety is a peptide or oligonucleotide that adequately binds to the protein in such a manner that it can be covalently modified. In certain embodiments the Protein Recognition Moiety is a residue of a pharmaceutically active compound that binds to the Target Protein (for example but not limited to a compound of the sort that would be reviewed as a drug by CDER of the FDA, or an approved or clinical stage drug) or a peptide, protein or biologic or a binding fragment thereof that adequately binds to the protein in such a manner that it can be covalently modified. A plethora of illustrative nonlimiting examples of Protein Recognition Moieties is provided in the Figures and additional Protein Recognition Moieties are readily apparent to the skilled artisan.

The present invention focuses on the covalent modification of a selected protein that mediates disease, for example, abnormal cellular proliferation such as a tumor or cancer. In certain embodiments, a method of treating a disorder mediated by a Target Protein is provided comprising administering an effective amount of a heteroaryl sulfonyl compound of Formula I, Formula II, Formula III, Formula IV, Formula V, Formula VI, Formula VII, or Formula VIII, to a patient in need thereof, for example a human, or a pharmaceutically acceptable salt thereof optionally in a pharmaceutically acceptable carrier. For example, in one embodiment, a heteroaryl sulfonyl compound of Formula I, Formula II, Formula III, Formula IV, Formula V, Formula VI, Formula VII, or Formula VIII, is administered to a human to treat a cancer or tumor where the heteroaryl sulfonyl compound has a Protein Recognition Moiety that targets a protein that mediates the cancer or tumor.

In certain embodiments, a heteroaryl sulfonyl compound described herein does not have to be administered in as high of a dose or as frequently as the Protein Recognition Moiety alone for treatment of a disorder. In certain embodiments, a heteroaryl sulfonyl compound of the present invention has fewer or less severe side effects in the treatment of a disorder mediated by the Target Protein, than the Protein Recognition Moiety alone. In certain embodiments, the heteroaryl sulfonyl compound of the present invention is more efficacious in the treatment of a disorder mediated by the Target Protein than the original protein binder corresponding to the Protein Recognition Moiety alone.

In certain embodiments, a heteroaryl sulfonyl compound described herein is useful to treat a disorder, for example abnormal cellular proliferation, such as a tumor or cancer, wherein the Target Protein is mutated. In other embodiments a heteroaryl sulfonyl compound described herein is useful to treat a disorder for example abnormal cellular proliferation, such as a tumor or cancer, wherein the Target Protein is not mutated. In certain embodiments a heteroaryl sulfonyl compound described herein is at least about 2-, 3-, 4-, 5-, 10-, 50-, 100-, 200-, 300-, 400-, 500-, or 1,000-fold more selective for a mutated Target Protein than the wild-type Target Protein.

In principle embodiments, the Protein Recognition Moiety is not a fluorophore, not a detectable labeling group, and not a moiety comprising an alkyne.

In principle embodiments, the heteroaryl sulfonyl compound of the present invention is also not a chemical probe used to perturb the function of a variety of proteins in a biological sample, but instead a focused Target Protein binder and covalent modifier.

In certain embodiments, a heteroaryl sulfonyl compound of the present invention is useful as a therapeutic agent, when administered in an effective amount to a patient, for the treatment of a medical disorder that can be treated with the Protein Recognition Moiety.

The heteroaryl sulfonyl compounds of the present invention can be administered in any manner that allows the heteroaryl sulfonyl compound to covalently modify the Target Protein. As such, examples of methods to deliver the heteroaryl sulfonyl compounds of the present invention include, but are not limited to, oral, parenteral, systemic, topical, transdermal, intravenous, buccal, sublingual, subcutaneous, and transnasal.

In certain embodiments, the heteroaryl sulfonyl compound of the present invention has at least one desired isotopic substitution of an atom, at an amount above the natural abundance of the isotope, i.e., enriched.

In one embodiment, the heteroaryl sulfonyl compound of the present invention includes a deuterium or multiple deuterium atoms. Deuterium is not considered or used herein as a detectable labelling group.

Another aspect of the present invention provides a heteroaryl sulfonyl compound as described herein, or an enantiomer, diastereomer, or stereoisomer thereof, or pharmaceutically acceptable salt, hydrate, or solvate thereof, or a pharmaceutical composition, for use in the manufacture of a medicament for treating or preventing a disease in which the Target Protein plays a role.

In one embodiment, the heteroaryl sulfonyl compound of the present invention is not fluorescent, including but not limited to not a fluorophore.

In other aspects a heteroaryl sulfonyl compound of Formula I′, Formula II′, Formula III′, Formula IV′, Formula V′, Formula VI′, Formula VII′, or Formula VIII′, is provided:

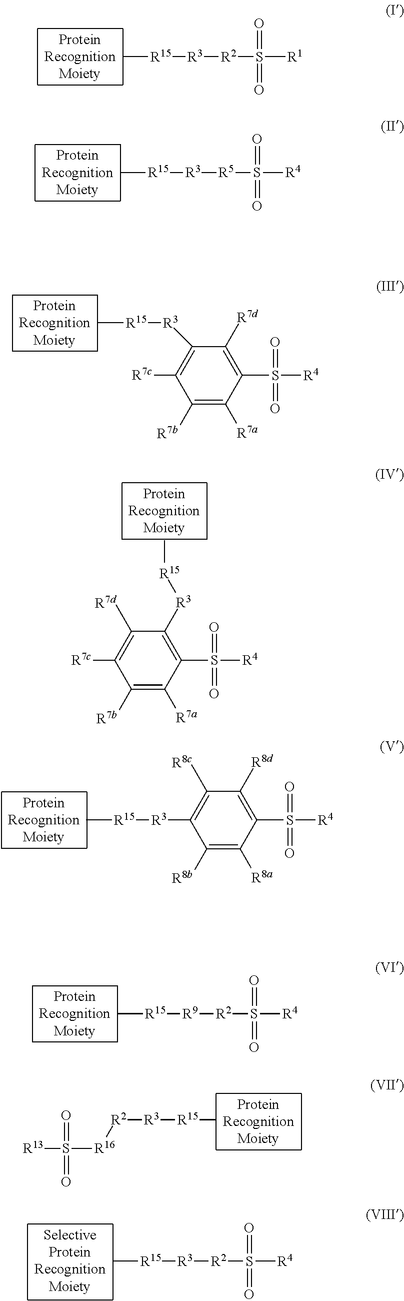

or a pharmaceutically acceptable salt, N-oxide, isotopic derivative, or prodrug thereof, optionally in a pharmaceutically acceptable carrier to form a pharmaceutical composition;
wherein R¹⁵ is a bivalent moiety selected from the group consisting of alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents independently selected from R⁷; and wherein all other variables are as defined herein.

Additional features and advantages of the present application will be apparent from the following detailed description.

The present invention thus includes at least the following features:
(a) A heteroaryl sulfonyl compound of Formula I, Formula II, Formula III, Formula IV, Formula V, Formula VI, Formula VII, or Formula VIII, as described herein, or a pharmaceutically acceptable salt or isotopic derivative (including a deuterated derivative) thereof;
(b) A method for treating a disorder mediated by the Target Protein, comprising administering an effective amount of a heteroaryl sulfonyl compound of Formula I, Formula II, Formula III, Formula IV, Formula V, Formula VI, Formula VII, or Formula VIII, or pharmaceutically acceptable salt thereof, as described herein, to a patient in need thereof,
(c) A heteroaryl sulfonyl compound of Formula I, Formula II, Formula III, Formula IV, Formula V, Formula VI, Formula VII, or Formula VIII, or a pharmaceutically acceptable salt thereof for use in the treatment of a disorder that is mediated by the Target Protein;
(d) Use of a heteroaryl sulfonyl compound of Formula I, Formula II, Formula III, Formula IV, Formula V, Formula VI, Formula VII, or Formula VIII, or a pharmaceutically acceptable salt thereof, in an effective amount in the treatment of a patient in need thereof, typically a human, with disorder mediated by the Target Protein;
(e) Use of a heteroaryl sulfonyl compound of Formula I, Formula II, Formula III, Formula IV, Formula V, Formula VI, Formula VII, or Formula VIII, or a pharmaceutically acceptable salt thereof in the manufacture of a medicament for the treatment of a disorder mediated by the Target Protein;
(f) A pharmaceutical composition comprising an effective patient-treating amount of a heteroaryl sulfonyl compound of Formula I, Formula II, Formula III, Formula IV, Formula V, Formula VI, Formula VII, or Formula VIII, or a pharmaceutically acceptable salt thereof; and a pharmaceutically acceptable carrier or diluent;
(g) A heteroaryl sulfonyl compound of Formula I, Formula II, Formula III, Formula IV, Formula V, Formula VI, Formula VII, or Formula VIII, as described herein as a mixture of enantiomers or diastereomers (as relevant), including as a racemate;
(h) A heteroaryl sulfonyl compound of Formula I, Formula II, Formula III, Formula IV, Formula V, Formula VI, Formula VII, or Formula VIII, as described herein in enantiomerically or diastereomerically (as relevant) enriched form, including an isolated enantiomer or diastereomer (i.e., greater than 85, 90, 95, 97, or 99% pure); and
(i) A process for the preparation of therapeutic products that contain an effective amount of a heteroaryl sulfonyl compound of Formula I, Formula II, Formula III, Formula IV, Formula V, Formula VI, Formula VII, or Formula VIII, or a pharmaceutically acceptable salt thereof, as described herein.
(j) A heteroaryl sulfonyl compound of Formula I′, Formula II′, Formula III′, Formula IV′, Formula V′, Formula VI′, Formula VII′, or Formula VIII′, as described herein, or a pharmaceutically acceptable salt or isotopic derivative (including a deuterated derivative) thereof;
(k) A method for treating a disorder mediated by the Target Protein, comprising administering an effective amount of a heteroaryl sulfonyl compound of Formula I′, Formula II′, Formula III′, Formula IV′, Formula V′, Formula VI′, Formula VII′, or Formula VIII′, or pharmaceutically acceptable salt thereof, as described herein, to a patient in need thereof;
(l) A heteroaryl sulfonyl compound of Formula I′, Formula II′, Formula III′, Formula IV′, Formula V′, Formula VI′, Formula VII′, or Formula VIII′, or a pharmaceutically acceptable salt thereof for use in the treatment of a disorder that is mediated by the Target Protein;
(m) Use of a heteroaryl sulfonyl compound of Formula I′, Formula II′, Formula III′, Formula IV′, Formula V′, Formula VI′, Formula VII′, or Formula VIII′, or a pharmaceutically acceptable salt thereof, in an effective amount in the treatment of a patient in need thereof, typically a human, with disorder mediated by the Target Protein;
(n) Use of a heteroaryl sulfonyl compound of Formula I′, Formula II′, Formula III′, Formula IV′, Formula V′, Formula VI′, Formula VII′, or Formula VIII′, or a pharmaceutically acceptable salt thereof in the manufacture of a medicament for the treatment of a disorder mediated by the Target Protein;
(o) A pharmaceutical composition comprising an effective patient-treating amount of a heteroaryl sulfonyl compound of Formula I′, Formula II′, Formula III′, Formula IV′, Formula V′, Formula VI′, Formula VII′, or Formula VIII′, or a pharmaceutically acceptable salt thereof; and a pharmaceutically acceptable carrier or diluent;
(p) A heteroaryl sulfonyl compound of Formula I′, Formula II′, Formula III′, Formula IV′, Formula V′, Formula VI′, Formula VII′, or Formula VIII′, as described herein as a mixture of enantiomers or diastereomers (as relevant), including as a racemate;
(q) A heteroaryl sulfonyl compound of Formula I′, Formula II′, Formula III′, Formula IV′, Formula V′, Formula VI′, Formula VII′, or Formula VIII′, as described herein in enantiomerically or diastereomerically (as relevant) enriched form, including an isolated enantiomer or diastereomer (i.e., greater than 85, 90, 95, 97, or 99% pure); and
(r) A process for the preparation of therapeutic products that contain an effective amount of a heteroaryl sulfonyl compound of Formula I′, Formula II′, Formula III′, Formula IV′, Formula V′, Formula VI′, Formula VII′, or Formula VIII′, or a pharmaceutically acceptable salt thereof, as described herein.

### BRIEF DESCRIPTION OF THE FIGURES

As used in the figures R²⁷ is independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl optionally substituted as allowed by valence with 1, 2, 3, or 4 substituents selected from R¹⁸;

As used in the figures n is 0, 1, 2, 3, or 4.

As used in the figures [symbol] is an Anchor Bond. Anchor Bond is the chemical bond between the Protein Recognition Moiety and the rest of the molecule for example a bond to R³, R⁹, or R¹⁶, as appropriate.

In the context of crystal structures three letter codes used below refer to specific ligands in the RCSB PDB database which is accessible on https://www.rcsb.org/.

FIGS. 1A, 1B and 1C present non-limiting examples of ligands that bind to isocitrate dehydrogenase cytoplasmic protein (IDH1), including the compounds NDP, 70Q, NAP, 59D, 70P, C81, 6VN, HJQ, 9UO, 1BX, DWP, DWM, DWG, DWJ, DWS, 6MX, 6N3, 7J2, VVS, 42V, 42W, LJY, LJV, PWV, AOU, QWM, 1K9, 69Q, and K32. For additional non-limiting examples and related ligands, see ligands identified by Xie et al., “Allosteric Mutant IDH1 Inhibitors Reveal Mechanisms for IDH1 Mutant and Isoform Selectivity”, Structure, 2017, 25: 506-513; Rendina et al., “Mutant IDH1 Enhances the Production of 2-Hydroxyglutarate Due to Its Kinetic Mechanism”, Biochemistry, 2013, 52: 4563-4577; Okoye-Okafor et al., “New IDH1 mutant inhibitors for treatment of acute myeloid leukemia”, Nat Chem Biol., 2015, 11: 878-886; Cho et al., “Discovery and Evaluation of Clinical Candidate IDH305, a Brain Penetrant Mutant IDH1 Inhibitor”, ACS Med Chem Lett., 2017, 8: 1116-1121; Pusch et al., “Pan-mutant IDH1 inhibitor BAY 1436032 for effective treatment of IDH1 mutant astrocytoma in vivo”, Acta Neuropathol., 2017, 133: 629-644; Chaturvedi et al., “In vivo efficacy of mutant IDH1 inhibitor HMS-101 and structural resolution of distinct binding site”, Leukemia, 2020, 34: 416-426; Ma et al., “Crystal structures of pan-IDH inhibitor AG-881 in complex with mutant human IDH1 and IDH2”, Biochem Biophys Res Commun., 2018, 503: 2912-2917; Zheng et al., “Crystallographic Investigation and Selective Inhibition of Mutant Isocitrate Dehydrogenase”, ACS Med Chem Lett., 2013, 4: 542-546; Jakob et al., “Novel Modes of Inhibition of Wild-Type Isocitrate Dehydrogenase 1 (IDH1): Direct Covalent Modification of His315”, J Med Chem., 2018, 61: 6647-6657; Jones et al., “Discovery and Optimization of Allosteric Inhibitors of Mutant Isocitrate Dehydrogenase 1 (R132H IDH1) Displaying Activity in Human Acute Myeloid Leukemia Cells”, J Med Chem., 2016, 59: 11120-11137; Yang et al., “Molecular mechanisms of “off-on switch” of activities of human IDH1 by tumor-associated mutation R132H”, Cell Res., 2010, 20: 1188-1200; Levell et al., “Optimization of 3-Pyrimidin-4-yl-oxazolidin-2-ones as Allosteric and Mutant Specific Inhibitors of IDH1”, ACS Med Chem Lett., 2017, 8: 151-156; Deng et al., “Selective Inhibition of Mutant Isocitrate Dehydrogenase 1 (Idh1) Via Disruption of a Metal Binding Network by an Allosteric Small Molecule”, J Biol Chem., 2015, 290: 762; Wu et al., “Inhibition of Cancer-Associated Mutant Isocitrate Dehydrogenases by 2-Thiohydantoin Compounds”, J Med Chem., 2015, 58: 6899-6908; Lin et al., “Discovery and Optimization of Quinolinone Derivatives as Potent, Selective, and Orally Bioavailable Mutant Isocitrate Dehydrogenase 1 (mIDH1) Inhibitors”, J Med Chem., 2019, 62: 6575-6596; Caravella et al., “Structure-Based Design and Identification of FT-2102 (Olutasidenib), a Potent Mutant-Selective IDH1 Inhibitor”, J Med Chem., 2020, 63: 1612-1623; Machida et al., “A Potent Blood-Brain Barrier-Permeable Mutant IDH1 Inhibitor Suppresses the Growth of Glioblastoma with IDH1 Mutation in a Patient-Derived Orthotopic Xenograft Model”, Mol Cancer Ther., 2020, 19: 375-383; Dang et al., “Cancer-associated IDH1 mutations produce 2-hydroxyglutarate”, Nature, 2009, 462: 739-744; Konteastis et al., “Vorasidenib (AG-881): A First-in-Class, Brain-Penetrant Dual Inhibitor of Mutant IDH1 and 2 for Treatment of Glioma”, ACS Med Chem Lett., 2020, 11: 101-107; “Targeted inhibition of mutant IDH2 in leukemia cells induces cellular differentiation”, Science, 2013, 340: 622-626; and Yen et al., “AG-221, a First-in-Class Therapy Targeting Acute Myeloid Leukemia Harboring Oncogenic IDH2 Mutations”, Cancer Discov., 2017, 7: 478-493.

FIG. 2 presents non-limiting examples of ligands that bind to phosphoglycerate mutase 1 (PGAM1), including the compounds AZN, 8KX, AW6, 8LF, HKB, 9HU, and 9JF. For additional non-limiting examples and related ligands, see ligands identified by Zhou et al., “X-ray structure of Phosphoglycerate Mutase 1 (PGAM1) complexed with a small molecule”, to be published; Jiang et al., “Phosphoglycerate mutase 1 (PGAM1) complexed with its inhibitor PGMI-004A”, to be published; Jiang et al., “Phosphoglycerate mutase 1 complexed with a small molecule inhibitor”, to be published; Jiang et al., “Phosphoglycerate mutase 1 complexed with a small molecule inhibitor KH1”, to be published; Jiang et al., “Phosphoglycerate mutase 1 complexed with a small molecule inhibitor KH2”, to be published; Jiang et al., “Phosphoglycerate mutase 1 complexed with a small molecule inhibitor In-AC”, to be published; and Jiang et al., “Acetylation of lysine 100 of Phosphoglycerate mutase 1 complexed with KH_ol”, to be published.

FIGS. 3A, 3B, and 3C present non-limiting examples of ligands that bind to glutathione S-transferase P (GSTP1), including the compounds GTX, GTB, GSH, GTS, GPR, GDN, OHH, VWW, BSP, SAS, CBD, GF5, LEE, GTD, OHG, LZ6, GBX, GSN, and 07Z. For additional non-limiting examples and related ligands, see ligands identified by Kyrieleis et al., “Mouse C14A Glutathione-S-Transferase Mutant in Complex with S-hexyl glutathione”, to be published; Kyrieleis et al., “Structural and Kinetic Analyses of Glutathione S-Transferase Mutants”, to be published; Garcia-Saez et al., “Molecular structure at 1.8 A of mouse liver class pi glutathione S-transferase complexed with S-(p-nitrobenzyl)glutathione and other inhibitors”, J Mol Biol., 1994, 237: 298-314; Parraga et al., “The three-dimensional structure of a class-Pi glutathione S-transferase complexed with glutathione: the active-site hydration provides insights into the reaction mechanism”, Biochem J., 1998, 333 (Pt 3): 811-816; Vega et al., “The three-dimensional structure of Cys-47-modified mouse liver glutathione S-transferase P1-1. Carboxymethylation dramatically decreases the affinity for glutathione and is associated with a loss of electron density in the alphaB-310B region”, J Biol Chem., 1998 273: 2844-2850; Ji et al., “Structure and function of the xenobiotic substrate-binding site and location of a potential non-substrate-binding site in a class pi glutathione S-transferase”, Biochemistry, 1997, 36: 9690-9702; Oakley et al., “The ligandin (non-substrate) binding site of human Pi class glutathione transferase is located in the electrophile binding site (H-site)”, J Mol Biol., 1999, 291: 913-926; Oakley et al., “The structures of human glutathione transferase P1-1 in complex with glutathione and various inhibitors at high resolution”, J Mol Biol., 1997, 274: 84-100; Shishido et al., “A covalent G-site inhibitor for glutathione S-transferase Pi (GSTP1-1)”, Chem Commun (Camb)., 2017, 53: 11138-11141; Reinemer et al., “Three-dimensional structure of class pi glutathione S-transferase from human placenta in complex with S-hexylglutathione at 2.8 A resolution”, J Mol Biol., 1992, 227: 214-226; Prade et al., “Structures of class pi glutathione S-transferase from human placenta in complex with substrate, transition-state analogue and inhibitor”, Structure, 1997, 5: 1287-1295; Parket et al., “The anti-cancer drug chlorambucil as a substrate for the human polymorphic enzyme glutathione transferase P1-1: kinetic properties and crystallographic characterization of allelic variants”, J Mol Biol., 2008, 380: 131-144; Ji et al., “Structure and function of residue 104 and water molecules in the xenobiotic substrate-binding site in human glutathione S-transferase P1-1”, Biochemistry, 1999, 38: 10231-10238; Tellez-Sanz et al., “Calorimetric and structural studies of the nitric oxide carrier S-nitrosoglutathione bound to human glutathione transferase P1-1”, Protein Sci., 2006, 15: 1093-1105; and Worth et al., “The interaction of IAA-94 with the soluble conformation of the CLIC1 protein and its structural homolog hGSTP1-1”, to be published.

FIG. 4 presents non-limiting examples of ligands that bind to nucleoside diphosphate kinase B (NME2), including the compounds DG, DA, and GDP. For additional non-limiting examples and related ligands, see ligands identified by Dexheimer et al., “NM23-H2 may play an indirect role in transcriptional activation of c-myc gene expression but does not cleave the nuclease hypersensitive element III1”, Mol Cancer Ther., 2009, 8: 1363-1377; Morera et al., “X-ray structure of human nucleoside diphosphate kinase B complexed with GDP at 2 A resolution”, Structure, 1995, 3: 1307-1314.

FIG. 5 presents non-limiting examples of ligands that bind to Biliverdin reductase A (BLVRA), including the compounds NAP and NAD. For additional non-limiting examples and related ligands, see ligands identified by Kavanagh et al., “Crystal Structure of Human Biliverdin Reductase A”, to be published; Whitby et al., “Crystal structure of a biliverdin IXalpha reductase enzyme-cofactor complex”, J Mol Biol., 2002, 319: 1199-1210.

FIG. 6 presents non-limiting examples of ligands that bind to Ras-related C3 botulinum toxin substrate 3 (RAC3), including the compounds GDP. For additional non-limiting examples and related ligands, see ligands identified by Debreczeni et al., “Crystal Structure of the Human Rac3 in Complex with Gdp”, to be published.

FIG. 7 presents non-limiting examples of ligands that bind to Thymidine kinase, cytosolic (TK1), including the compounds TTP. For additional non-limiting examples and related ligands, see ligands identified by Welin et al., “Structures of thymidine kinase 1 of human and mycoplasmic origin”, Proc Natl Acad Sci USA., 2004, 101: 17970-17975.

FIG. 8 presents non-limiting examples of ligands that bind to Glutamine synthetase (GLUL or GS), including the compounds ADP. For additional non-limiting examples and related ligands, see ligands identified by Krajewski et al., “Crystal structures of mammalian glutamine synthetases illustrate substrate-induced conformational changes and provide opportunities for drug and herbicide design”, J Mol Biol., 2008 375: 217-228.

FIG. 9 presents non-limiting examples of ligands that bind to Eukaryotic initiation factor 4A-III (EIF4A3), including the compounds ANP. For additional non-limiting examples and related ligands, see ligands identified by Bono et al., “The crystal structure of the exon junction complex reveals how it maintains a stable grip on Mrna”, Cell, 2006, 126: 713; Anderson et al., “Structure of the exon junction core complex with a trapped DEAD-box ATPase bound to RNA”, Science, 2006, 313: 1968-1972.

FIG. 10 presents non-limiting examples of ligands that bind to Hypoxanthine-guanine phosphoribosyltransferase (HPRT or HPRT1), including the compounds IMU. For additional non-limiting examples and related ligands, see ligands identified by Shi et al., “The 2.0 A structure of human hypoxanthine-guanine phosphoribosyltransferase in complex with a transition-state analog inhibitor”, Nat Struct Biol., 1999, 6: 588-593.

FIG. 11 presents non-limiting examples of ligands that bind to Glycogen phosphorylase, brain form (PYGB), including the compounds AMP. For additional non-limiting examples and related ligands, see ligands identified by Mathieu et al., “Insights into Brain Glycogen Metabolism: the structure of human brain glycogen phosphorylase”, J Biol Chem., 2016, 291: 18072-18083.

FIG. 12 presents non-limiting examples of ligands that bind to Vinculin (VCL) including the compounds PIO For additional non-limiting examples and related ligands, see ligands identified by Chinthalapudi et al., “Differential lipid binding of vinculin isoforms promotes quasi-equivalent dimerization”, Proc Natl Acad Sci USA., 2016, 113: 9539-9544.

FIG. 13 presents non-limiting examples of ligands that bind to cytosolic Branched-chain-amino-acid aminotransferase (BCAT1), including the compounds PLP, GBN and CBC. For additional non-limiting examples and related ligands, see ligands identified by Goto et al., “Structural determinants for branched-chain aminotransferase isozyme-specific inhibition by the anticonvulsant drug gabapentin”, J Biol Chem., 2005, 280: 37246-37256; Hu et al., “The design and synthesis of human branched-chain amino acid aminotransferase inhibitors for treatment of neurodegenerative diseases”, Bioorg Med Chem Lett., 2006, 16: 2337-2340.

FIG. 14 presents non-limiting examples of ligands that bind to Nucleoside diphosphate kinase A (NME1), including the compounds ADP and A7Z. For additional non-limiting examples and related ligands, see ligands identified by Giraud et al., “Crystal Structures of S120G Mutant and Wild Type of Human Nucleoside Diphosphate Kinase A in Complex with ADP”, J Bioenerg Biomembr., 2006, 38: 261-264; Mortenson et al., “Inverse drug discovery” strategy to identify proteins that are targeted by latent electrophiles as exemplified by Aryl Fluorosulfates”, J Am Chem Soc., 2018, 140: 200-210.

FIG. 15 presents non-limiting examples of ligands that bind to Adenylosuccinate lyase (ADSL), including the compounds AMP and 2SA. For additional non-limiting examples and related ligands, see ligands identified by Stenmark et al., “Crystal Structure of Human Adenylosuccinate Lyase”, to be published; Stenmark et al., “Human Adenylosuccinate Lyase in Complex with its Substrate N6-(1,2-Dicarboxyethyl)-AMP, and its Products AMP and Fumarate”, to be published.

FIG. 16 presents non-limiting examples of ligands that bind to ADP-ribose pyrophosphatase, mitochondrial (NUDT9), including the compounds RP5 and BGC. For additional non-limiting examples and related ligands, see ligands identified by Shen et al., “The crystal structure and mutational analysis of human NUDT9”, J Mol Biol., 2003, 332: 385-398.

FIG. 17 presents non-limiting examples of ligands that bind to Peptidyl-prolyl cis-trans isomerase NIMA-interacting 1 (PIN1), including the compound GIA. For additional non-limiting examples and related ligands, see ligands identified by Zhang et al., “Structural basis for high-affinity peptide inhibition of human Pin1”, ACS Chem Biol., 2007, 2: 320-328; Dong et al., “Structure-based design of novel human Pin1 inhibitors (II)”, Bioorg Med Chem Lett., 2010, 20: 2210-2214.

FIG. 18 presents non-limiting examples of ligands that bind to 14-3-3 protein beta/alpha (YWHAB), including the compound NAG. For additional non-limiting examples and related ligands, see ligands identified by De Vink et al., “A Binary Bivalent Supramolecular Assembly Platform Based on Cucurbit[8]uril and Dimeric Adapter Protein 14-3-3”, Angew Chem Int Ed Engl., 2017, 56: 8998-9002; Toleman et al., “Structural basis of O-GlcNAc recognition by mammalian 14-3-3 proteins”, Proc Natl Acad Sci USA., 2018, 115: 5956-5961.

FIGS. 19A and 19B present non-limiting examples of ligands that bind to Bifunctional purine biosynthesis protein ATIC (ATIC), including the compounds BW2, XMP, AMZ, 8US, and 8UM. For additional non-limiting examples and related ligands, see ligands identified by Cheong et al., “Crystal Structures of Human Bifunctional Enzyme Aminoimidazole-4-carboxamide Ribonucleotide Transformylase/IMP Cyclohydrolase in Complex with Potent Sulfonyl-containing Antifolates”, J Biol Chem., 2004, 279: 18034-18045; Fales et al., “Discovery of N-(6-Fluoro-1-oxo-1,2-dihydroisoquinolin-7-yl)-5-[(3R)-3-hydroxypyrrolidin-1-yl]thiophene-2-sulfonamide (LSN 3213128), a Potent and Selective Nonclassical Antifolate Aminoimidazole-4-carboxamide Ribonucleotide Formyltransferase (AICARFT) Inhibitor Effective at Tumor Suppression in a Cancer Xenograft Model”, J Med Chem., 2017, 60: 9599-9616.

FIG. 20 presents non-limiting examples of ligands that bind to Glucose-6-phosphate 1-dehydrogenase (G6PD), including the compounds BG6 and NAP. For additional non-limiting examples and related ligands, see ligands identified by Ranzani et al., “Mutations in the tetramer interface of human glucose-6-phosphate dehydrogenase reveals kinetic differences between oligomeric states”, FEBS Lett., 2017, 591: 1278-1284; Au et al., “Human Glucose-6-Phosphate Dehydrogenase: The Crystal Structure Reveals a Structural Nadp+ Molecule and Provides Insights Into Enzyme Deficiency”, Structure, 2000, 8: 293; Kotaka et al., “Structural Studies of Glucose-6-Phosphate and Nadp+ Binding to Human Glucose-6-Phosphate Dehydrogenase”, Acta Crystallogr D Biol Crystallogr., 2005, 61: 495; Au et al., “Crystal structure of Human G6PD Canton”, to be published.

FIG. 21 presents non-limiting examples of ligands that bind to Glycogen phosphorylase, liver form (PYGL), including the compounds AMP, PLP, 25D, 26B, 055, AVE, AVF and NBG. For additional non-limiting examples and related ligands, see ligands identified by Rath et al., “Activation of human liver glycogen phosphorylase by alteration of the secondary structure and packing of the catalytic core”, Mol Cell, 2000, 6: 139-148; Thomson et al., “Anthranilimide based glycogen phosphorylase inhibitors for the treatment of type 2 diabetes. Part 3: X-ray crystallographic characterization, core and urea optimization and in vivo efficacy”, Bioorg Med Chem Lett., 2009, 19: 1177-1182; Pautsch et al., “Molecular recognition of the protein phosphatase 1 glycogen targeting subunit by glycogen phosphorylase”, J Biol Chem., 2008, 283: 8913-8918; Anderka et al., “Thermodynamic characterization of allosteric glycogen phosphorylase inhibitors”, Biochemistry, 2008, 47: 4683-4691.

FIG. 22 presents non-limiting examples of ligands that bind to GDP-mannose 4,6 dehydratase (GMDS), including the compounds NAP, GDD, FZE, NDP and GDP. For additional non-limiting examples and related ligands, see ligands identified by Pfeiffer et al., “A Parsimonious Mechanism of Sugar Dehydration by Human GDP-Mannose-4,6-dehydratase”, ACS Catal., 2019, 9: 2962-2968; Vedadi et al., “Crystal Structure and Biophysical Characterization of Human GDP-D-mannose 4,6-dehydratase”, to be published.

FIGS. 23A, 23B, and 23C present non-limiting examples of ligands that bind to Peptidyl-prolyl cis-trans isomerase FKBPlA (FKBPlA), including the compounds GPI, TST, 818, FK5, SUB, AP1, SB3, SBX, SB1, B7G, 858, ARD, RAD, RAP, 001, FKA, and 587. For additional non-limiting examples and related ligands, see ligands identified by Burkhard et al., “X-ray structures of small ligand-FKBP complexes provide an estimate for hydrophobic interaction energies”, J Mol Biol., 2000, 295: 953-962; Sich et al., “Solution structure of a neurotrophic ligand bound to FKBP12 and its effects on protein dynamics”, Eur J Biochem., 2000, 267: 5342-5354; Becker et al., “FK-506-binding protein: three-dimensional structure of the complex with the antagonist L-685,818”, J Biol Chem., 1993, 268: 11335-11339; Van Duyne et al., “Atomic structure of FKBP-FK506, an immunophilin-immunosuppressant complex”, Science, 1991, 252: 839-842; Wilson et al., “Comparative X-ray structures of the major binding protein for the immunosuppressant FK506 (tacrolimus) in unliganded form and in complex with FK506 and rapamycin”, Acta Crystallogr D Biol Crystallogr., 1995, 51: 511-521; Sun et al., “Design and structure-based study of new potential FKBP12 inhibitors”, Biophys J., 2003, 85: 3194-3201; Clarkson et al., “Redesigning an FKBP-ligand interface to generate chemical dimerizers with novel specificity”, Proc Natl Acad Sci USA., 1998, 95: 10437-10442; Holt et al., “Design, synthesis, and kinetic evaluation of high-affinity FKBP ligands and the x-ray crystal-structures of their complexes with FKBP12”, J Am Chem Soc., 1993, 115: 9925-9938; Becket et al., “32-Indolyl ether derivatives of ascomycin: three-dimensional structures of complexes with FK506-binding protein”, J Med Chem., 1999, 42: 2798-2804; Liang et al., “Refined structure of the FKBP12-rapamycin-FRB ternary complex at 2.2 A resolution”, Acta Crystallogr D Biol Crystallogr., 1999, 55: 736-744; Wu et al., “Rational design and implementation of a chemically inducible heterotrimerization system”, Nat Methods, 2020, 17: 928-936; Choi et al., “Structure of the FKBP12-rapamycin complex interacting with the binding domain of human FRAP”, Science, 1996, 273: 239-242; Dubowchik et al., “2-Aryl-2,2-difluoroacetamide FKBP12 ligands: synthesis and X-ray structural studies”, Org Lett., 2001, 3: 3987-3990; Schultz et al., “Chemical inducers of dimerization: the atomic structure of FKBP12-FK1012A-FKBP12”, Bioorg Med Chem Lett., 1998, 8: 1-6; Van Duyne et al., “Atomic Structure of the Rapamycin Human Immunophilin Fkbp-12 Complex”, J Am Chem Soc., 1991, 113: 7433; and Fulton et al., “Energetic and structural analysis of the role of tryptophan 59 in FKBP12”, Biochemistry, 2003, 42: 2364-2372.

FIG. 24 is a non-limiting example of a Formula described herein.

### DETAILED DESCRIPTION OF THE INVENTION

Heteroaryl sulfonyl compounds and their use and manufacture are provided that covalently modify a Target Protein to treat a disease that is mediated by the Target Protein in a host, typically a human. In one aspect of the invention a heteroaryl sulfonyl compound described herein reacts with a tyrosine residue on the Target Protein to form a covalent bond. In another aspect a heteroaryl sulfonyl compound described herein reacts with a lysine residue on the Target Protein to form a covalent bond. The invention provides a heteroaryl sulfonyl compound of Formula I, Formula II, Formula III, Formula IV, Formula V, Formula VI, Formula VII, or Formula VIII, or a pharmaceutically acceptable salt thereof that includes a Protein Recognition Moiety that provides specificity to the heteroaryl sulfonyl compound, and an electrophilic sulfonyl (SO₂) that reacts with the target tyrosine or lysine to create a covalent bond between the Target Protein and the presently described inhibitor.

The heteroaryl sulfonyl compound as described herein in principle embodiments has a stable shelf life for at least 2 months, 3 months, 6 months or 1 year or more neat or as part of a pharmaceutically acceptable dosage form, and itself is pharmaceutically acceptable.

In one aspect a heteroaryl sulfonyl compound of Formula I, Formula II, Formula III, Formula IV, Formula V, Formula VI, Formula VII, or Formula VIII, or a pharmaceutically acceptable salt thereof, is provided:

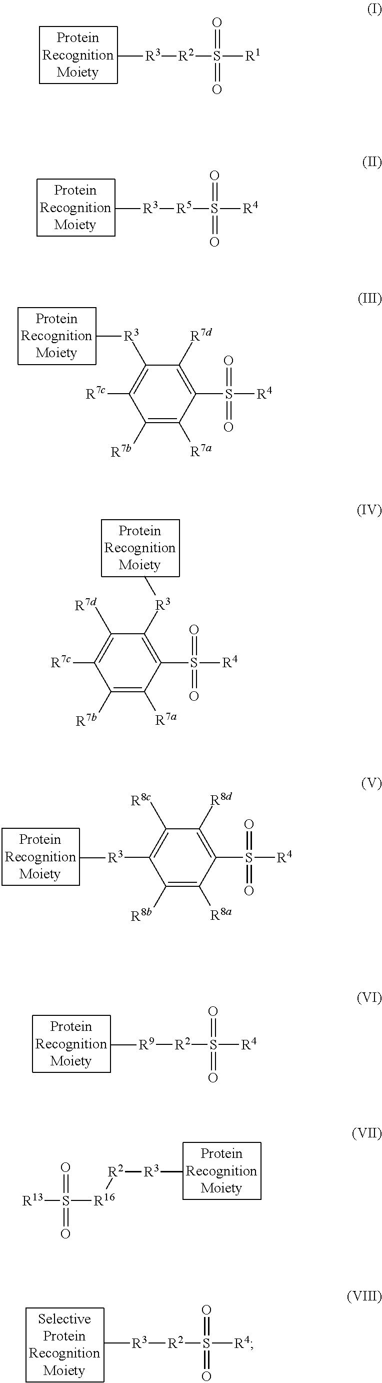

wherein the variables are as defined herein.

### Embodiments of Formula I

In certain embodiments the heteroaryl sulfonyl compound of Formula I is selected from:

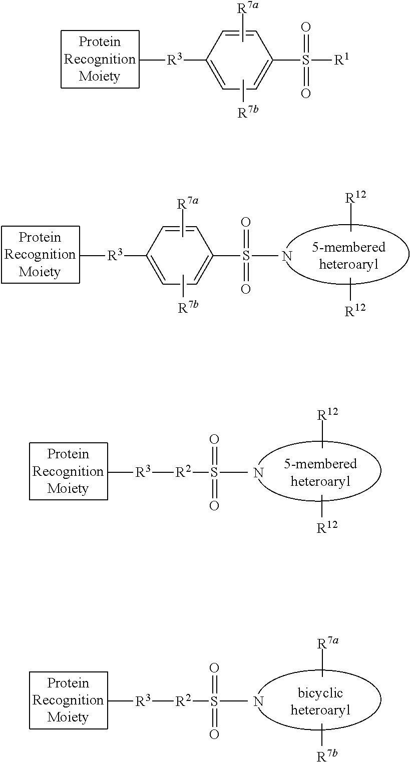

or a pharmaceutically acceptable salt thereof.

### Embodiments of Formula II

In certain embodiments the heteroaryl sulfonyl compound of Formula II is selected from:

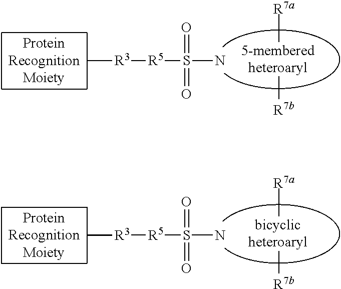

or a pharmaceutically acceptable salt thereof.

### Embodiments of Formula III

In certain embodiments the heteroaryl sulfonyl compound of Formula III is selected from:

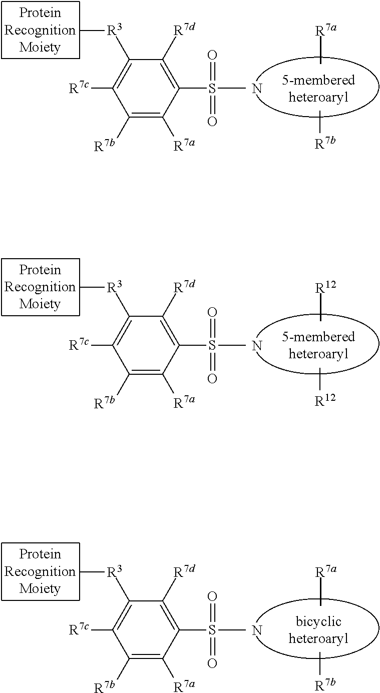

or a pharmaceutically acceptable salt thereof.

### Embodiments of Formula IV

In certain embodiments the heteroaryl sulfonyl compound of Formula IV is selected from:

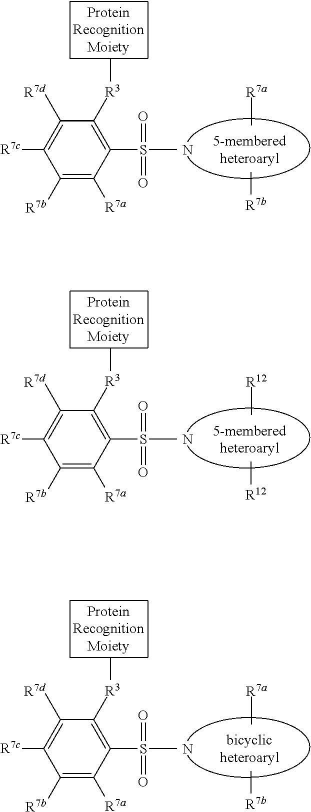

or a pharmaceutically acceptable salt thereof.

### Embodiments of Formula V

In certain embodiments the heteroaryl sulfonyl compound of Formula V is selected from:

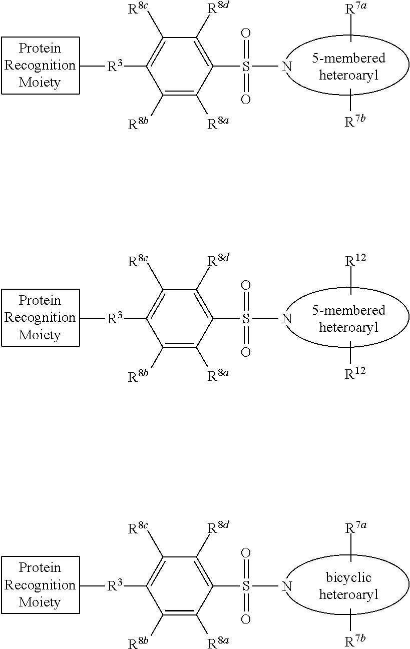

or a pharmaceutically acceptable salt thereof.

### Embodiments of Formula VI

In certain embodiments the heteroaryl sulfonyl compound of Formula VI is selected from:

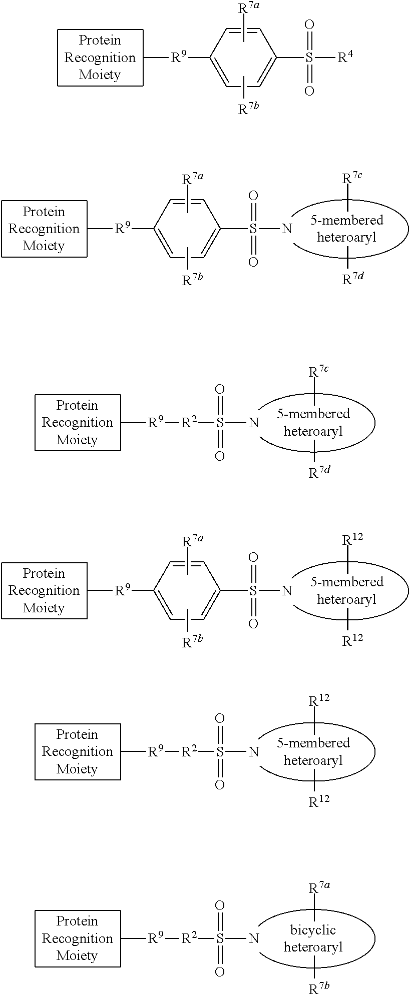

or a pharmaceutically acceptable salt thereof.

### Embodiments of Formula VII

In certain embodiments the heteroaryl sulfonyl compound of Formula VII is selected from:

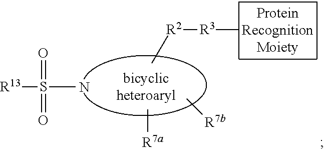

or a pharmaceutically acceptable salt thereof.

In certain embodiments the heteroaryl sulfonyl compound of Formula VII is selected from:

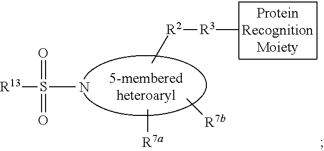

or a pharmaceutically acceptable salt thereof.

### Embodiments of Formula VIII

In certain embodiments the heteroaryl sulfonyl compound of Formula VIII is selected from:

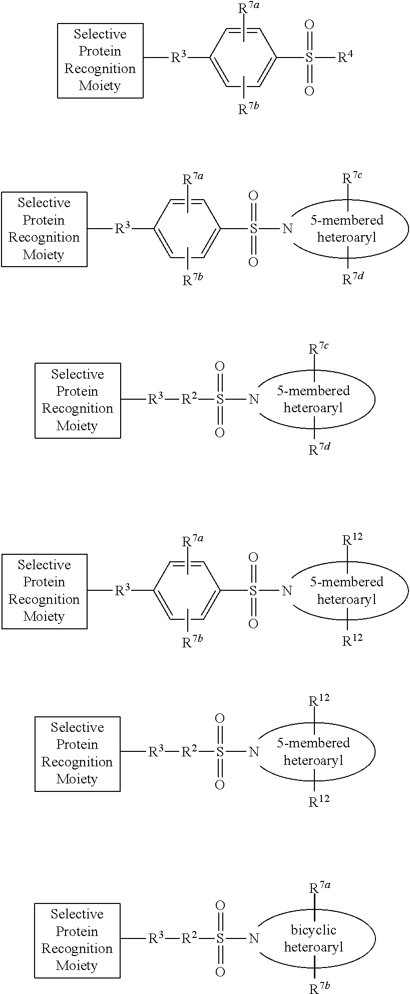

or a pharmaceutically acceptable salt thereof.

### Embodiments of Formula I′

In certain embodiments the heteroaryl sulfonyl compound of Formula I′ is selected from:

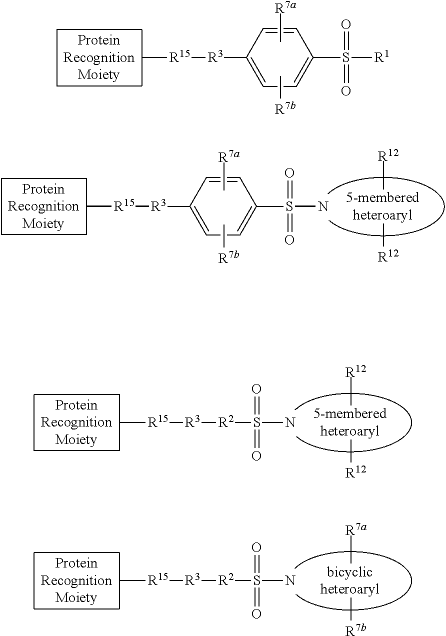

or a pharmaceutically acceptable salt thereof.

### Embodiments of Formula II′

In certain embodiments the heteroaryl sulfonyl compound of Formula II′ is selected from:

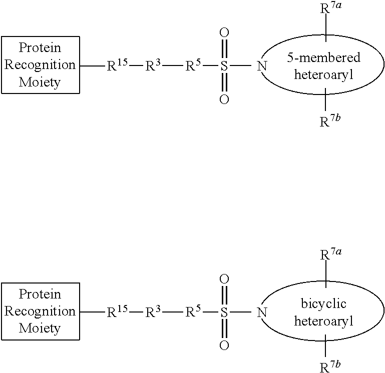

or a pharmaceutically acceptable salt thereof.

### Embodiments of Formula III′

In certain embodiments the heteroaryl sulfonyl compound of Formula III′ is selected from:

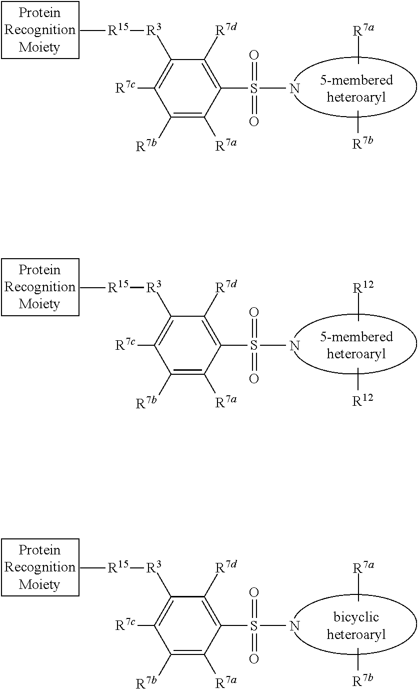

or a pharmaceutically acceptable salt thereof.

### Embodiments of Formula IV′

In certain embodiments the heteroaryl sulfonyl compound of Formula IV′ is selected from:

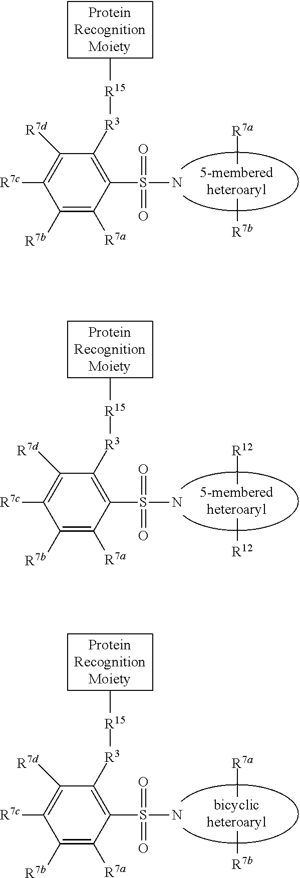

or a pharmaceutically acceptable salt thereof.

### Embodiments of Formula V′

In certain embodiments the heteroaryl sulfonyl compound of Formula V′ is selected from:

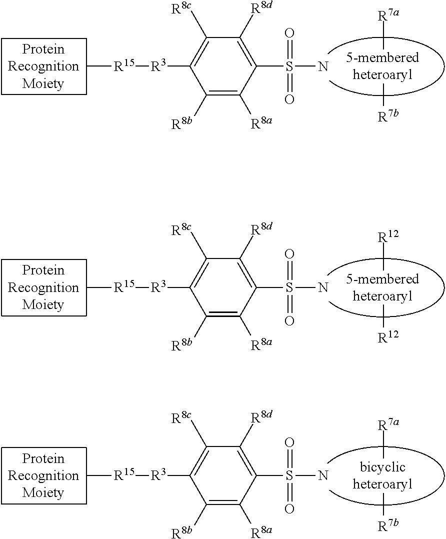

or a pharmaceutically acceptable salt thereof.

### Embodiments of Formula VI′

In certain embodiments the heteroaryl sulfonyl compound of Formula VI′ is selected from:

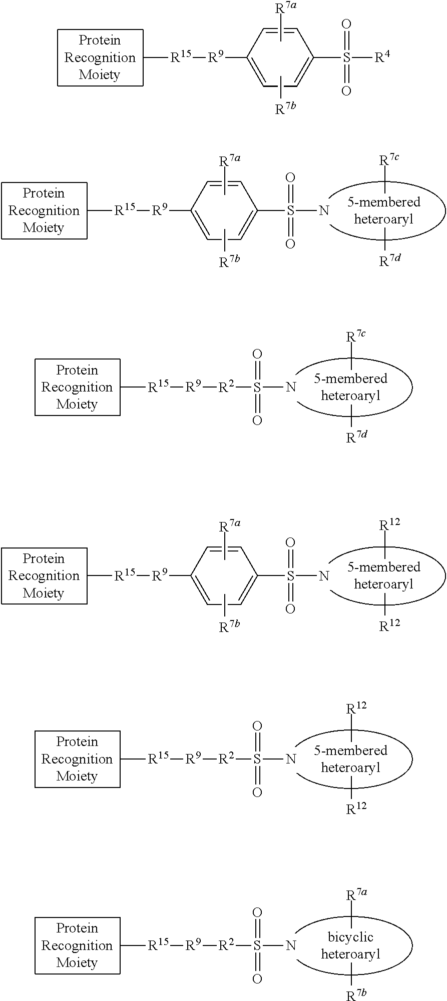

or a pharmaceutically acceptable salt thereof.

### Embodiments of Formula VII′

In certain embodiments the heteroaryl sulfonyl compound of Formula VII′ is selected from:

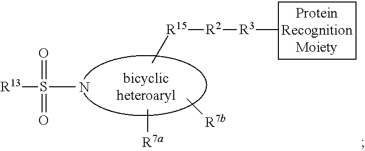

or a pharmaceutically acceptable salt thereof.

In certain embodiments the heteroaryl sulfonyl compound of Formula VII′ is selected from:

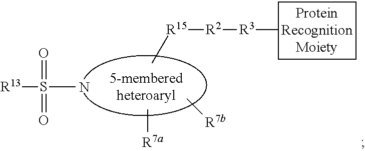

or a pharmaceutically acceptable salt thereof.

### Embodiments of Formula VIII′

In certain embodiments the heteroaryl sulfonyl compound of Formula VIII′ is selected

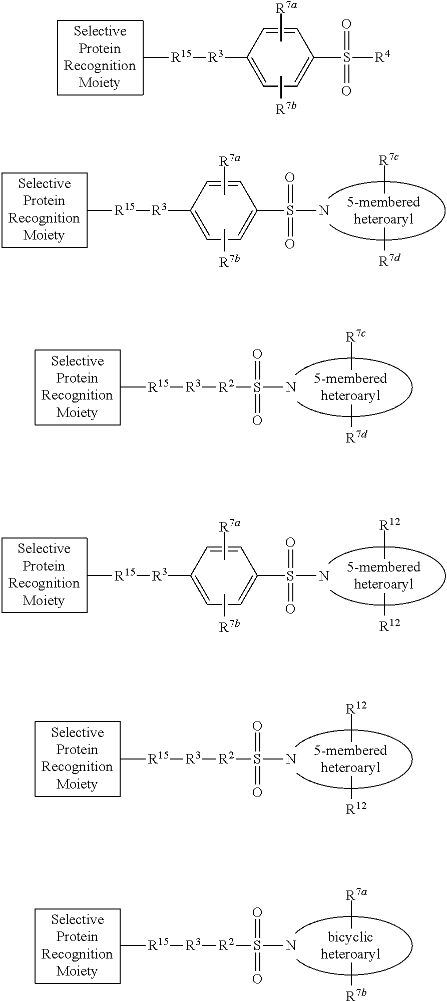

or a pharmaceutically acceptable salt thereof.

### Additional Embodiments

In certain embodiments the heteroaryl sulfonyl compound of the present invention is selected from:

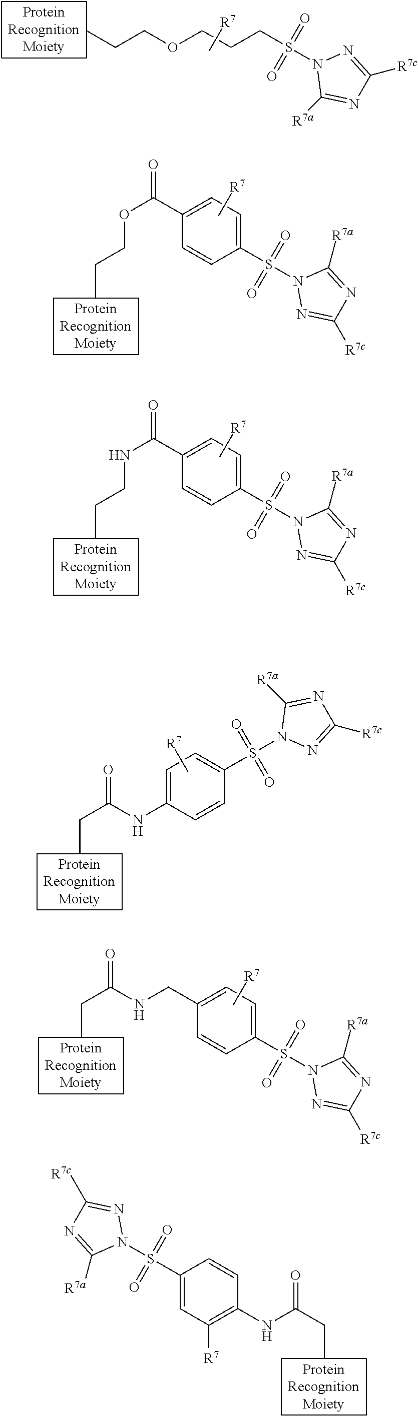

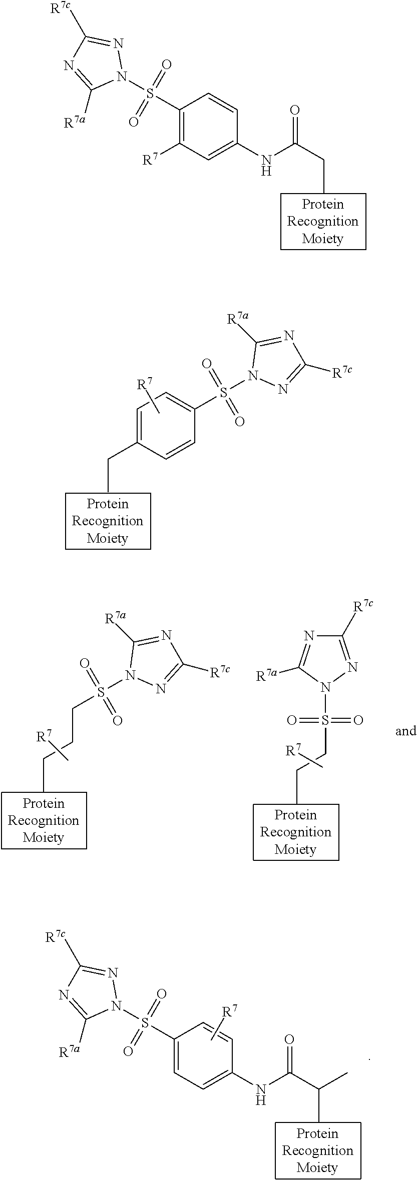

In certain embodiments the heteroaryl sulfonyl compound of the present invention is selected from:

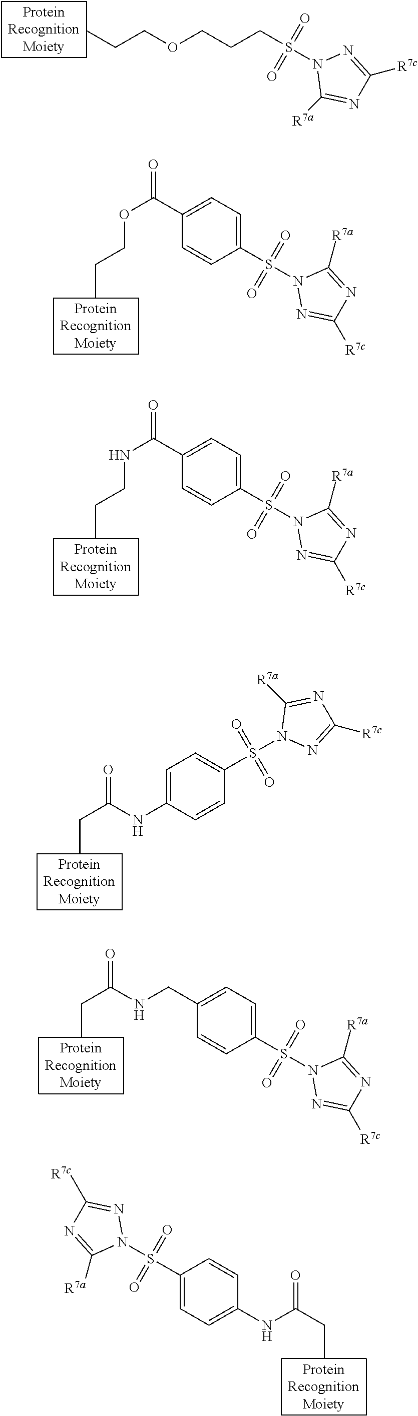

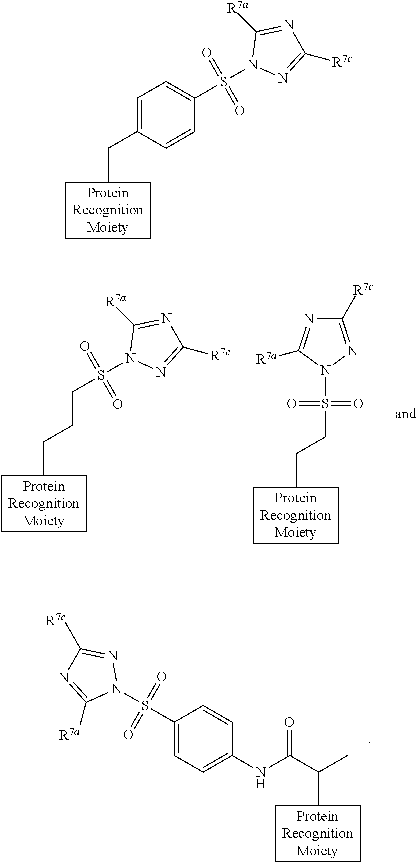

In certain embodiments the heteroaryl sulfonyl compound of the present invention is selected from:

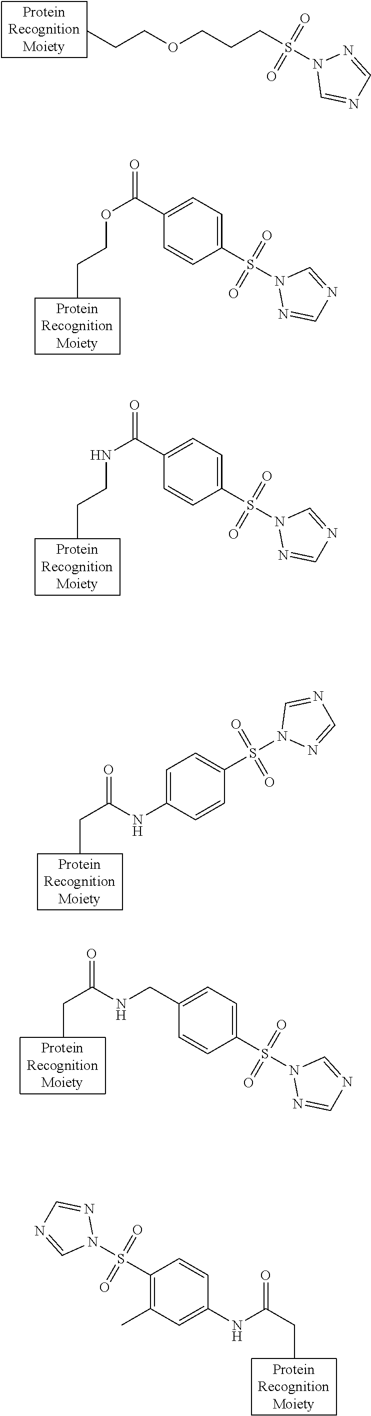 [SMILES: CCCOCCCS(=O)(=O)n1cncn1 ; CCCOC(=O)c1ccc(S(=O)(=O)n2cncn2)cc1 ; CCCNC(=O)c1ccc(S(=O)(=O)n2cncn2)cc1 ; CCC(=O)Nc1ccc(S(=O)(=O)n2cncn2)cc1 ; CCC(=O)NCc1ccc(S(=O)(=O)n2cncn2)cc1 ; CCC(=O)Nc1ccc(S(=O)(=O)n2cncn2)c(C)c1]

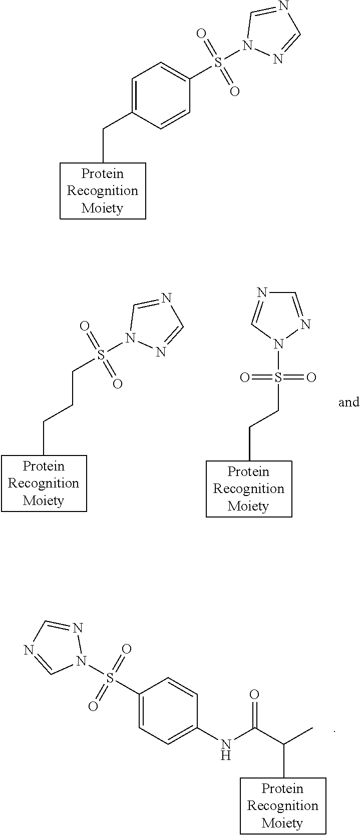 [SMILES: CCc1ccc(S(=O)(=O)n2cncn2)cc1 ; CCCCS(=O)(=O)n1cncn1 ; CCCS(=O)(=O)n1cncn1 ; CC(C)C(=O)Nc1ccc(S(=O)(=O)n2cncn2)cc1]

In certain embodiments the heteroaryl sulfonyl compound of the present invention is selected from:

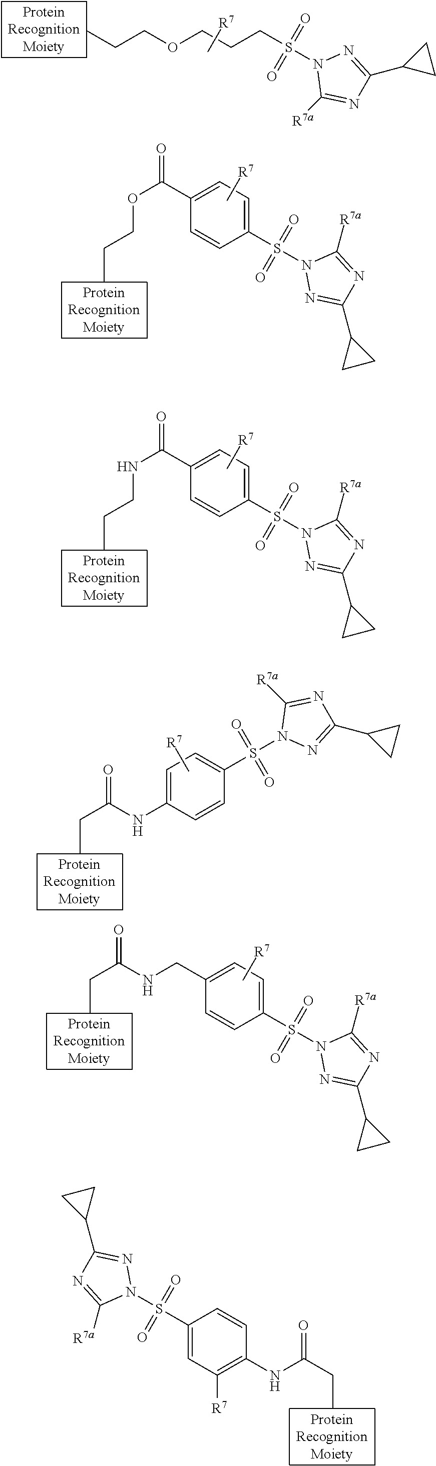

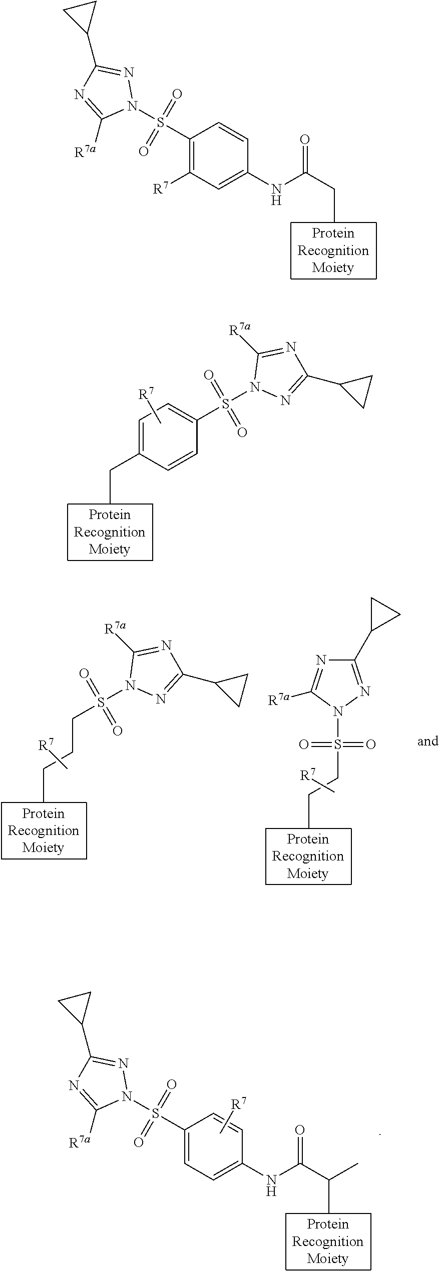

In certain embodiments the heteroaryl sulfonyl compound of the present invention is selected from:

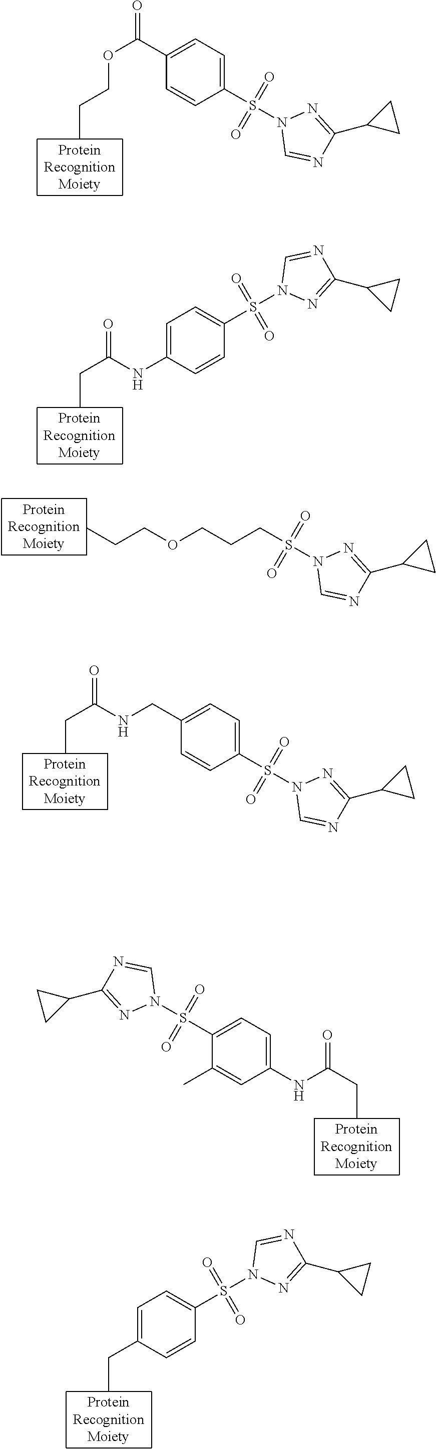 [SMILES: CCCOC(=O)c1ccc(S(=O)(=O)n2cnc(C3CC3)n2)cc1 ; CCC(=O)Nc1ccc(S(=O)(=O)n2cnc(C3CC3)n2)cc1 ; CCCOCCCS(=O)(=O)n1cnc(C2CC2)n1 ; CCC(=O)NCc1ccc(S(=O)(=O)n2cnc(C3CC3)n2)cc1 ; CCC(=O)Nc1ccc(S(=O)(=O)n2cnc(C3CC3)n2)c(C)c1 ; CCc1ccc(S(=O)(=O)n2cnc(C3CC3)n2)cc1]

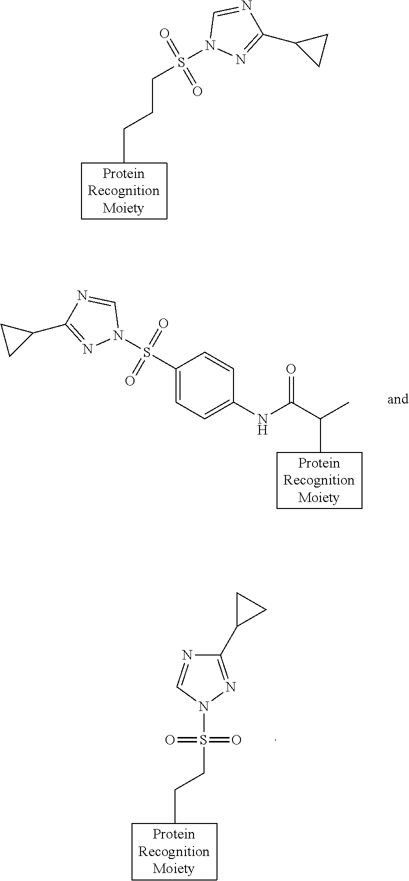 [SMILES: CCCCS(=O)(=O)n1cnc(C2CC2)n1 ; CC(C)C(=O)Nc1ccc(S(=O)(=O)n2cnc(C3CC3)n2)cc1 ; CCCS(=O)(=O)n1cnc(C2CC2)n1]

In certain embodiments the heteroaryl sulfonyl compound of the present invention is selected from:

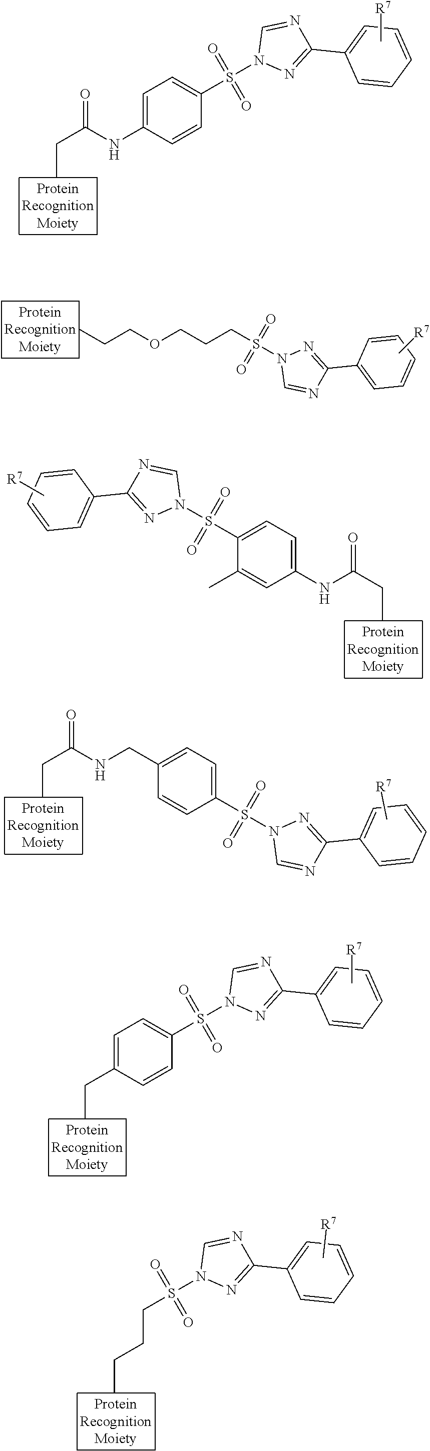

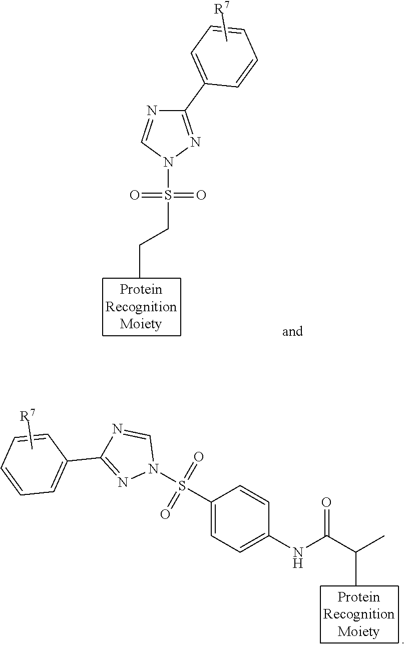

In certain embodiments the heteroaryl sulfonyl compound of the present invention is selected from:

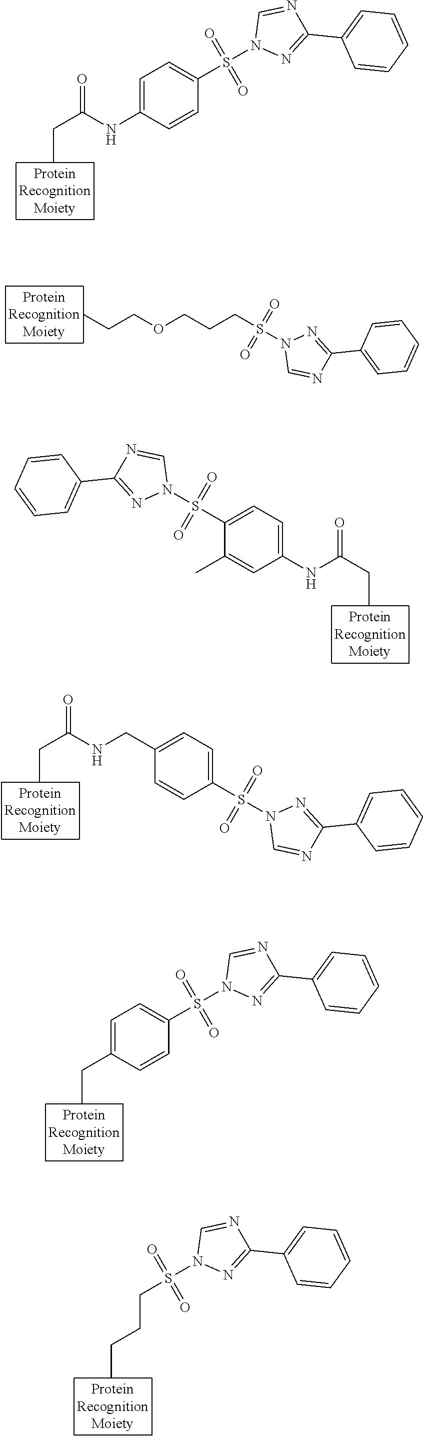

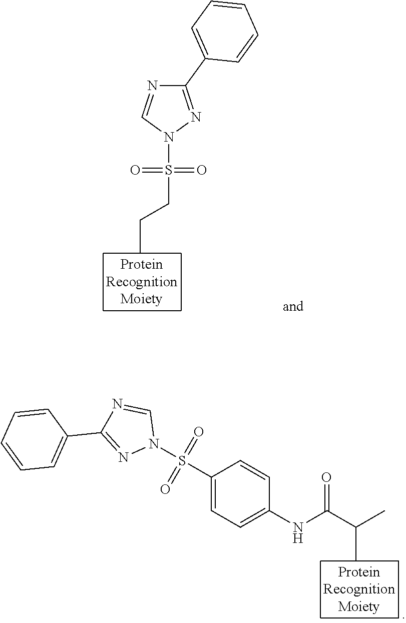 [SMILES: CCCS(=O)(=O)n1cnc(-c2ccccc2)n1 ; CC(C)C(=O)Nc1ccc(S(=O)(=O)n2cnc(-c3ccccc3)n2)cc1]

In certain embodiments the heteroaryl sulfonyl compound of the present invention is selected from:

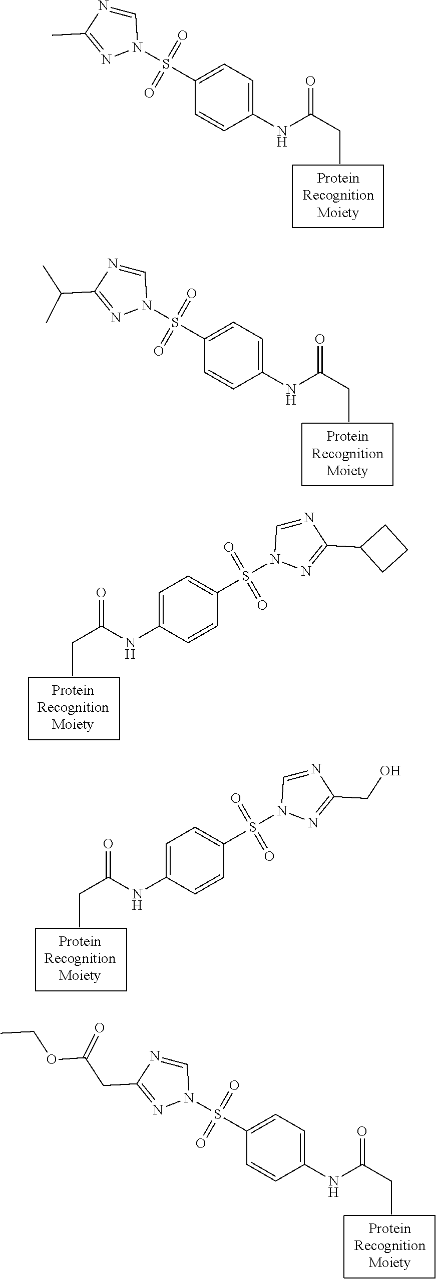

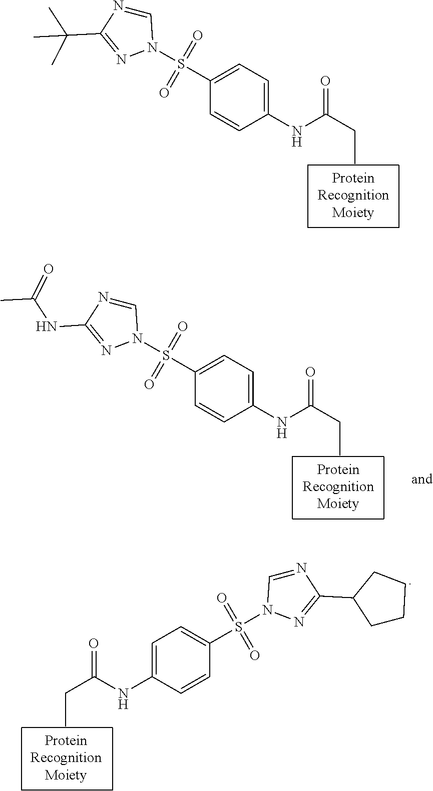

In certain embodiments, the heteroaryl sulfonyl compound of the present invention is selected from:

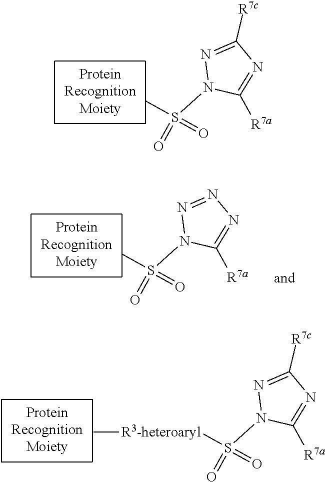

In certain embodiments, the heteroaryl sulfonyl compound of the present invention is selected from:

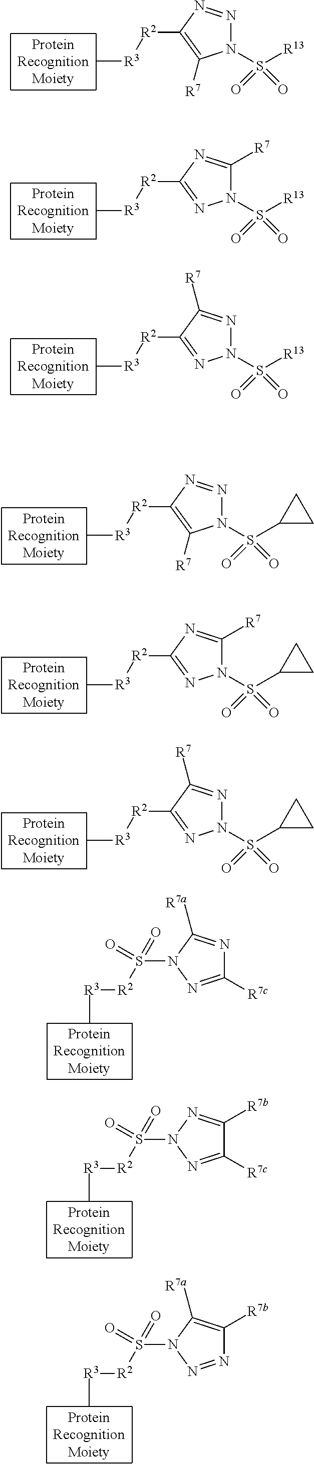

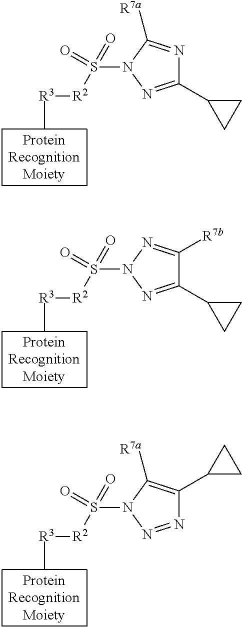

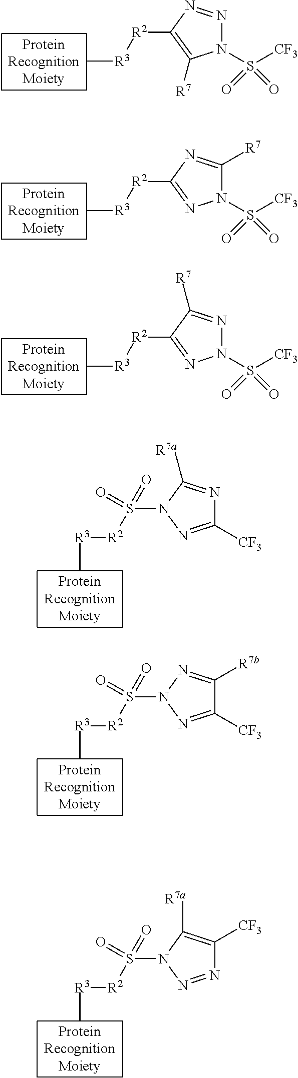

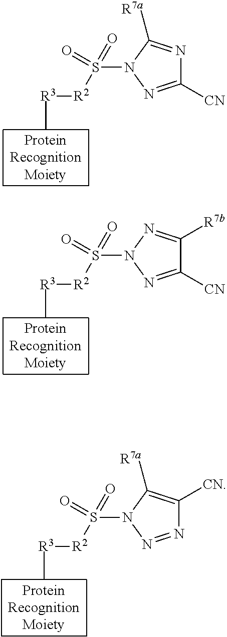

In certain embodiments, the heteroaryl sulfonyl compound of the present invention is selected from:

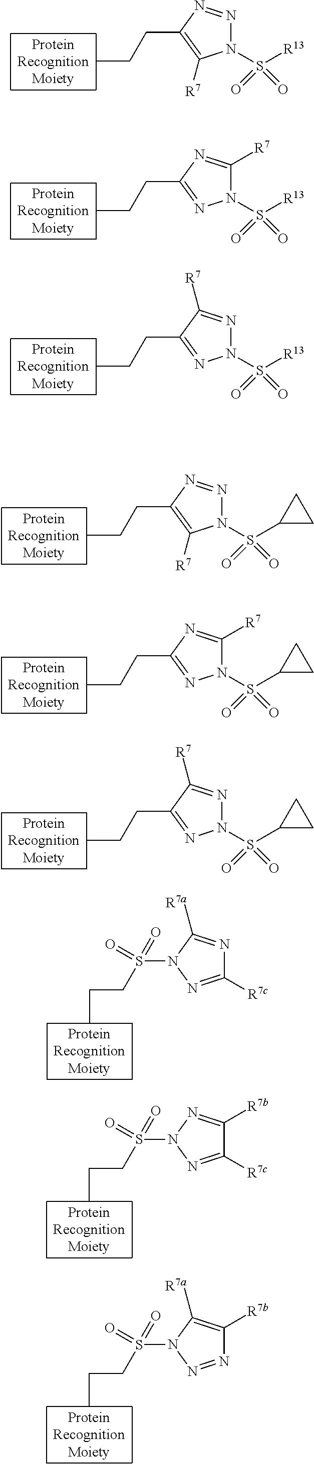

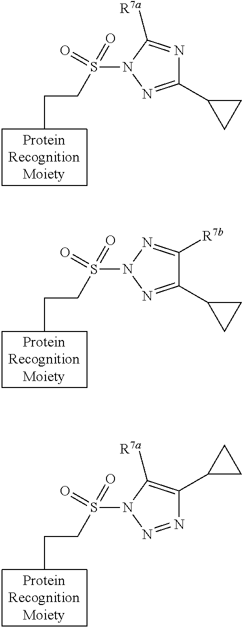

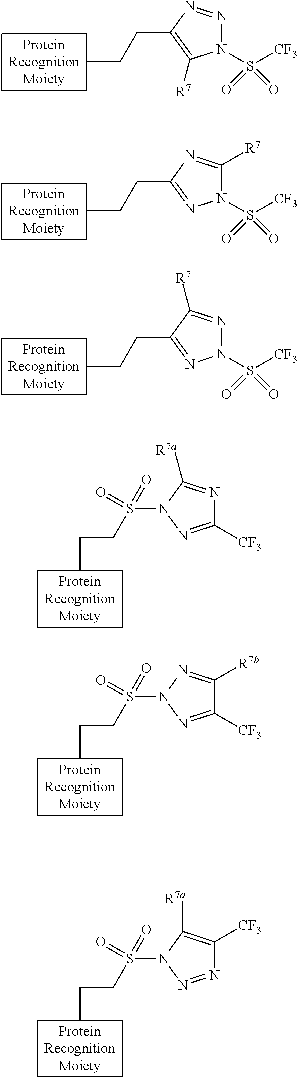

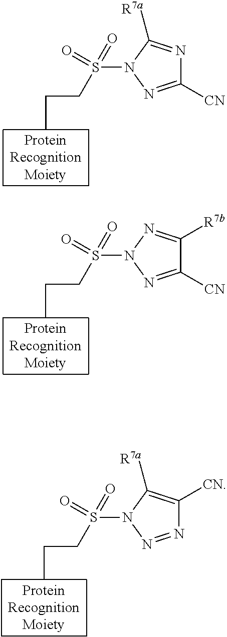

In certain embodiments, the heteroaryl sulfonyl compound of the present invention is selected from:

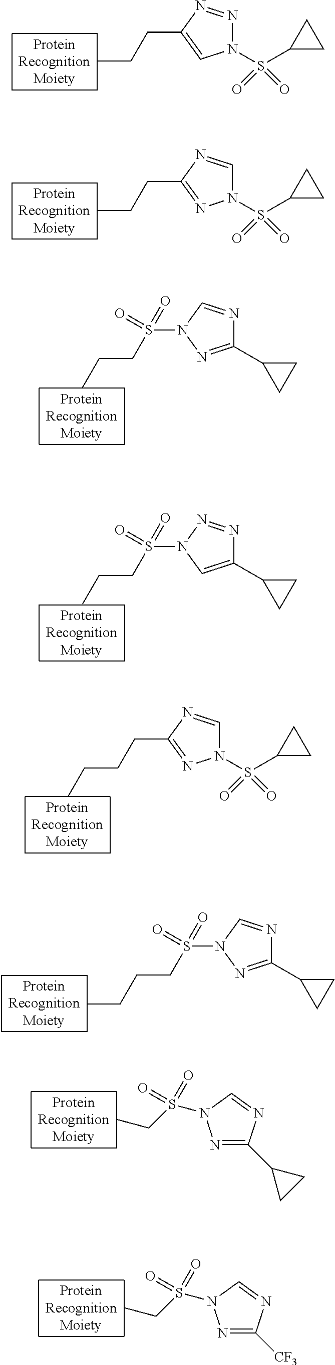

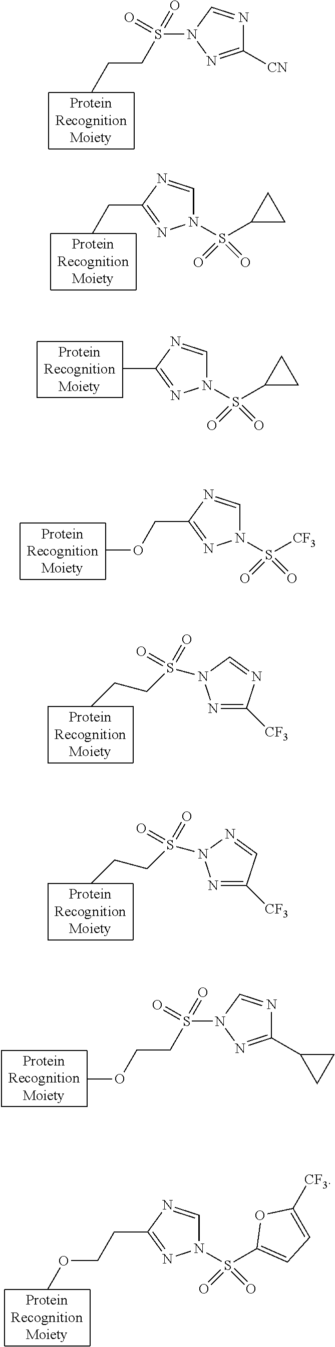

In certain embodiments, the heteroaryl sulfonyl compound of the present invention is selected from:

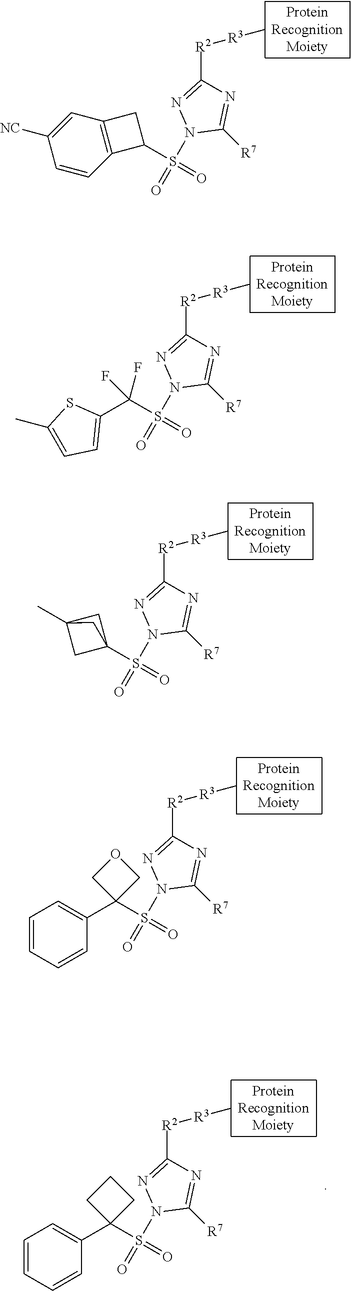

In certain embodiments, the heteroaryl sulfonyl compound of the present invention is selected from:

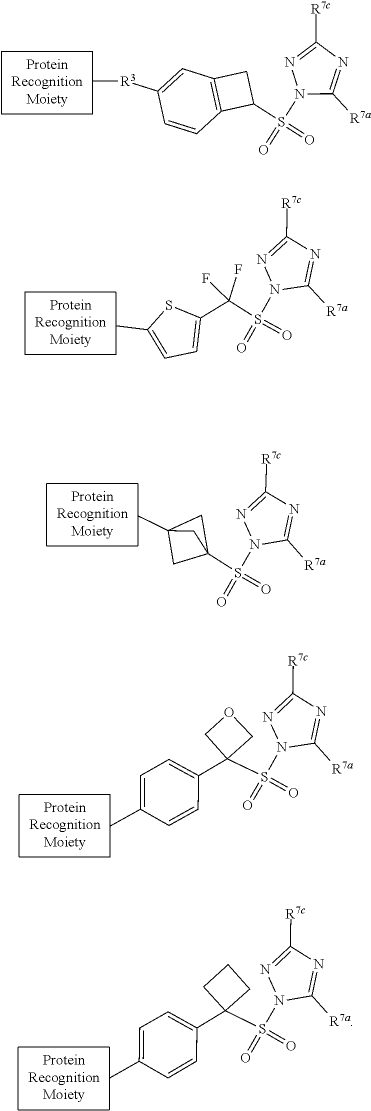

In certain embodiments, the heteroaryl sulfonyl compound of the present invention is selected from:

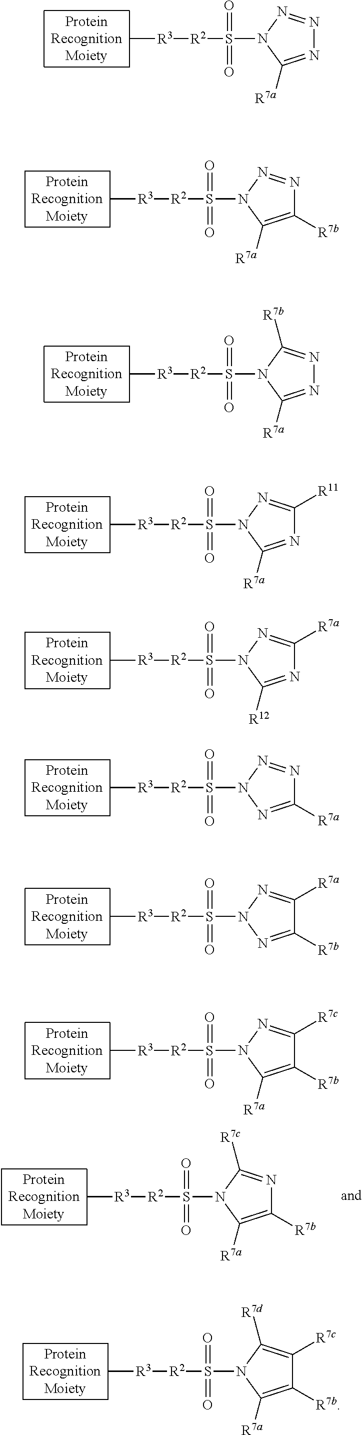

In certain embodiments, the heteroaryl sulfonyl compound of the present invention is selected from:

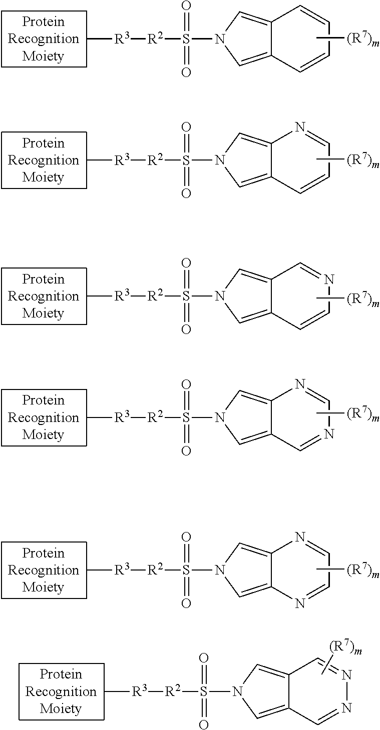

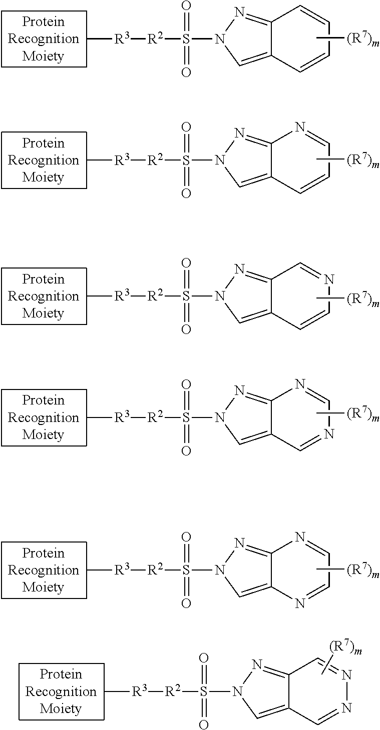

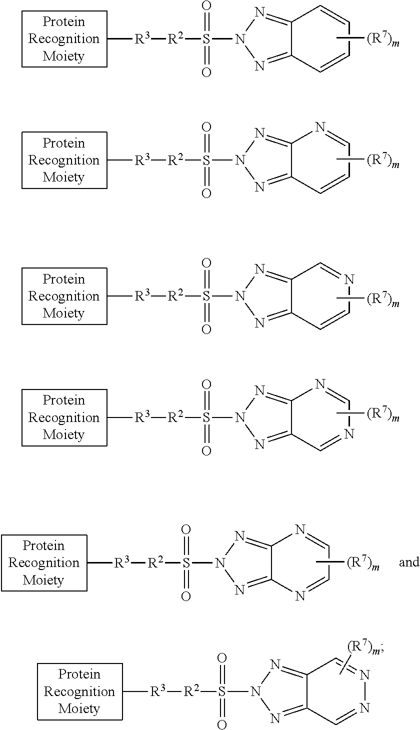

wherein m is independently selected from 1, 2, 3, and 4; and a floating bond on one ring of a bicyclic system means the substituent or substituents are optionally placed on any ring of the system.

For example,

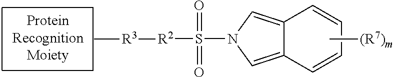

represents, but is not limited to:

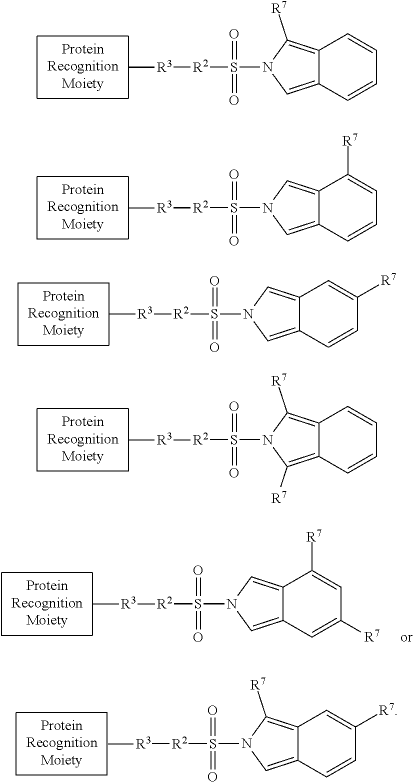

In certain embodiments, the heteroaryl sulfonyl compound of the present invention is selected from:

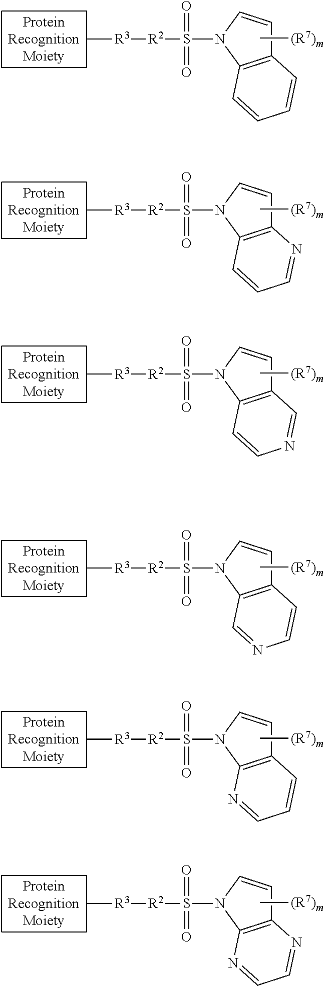

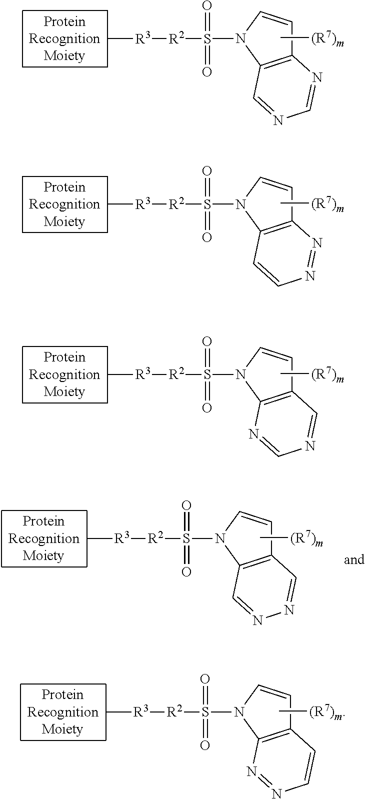

In certain embodiments, the heteroaryl sulfonyl compound of the present invention is selected from:

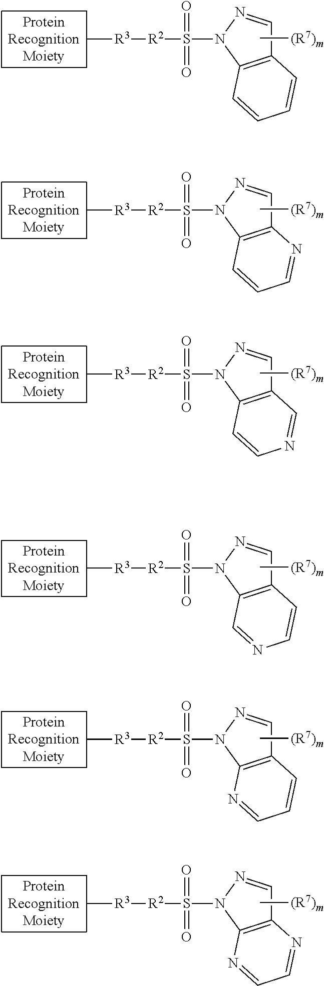

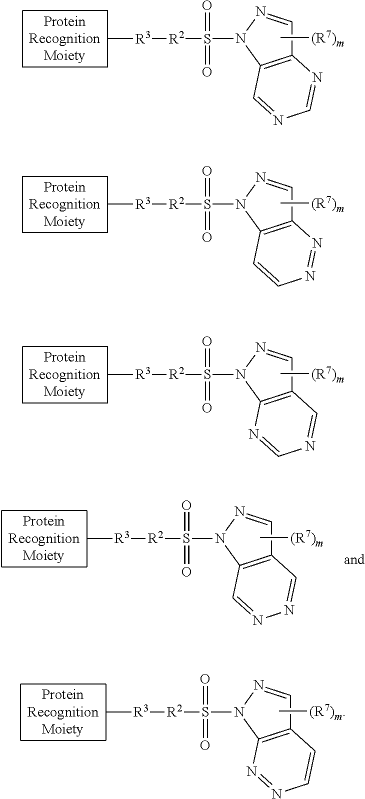

In certain embodiments, the heteroaryl sulfonyl compound of the present invention is selected from:

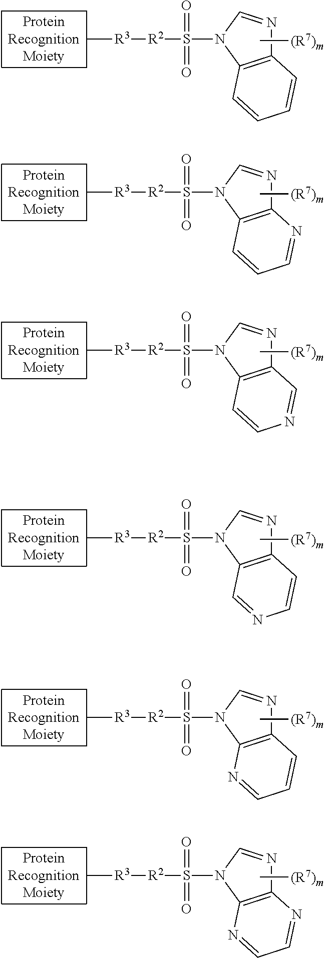

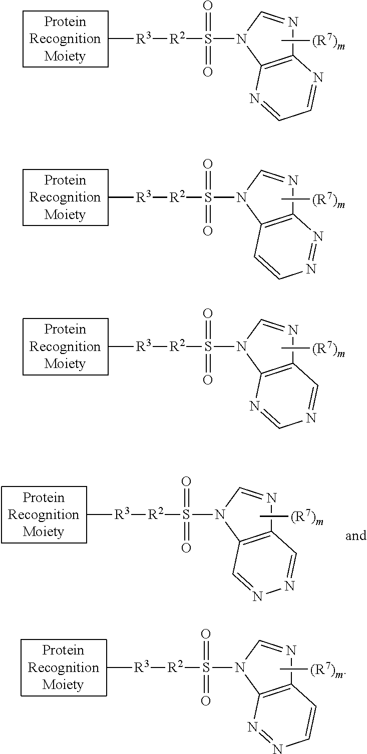

In certain embodiments, the heteroaryl sulfonyl compound of the present invention is selected from:

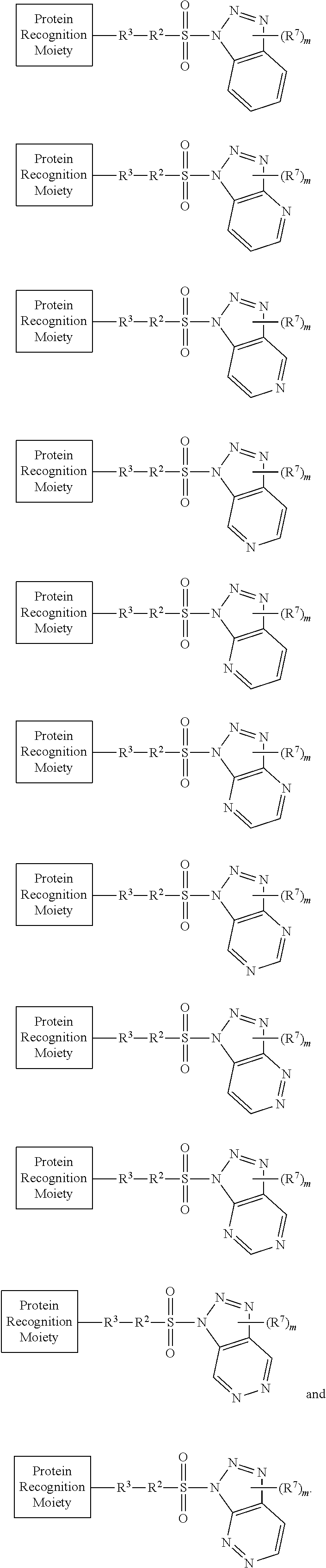

In certain embodiments, the heteroaryl sulfonyl compound of the present invention is selected from:

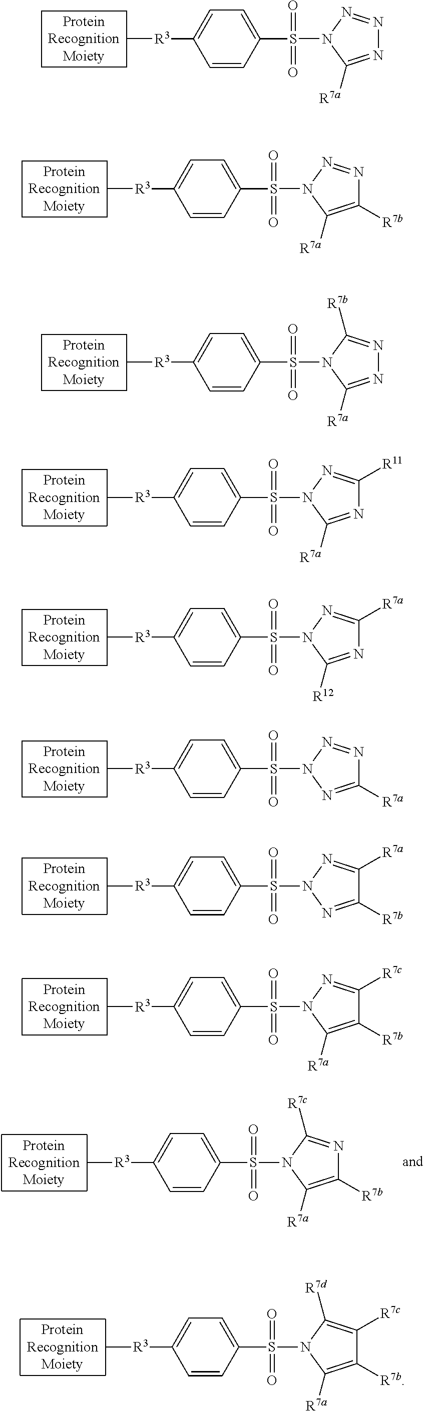

In certain embodiments, the heteroaryl sulfonyl compound of the present invention is selected from:

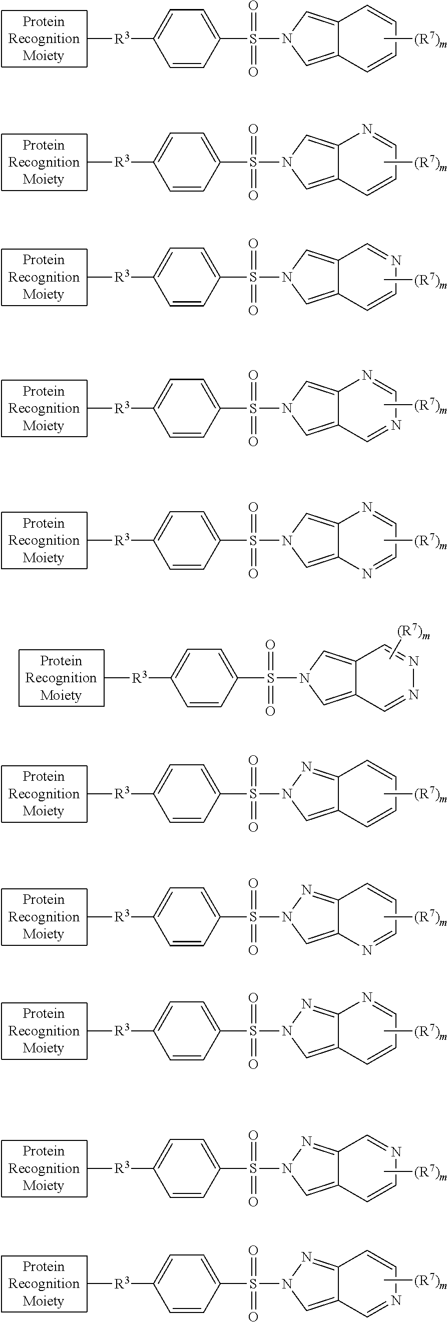

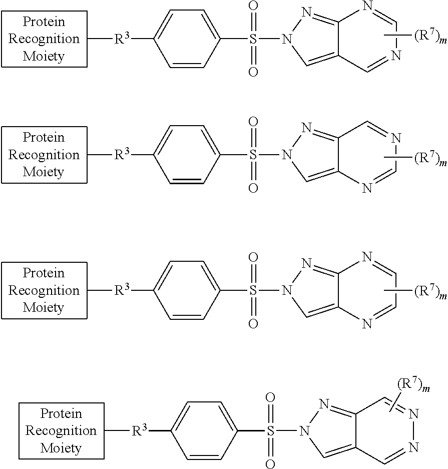

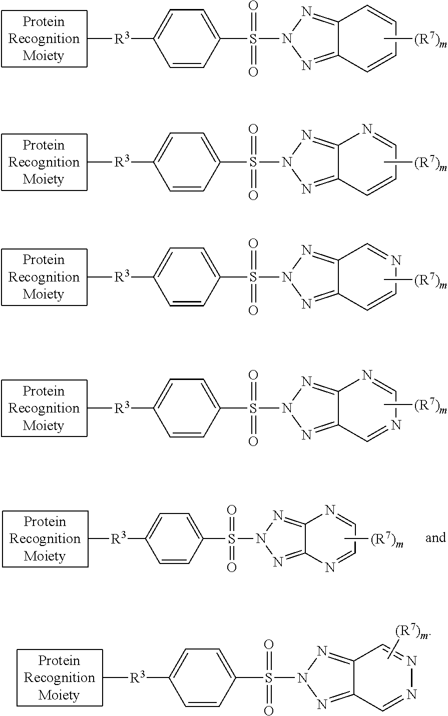

In certain embodiments, the heteroaryl sulfonyl compound of the present invention is selected from:

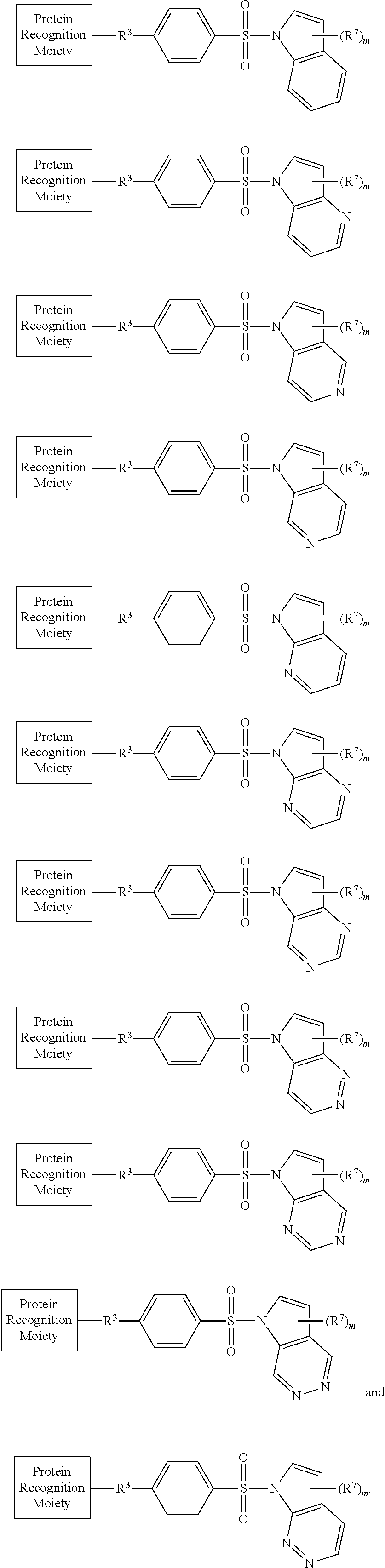

In certain embodiments, the heteroaryl sulfonyl compound of the present invention is selected from:

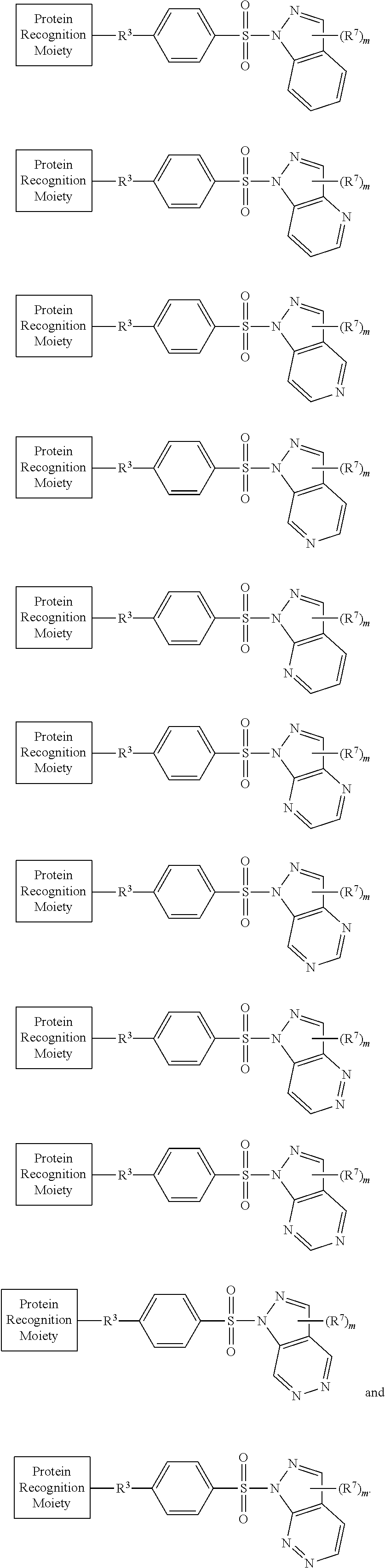

In certain embodiments, the heteroaryl sulfonyl compound of the present invention is selected from:

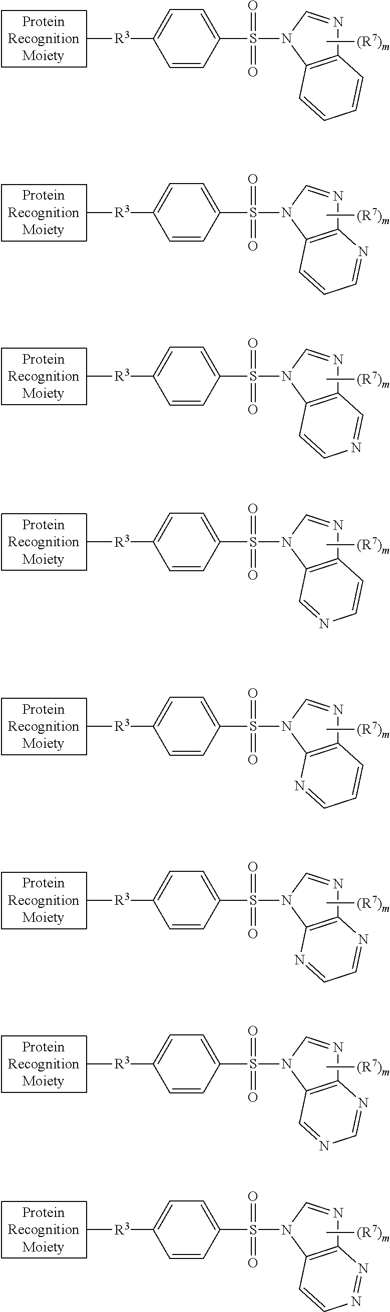

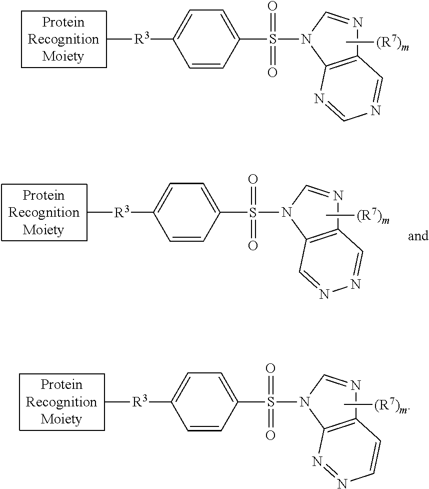

In certain embodiments, the heteroaryl sulfonyl compound of the present invention is selected from:

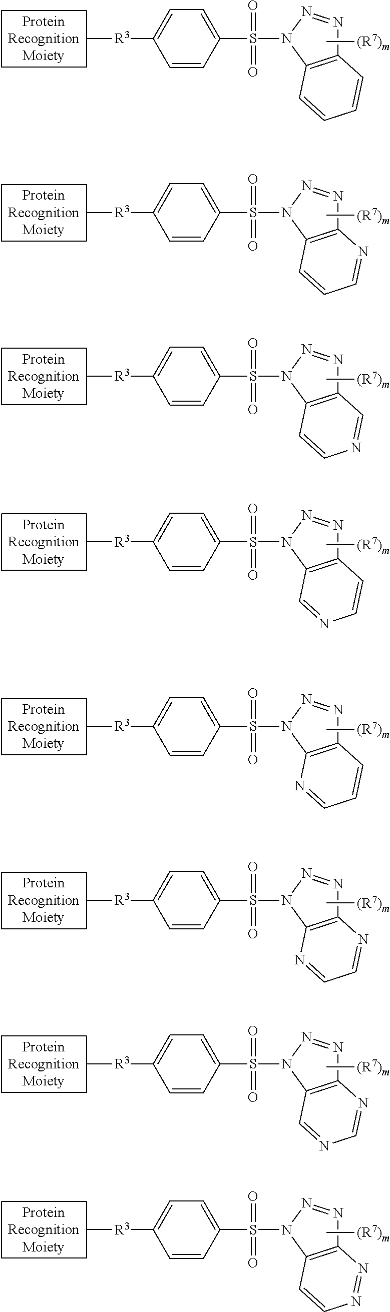

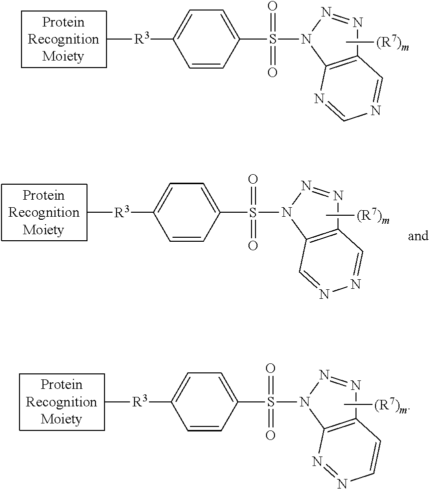

In certain embodiments, the heteroaryl sulfonyl compound of the present invention is selected from:

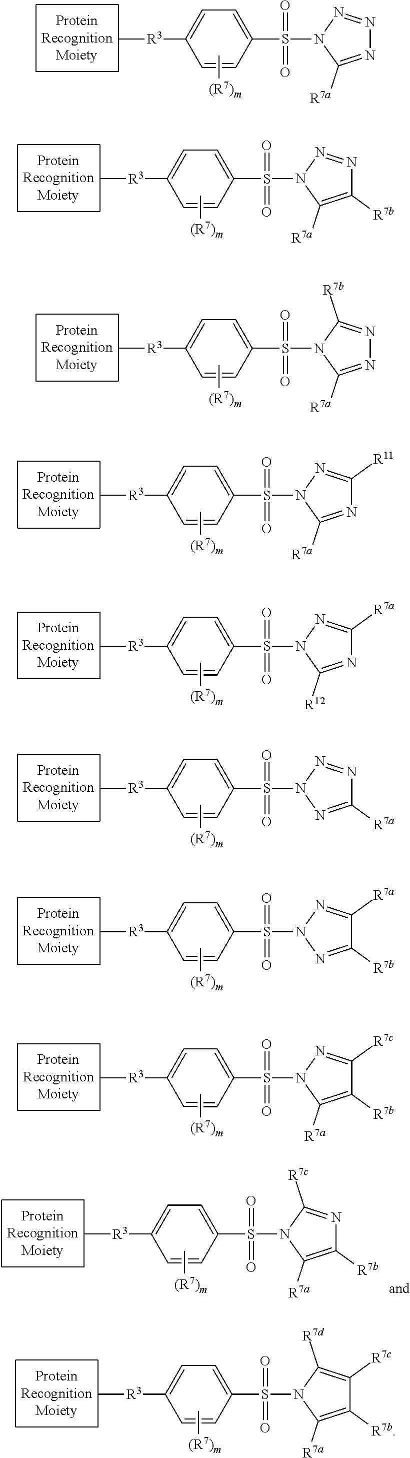

In certain embodiments, the heteroaryl sulfonyl compound of the present invention is selected from:

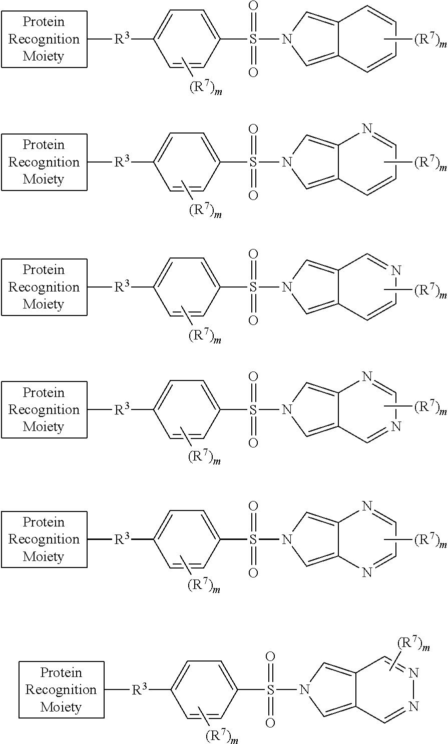

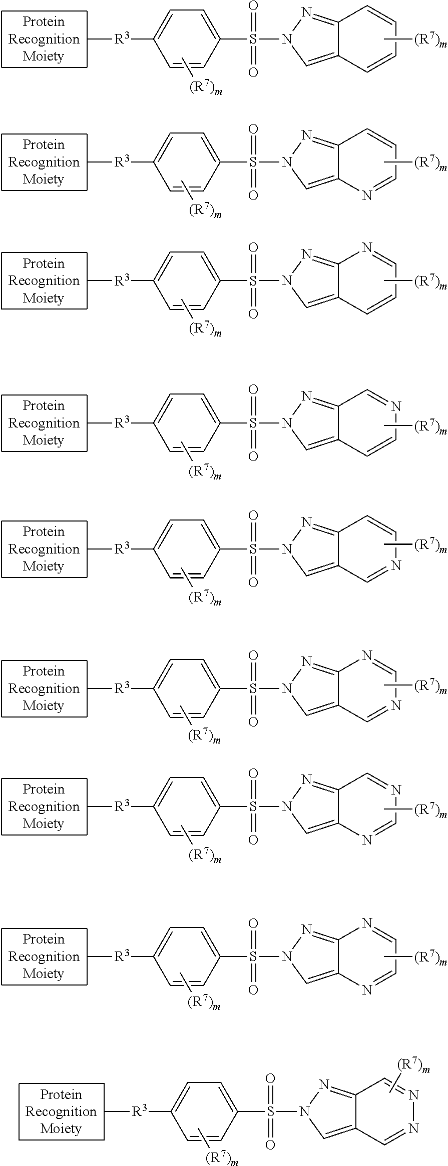

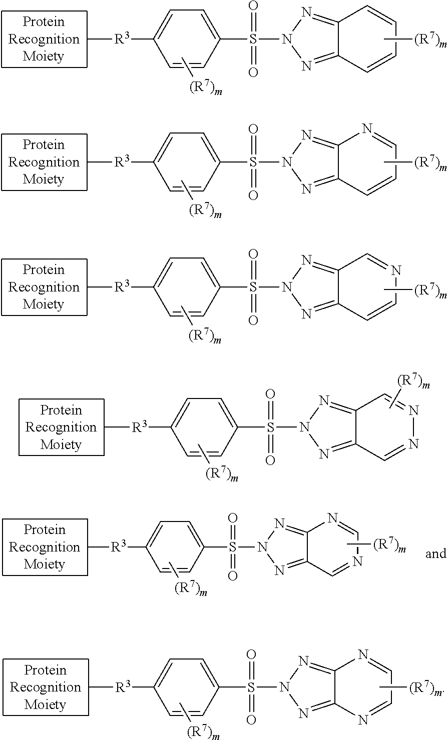

In certain embodiments, the heteroaryl sulfonyl compound of the present invention is selected from:

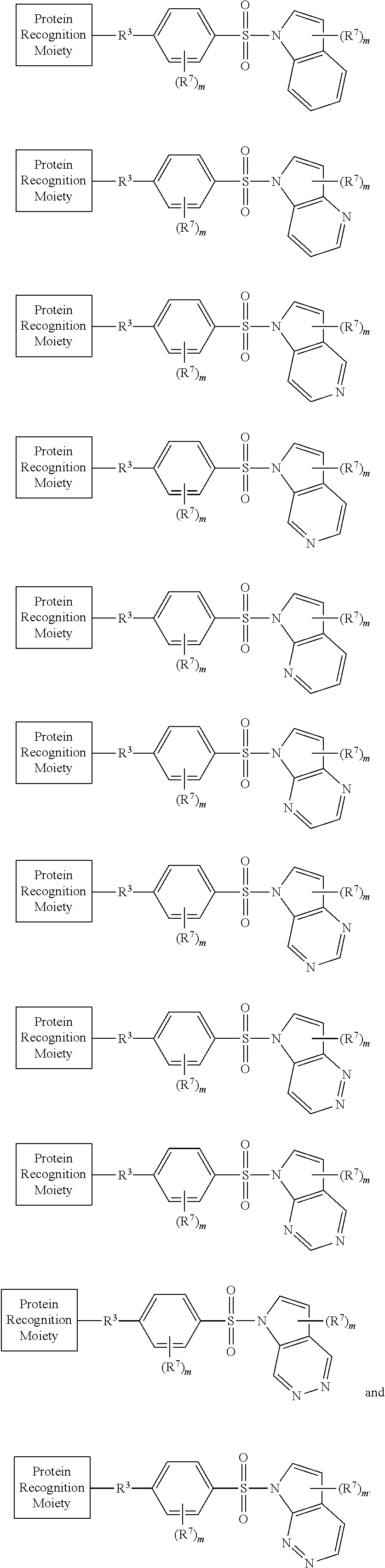

In certain embodiments, the heteroaryl sulfonyl compound of the present invention is selected from:

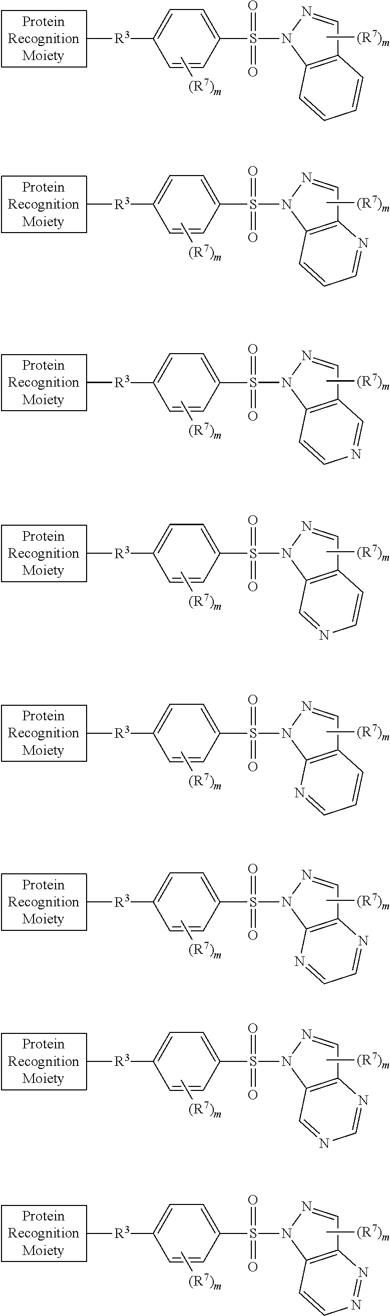

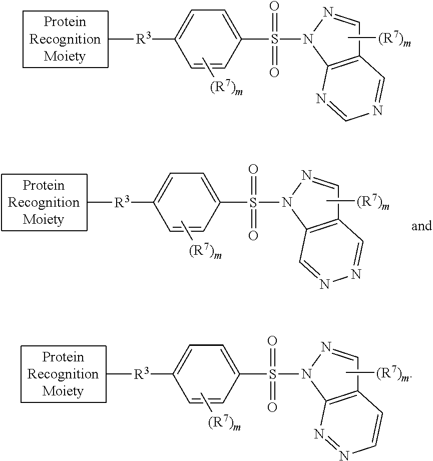

In certain embodiments, the heteroaryl sulfonyl compound of the present invention is selected from:

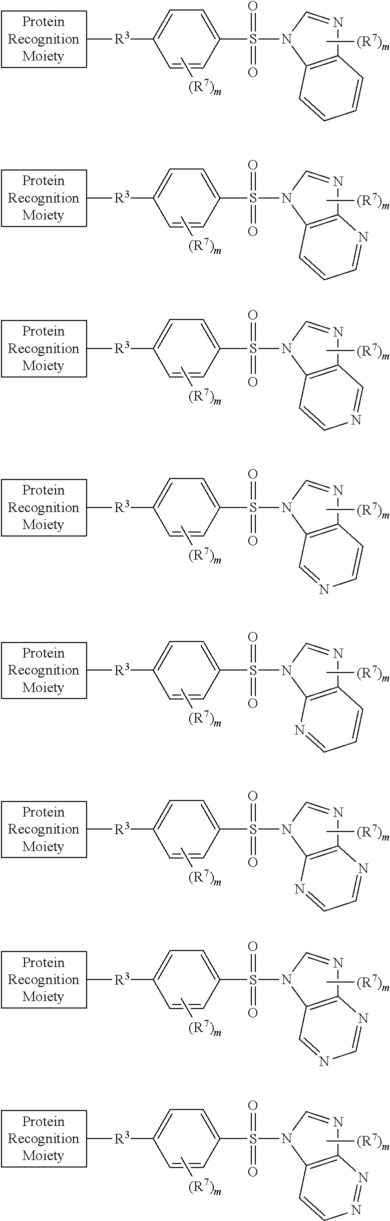

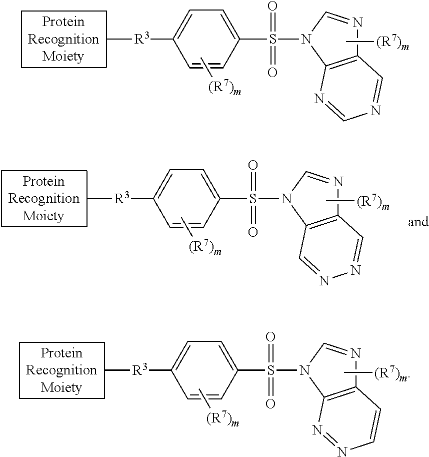

In certain embodiments, the heteroaryl sulfonyl compound of the present invention is selected from:

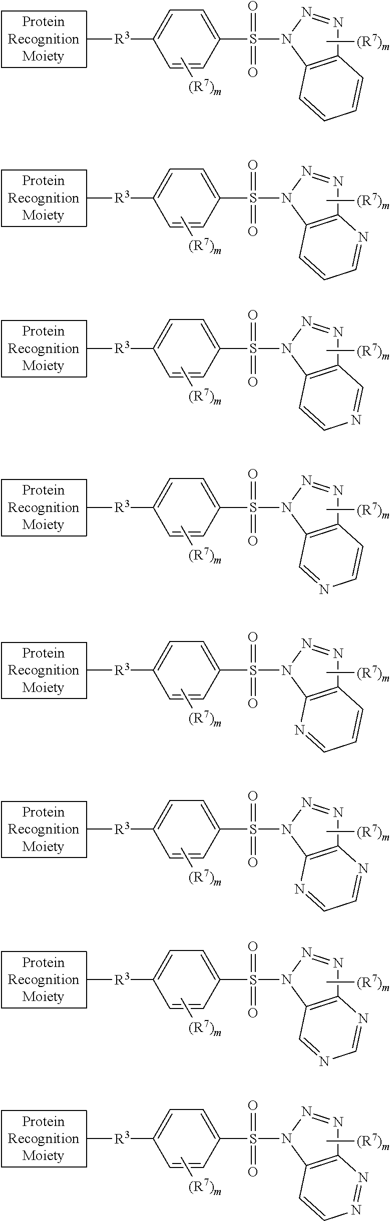

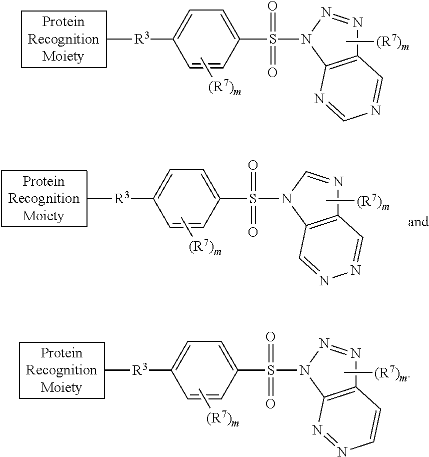

In certain embodiments, the heteroaryl sulfonyl compound of the present invention is selected from:

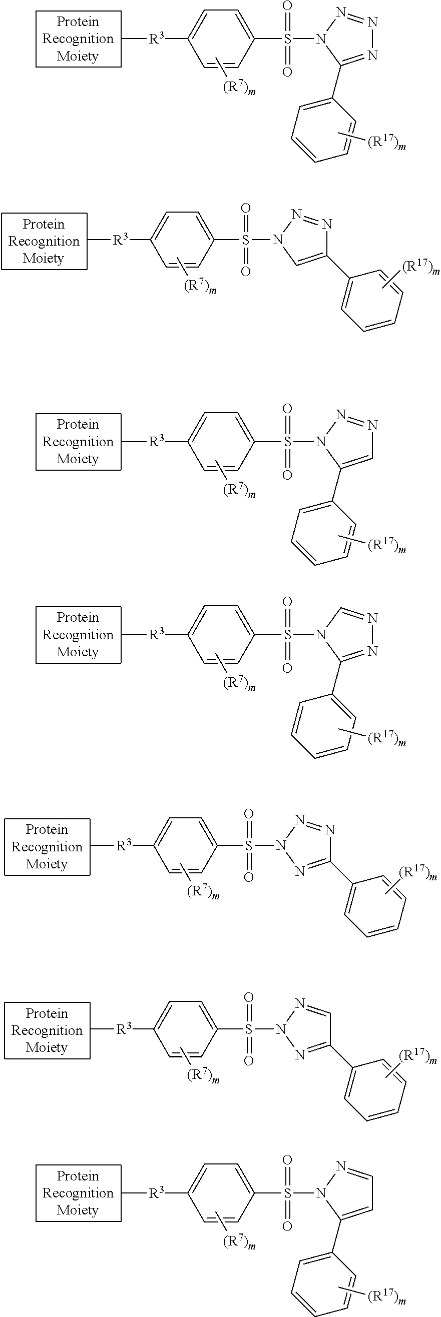

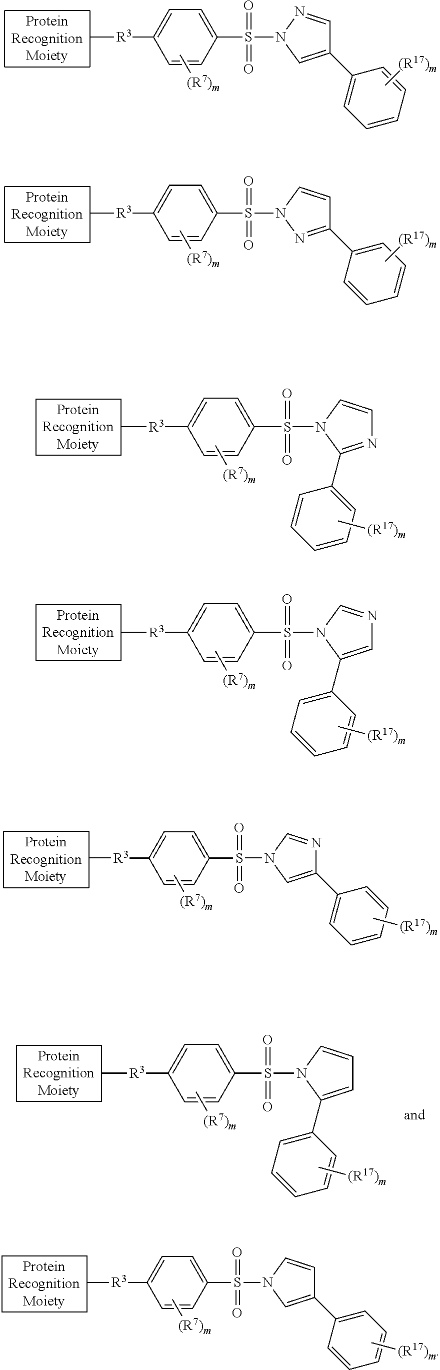

In certain embodiments, the heteroaryl sulfonyl compound of the present invention is selected from:

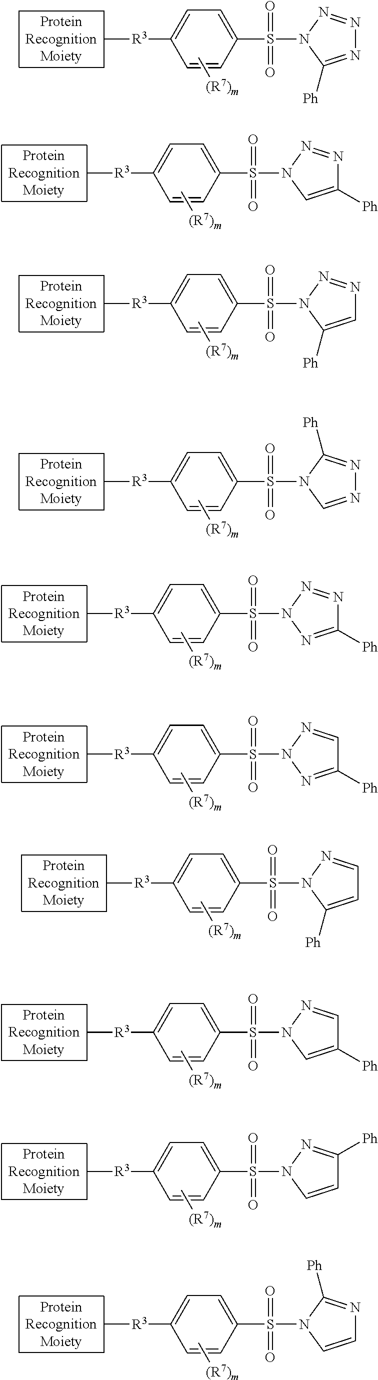

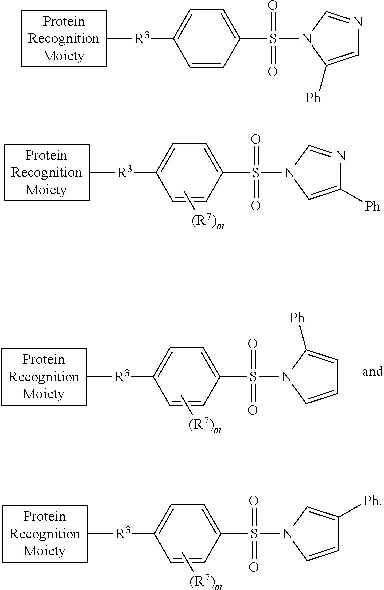

In certain embodiments, the heteroaryl sulfonyl compound of the present invention is selected from:

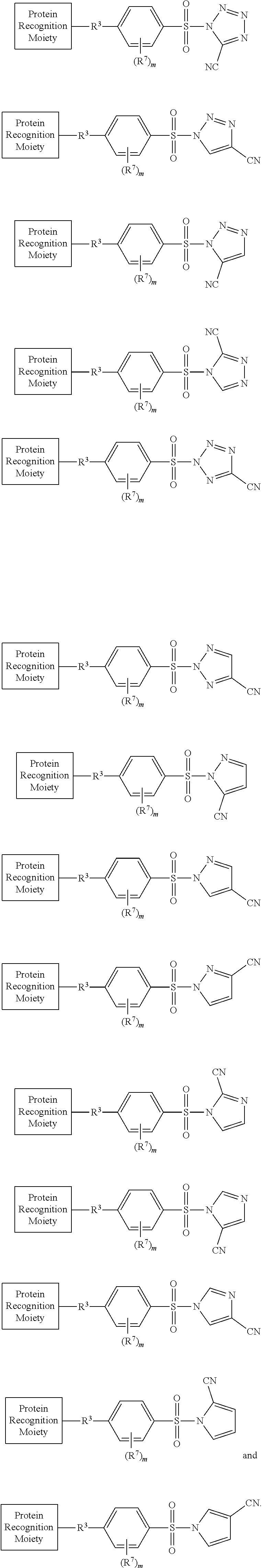

### Embodiments of R¹

In certain embodiments R¹ is

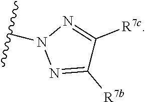

In certain embodiments R¹ is

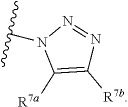

In certain embodiments R¹ is

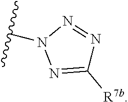

In certain embodiments R¹ is

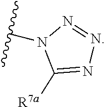

In certain embodiments R¹ is

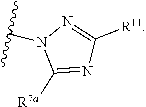

In certain embodiments R¹ is

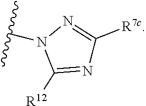

In certain embodiments R¹ is

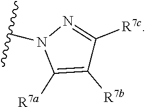

In certain embodiments R¹ is

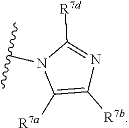

In certain embodiments R¹ is a fused bicyclic heteroaryl.

In certain embodiments R¹ or R⁴ is

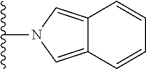 [SMILES: *n1cc2ccccc2c1]

optionally substituted with 1 2, 3, or 4 R⁷ substituents.

In certain embodiments R¹ or R⁴ is

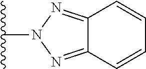 [SMILES: *n1nc2ccccc2n1]

optionally substituted with 1, 2, 3, or 4 R⁷ substituents.

In certain embodiments R¹ or R⁴ is

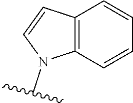 [SMILES: *n1ccc2ccccc21]

optionally substituted with 1, 2, 3, or 4 R⁷ substituents.

In certain embodiments R¹ or R⁴ is

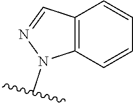 [SMILES: *n1ncc2ccccc21]

optionally substituted with 1, 2, 3, or 4 R⁷ substituents.

In certain embodiments R¹ or R⁴ is

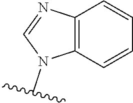 [SMILES: *n1cnc2ccccc21]

optionally substituted with 1, 2, 3, or 4 R⁷ substituents.

In certain embodiments R¹ or R⁴ is

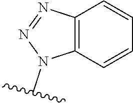 [SMILES: *n1nnc2ccccc21]

optionally substituted with 1, 2, 3, or 4 R⁷ substituents.

In certain embodiments R¹ or R⁴ is

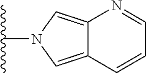 [SMILES: *n1cc2cccnc2c1]

optionally substituted with 1, 2, 3, or 4 R⁷ substituents.

In certain embodiments R¹ or R⁴ is

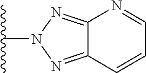 [SMILES: *n1nc2cccnc2n1]

optionally substituted with 1, 2, or 3 R⁷ substituents.

In certain embodiments R¹ or R⁴ is

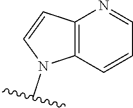 [SMILES: *n1ccc2ncccc21]

optionally substituted with 1, 2, 3, or 4 R⁷ substituents.

In certain embodiments R¹ or R⁴ is

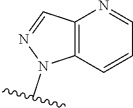 [SMILES: *n1ncc2ncccc21]

optionally substituted with 1, 2, 3, or 4 R⁷ substituents.

In certain embodiments R¹ or R⁴ is

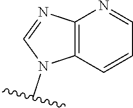 [SMILES: *n1cnc2ncccc21]

optionally substituted with 1, 2, 3, or 4 R⁷ substituents.

In certain embodiments R¹ or R⁴ is

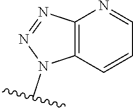 [SMILES: *n1nnc2ncccc21]

optionally substituted with 1, 2, or 3 R⁷ substituents.

In certain embodiments R¹ or R⁴ is

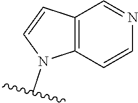 [SMILES: *n1ccc2cnccc21]

optionally substituted with 1, 2, 3, or 4 R⁷ substituents.

In certain embodiments R¹ or R⁴ is

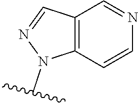 [SMILES: *n1ncc2cnccc21]

optionally substituted with 1, 2, 3, or 4 R⁷ substituents.

In certain embodiments R¹ or R⁴ is

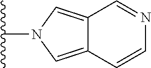 [SMILES: *n1cc2ccncc2c1]

optionally substituted with 1, 2, 3, or 4 R⁷ substituents.

In certain embodiments R¹ or R⁴ is

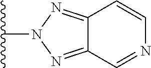 [SMILES: *n1nc2ccncc2n1]

optionally substituted with 1, 2, or 3, R⁷ substituents.

In certain embodiments R¹ or R⁴ is

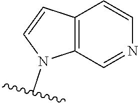 [SMILES: *n1ccc2ccncc21]

optionally substituted with 1, 2, 3, or 4 R⁷ substituents.

In certain embodiments R¹ or R⁴ is

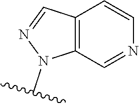 [SMILES: *n1ncc2ccncc21]

optionally substituted with 1, 2, 3, or 4 R⁷ substituents.

In certain embodiments R¹ or R⁴ is

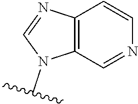 [SMILES: *n1cnc2ccncc21]

optionally substituted with 1, 2, 3, or 4 R⁷ substituents.

In certain embodiments R¹ or R⁴ is

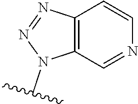 [SMILES: *n1nnc2ccncc21]

optionally substituted with 1, 2, or 3 R⁷ substituents.

In certain embodiments R¹ or R⁴ is

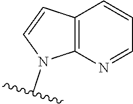 [SMILES: *n1ccc2cccnc21]

optionally substituted with 1, 2, 3, or 4 R⁷ substituents.

In certain embodiments R¹ or R⁴ is

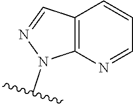 [SMILES: *n1ncc2cccnc21]

optionally substituted with 1, 2, 3, or 4 R⁷ substituents.

In certain embodiments R¹ or R⁴ is

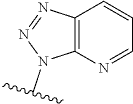 [SMILES: *n1nnc2cccnc21]

optionally substituted with 1, 2, or 3 R⁷ substituents.

In certain embodiments R¹ or R⁴ is

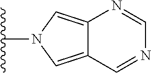 [SMILES: *n1cc2cncnc2c1]

optionally substituted with 1, 2, 3, or 4 R⁷ substituents.

In certain embodiments R¹ or R⁴ is

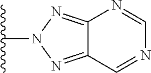 [SMILES: *n1nc2cncnc2n1]

optionally substituted with 1 or 2 R⁷ substituents.

In certain embodiments R¹ or R⁴ is

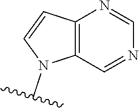 [SMILES: *n1ccc2ncncc21]

optionally substituted with 1, 2, 3, or 4 R⁷ substituents.

In certain embodiments R¹ or R⁴ is

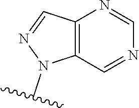 [SMILES: *n1ncc2ncncc21]

optionally substituted with 1, 2, or 3 R⁷ substituents.

In certain embodiments R¹ or R⁴ is

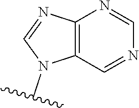 [SMILES: *n1cnc2ncncc21]

optionally substituted with 1, 2, or 3 R⁷ substituents.

In certain embodiments R¹ or R⁴ is

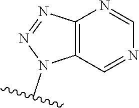 [SMILES: *n1nnc2ncncc21]

optionally substituted with 1 or 2 R⁷ substituents.

In certain embodiments R¹ or R⁴ is

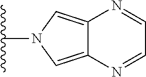 [SMILES: *n1cc2nccnc2c1]

optionally substituted with 1, 2, 3, or 4 R⁷ substituents.

In certain embodiments R¹ or R⁴ is

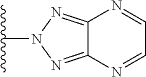 [SMILES: *n1nc2nccnc2n1]

optionally substituted with 1 or 2 R⁷ substituents.

In certain embodiments R¹ or R⁴ is

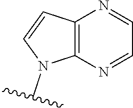 [SMILES: *n1ccc2nccnc21]

optionally substituted with 1, 2, 3, or 4 R⁷ substituents.

In certain embodiments R¹ or R⁴ is

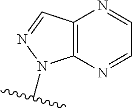 [SMILES: *n1ncc2nccnc21]

optionally substituted with 1, 2, or 3 R⁷ substituents.

In certain embodiments R¹ or R⁴ is

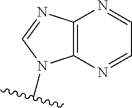 [SMILES: *n1cnc2nccnc21]

optionally substituted with 1, 2, or 3 R⁷ substituents.

In certain embodiments R¹ or R⁴ is

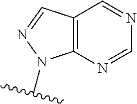 [SMILES: *n1ncc2cncnc21]

optionally substituted with 1, 2, or 3 R⁷ substituents.

In certain embodiments R¹ or R⁴ is

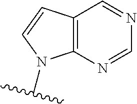 [SMILES: *n1ccc2cncnc21]

optionally substituted with 1, 2, 3, or 4 R⁷ substituents.

In certain embodiments R¹ or R⁴ is

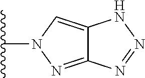 [SMILES: *n1cc2[nH]nnc2n1]

optionally substituted with 1 or 2 R⁷ substituents.

In certain embodiments R¹ or R⁴ is

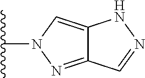 [SMILES: *n1cc2[nH]ncc2n1]

optionally substituted with 1, 2, or 3 R⁷ substituents.

In certain embodiments R¹ or R⁴ is

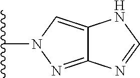 [SMILES: *n1cc2[nH]cnc2n1]

optionally substituted with 1, 2, or 3 R⁷ substituents.

In certain embodiments R¹ or R⁴ is

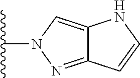 [SMILES: *n1cc2[nH]ccc2n1]

optionally substituted with 1, 2, 3, or 4 R⁷ substituents.

In certain embodiments R¹ or R⁴ is

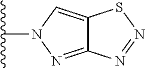 [SMILES: *n1cc2snnc2n1]

optionally substituted with 1 R⁷ substituents.

In certain embodiments R¹ or R⁴ is

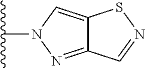 [SMILES: *n1cc2sncc2n1]

optionally substituted with 1 or 2 R⁷ substituents.

In certain embodiments R¹ or R⁴ is

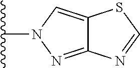 [SMILES: *n1cc2scnc2n1]

optionally substituted with 1 or 2 R⁷ substituents.

In certain embodiments R¹ or R⁴ is

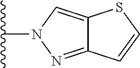 [SMILES: *n1cc2sccc2n1]

optionally substituted with 1, 2, or 3 R⁷ substituents.

In certain embodiments R¹ or R⁴ is

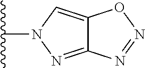 [SMILES: *n1cc2onnc2n1]

optionally substituted with 1 R⁷ substituents.

In certain embodiments R¹ or R⁴ is

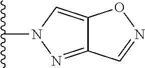 [SMILES: *n1cc2oncc2n1]

optionally substituted with 1 or 2 R⁷ substituents.

In certain embodiments R¹ or R⁴ is

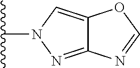 [SMILES: *n1cc2ocnc2n1]

optionally substituted with 1 or 2 R⁷ substituents.

In certain embodiments R¹ or R⁴ is

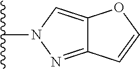 [SMILES: *n1cc2occc2n1]

optionally substituted with 1, 2, or 3 R⁷ substituents.

In certain embodiments R¹ or R⁴ is

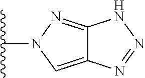 [SMILES: *n1cc2nn[nH]c2n1]

optionally substituted with 1 or 2 R⁷ substituents.

In certain embodiments R¹ or R⁴ is

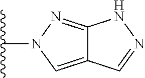 [SMILES: *n1cc2cn[nH]c2n1]

optionally substituted with 1, 2, or 3 R⁷ substituents.

In certain embodiments R¹ or R⁴ is

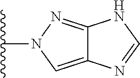 [SMILES: *n1cc2nc[nH]c2n1]

optionally substituted with 1, 2, or 3 R⁴ substituents.

In certain embodiments R¹ or R⁴ is

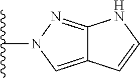 [SMILES: *n1cc2cc[nH]c2n1]

optionally substituted with 1, 2, 3 or 4 R⁷ substituents.

In certain embodiments R¹ or R⁴ is

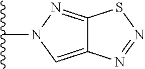 [SMILES: *n1cc2nnsc2n1]

optionally substituted with 1 R⁷ substituents.

In certain embodiments R¹ or R⁴ is

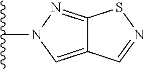 [SMILES: *n1cc2cnsc2n1]

optionally substituted with 1 or 2 R⁷ substituents.

In certain embodiments R¹ or R⁴ is

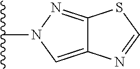 [SMILES: *n1cc2ncsc2n1]

optionally substituted with 1 or 2 R⁷ substituents.

In certain embodiments R¹ or R⁴ is

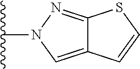 [SMILES: *n1cc2ccsc2n1]

optionally substituted with 1, 2 or 3 R⁷ substituents.

In certain embodiments R¹ or R⁴ is

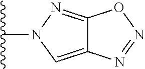 [SMILES: *n1cc2nnoc2n1]

optionally substituted with 1 R⁷ substituents.

In certain embodiments R¹ or R⁴ is

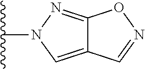 [SMILES: *n1cc2cnoc2n1]

optionally substituted with 1 or 2 R⁷ substituents.

In certain embodiments R¹ or R⁴ is

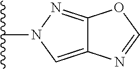 [SMILES: *n1cc2ncoc2n1]

optionally substituted with 1 or 2 R⁷ substituents.

In certain embodiments R¹ or R⁴ is

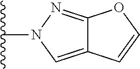 [SMILES: *n1cc2ccoc2n1]

optionally substituted with 1, 2, or 3 R⁷ substituents.

### Embodiments of R² and R³

Bivalent substituents described herein can be either attached in a left to right fashion or a right to left fashion except as excluded by context. For example, where R² is -aryl-C(O)—NR⁶— either the aryl or nitrogen side is attached to the sulfonyl group. For example, when R² is -aryl-C(O)—NR⁶—, Formula I can be

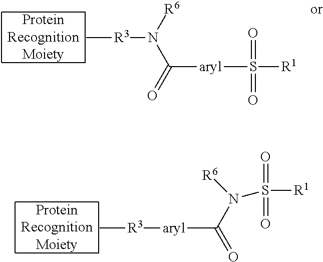

similarly, when —R³— is

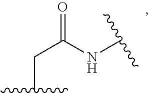 [SMILES: *CC(=O)N*]

### Formula I can be

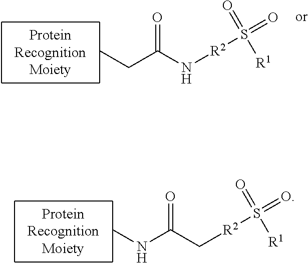

In certain embodiments —R²—R³— and —R³—R²— are selected from:

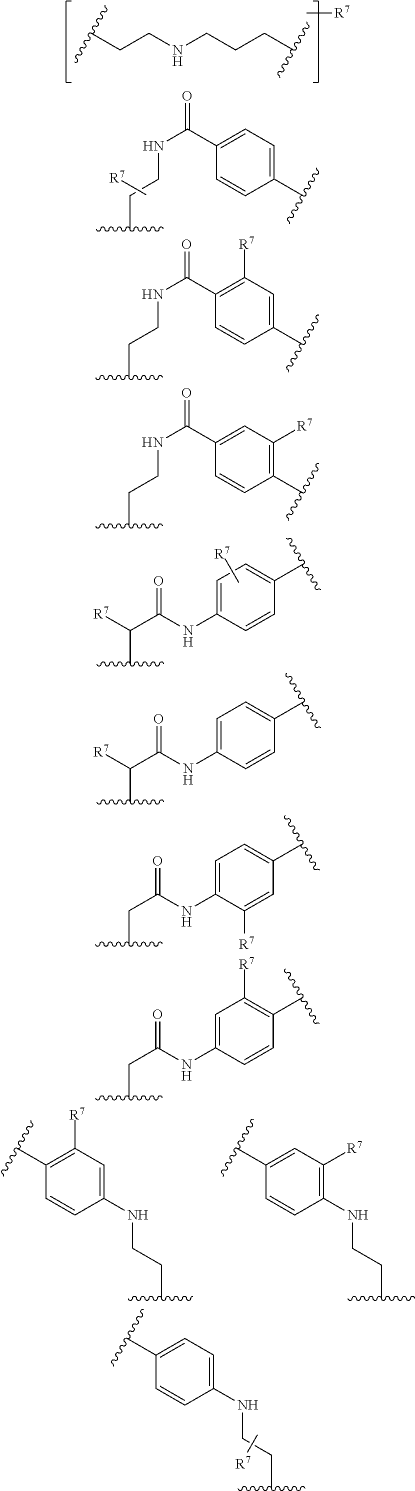

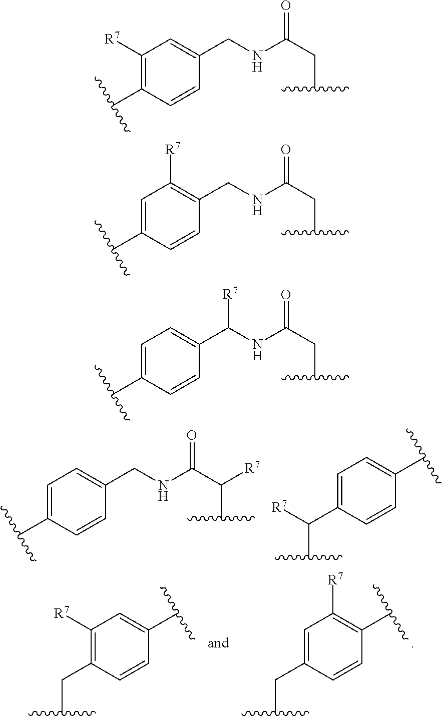

In certain embodiments —R²—R³— and —R³—R²— are selected from:

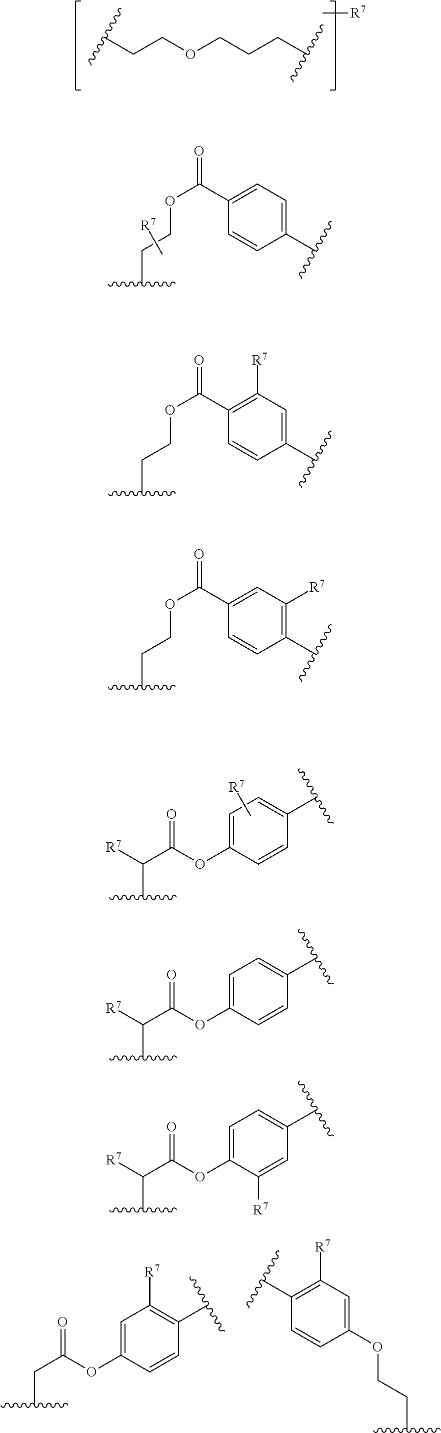

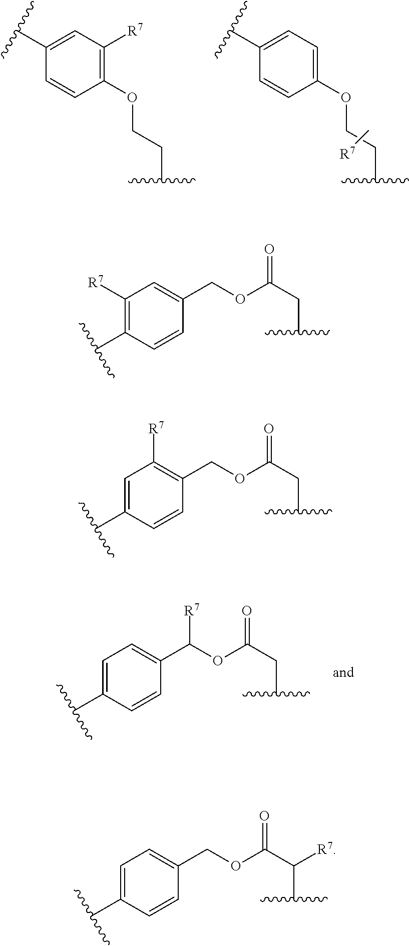

In certain embodiments —R²—R³— and —R³—R²— are selected from:

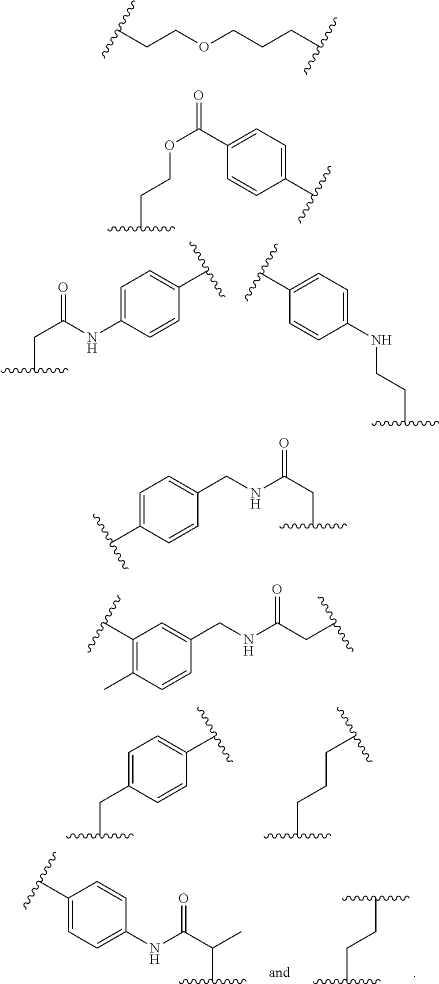

In certain embodiments R² is selected from:

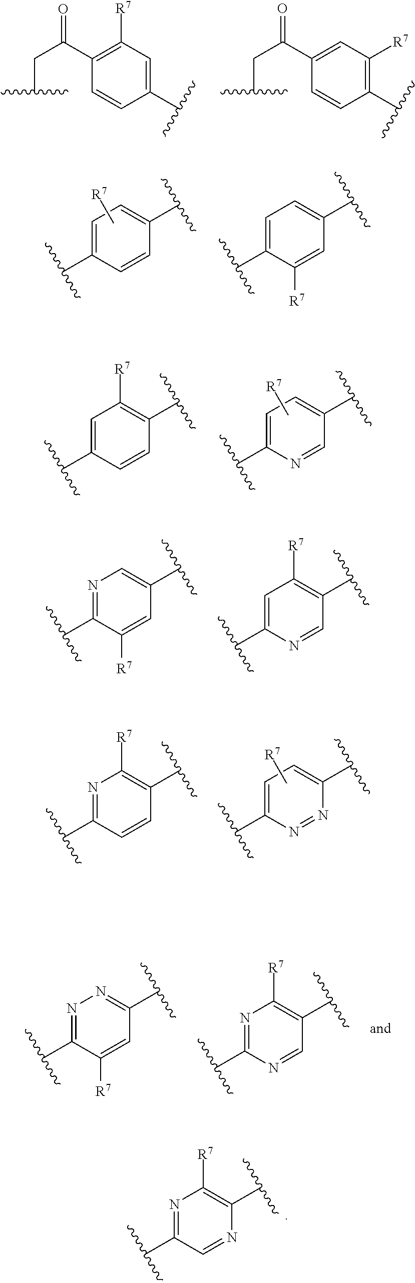

In certain embodiments R² is selected from

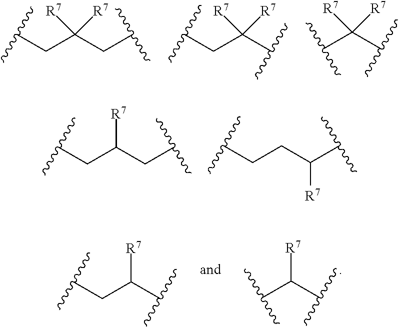

In certain embodiments R² is selected from:

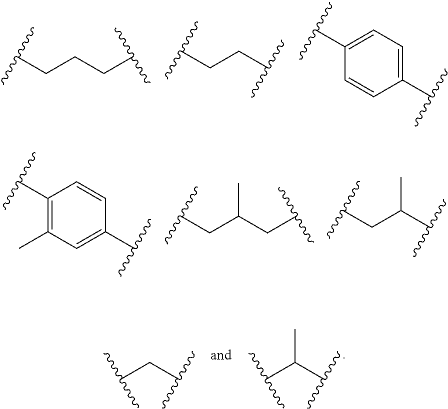

In certain embodiments R² is selected from:

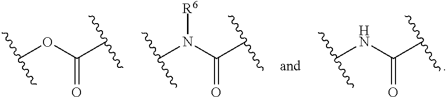

In certain embodiments R³ is selected from

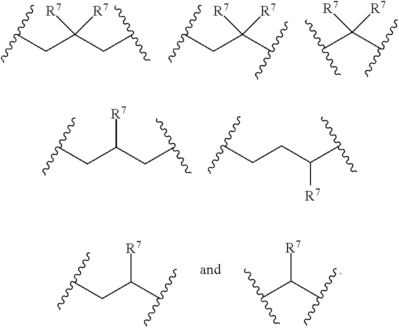

In certain embodiments R³ is selected from:

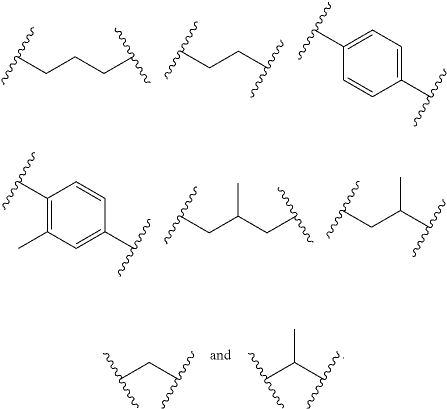

In certain embodiments R³ is selected from:

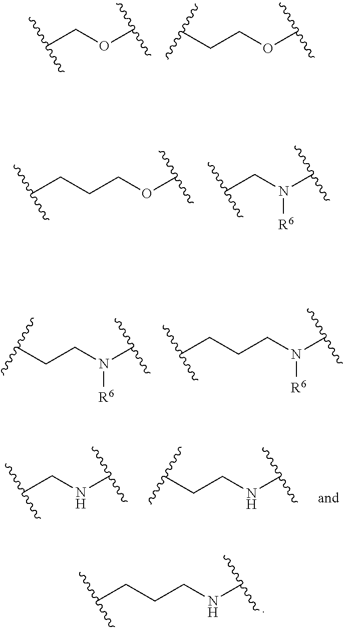

In certain embodiments R³ is selected from:

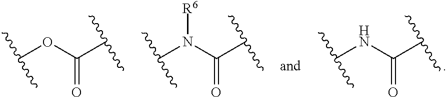

In certain embodiments R³ is selected from:

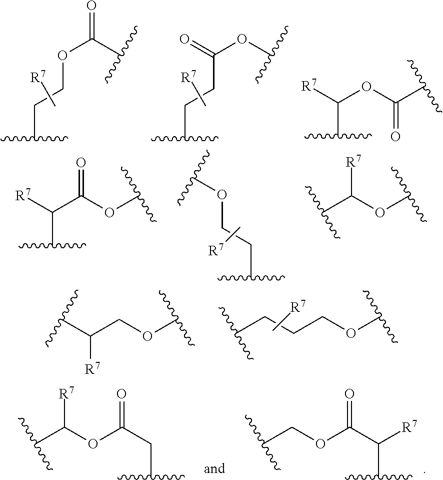

In certain embodiments R³ is selected from:

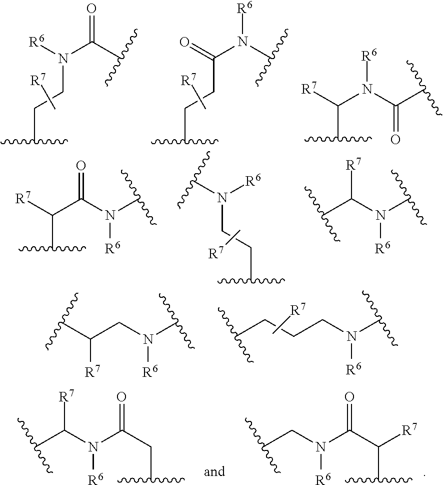

In certain embodiments R³ is selected from:

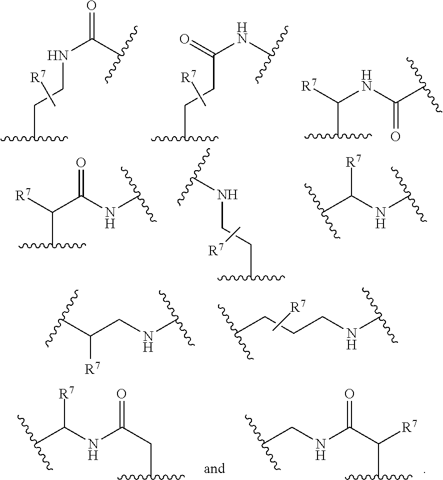

In certain embodiments R² is bond.

In certain embodiments R³ is bond.

In certain embodiments R² and R³ are both bond.

In certain embodiments one of R² and R³ is bond and the other is selected from alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ, (OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl, aryl-C(O)—NR⁶—, heteroaryl-C(O)—NR⁶—, and heteroaryl, each of which is optionally substituted as allowed by valence with 1, 2, 3, or 4 substituents selected from R⁷.

In certain embodiments p is 1.

In certain embodiments p is 2.

In certain embodiments p is 3.

In certain embodiments p is 4.

In certain embodiments p is 5.

In certain embodiments p is 6.

In certain embodiments R² is phenyl.

In certain embodiments R² is phenyl substituted with 1 substituent selected from R⁷.

In certain embodiments R² is phenyl substituted with 2 substituents selected from R⁷.

In certain embodiments R² is phenyl substituted with 3 substituents selected from R⁷.

In certain embodiments R² is phenyl substituted with 4 substituents selected from R⁷.

In certain embodiments R² is phenyl substituted with 1 substituent selected from R^{7EWG}.

In certain embodiments R² is phenyl substituted with 2 substituents selected from R^{7EWG}.

In certain embodiments R² is phenyl substituted with 3 substituents selected from R^{7EWG}.

In certain embodiments R² is phenyl substituted with 4 substituents selected from R^{7EWG}.

R^{7EWG} is independently selected at each instance from halogen, haloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, with each haloalkyl, heterocycle, aryl, and heteroaryl optionally substituted as allowed by valence with 1, 2, 3, or 4 substituents selected from R¹⁷.

In certain embodiments R³ is phenyl substituted with 1 substituent selected from R⁷.

In certain embodiments R³ is phenyl substituted with 2 substituents selected from R⁷.

In certain embodiments R³ is phenyl substituted with 3 substituents selected from R⁷.

In certain embodiments R³ is phenyl substituted with 4 substituents selected from R⁷.

In certain embodiments R² is heteroaryl.

In certain embodiments R² is heteroaryl substituted with 1 substituent selected from R⁷.

In certain embodiments R² is heteroaryl substituted with 2 substituents selected from R⁷.

In certain embodiments R² is heteroaryl substituted with 3 substituents selected from R⁷.

In certain embodiments R² is heteroaryl substituted with 4 substituents selected from R⁷.

### Embodiments of R⁴

In certain embodiments R⁴ is

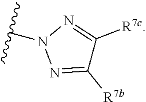

In certain embodiments R⁴ is

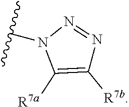

In certain embodiments R⁴ is

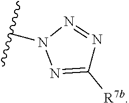

In certain embodiments R⁴ is

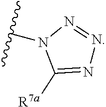

In certain embodiments R⁴ is

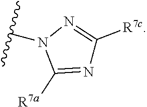

In certain embodiments R⁴ is

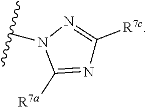

In certain embodiments R⁴ is

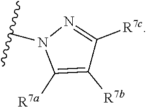

In certain embodiments R⁴ is

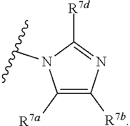

In certain embodiments R⁴ is a 5-membered heteroaryl.

In certain embodiments R⁴ is a fused bicyclic heteroaryl.

In certain embodiments each R⁷ is independently selected from R^{7a}, R^{7b}, R^{7c} and R^{7d}.

In certain embodiments R⁴ is a bicyclic heteroaryl optionally substituted with 1, 2, 3, or 4 R⁷ substituents.

### Embodiments of R⁵

In certain embodiments R⁵ is selected from

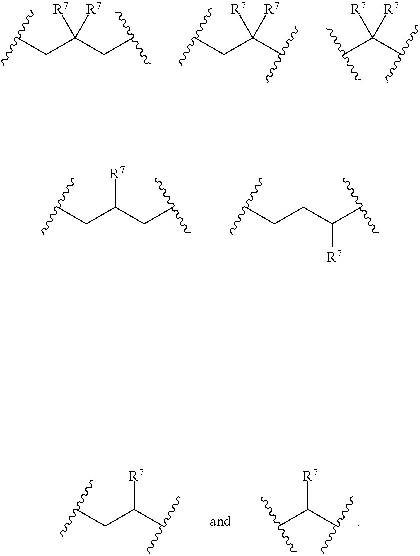

In certain embodiments R⁵ is selected from:

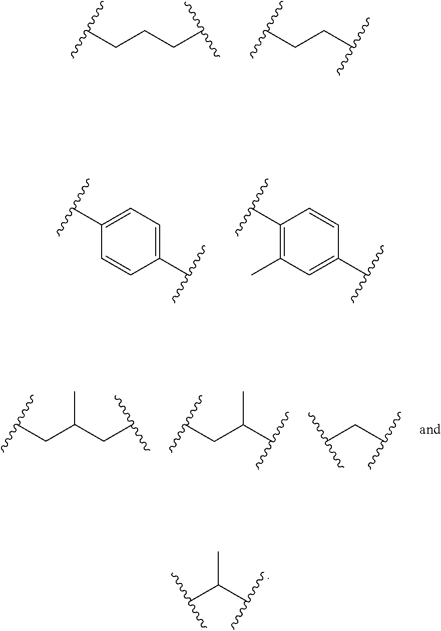

In certain embodiments R⁵ is bond.

In certain embodiments R⁵ and R³ are both bond.

In certain embodiments one of R⁵ and R³ is bond and the other is selected from alkyl, alkenyl, haloalkyl, cycloalkyl, heterocycle, naphthyl, —S—, —O—, —NR⁶—, —(CH₂)ₚ—C(O)—, —(CH₂)ₚ—C(O)—NR⁶—, —(CH₂CH₂O)ₚ, (OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl, heteroaryl-C(O)—NR⁶—, and heteroaryl, each of which except bond is optionally substituted as allowed by valence with 1, 2, 3, or 4 substituents selected from R⁷.

### Embodiments of R⁶

In certain embodiments one R⁶ is hydrogen.

In certain embodiments one R⁶ is alkyl.

In certain embodiments one R⁶ is haloalkyl.

In certain embodiments one R⁶ is cycloalkyl.

In certain embodiments one R⁶ is aryl.

In certain embodiments one R⁶ is heterocycle.

In certain embodiments one R⁶ is heteroaryl.

### Embodiments of R⁷

In certain embodiments R⁷ is independently selected at each instance from R^{7EWG}.

In certain embodiments R^{7a} is independently selected at each instance from R^{7EWG}.

In certain embodiments R^{7b} is independently selected at each instance from R^{7EWG}.

In certain embodiments R^{7c} is independently selected at each instance from R^{7EWG}.

In certain embodiments R^{7d} is independently selected at each instance from R^{7EWG}.

R^{7EWG} is independently selected at each instance from halogen, haloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, with each haloalkyl, heterocycle, aryl, and heteroaryl optionally substituted as allowed by valence with 1, 2, 3, or 4 substituents selected from R¹⁷.

In certain embodiments R⁷ is hydrogen.

In certain embodiments R⁷ is cyano.

In certain embodiments R⁷ is halogen.

In certain embodiments R⁷ is fluoro.

In certain embodiments R⁷ is haloalkyl.

In certain embodiments R⁷ is —CF₃.

In certain embodiments R⁷ is aryl optionally substituted as allowed by valence with 1, 2, 3, or 4 substituents selected from R¹⁷.

In certain embodiments R⁷ is phenyl optionally substituted as allowed by valence with 1, 2, 3, or 4 substituents selected from R¹⁷.

In certain embodiments R⁷ is aryl.

In certain embodiments R⁷ is phenyl.

In certain embodiments R^{7a} is hydrogen.

In certain embodiments R^{7a} is cyano.

In certain embodiments R^{7a} is halogen.

In certain embodiments R^{7a} is fluoro.

In certain embodiments R^{7a} is haloalkyl.

In certain embodiments R^{7a} is —CF₃.

In certain embodiments R^{7a} is aryl optionally substituted as allowed by valence with 1, 2, 3, or 4 substituents selected from R¹⁷.

In certain embodiments R^{7a} is phenyl optionally substituted as allowed by valence with 1, 2, 3, or 4 substituents selected from R¹⁷.

In certain embodiments R^{7a} is aryl.

In certain embodiments R^{7a} is phenyl.

In certain embodiments R^{7b} is hydrogen.

In certain embodiments R^{7b} is cyano.

In certain embodiments R^{7b} is halogen.

In certain embodiments R^{7b} is fluoro.

In certain embodiments R^{7b} is haloalkyl.

In certain embodiments R^{7b} is —CF₃.

In certain embodiments R^{7b} is aryl optionally substituted as allowed by valence with 1, 2, 3, or 4 substituents selected from R¹⁷.

In certain embodiments R^{7b} is phenyl optionally substituted as allowed by valence with 1, 2, 3, or 4 substituents selected from R¹⁷.

In certain embodiments R^{7b} is aryl.

In certain embodiments R^{7b} is phenyl.

In certain embodiments R^{7c} is hydrogen.

In certain embodiments R^{7c} is cyano.

In certain embodiments R^{7c} is halogen.

In certain embodiments R^{7c} is fluoro.

In certain embodiments R^{7c} is haloalkyl.

In certain embodiments R^{7c} is —CF₃.

In certain embodiments R^{7c} is aryl optionally substituted as allowed by valence with 1, 2, 3, or 4 substituents selected from R¹⁷.

In certain embodiments R^{7c} is phenyl optionally substituted as allowed by valence with 1, 2, 3, or 4 substituents selected from R¹⁷.

In certain embodiments R^{7c} is aryl.

In certain embodiments R^{7c} is phenyl.

In certain embodiments R^{7d} is hydrogen.

In certain embodiments R^{7d} is cyano.

In certain embodiments R^{7d} is halogen.

In certain embodiments R^{7d} is fluoro.

In certain embodiments R^{7d} is haloalkyl.

In certain embodiments R^{7d} is —CF₃.

In certain embodiments R^{7d} is aryl optionally substituted as allowed by valence with 1, 2, 3, or 4 substituents selected from R¹⁷.

In certain embodiments R^{7d} is phenyl optionally substituted as allowed by valence with 1, 2, 3, or 4 substituents selected from R¹⁷.

In certain embodiments R^{7d} is aryl.

In certain embodiments R^{7d} is phenyl.

### Embodiments of R^{8a}, R^{8b}, R^{8c}, and R^{8d}

In certain embodiments R^{8a} is R¹².

In certain embodiments R^{8b} is R¹².

In certain embodiments R^{8c} is R¹².

In certain embodiments R^{8d} is R¹².

In certain embodiments R^{8a} is R¹² and R^{8b}, R^{8c}, and R^{8d} are hydrogen.

In certain embodiments R^{8b} is R¹² and R^{8c}, R^{8d}, and R^{8a} are hydrogen.

In certain embodiments R^{8c} is R¹² and R^{8b}, R^{8d}, and R^{8a} are hydrogen.

In certain embodiments R^{8d} is R¹² and R^{8b}, R^{8c}, and R^{8a} are hydrogen.

In certain embodiments R^{8a} is cyano.

In certain embodiments R^{8a} is halogen.

In certain embodiments R^{8a} is fluoro.

In certain embodiments R^{8a} is haloalkyl.

In certain embodiments R^{8a} is —CF₃.

In certain embodiments R^{8a} is aryl optionally substituted as allowed by valence with 1, 2, 3, or 4 substituents selected from R¹⁷.

In certain embodiments R^{8a} is phenyl optionally substituted as allowed by valence with 1, 2, 3, or 4 substituents selected from R¹⁷.

In certain embodiments R^{8a} is aryl.

In certain embodiments R^{8a} is phenyl.

In certain embodiments R^{8a} is OR⁶.

In certain embodiments R^{8a} is N(R⁶)₂.

In certain embodiments R^{8b} is cyano.

In certain embodiments R^{8b} is halogen.

In certain embodiments R^{8b} is fluoro.

In certain embodiments R^{8b} is haloalkyl.

In certain embodiments R^{8b} is —CF₃.

In certain embodiments R^{8b} is aryl optionally substituted as allowed by valence with 1, 2, 3, or 4 substituents selected from R¹⁷.

In certain embodiments R^{8b} is phenyl optionally substituted as allowed by valence with 1, 2, 3, or 4 substituents selected from R¹⁷.

In certain embodiments R^{8b} is aryl.

In certain embodiments R^{8b} is phenyl.

In certain embodiments R^{8b} is OR⁶.

In certain embodiments R^{8b} is N(R⁶)₂.

In certain embodiments R^{8c} is cyano.

In certain embodiments R^{8c} is halogen.

In certain embodiments R^{8c} is fluoro.

In certain embodiments R^{8c} is haloalkyl.

In certain embodiments R^{8c} is —CF₃.

In certain embodiments R^{8c} is aryl optionally substituted as allowed by valence with 1, 2, 3, or 4 substituents selected from R¹⁷.

In certain embodiments R^{8c} is phenyl optionally substituted as allowed by valence with 1, 2, 3, or 4 substituents selected from R¹⁷.

In certain embodiments R^{8c} is aryl.

In certain embodiments R^{8c} is phenyl.

In certain embodiments R^{8c} is OR⁶.

In certain embodiments R^{8c} is N(R⁶)₂.

In certain embodiments R^{8d} is cyano.

In certain embodiments R^{8d} is halogen.

In certain embodiments R^{8d} is fluoro.

In certain embodiments R^{8d} is haloalkyl.

In certain embodiments R^{8d} is —CF₃.

In certain embodiments R^{8d} is aryl optionally substituted as allowed by valence with 1, 2, 3, or 4 substituents selected from R¹⁷.

In certain embodiments R^{8d} is phenyl optionally substituted as allowed by valence with 1, 2, 3, or 4 substituents selected from R¹⁷.

In certain embodiments R^{8d} is aryl.

In certain embodiments R^{8d} is phenyl.

In certain embodiments R^{8d} is OR⁶.

In certain embodiments R^{8d} is N(R⁶)₂.

### Embodiments of R⁹

In certain embodiments —R²—R⁹— and —R⁹—R²— are selected from:

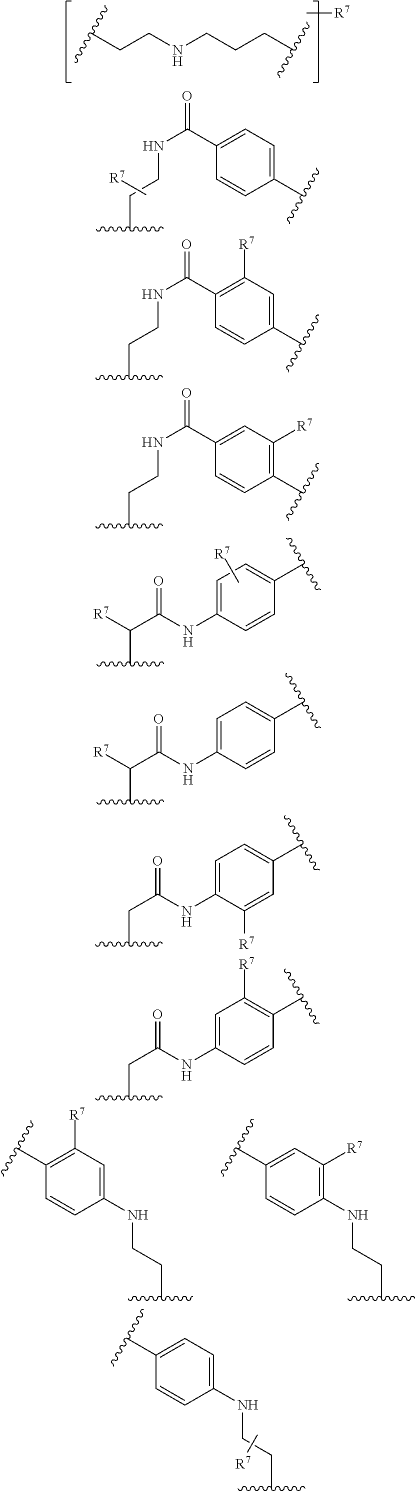

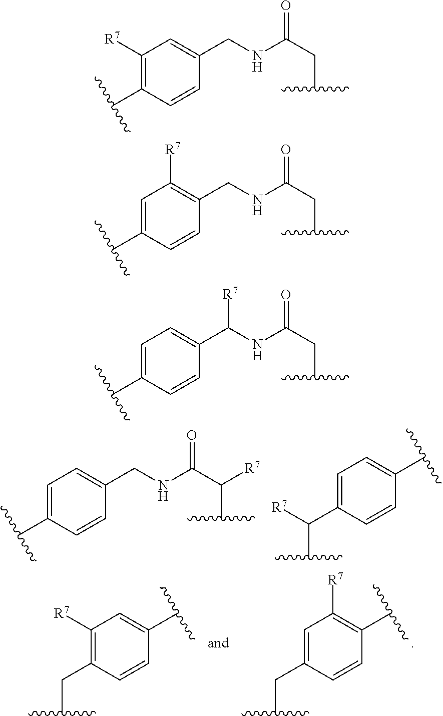

In certain embodiments —R²—R⁹— and —R⁹—R²— are selected from:

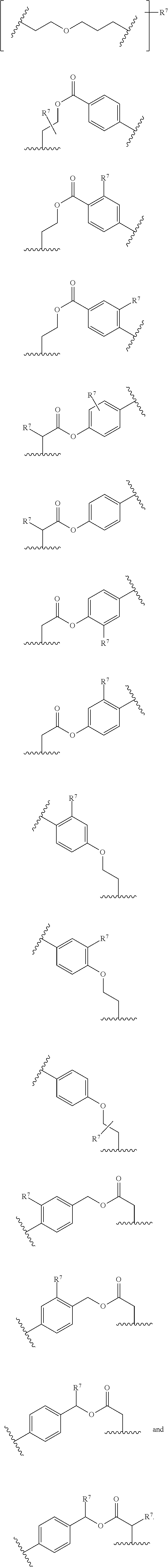

In certain embodiments —R⁹—R³— and —R⁹—R²— are selected from:

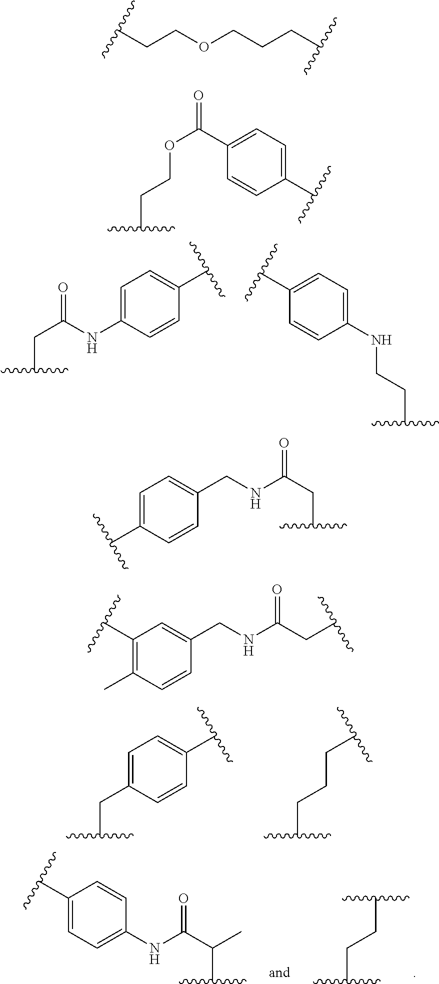

In certain embodiments R⁹ is selected from

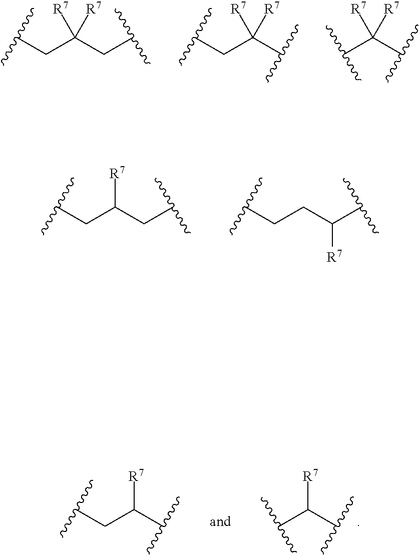

In certain embodiments R⁹ is selected from:

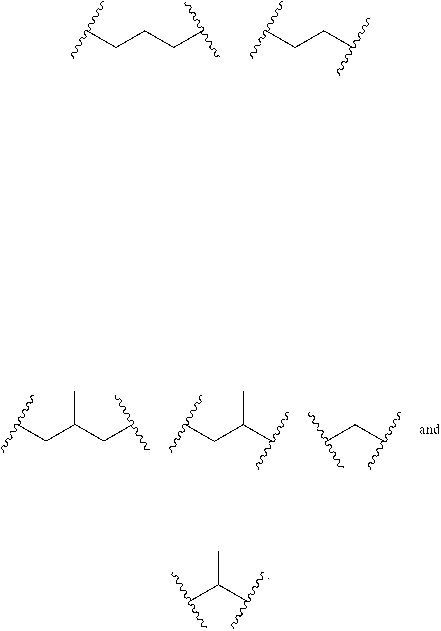

In certain embodiments R⁹ is selected from:

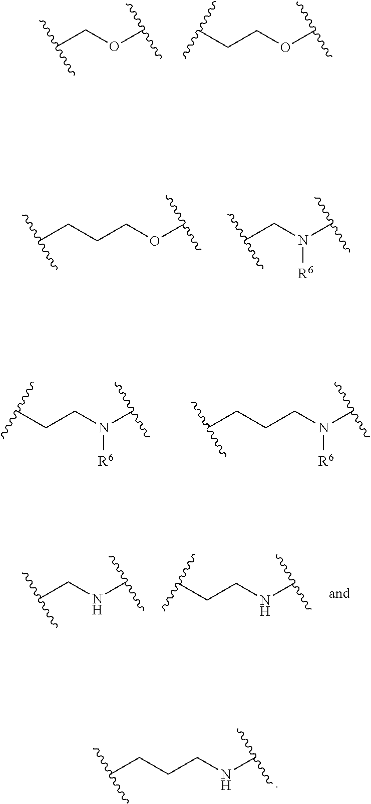

In certain embodiments R⁹ is selected from:

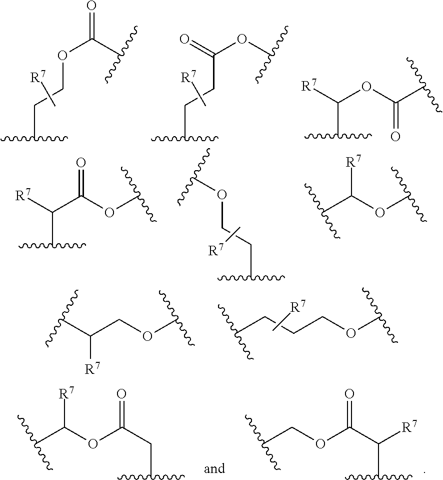

In certain embodiments R⁹ is selected from:

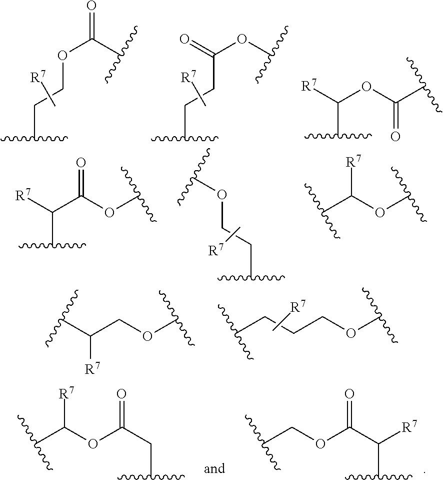

In certain embodiments R² is bond and R⁹ is selected from alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, and heteroaryl, each of which is optionally substituted as allowed by valence with 1, 2, 3, or 4 substituents selected from R⁷.

### Embodiments of R¹¹

In certain embodiments R¹¹ is hydrogen.

In certain embodiments R¹¹ is cyano.

In certain embodiments R¹¹ is halogen.

In certain embodiments R¹¹ is fluoro.

In certain embodiments R¹¹ is haloalkyl.

In certain embodiments R¹¹ is —CF₃.

In certain embodiments R¹¹ is naphthyl optionally substituted as allowed by valence with 1, 2, 3, or 4 substituents selected from R¹⁷.

In certain embodiments R¹¹ is naphthyl.

### Embodiments of R²

In certain embodiments R¹² is cyano.

In certain embodiments R¹² is halogen.

In certain embodiments R¹² is fluoro.

In certain embodiments R¹² is haloalkyl.

In certain embodiments R¹² is —CF₃.

In certain embodiments R¹² is aryl optionally substituted as allowed by valence with 1, 2, 3, or 4 substituents selected from R¹⁷.

In certain embodiments R¹² is aryl.

In certain embodiments R¹² is phenyl optionally substituted as allowed by valence with 1, 2, 3, or 4 substituents selected from R¹⁷.

In certain embodiments R¹² is phenyl.

### Embodiments of R³

In certain embodiments R¹³ is cycloalkyl.

In certain embodiments R¹³ is cyclopropyl.

In certain embodiments R¹³ is aryl optionally substituted as allowed by valence with 1, 2, 3, or 4 substituents selected from R⁷.

In certain embodiments R¹³ is phenyl optionally substituted as allowed by valence with 1, 2, 3, or 4 substituents selected from R⁷.

In certain embodiments R¹³ is heteroaryl optionally substituted as allowed by valence with 1, 2, 3, or 4 substituents selected from R⁷.

In certain embodiments R¹³ is aryl.

In certain embodiments R¹³ is phenyl.

In certain embodiments R¹³ is heteroaryl.

### Embodiments of R¹¹

In certain embodiments R¹⁵ is selected from

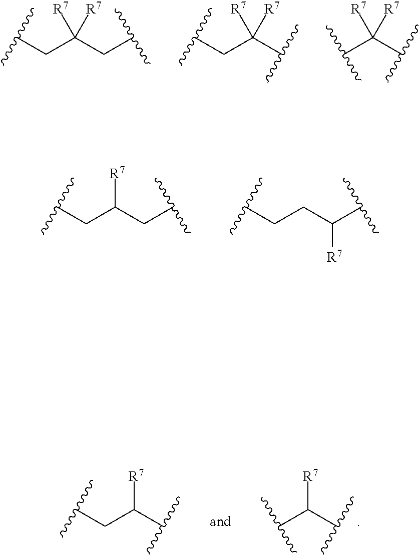

In certain embodiments R¹⁵ is selected from:

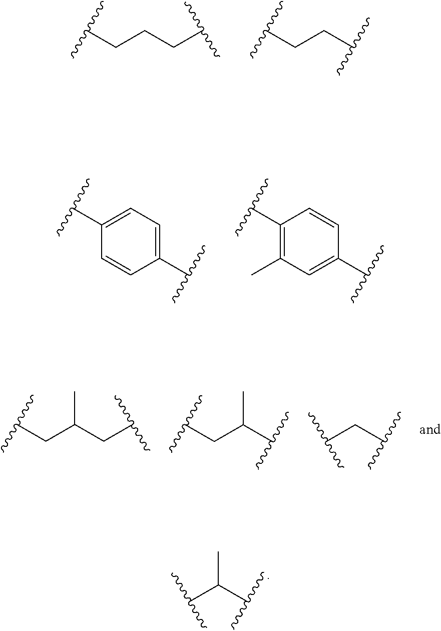

In certain embodiments R¹⁵ is selected from:

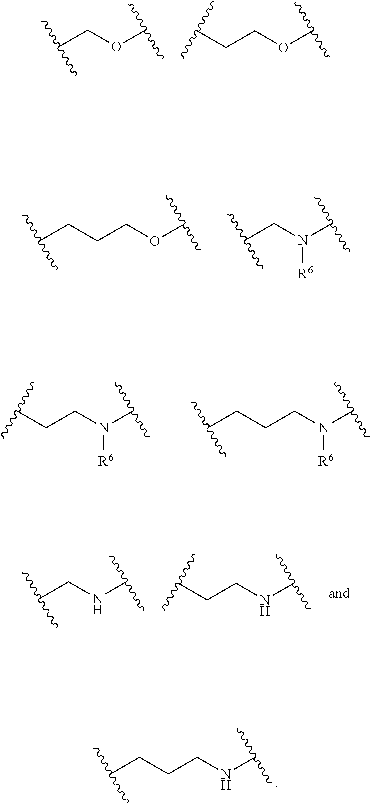

In certain embodiments R¹⁵ is selected from:

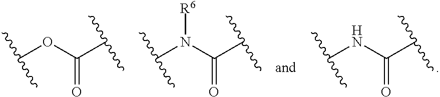

In certain embodiments R¹⁵ is selected from:

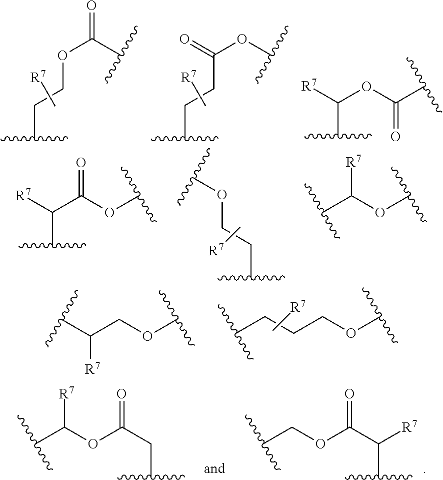

In certain embodiments R¹⁵ is selected from:

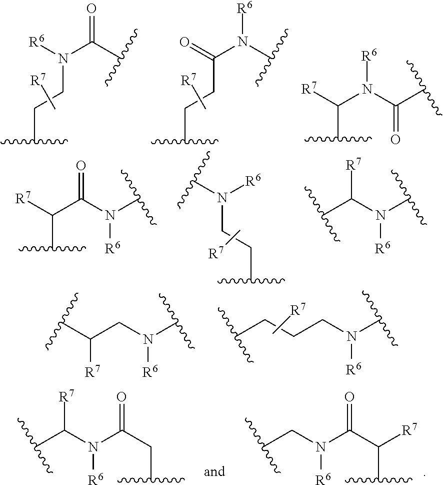

In certain embodiments R¹⁵ is selected from:

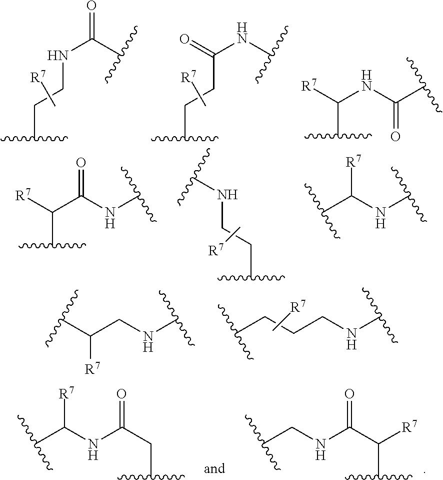

In certain embodiments R¹⁵ is phenyl.

In certain embodiments R¹⁵ is phenyl substituted with 1 substituent selected from R⁷.

In certain embodiments R¹⁵ is phenyl substituted with 2 substituents selected from R⁷.

In certain embodiments R¹⁵ is phenyl substituted with 3 substituents selected from R⁷.

In certain embodiments R¹⁵ is phenyl substituted with 4 substituents selected from R⁷.

In certain embodiments R¹⁵ is heteroaryl.

In certain embodiments R¹⁵ is heteroaryl substituted with 1 substituent selected from R⁷.

In certain embodiments R¹⁵ is heteroaryl substituted with 2 substituents selected from R⁷.

In certain embodiments R¹⁵ is heteroaryl substituted with 3 substituents selected from R⁷.

In certain embodiments R¹⁵ is heteroaryl substituted with 4 substituents selected from R⁷.

### Embodiments of R¹⁶

In certain embodiments R¹⁶ is

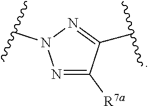

In certain embodiments R¹⁶ is

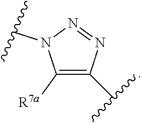

In certain embodiments R¹⁶ is

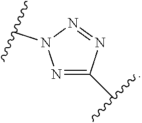 [SMILES: *c1nnn(*)n1]

In certain embodiments R¹⁶ is

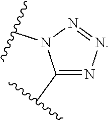 [SMILES: *c1nnnn1*]

In certain embodiments R¹⁶ is

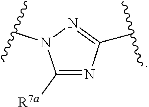

In certain embodiments R¹⁶ is

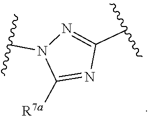

In certain embodiments R¹⁶ is

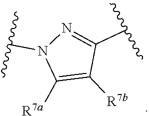

In certain embodiments R¹⁶ is

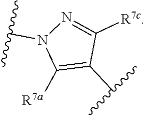

In certain embodiments R¹⁶ is

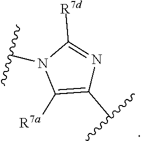

In certain embodiments R¹⁶ is a fused bicyclic heteroaryl.

### Embodiments of R⁷

In certain embodiments R¹⁷ is hydrogen.

In certain embodiments R¹⁷ is cyano.

In certain embodiments R¹⁷ is halogen.

In certain embodiments R¹⁷ is fluoro.

In certain embodiments R¹⁷ is haloalkyl.

In certain embodiments R¹⁷ is —CF₃.

In certain embodiments R¹⁷ is aryl.

In certain embodiments R¹⁷ is phenyl.

In certain embodiments one R¹⁷ is hydrogen.

In certain embodiments one R¹⁷ is cyano.

In certain embodiments one R¹⁷ is halogen.

In certain embodiments one R¹⁷ is fluoro.

In certain embodiments one R¹⁷ is haloalkyl.

In certain embodiments one R¹⁷ is —CF₃.

In certain embodiments one R¹⁷ is aryl.

In certain embodiments one R¹⁷ is phenyl.

### Embodiments of R⁸

In certain embodiments R¹⁸ is hydrogen.

In certain embodiments R¹⁸ is halogen.

In certain embodiments R¹⁸ is alkyl.

In certain embodiments R¹⁸ is haloalkyl.

In certain embodiments R¹⁸ is alkenyl.

In certain embodiments R¹⁸ is cycloalkyl.

In certain embodiments R¹⁸ is heterocycle.

In certain embodiments R¹⁸ is aryl.

In certain embodiments R¹⁸ is heteroaryl.

In certain embodiments R¹⁸ is cyano.

In certain embodiments R¹⁸ is nitro.

In certain embodiments one R¹⁸ is hydrogen.

In certain embodiments one R¹⁸ is halogen.

In certain embodiments one R¹⁸ is alkyl.

In certain embodiments one R¹⁸ is haloalkyl.

In certain embodiments one R¹⁸ is alkenyl.

In certain embodiments one R¹⁸ is cycloalkyl.

In certain embodiments one R¹⁸ is heterocycle.

In certain embodiments one R¹⁸ is aryl.

In certain embodiments one R¹⁸ is heteroaryl.

In certain embodiments one R¹⁸ is cyano.

In certain embodiments one R¹⁸ is nitro.

### Embodiments of R¹⁹

In certain embodiments R¹⁹ is hydrogen.

In certain embodiments R¹⁹ is alkyl.

In certain embodiments R¹⁹ is haloalkyl.

In certain embodiments R¹⁹ is cycloalkyl.

In certain embodiments R¹⁹ is heterocycle.

In certain embodiments R¹⁹ is aryl.

In certain embodiments R¹⁹ is heteroaryl.

In certain embodiments one R¹⁹ is hydrogen.

In certain embodiments one R¹⁹ is alkyl.

In certain embodiments one R¹⁹ is haloalkyl.

In certain embodiments one R¹⁹ is cycloalkyl.

In certain embodiments one R¹⁹ is heterocycle.

In certain embodiments one R¹⁹ is aryl.

In certain embodiments one R¹⁹ is heteroaryl.

### Embodiments of R²⁷

In certain embodiments R²⁷ is hydrogen.

In certain embodiments R²⁷ is cyano.

In certain embodiments R²⁷ is halogen.

In certain embodiments R²⁷ is fluoro.

In certain embodiments R²⁷ is haloalkyl.

In certain embodiments R²⁷ is —CF₃.

In certain embodiments R²⁷ is aryl.

In certain embodiments R²⁷ is phenyl.

In certain embodiments one R²⁷ is hydrogen.

In certain embodiments one R²⁷ is cyano.

In certain embodiments one R²⁷ is halogen.

In certain embodiments one R²⁷ is fluoro.

In certain embodiments one R²⁷ is haloalkyl.

In certain embodiments one R²⁷ is —CF₃.

In certain embodiments one R²⁷ is aryl.

In certain embodiments one R²⁷ is phenyl.

### Embodiments of “Alkyl”

In one embodiment “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In one embodiment “alkyl” has one carbon.

In one embodiment “alkyl” has two carbons.

In one embodiment “alkyl” has three carbons.

In one embodiment “alkyl” has four carbons.

In one embodiment “alkyl” has five carbons.

In one embodiment “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In an alternative embodiment the “alkyl” group is optionally substituted.

In an alternative embodiment the “alkenyl” group is optionally substituted.

### Embodiments of “Haloalkyl”

In one embodiment “haloalkyl” is a C₁-C₁₀haloalkyl, C₁-C₉haloalkyl, C₁-C₈haloalkyl, C₁-C₇haloalkyl, C₁-C₆haloalkyl, C₁-C₈haloalkyl, C₁-C₄haloalkyl, C₁-C₃haloalkyl, and C₁-C₂haloalkyl.

In one embodiment “haloalkyl” has one carbon.

In one embodiment “haloalkyl” has one carbon and one halogen.

In one embodiment “haloalkyl” has one carbon and two halogens.

In one embodiment “haloalkyl” has one carbon and three halogens.

In one embodiment “haloalkyl” has two carbons.

In one embodiment “haloalkyl” has three carbons.

In one embodiment “haloalkyl” has four carbons.

In one embodiment “haloalkyl” has five carbons.

In one embodiment “haloalkyl” has six carbons.

Non-limiting examples of “haloalkyl” include:

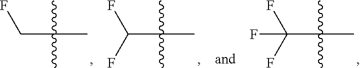

Additional non-limiting examples of “haloalkyl” include:

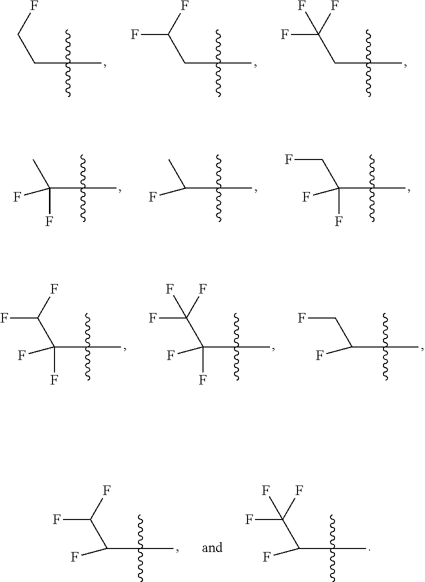

Additional non-limiting examples of “haloalkyl” include

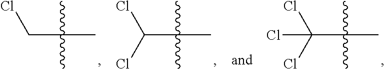

Additional non-limiting examples of “haloalkyl” include:

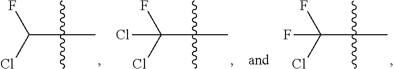

### Embodiments of “Heteroaryl”

Non-limiting examples of 5 membered “heteroaryl” groups include pyrrole, furan, thiophene, pyrazole, imidazole, triazole, isoxazole, oxazole, oxadiazole, oxatriazole, isothiazole, thiazole, thiadiazole, and thiatriazole.

Additional non-limiting examples of 5 membered “heteroaryl” groups include:

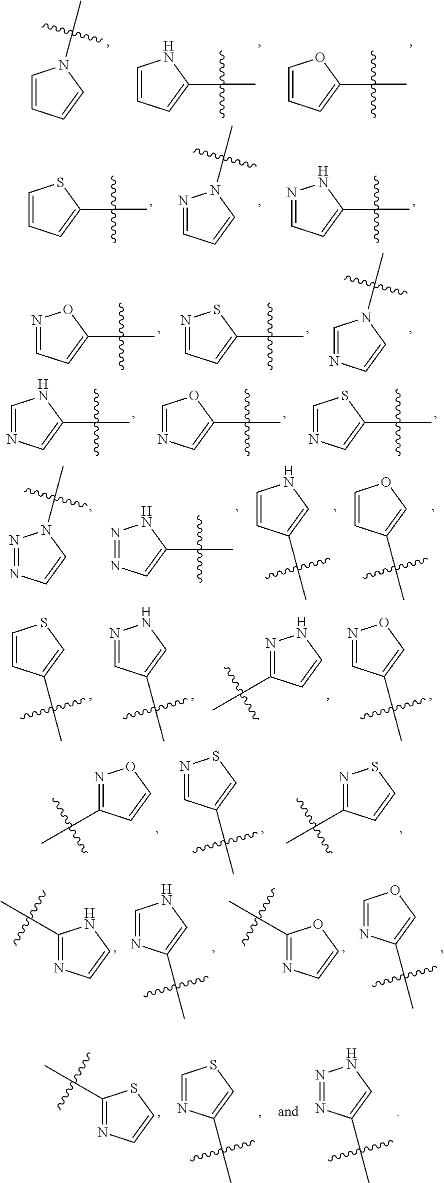

In one embodiment “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

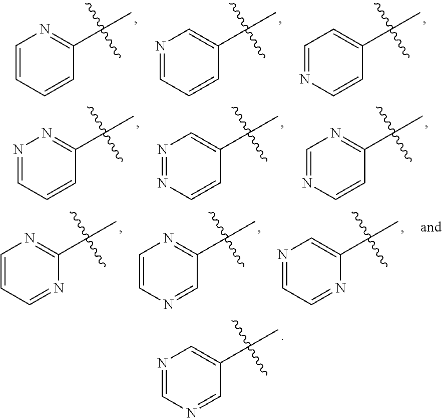

In one embodiment “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

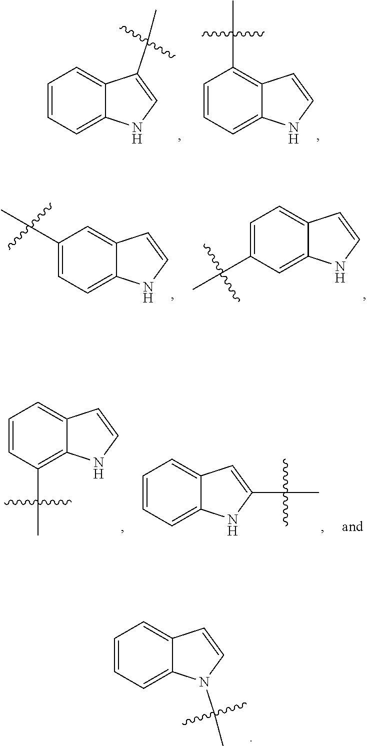

Additional non-limiting examples of “heteroaryl” groups that are bicyclic include:

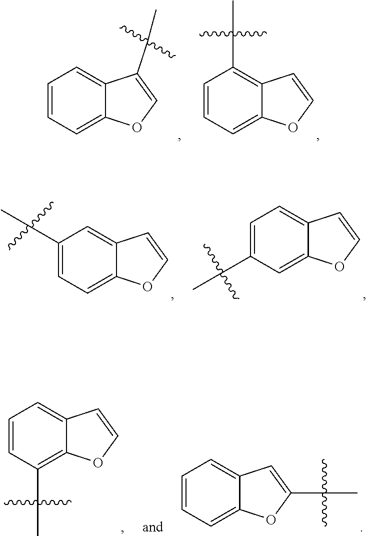 [SMILES: *c1coc2ccccc12 ; *c1cccc2occc12 ; *c1ccc2occc2c1 ; *c1ccc2ccoc2c1 ; *c1cccc2ccoc12 ; *c1cc2ccccc2o1]

Additional non-limiting examples of “heteroaryl” groups that are bicyclic include:

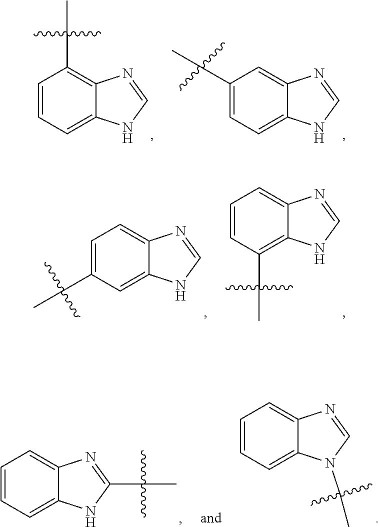

In one embodiment “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

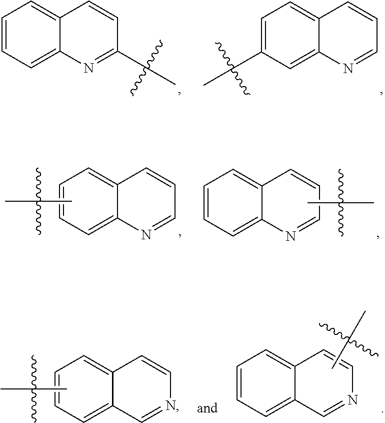

### Embodiments of “Heterocycle”

In one embodiment “heterocycle” refers to a cyclic ring with one nitrogen and 3, 4, 5, 6, 7, or 8 carbon atoms.

In one embodiment “heterocycle” refers to a cyclic ring with one nitrogen and one oxygen and 3, 4, 5, 6, 7, or 8 carbon atoms.

In one embodiment “heterocycle” refers to a cyclic ring with two nitrogens and 3, 4, 5, 6, 7, or 8 carbon atoms.

In one embodiment “heterocycle” refers to a cyclic ring with one oxygen and 3, 4, 5, 6, 7, or 8 carbon atoms.

In one embodiment “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

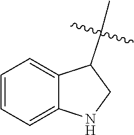 [SMILES: *C1CNc2ccccc21]

is a “heterocycle” group.

However,

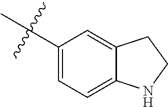 [SMILES: *c1ccc2c(c1)CCN2]

is an “aryl” group.

Non-limiting examples of “heterocycle” also include:

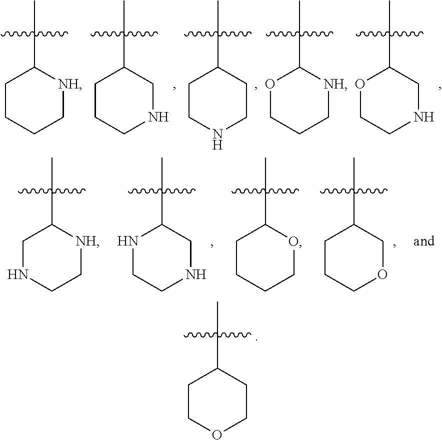

Additional non-limiting examples of “heterocycle” include:

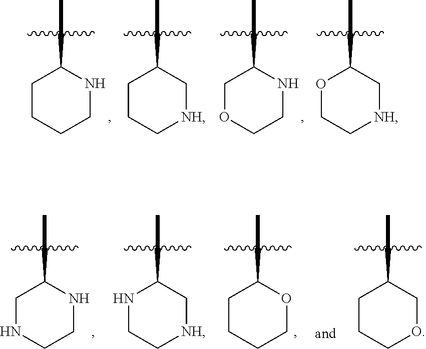

Additional non-limiting examples of “heterocycle” include:

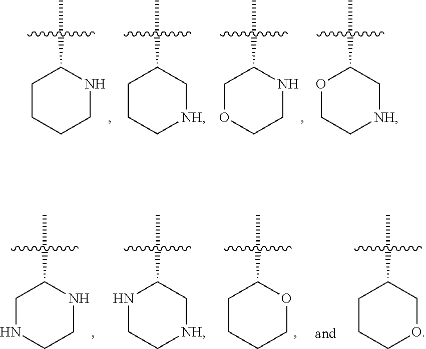

Non-limiting examples of “heterocycle” also include:

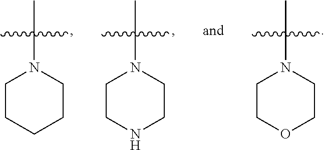

Non-limiting examples of “heterocycle” also include:

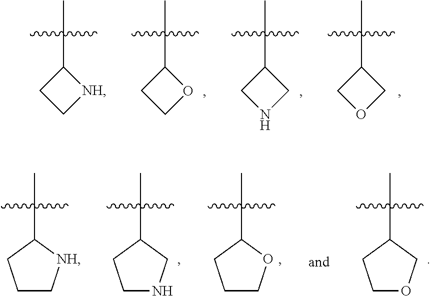

Additional non-limiting examples of “heterocycle” include:

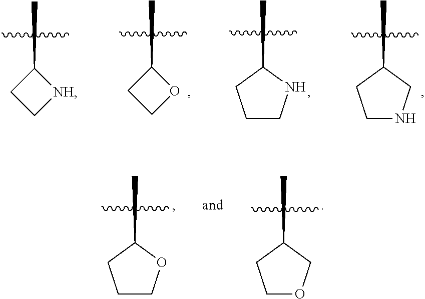

Additional non-limiting examples of “heterocycle” include:

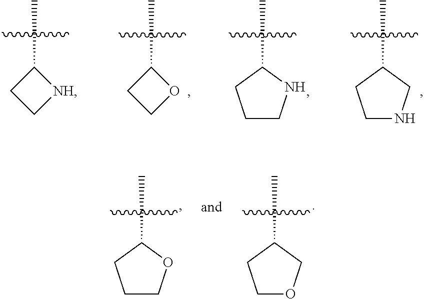

### Embodiments of “Aryl”

In one embodiment “aryl” is a 6 carbon aromatic group (phenyl).

In one embodiment “aryl” is a 10 carbon aromatic group (naphthyl).

In one embodiment “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example

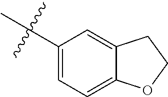 [SMILES: *c1ccc2c(c1)CCO2]

is an “aryl” group.

However,

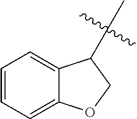 [SMILES: *C1COc2ccccc21]

is a “heterocycle” group.

### Embodiments of “Arylalkyl”

Non-limiting examples of “arylalkyl” include:

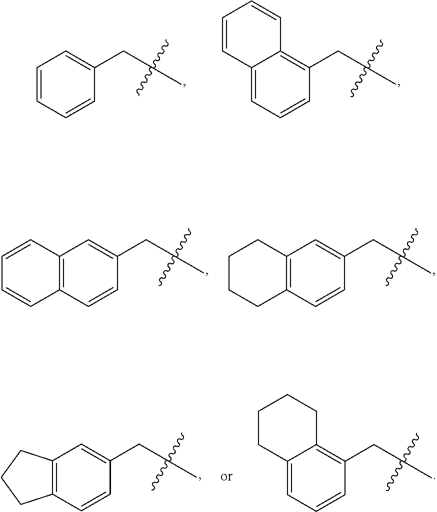 [SMILES: *Cc1ccccc1 ; *Cc1cccc2ccccc12 ; *Cc1ccc2ccccc2c1 ; *Cc1ccc2c(c1)CCCC2 ; *Cc1ccc2c(c1)CCC2 ; *Cc1cccc2c1CCCC2]

In one embodiment “arylalkyl” is

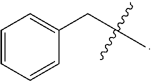 [SMILES: *Cc1ccccc1]

In one embodiment the “arylalkyl” refers to a 2 carbon alkyl group substituted with an aryl group.

Non-limiting examples of “arylalkyl” include:

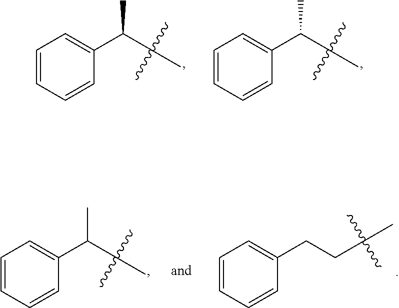

### Additional Heteroaryl Sulfonyl Compounds of the Present Invention

In certain embodiments the heteroaryl sulfonyl compound of the present invention is selected from:

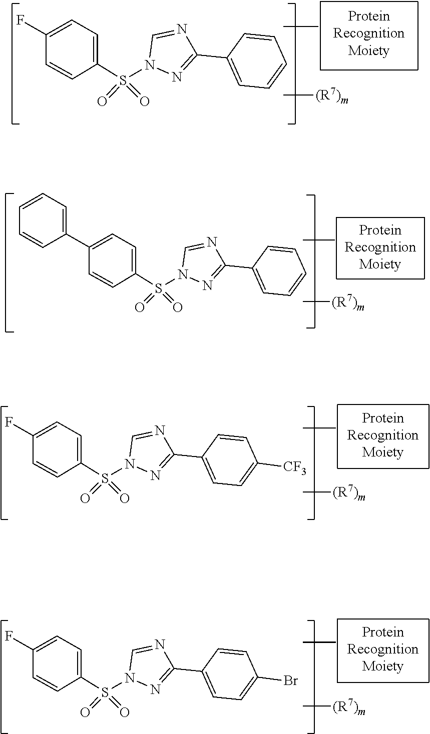

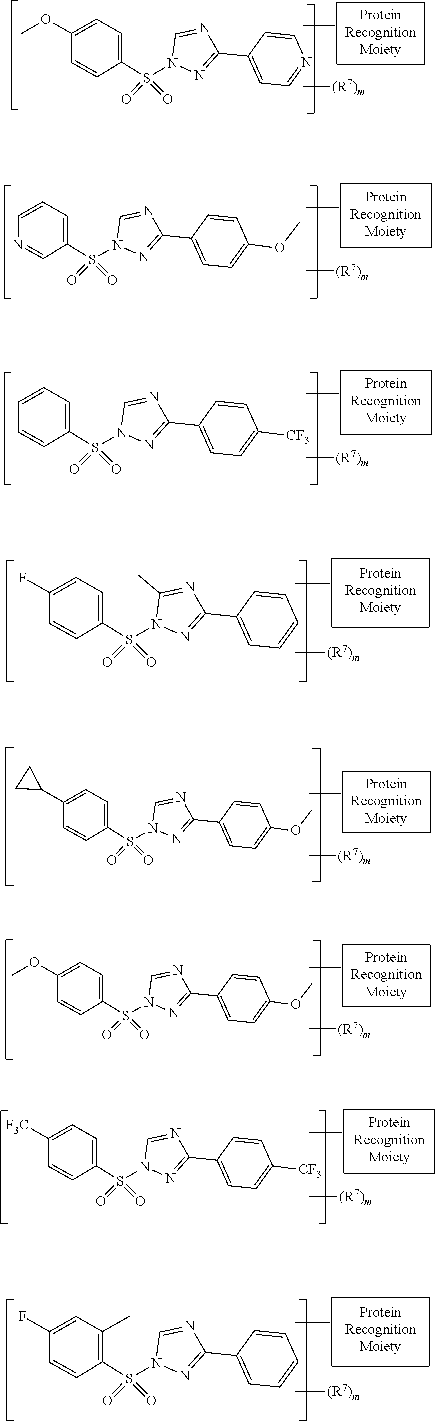

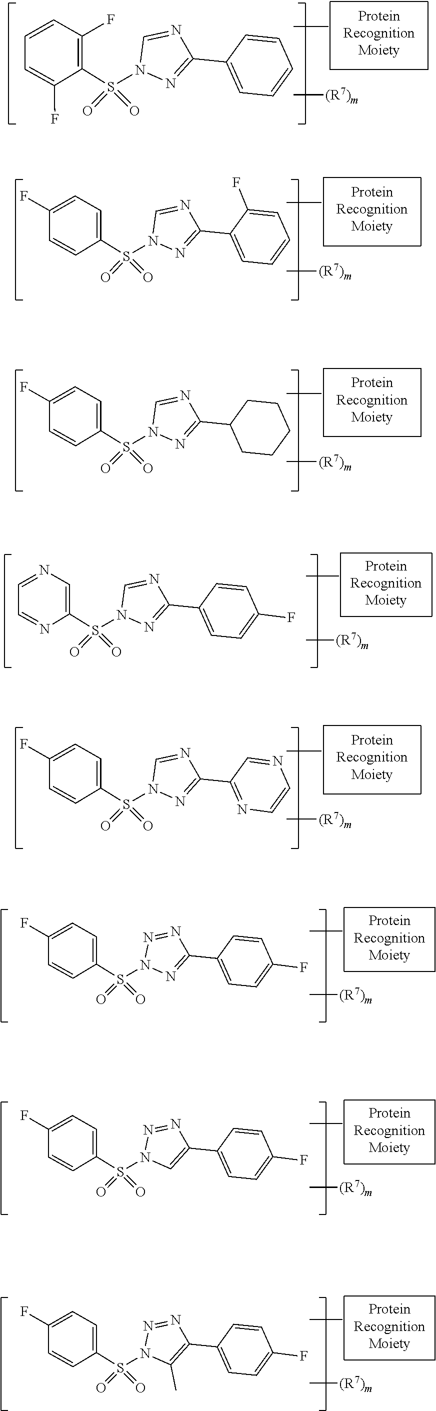

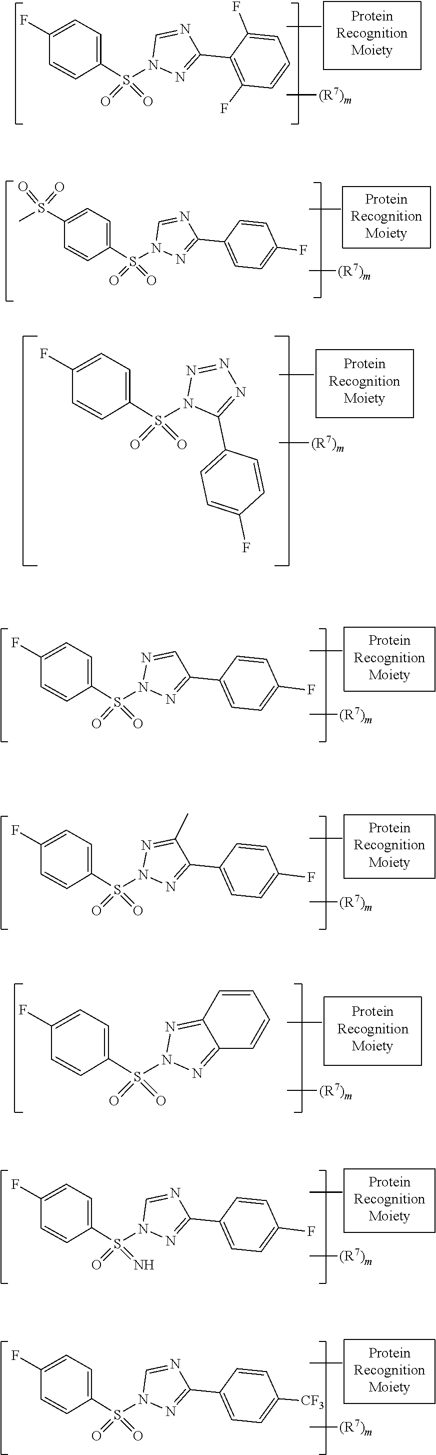

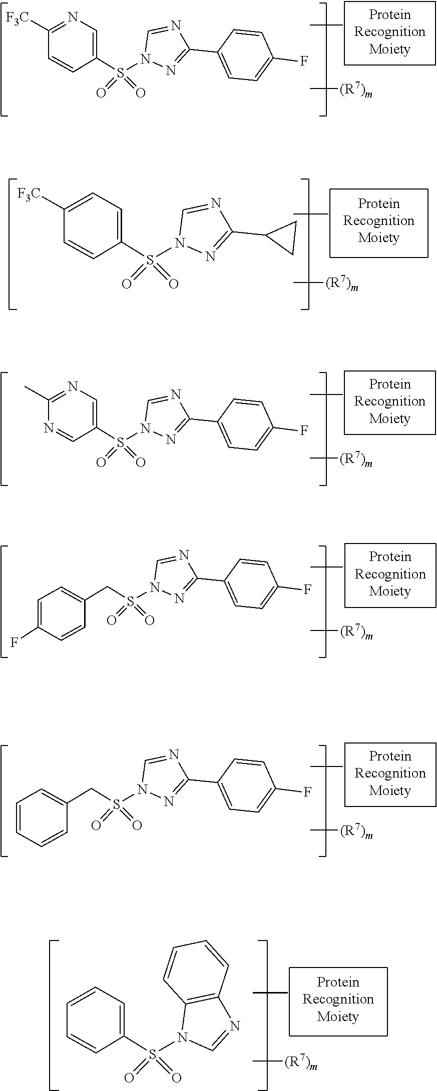

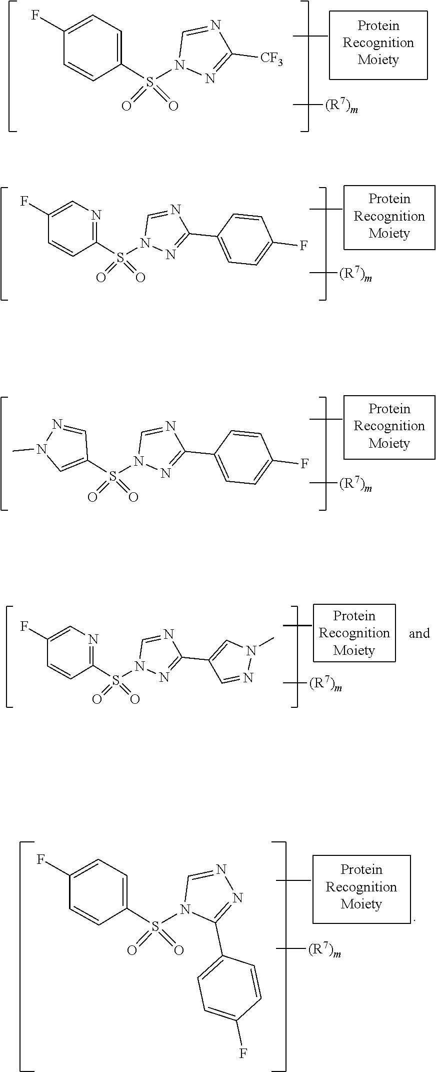

In certain embodiments the heteroaryl sulfonyl compound of the present invention is selected from:

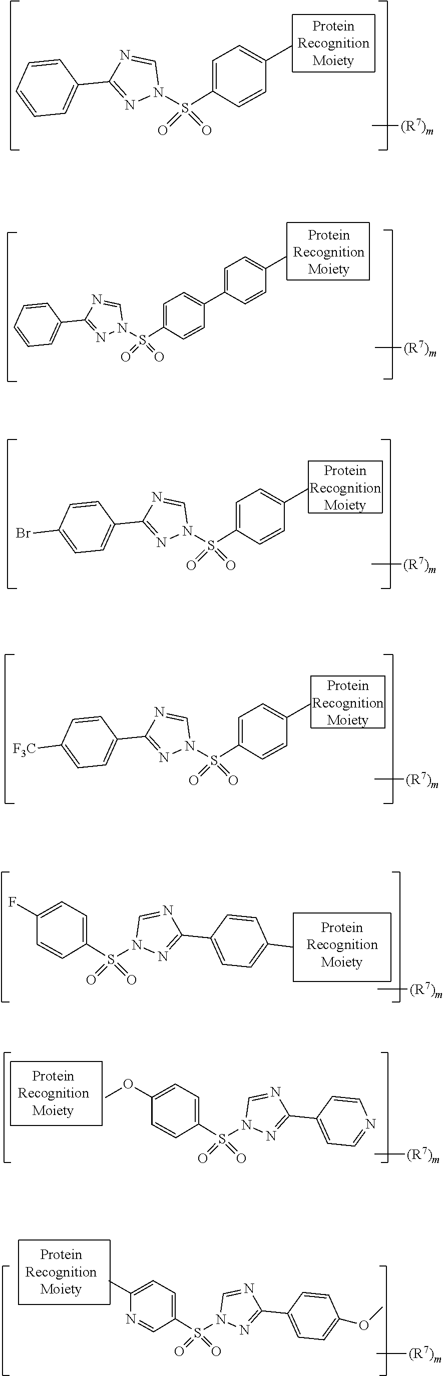

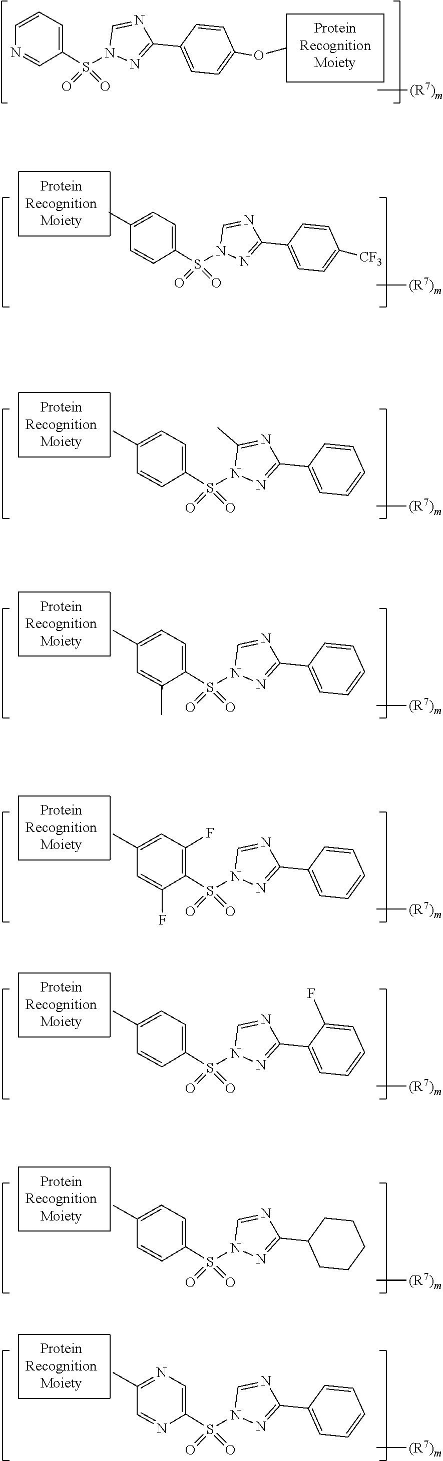

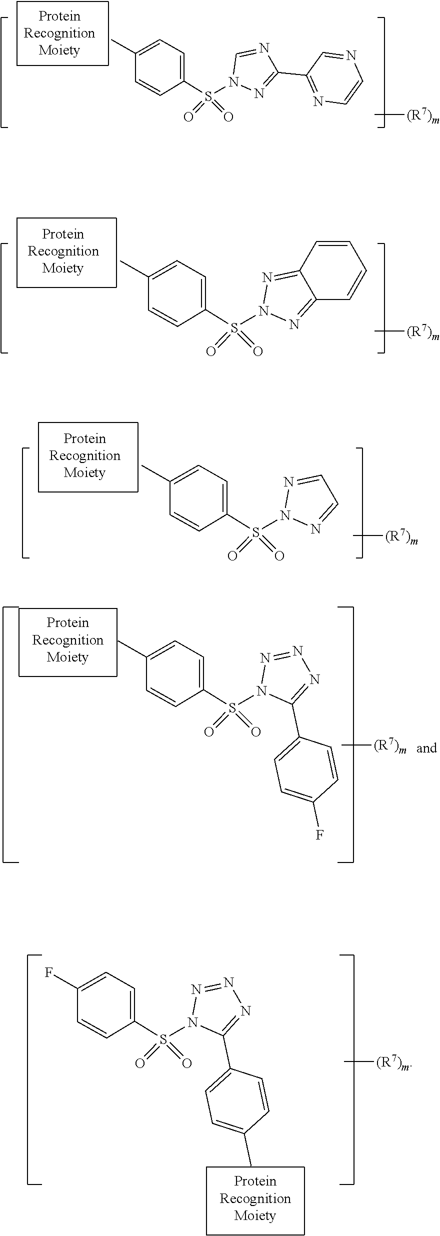

1. In one embodiment a compound selected from Formula I, Formula II, Formula III, Formula IV, Formula V, Formula VI, Formula VII, and Formula VIII is provided:

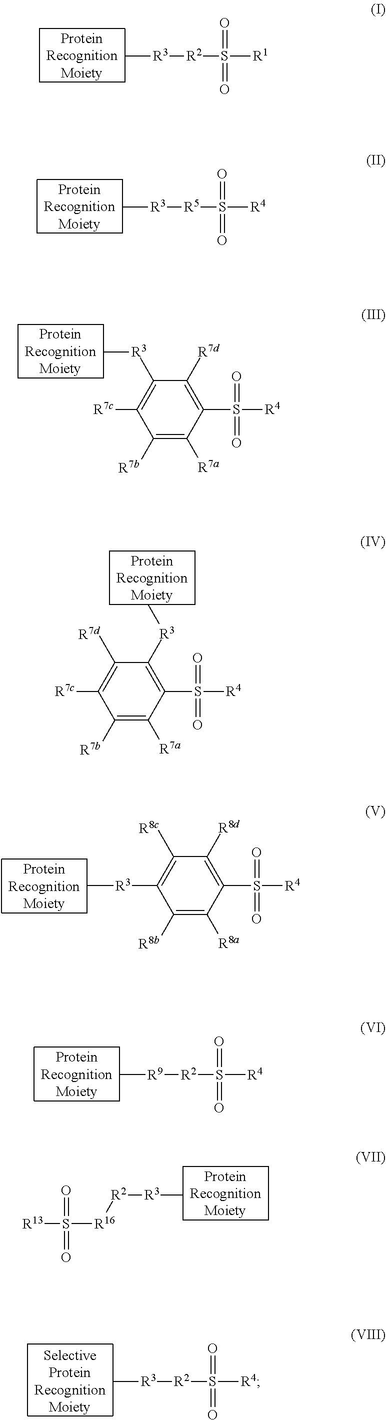

or a pharmaceutically acceptable salt, N-oxide, isotopic derivative, or prodrug thereof, wherein:
R¹ is selected from:
a)

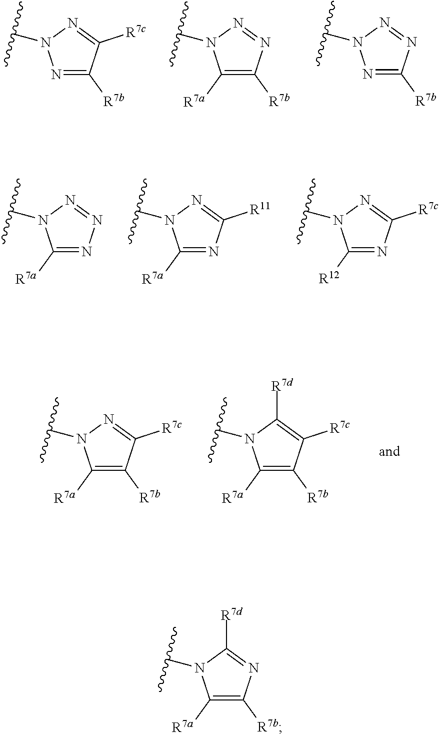

b) a bicyclic heteroaryl or tricyclic heteroaryl, each of which is optionally substituted as allowed by valence with 1, 2, 3, or 4 substituents selected from R⁷;
R² is independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which except bond is optionally substituted as allowed by valence with 1, 2, 3, or 4 substituents selected from R⁷;
R³ is independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which except bond is optionally substituted as allowed by valence with 1, 2, 3, or 4 substituents selected from R⁷;
and wherein R² and R³ are selected such that a suitably stable and suitably nontoxic compound for in vivo administration in a host is achieved, and in certain embodiments, in a way that avoids undesired repetition of atoms or moieties, such as in nonlimiting examples, S, O, or a combination thereof (i.e., that would otherwise form a disulfide or peroxide bond), as well known to skilled artisans;
p is independently selected from 1, 2, 3, 4, 5, and 6;
R⁴ is a heteroaryl group, where the bond to the sulfur atom is through one of the heteroatoms present in the cycle, and each heteroaryl is optionally substituted as allowed by valence with 1, 2, 3, or 4 substituents selected from R⁷;
R⁵ is selected from alkyl, alkenyl, haloalkyl, cycloalkyl, naphthyl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl-C(O)—NR⁶—, bicycle, tricycle, and heteroaryl, each of which is optionally substituted as allowed by valence with 1, 2, 3, or 4 substituents selected from R⁷;
and wherein R³ and R⁵ are selected such that a suitably stable and suitably nontoxic compound for in vivo administration in a host is achieved, and in certain embodiments, in a way that avoids undesired repetition of atoms or moieties, such as in nonlimiting examples, S, O, or a combination thereof (i.e., that would otherwise form a disulfide or peroxide bond), as well known to skilled artisans;
R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;
R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;
R¹⁷ is independently selected in each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶;
R¹⁸ is independently selected in each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, ═O, and —SR¹⁹;
R¹⁹ is independently selected at each instance from hydrogen, alkyl, haloalkyl, cycloalkyl, aryl, heterocycle, and heteroaryl;
R^{8a}, R^{8b}, R^{8c}, and R^{8d} are independently selected at each instance from R⁷ and R¹² wherein at least one of R^{8a}, R^{8b}, R^{8c}, and R^{8d} is R¹²;
R⁹ is selected from alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which is optionally substituted as allowed by valence with 1, 2, 3, or 4 substituents selected from R⁷;
R¹¹ is selected from hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, and —SR⁶, wherein each alkyl, haloalkyl, alkenyl, cycloalkyl, heterocycle, aryl, and heteroaryl optionally substituted as allowed by valence with 1, 2, 3, or 4 substituents selected from R¹⁷; and
R¹² is independently selected from halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷; and
R¹³ is selected from hydrogen, alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, and —NR⁶C(O)N(R⁶)₂, each of which except hydrogen is optionally substituted as allowed by valence with 1, 2, 3, or 4 substituents selected from R⁷;
R¹⁶ is a heteroaryl group, where the bond to the sulfur atom is through one of the heteroatoms present in the cycle, and R¹⁶ is optionally substituted as allowed by valence with 1, 2, 3, or 4 substituents selected from R⁷;
Protein Recognition Moiety is a molecule, for example a small molecule, peptide, protein, oligonucleotide, nucleotide, RNA, DNA, SiRNA, a biologic, an antibody or a fragment thereof, which can bind to or otherwise interact with a Target Protein;
Target Protein is a mediator of disease; and
Selective Protein Recognition Moiety is a Protein Recognition Moiety as defined herein wherein at least one of the following is satisfied:
i. there are fewer than 80, 70, 60, 50, 40, 30, 20, 15, 10, or 5 endogenous protein kinases to which the Selective Protein Recognition Moiety binds with an KD50 of 2 μM or less;
ii. the Selective Protein Recognition Moiety has an KD50 greater than 1 μM against aurora B kinase, c-Src kinase domain, human serine/threonine-protein kinase MST4, activin receptor type-IIA (ACVR2A), human calcium calmodulin dependent protein kinase II delta isoform 1 (CAMKD), and/or human ste20-like kinase;
2. The compound of embodiment 1, wherein the compound is of Formula:

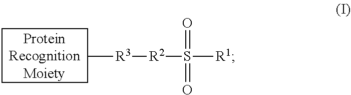

or a pharmaceutically acceptable salt thereof.
3. The compound of embodiment 1, wherein the compound is of Formula:

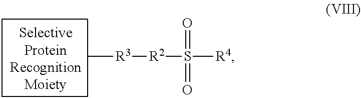

or a pharmaceutically acceptable salt thereof.
4. The compound of embodiment 1, wherein the compound is of Formula:

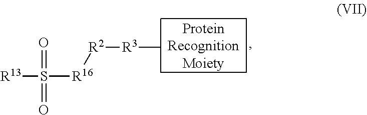

or a pharmaceutically acceptable salt thereof.
5. The compound of embodiment 4, wherein R¹³ is phenyl optionally substituted as allowed by valence with 1, 2, 3, or 4 substituents selected from R⁷.
6. The compound of embodiment 4, wherein R¹³ is alkyl optionally substituted as allowed by valence with 1, 2, 3, or 4 substituents selected from R⁷.
7. The compound of embodiment 4, wherein R¹³ is cycloalkyl optionally substituted as allowed by valence with 1, 2, 3, or 4 substituents selected from R⁷.
8. The compound of embodiment 4, wherein R¹³ is aryl optionally substituted as allowed by valence with 1, 2, 3, or 4 substituents selected from R⁷.
9. The compound of embodiment 4, wherein R¹³ is cyclopropyl optionally substituted as allowed by valence with 1, 2, 3, or 4 substituents selected from R⁷.
10. The compound of embodiment 4, wherein R¹³ is heterocycle optionally substituted as allowed by valence with 1, 2, 3, or 4 substituents selected from R⁷.
11. The compound of embodiment 4, wherein R¹³ is haloalkyl optionally substituted as allowed by valence with 1, 2, 3, or 4 substituents selected from R⁷.
12. The compound of any one of embodiments 5-11, wherein R¹⁶ is a triazole.
13. The compound of any one of embodiments 5-12, wherein R¹⁶ is

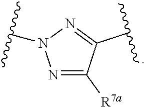

14. The compound of any one of embodiments 5-12, wherein R¹⁶ is

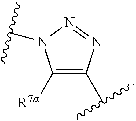

15. The compound of any one of embodiments 5-11, wherein R¹⁶ is

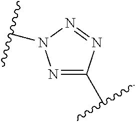 [SMILES: *c1nnn(*)n1]

16. The compound of any one of embodiments 5-11, wherein R¹⁶ is

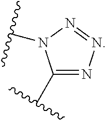 [SMILES: *c1nnnn1*]

17. The compound of any one of embodiments 5-12, wherein R¹⁶ is

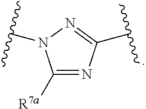

18. The compound of any one of embodiments 5-11, wherein R¹⁶ is R^{7b}

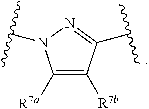

19. The compound of any one of embodiments 5-11, wherein R¹⁶ is C

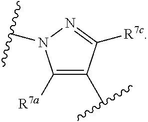

20. The compound of any one of embodiments 5-11, wherein R¹⁶ is

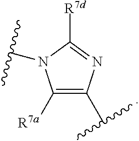

21. The compound of embodiment 1, wherein the compound is of Formula:

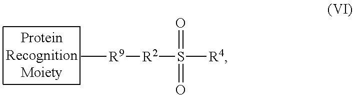

or a pharmaceutically acceptable salt thereof.
22. The compound of embodiment 21, wherein R⁹ is selected alkyl, alkenyl, haloalkyl, cycloalkyl, bicycle, tricycle, and heteroaryl, each of which except bond is optionally substituted as allowed by valence with 1, 2, 3, or 4 substituents selected from R⁷.
23. The compound of embodiment 22, wherein R⁹ is alkyl optionally substituted as allowed by valence with 1, 2, 3, or 4 substituents selected from R⁷.
24. The compound of embodiment 22, wherein R⁹ is alkenyl optionally substituted as allowed by valence with 1, 2, 3, or 4 substituents selected from R⁷.
25. The compound of embodiment 22, wherein R⁹ is haloalkyl optionally substituted as allowed by valence with 1, 2, 3, or 4 substituents selected from R⁷.
26. The compound of embodiment 22, wherein R⁹ is cycloalkyl optionally substituted as allowed by valence with 1, 2, 3, or 4 substituents selected from R⁷.
27. The compound of embodiment 22, wherein R⁹ is heteroaryl optionally substituted as allowed by valence with 1, 2, 3, or 4 substituents selected from R⁷.
28. The compound of embodiment 22, wherein R⁹ is bicycle optionally substituted as allowed by valence with 1, 2, 3, or 4 substituents selected from R⁷.
29. The compound of any one of embodiments 1-20, wherein R³ is bond.
30. The compound of any one of embodiments 1-20, wherein R³ is phenyl optionally substituted as allowed by valence with 1, 2, 3, or 4 substituents selected from R⁷.
31. The compound of any one of embodiments 1-20, wherein R³ is alkyl optionally substituted as allowed by valence with 1, 2, 3, or 4 substituents selected from R⁷.
32. The compound of any one of embodiments 1-20, wherein R³ is alkenyl optionally substituted as allowed by valence with 1, 2, 3, or 4 substituents selected from R⁷.
33. The compound of any one of embodiments 1-20, wherein R³ is haloalkyl optionally substituted as allowed by valence with 1, 2, 3, or 4 substituents selected from R⁷.
34. The compound of any one of embodiments 1-20, wherein R³ is cycloalkyl optionally substituted as allowed by valence with 1, 2, 3, or 4 substituents selected from R⁷.
35. The compound of any one of embodiments 1-20, wherein R³ is aryl optionally substituted as allowed by valence with 1, 2, 3, or 4 substituents selected from R⁷.
36. The compound of any one of embodiments 1-20, wherein R³ is heteroaryl optionally substituted as allowed by valence with 1, 2, 3, or 4 substituents selected from R⁷.
37. The compound of any one of embodiments 1-20, wherein R³ is bicycle optionally substituted as allowed by valence with 1, 2, 3, or 4 substituents selected from R⁷.
38. The compound of any one of embodiments 1-37, wherein R² is selected from bond, alkyl, alkenyl, haloalkyl, cycloalkyl, aryl, bicycle, tricycle, and heteroaryl, each of which except bond is optionally substituted as allowed by valence with 1, 2, 3, or 4 substituents selected from R⁷.
39. The compound of any one of embodiments 1-38, wherein R² is bond.
40. The compound of any one of embodiments 1-38, wherein R² is phenyl optionally substituted as allowed by valence with 1, 2, 3, or 4 substituents selected from R⁷.
41. The compound of any one of embodiments 1-38, wherein R² is alkyl optionally substituted as allowed by valence with 1, 2, 3, or 4 substituents selected from R⁷.
42. The compound of any one of embodiments 1-38, wherein R² is alkenyl optionally substituted as allowed by valence with 1, 2, 3, or 4 substituents selected from R⁷.
43. The compound of any one of embodiments 1-38, wherein R² is haloalkyl optionally substituted as allowed by valence with 1, 2, 3, or 4 substituents selected from R⁷.
44. The compound of any one of embodiments 1-38, wherein R² is cycloalkyl optionally substituted as allowed by valence with 1, 2, 3, or 4 substituents selected from R⁷.
45. The compound of any one of embodiments 1-38, wherein R² is aryl optionally substituted as allowed by valence with 1, 2, 3, or 4 substituents selected from R⁷.
46. The compound of any one of embodiments 1-38, wherein R² is heteroaryl optionally substituted as allowed by valence with 1, 2, 3, or 4 substituents selected from R⁷.
47. The compound of any one of embodiments 1-38, wherein R² is bicycle optionally substituted as allowed by valence with 1, 2, 3, or 4 substituents selected from R⁷.
48. The compound of any one of embodiments 1-47, wherein R² is not substituted.
49. The compound of any one of embodiments 1-47, wherein R² is substituted as allowed by valence with 1 substituent selected from R⁷.
50. The compound of any one of embodiments 1-47, wherein R² is substituted as allowed by valence with 2 substituents selected from R⁷.
51. The compound of any one of embodiments 1-47, wherein R² is substituted as allowed by valence with 3 substituents selected from R⁷.
52. The compound of embodiment 1, wherein the compound is of Formula:

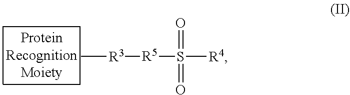

or a pharmaceutically acceptable salt thereof.
53. The compound of embodiment 1 wherein the compound is Formula:

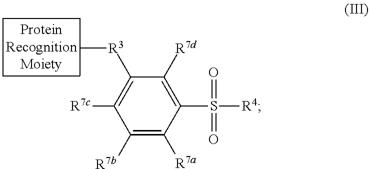

or a pharmaceutically acceptable salt thereof.
54. The compound of embodiment 1 wherein the compound is Formula:

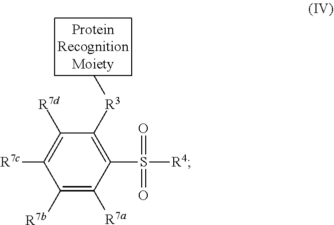

or a pharmaceutically acceptable salt thereof.
55. The compound of embodiment 1 wherein the compound is Formula:

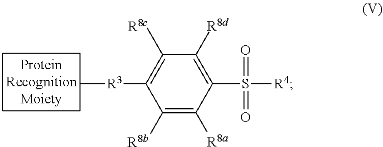

or a pharmaceutically acceptable salt thereof.
56. The compound of any one of embodiments 52-55, wherein R³ is bond.
57. The compound of any one of embodiments 52-55, wherein R³ is phenyl optionally substituted as allowed by valence with 1, 2, 3, or 4 substituents selected from R⁷.
58. The compound of any one of embodiments 52-55, wherein R³ is alkyl optionally substituted as allowed by valence with 1, 2, 3, or 4 substituents selected from R⁷.
59. The compound of any one of embodiments 52-55, wherein R³ is alkenyl optionally substituted as allowed by valence with 1, 2, 3, or 4 substituents selected from R⁷.
60. The compound of any one of embodiments 52-55, wherein R³ is haloalkyl optionally substituted as allowed by valence with 1, 2, 3, or 4 substituents selected from R⁷.
61. The compound of any one of embodiments 52-55, wherein R³ is cycloalkyl optionally substituted as allowed by valence with 1, 2, 3, or 4 substituents selected from R⁷.
62. The compound of any one of embodiments 52-55, wherein R³ is aryl optionally substituted as allowed by valence with 1, 2, 3, or 4 substituents selected from R⁷.
63. The compound of any one of embodiments 52-55, wherein R³ is heteroaryl optionally substituted as allowed by valence with 1, 2, 3, or 4 substituents selected from R⁷.
64. The compound of any one of embodiments 52-55, wherein R³ is bicycle optionally substituted as allowed by valence with 1, 2, 3, or 4 substituents selected from R⁷.
65. The compound of any one of embodiments 1-3, or 21-64, wherein R¹ and R⁴ are

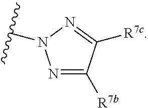

66. The compound of any one of embodiments 1-3, or 21-64, wherein R¹ and R⁴ are

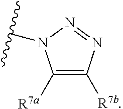

67. The compound of any one of embodiments 1-3, or 21-64, wherein R¹ and R⁴ are

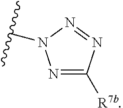

68 The compound of any one of embodiments 1-3, or 21-64, wherein R¹ and R⁴ are

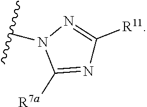

69. The compound of any one of embodiments 1-3, or 21-64, wherein R¹ and R⁴ are

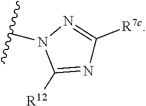

70. The compound of any one of embodiments 1-3, or 21-64, wherein R¹ and R⁴ are

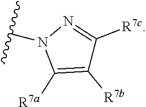

71. The compound of any one of embodiments 1-3, or 21-64, wherein R¹ and R⁴ are

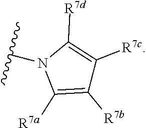

72. The compound of any one of embodiments 1-3, or 21-64, wherein R¹ and R⁴ are

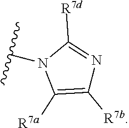

73. The compound of any one of embodiments 1-3, or 21-64, wherein R¹ and R⁴ are a bicyclic heteroaryl which is optionally substituted as allowed by valence with 1, 2, 3, or 4 substituents selected from R⁷.
74. The compound of any one of embodiments 1-3, or 21-64, wherein R⁴ is a heteroaryl group, where the bond to the sulfur atom is through one of the nitrogen present in the cycle, and each heteroaryl is optionally substituted as allowed by valence with 1, 2, 3, or 4 substituents selected from R⁷.
75. The compound of any one of embodiments 1-74, wherein R⁶ is independently selected at each instance from alkyl, haloalkyl, cycloalkyl, aryl, heterocycle, and heteroaryl; each of which is optionally substituted as allowed by valence with 1, 2, 3, or 4 substituents selected from R¹⁸.
76. The compound of embodiment 75, wherein R⁶ is alkyl optionally substituted as allowed by valence with 1, 2, 3, or 4 substituents selected from R¹⁸.
77. The compound of embodiment 75, wherein R⁶ is haloalkyl optionally substituted as allowed by valence with 1, 2, 3, or 4 substituents selected from R¹⁸.
78. The compound of embodiment 75, wherein R⁶ is cycloalkyl optionally substituted as allowed by valence with 1, 2, 3, or 4 substituents selected from R¹⁸.
79. The compound of embodiment 75, wherein R⁶ is aryl optionally substituted as allowed by valence with 1, 2, 3, or 4 substituents selected from R¹⁸.
80. The compound of embodiment 75, wherein R⁶ is heterocycle optionally substituted as allowed by valence with 1, 2, 3, or 4 substituents selected from R¹⁸.
81. The compound of embodiment 75, wherein R⁶ is heteroaryl optionally substituted as allowed by valence with 1, 2, 3, or 4 substituents selected from R¹⁸.
82. The compound of any one of embodiments 75-81, wherein R⁶ is not substituted with R¹⁸.
83. The compound of any one of embodiments 75-81, wherein R⁶ is substituted with 1 substituent selected from R¹⁸.
84. The compound of any one of embodiments 75-81, wherein R⁶ is substituted with 2 substituents independently selected from R¹⁸.
85. The compound of any one of embodiments 75-81, wherein R⁶ is substituted with 3 substituents independently selected from R¹⁸.
86. The compound of any one of embodiments 1-85, wherein R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, and nitro, wherein each alkyl, haloalkyl, alkenyl, cycloalkyl, heterocycle, aryl, and heteroaryl is optionally substituted as allowed by valence with 1, 2, 3, or 4 substituents selected from R¹⁷
87. The compound of any one of embodiments 1-86, wherein one of R⁷, R^{7a}, R^{7b}, R^{7c}, and R^{7d} is alkyl optionally substituted as allowed by valence with 1, 2, 3, or 4 substituents selected from R¹⁷.
88. The compound of any one of embodiments 1-86, wherein one of R⁷, R^{7a}, R^{7b}, R^{7c}, and R^{7d} is haloalkyl optionally substituted as allowed by valence with 1, 2, 3, or 4 substituents selected from R¹⁷.
89. The compound of any one of embodiments 1-86, wherein one of R⁷, R^{7a}, R^{7b}, R^{7c}, and R^{7d} is phenyl optionally substituted as allowed by valence with 1, 2, 3, or 4 substituents selected from R¹⁷.
90. The compound of any one of embodiments 1-86, wherein one of R⁷, R^{7a}, R^{7b}, R^{7c}, and R^{7d} is heteroaryl optionally substituted as allowed by valence with 1, 2, 3, or 4 substituents selected from R¹⁷.
91. The compound of any one of embodiments 1-86, wherein one of R⁷, R^{7a}, R^{7b}, R^{7c}, and R^{7d} is haloalkyl optionally substituted as allowed by valence with 1, 2, 3, or 4 substituents selected from R¹⁷.
92. The compound of any one of embodiments 1-86, wherein one of R⁷, R^{7a}, R^{7b}, R^{7c}, and R^{7d} is heterocycle optionally substituted as allowed by valence with 1, 2, 3, or 4 substituents selected from R¹⁷.
93. The compound of any one of embodiments 1-86, wherein one of R⁷, R^{7a}, R^{7b}, R^{7c}, and R^{7d} is cycloalkyl optionally substituted as allowed by valence with 1, 2, 3, or 4 substituents selected from R¹⁷.
94. The compound of any one of embodiments 87-93, wherein R⁷, R^{7a}, R^{7b}, R^{7c}, and R^{7d} are not substituted.
95. The compound of any one of embodiments 87-93, wherein R⁷, R^{7a}, R^{7b}, R^{7c}, and R^{7d} are optionally substituted with 1 or 2 substituents selected from R¹⁷.
96. The compound of any one of embodiments 1-86, wherein one of R⁷, R^{7a}, R^{7b}, R^{7c}, and R^{7d} is halogen.
97. The compound of any one of embodiments 1-86, wherein one of R⁷, R^{7a}, R^{7b}, R^{7c}, and R^{7d} is —OR⁶.
98. The compound of any one of embodiments 1-86, wherein one of R⁷, R^{7a}, R^{7b}, R^{7c}, and R^{7d} is ═O.
99. The compound of any one of embodiments 1-86, wherein one of R⁷, R^{7a}, R^{7b}, R^{7c}, and R^{7d} is cyano.
100. The compound of any one of embodiments 1-86, wherein one of R⁷, R^{7a}, R^{7b}, R^{7c}, and R^{7d} is nitro.
101. The compound of any one of embodiments 1-100, wherein R¹⁷ is selected in each instance from halogen, alkyl, haloalkyl, alkenyl, and cyano.
102. In one embodiment a pharmaceutical composition comprising a compound of any one of embodiments 1-101 or a pharmaceutically acceptable salt thereof and a pharmaceutically acceptable excipient is provided.
103. In one embodiment a method of treating a disorder mediated by the Target Protein comprising administering an effective amount of a compound of any one of embodiments 1-102 or a pharmaceutically acceptable salt thereof to a patient in need thereof, wherein the Protein Recognition Moiety binds the Target Protein, is provided.
104. The method of embodiment 103 wherein the patient is a human.

In other embodiments a compound, pharmaceutical composition, or method is provided as described below:
1. A compound of Formula:

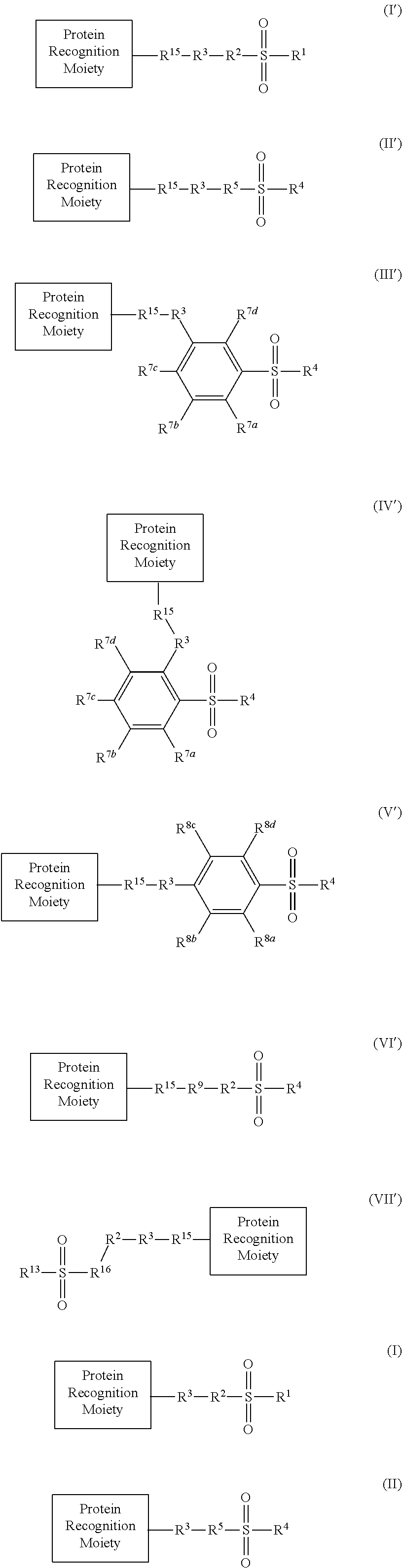

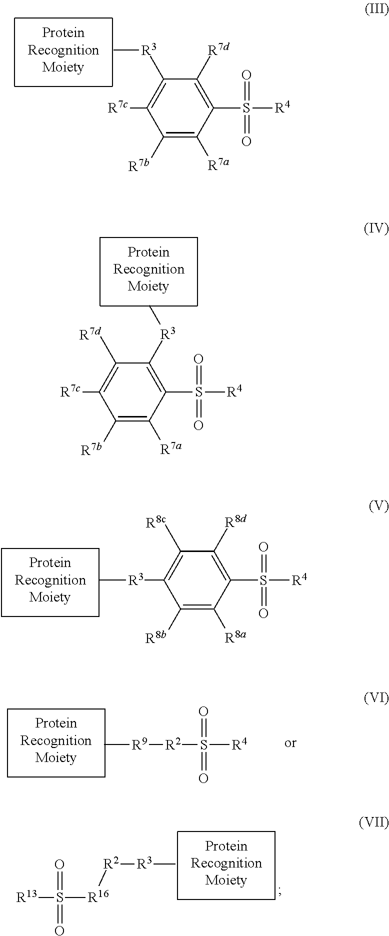

or a pharmaceutically acceptable salt thereof;
wherein:
R¹ is selected from:

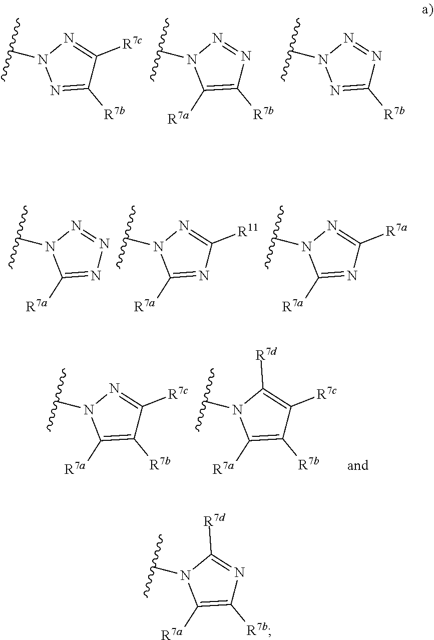

or
b) a bicyclic heteroaryl or tricyclic heteroaryl, optionally substituted as allowed by valence with 1, 2, 3, or 4 substituents selected from R⁷;
R² is a bivalent moiety independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;
R³ is a bivalent moiety independently selected at each instance from bond, alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents selected from R⁷;
and wherein R² and R³ are selected such that a suitably stable and suitably nontoxic compound for in vivo administration in a host is achieved;
each p is independently selected from 1, 2, 3, 4, 5, and 6;
R⁴ is a heteroaryl group, where the bond to the sulfur atom is through one of the nitrogen atoms present in the cycle, and each heteroaryl is optionally substituted as allowed by valence with 1, 2, 3, or 4 substituents selected from R⁷;
R⁵ is a bivalent moiety selected from alkyl, alkenyl, haloalkyl, cycloalkyl, naphthyl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl-C(O)—NR⁶—, bicycle, tricycle, and heteroaryl, each of which is optionally substituted as allowed by valence with 1, 2, 3, or 4 substituents selected from R⁷;
and wherein R³ and R⁵ are selected such that a suitably stable and suitably nontoxic compound for in vivo administration in a host is achieved;
R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;
R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;
R¹⁵ is a bivalent moiety selected from the group consisting of alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which except bond is optionally substituted as allowed by valence with 1, 2, 3, or 4 substituents independently selected from R⁷;
R¹⁷ is independently selected in each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶;
R¹⁸ is independently selected in each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, ═O, and —SR¹⁹;
R¹⁹ is independently selected at each instance from hydrogen, alkyl, haloalkyl, cycloalkyl, aryl, heterocycle, and heteroaryl;
R^{8a}, R^{8b}, R^{8c}, and R^{8d} are independently selected at each instance from R⁷ and R¹² wherein at least one of R^{8a}, R^{8b}, R^{8c}, and R^{8d} is R¹²;
R⁹ is a bivalent moiety selected from alkyl, alkenyl, haloalkyl, cycloalkyl, heterocycle, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which is optionally substituted as allowed by valence with 1, 2, 3, or 4 substituents selected from R⁷;
R¹¹ is selected from hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷; and
R¹² is independently selected from halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;
R¹³ is selected from hydrogen, alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, and —NR⁶C(O)N(R⁶)₂, each of which except hydrogen is optionally substituted as allowed by valence with 1, 2, 3, or 4 substituents selected from R⁷;
R¹⁶ is a heteroaryl group, where the bond to the sulfur atom is through one of the nitrogen atoms present in the cycle, and R¹⁶ is optionally substituted as allowed by valence with 1, 2, 3, or 4 substituents selected from R⁷; and
Protein Recognition Moiety is a molecule which can bind to or otherwise interact with a Target Protein; and
Target Protein is a mediator of disease.
2. A compound of Formula:

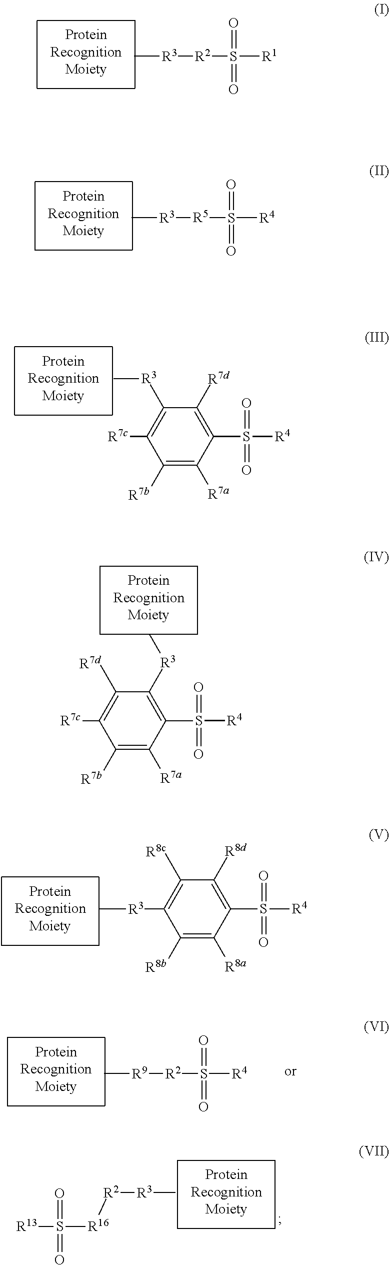

or a pharmaceutically acceptable salt thereof,
wherein:
R¹ is selected from:

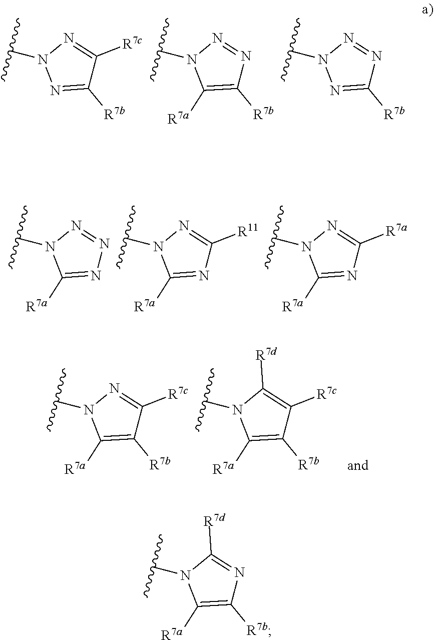

or
b) a bicyclic heteroaryl or tricyclic heteroaryl, each of which is optionally substituted as allowed by valence with 1, 2, 3, or 4 substituents selected from R⁷;
R² is a bivalent moiety independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;
R³ is a bivalent moiety independently selected at each instance from bond, alkyl, alkenyl, haloalkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, tricycle, and heteroaryl, each of which is optionally substituted as allowed by valence with 1, 2, 3, or 4 substituents selected from R⁷;
and wherein R² and R³ are selected such that a suitably stable and suitably nontoxic compound for in vivo administration in a host is achieved;
p is independently selected from 1, 2, 3, 4, 5, and 6;
R⁴ is a heteroaryl group, where the bond to the sulfur atom is through one of the nitrogen atoms present in the cycle, and each heteroaryl is optionally substituted as allowed by valence with 1, 2, 3, or 4 substituents selected from R⁷;
R⁵ is a bivalent moiety selected from alkyl, alkenyl, haloalkyl, cycloalkyl, naphthyl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, heteroaryl-C(O)—NR⁶—, bicycle, tricycle, and heteroaryl, each of which is optionally substituted as allowed by valence with 1, 2, 3, or 4 substituents selected from R⁷;
and wherein R³ and R⁵ are selected such that a suitably stable and suitably nontoxic compound for in vivo administration in a host is achieved;
R⁶ is independently selected at each instance from hydrogen, alkyl, haloalkyl, cycloalkyl, aryl, heterocycle, and heteroaryl; each of which except hydrogen is optionally substituted as allowed by valence with 1, 2, 3, or 4 substituents selected from R¹⁸;
R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷;
R¹⁷ is independently selected in each instance from hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶;
R¹⁸ is independently selected in each instance from hydrogen, halogen, alkyl, haloalkyl, alkenyl, cycloalkyl, heterocycle, aryl, heteroaryl, cyano, nitro, —C(O)R¹⁹, —OC(O)R¹⁹, —NR¹⁹C(O)R¹⁹, —C(O)OR¹⁹, —OC(O)OR¹⁹, —NR¹⁹C(O)OR¹⁹, —C(O)N(R¹⁹)₂, —OC(O)N(R¹⁹)₂, —NR¹⁹C(O)N(R¹⁹)₂, —OR¹⁹, —N(R¹⁹)₂, —S(O)R¹⁹, —S(O)₂R¹⁹, —S(O)OR¹⁹, —S(O)₂OR¹⁹, —S(O)N(R¹⁹)₂, S(O)₂N(R¹⁹)₂, ═O, and —SR¹⁹;
R¹⁹ is independently selected at each instance from hydrogen, alkyl, haloalkyl, cycloalkyl, aryl, heterocycle, and heteroaryl;
R^{8a}, R^{8b}, R^{8c}, and R^{8d} are independently selected at each instance from R⁷ and R¹² wherein at least one of R^{8a}, R^{8b}, R^{8c}, and R^{8d} is R¹²;
R⁹ is a bivalent moiety selected from alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, tricycle, and heteroaryl, each of which is optionally substituted as allowed by valence with 1, 2, 3, or 4 substituents selected from R⁷;
R¹¹ is selected from hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, and —SR⁶, wherein each alkyl, haloalkyl, alkenyl, cycloalkyl, heterocycle, aryl, and heteroaryl optionally substituted as allowed by valence with 1, 2, 3, or 4 substituents selected from R¹⁷; and
R¹² is independently selected from halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, ═O, and —SR⁶, wherein each alkyl, haloalkyl, alkenyl, cycloalkyl, heterocycle, aryl, and heteroaryl is optionally substituted as allowed by valence with 1, 2, 3, or 4 substituents selected from R¹⁷; and
R¹³ is selected from hydrogen, alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, and —NR⁶C(O)N(R⁶)₂, each of which except hydrogen is optionally substituted as allowed by valence with 1, 2, 3, or 4 substituents selected from R⁷;
R¹⁶ is a heteroaryl group, where the bond to the sulfur atom is through one of the nitrogen atoms present in the cycle, and R¹⁶ is optionally substituted as allowed by valence with 1, 2, 3, or 4 substituents selected from R⁷;
Protein Recognition Moiety is a molecule which can bind to or otherwise interact with a Target Protein; and
Target Protein is a mediator of disease.
3. The compound of embodiment 1 or embodiment 2, wherein the compound is of Formula:

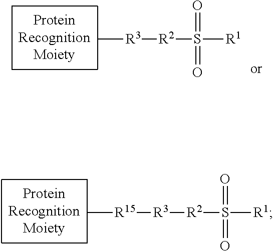

or a pharmaceutically acceptable salt thereof.
4. The compound of embodiment 1 or embodiment 2, wherein the compound is of Formula:

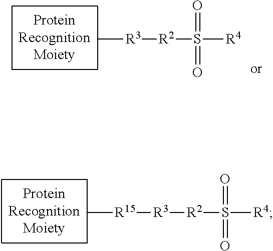

or a pharmaceutically acceptable salt thereof.
5. The compound of embodiment 1 or embodiment 2, wherein the compound is of Formula:

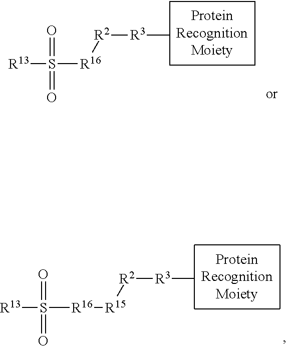

or a pharmaceutically acceptable salt thereof.
6. The compound of embodiment 5, wherein R¹³ is phenyl optionally substituted as allowed by valence with 1, 2, 3, or 4 substituents selected from R⁷.
7. The compound of embodiment 5, wherein R¹³ is alkyl optionally substituted as allowed by valence with 1, 2, 3, or 4 substituents selected from R⁷.
8. The compound of embodiment 5, wherein R¹³ is cycloalkyl optionally substituted as allowed by valence with 1, 2, 3, or 4 substituents selected from R⁷.
9. The compound of embodiment 5, wherein R¹³ is aryl optionally substituted as allowed by valence with 1, 2, 3, or 4 substituents selected from R⁷.
10. The compound of embodiment 5, wherein R¹³ is cyclopropyl optionally substituted as allowed by valence with 1, 2, 3, or 4 substituents selected from R⁷.
11. The compound of embodiment 5, wherein R¹³ is heterocycle optionally substituted as allowed by valence with 1, 2, 3, or 4 substituents selected from R⁷.
12. The compound of embodiment 5, wherein R¹³ is haloalkyl optionally substituted as allowed by valence with 1, 2, 3, or 4 substituents selected from R⁷.
13. The compound of any one of embodiments 5-12, wherein R¹⁶ is a triazole.
14. The compound of any one of embodiments 5-12, wherein R¹⁶ is

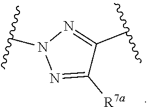

15. The compound of any one of embodiments 5-12, wherein R¹⁶ is

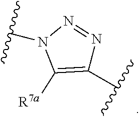

16. The compound of any one of embodiments 5-12, wherein R¹⁶ is

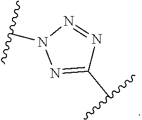 [SMILES: *c1nnn(*)n1]

17. The compound of any one of embodiments 5-12, wherein R¹⁶ is

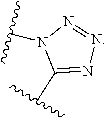 [SMILES: *c1nnnn1*]

18. The compound of any one of embodiments 5-12, wherein R¹⁶ is

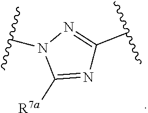

19. The compound of any one of embodiments 5-12, wherein R¹⁶ is

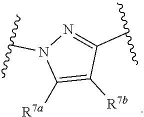

20. The compound of any one of embodiments 5-12, wherein R¹⁶ is

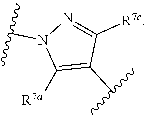

21. The compound of any one of embodiments 5-12, wherein R¹⁶ is

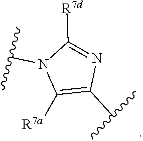

22. The compound of embodiment 1 or 2, wherein the compound is of Formula:

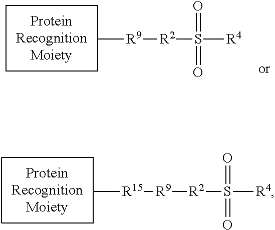

or a pharmaceutically acceptable salt thereof.
23. The compound of embodiment 22, wherein R⁹ is selected from alkyl, alkenyl, haloalkyl, cycloalkyl, bicycle, tricycle, and heteroaryl, each of which except bond is optionally substituted as allowed by valence with 1, 2, 3, or 4 substituents selected from R⁷.
24. The compound of embodiment 22, wherein R⁹ is alkyl optionally substituted as allowed by valence with 1, 2, 3, or 4 substituents selected from R⁷.
25. The compound of embodiment 22, wherein R⁹ is alkenyl optionally substituted as allowed by valence with 1, 2, 3, or 4 substituents selected from R⁷.
26. The compound of embodiment 22, wherein R⁹ is haloalkyl optionally substituted as allowed by valence with 1, 2, 3, or 4 substituents selected from R⁷.
27. The compound of embodiment 22, wherein R⁹ is cycloalkyl optionally substituted as allowed by valence with 1, 2, 3, or 4 substituents selected from R⁷.
28. The compound of embodiment 22, wherein R⁹ is heteroaryl optionally substituted as allowed by valence with 1, 2, 3, or 4 substituents selected from R⁷.
29. The compound of embodiment 22, wherein R⁹ is bicycle optionally substituted as allowed by valence with 1, 2, 3, or 4 substituents selected from R⁷.
30. The compound of any one of embodiments 1-29, wherein R³ is bond.
31. The compound of any one of embodiments 1-29, wherein R³ is phenyl optionally substituted as allowed by valence with 1, 2, 3, or 4 substituents selected from R⁷.
32. The compound of any one of embodiments 1-29, wherein R³ is alkyl optionally substituted as allowed by valence with 1, 2, 3, or 4 substituents selected from R⁷.
33. The compound of any one of embodiments 1-29, wherein R³ is alkenyl optionally substituted as allowed by valence with 1, 2, 3, or 4 substituents selected from R⁷.
34. The compound of any one of embodiments 1-29, wherein R³ is haloalkyl optionally substituted as allowed by valence with 1, 2, 3, or 4 substituents selected from R⁷.
35. The compound of any one of embodiments 1-29, wherein R³ is cycloalkyl optionally substituted as allowed by valence with 1, 2, 3, or 4 substituents selected from R⁷.
36. The compound of any one of embodiments 1-29, wherein R³ is aryl optionally substituted as allowed by valence with 1, 2, 3, or 4 substituents selected from R⁷.
37. The compound of any one of embodiments 1-29, wherein R³ is heteroaryl optionally substituted as allowed by valence with 1, 2, 3, or 4 substituents selected from R⁷.
38. The compound of any one of embodiments 1-29, wherein R³ is bicycle optionally substituted as allowed by valence with 1, 2, 3, or 4 substituents selected from R⁷.
39. The compound of any one of embodiments 1-38, wherein R² is selected from bond, alkyl, alkenyl, haloalkyl, cycloalkyl, aryl, bicycle, tricycle, and heteroaryl, each of which except bond is optionally substituted as allowed by valence with 1, 2, 3, or 4 substituents selected from R⁷.
40. The compound of any one of embodiments 1-38, wherein R² is bond.
41. The compound of any one of embodiments 1-38, wherein R² is phenyl optionally substituted as allowed by valence with 1, 2, 3, or 4 substituents selected from R⁷.
42. The compound of any one of embodiments 1-38, wherein R² is alkyl optionally substituted as allowed by valence with 1, 2, 3, or 4 substituents selected from R⁷.
43. The compound of any one of embodiments 1-38, wherein R² is alkenyl optionally substituted as allowed by valence with 1, 2, 3, or 4 substituents selected from R⁷.
44. The compound of any one of embodiments 1-38, wherein R² is haloalkyl optionally substituted as allowed by valence with 1, 2, 3, or 4 substituents selected from R⁷.
45. The compound of any one of embodiments 1-38, wherein R² is cycloalkyl optionally substituted as allowed by valence with 1, 2, 3, or 4 substituents selected from R⁷.
46. The compound of any one of embodiments 1-38, wherein R² is aryl optionally substituted as allowed by valence with 1, 2, 3, or 4 substituents selected from R⁷.
47. The compound of any one of embodiments 1-38, wherein R² is heteroaryl optionally substituted as allowed by valence with 1, 2, 3, or 4 substituents selected from R⁷.
48. The compound of any one of embodiments 1-38, wherein R² is bicycle optionally substituted as allowed by valence with 1, 2, 3, or 4 substituents selected from R⁷.
49. The compound of any one of embodiments 1-48, wherein R² is not substituted.
50. The compound of any one of embodiments 1-48, wherein R² is substituted as allowed by valence with 1 substituent selected from R⁷.
51. The compound of any one of embodiments 1-48, wherein R² is substituted as allowed by valence with 2 substituents selected from R⁷.
52. The compound of any one of embodiments 1-48, wherein R² is substituted as allowed by valence with 3 substituents selected from R⁷.
53. The compound of embodiment 1, wherein the compound is of Formula:

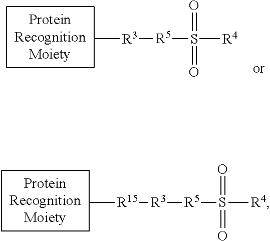

or a pharmaceutically acceptable salt thereof.
54. The compound of embodiment 1, wherein the compound is Formula:

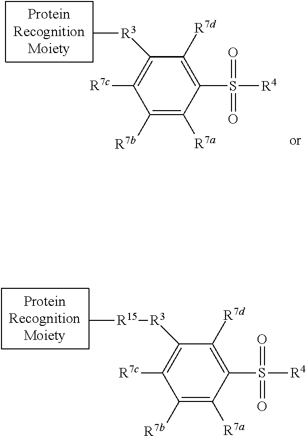

55. The compound of embodiment 1, wherein the compound is Formula:

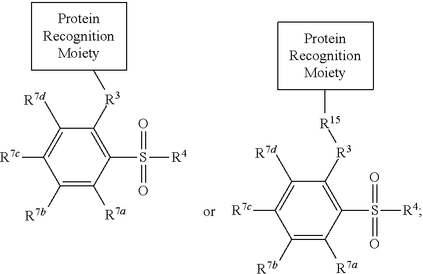

or a pharmaceutically acceptable salt thereof.
56. The compound of embodiment 1, wherein the compound is Formula:

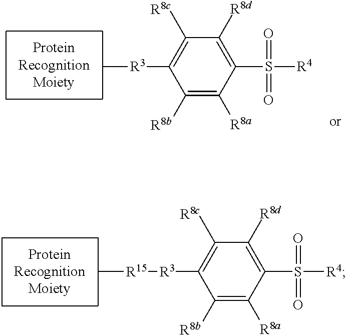

or a pharmaceutically acceptable salt thereof.
57. The compound of any one of embodiments 53-56, wherein R³ is bond.
58. The compound of any one of embodiments 53-56, wherein R³ is phenyl optionally substituted as allowed by valence with 1, 2, 3, or 4 substituents selected from R⁷.
59. The compound of any one of embodiments 53-56, wherein R³ is alkyl optionally substituted as allowed by valence with 1, 2, 3, or 4 substituents selected from R⁷.
60. The compound of any one of embodiments 53-56, wherein R³ is alkenyl optionally substituted as allowed by valence with 1, 2, 3, or 4 substituents selected from R⁷.
61. The compound of any one of embodiments 53-56, wherein R³ is haloalkyl optionally substituted as allowed by valence with 1, 2, 3, or 4 substituents selected from R⁷.
62. The compound of any one of embodiments 53-56, wherein R³ is cycloalkyl optionally substituted as allowed by valence with 1, 2, 3, or 4 substituents selected from R⁷.
63. The compound of any one of embodiments 53-56, wherein R³ is aryl optionally substituted as allowed by valence with 1, 2, 3, or 4 substituents selected from R⁷.
64. The compound of any one of embodiments 53-56, wherein R³ is heteroaryl optionally substituted as allowed by valence with 1, 2, 3, or 4 substituents selected from R⁷.
65. The compound of any one of embodiments 53-56, wherein R³ is bicycle optionally substituted as allowed by valence with 1, 2, 3, or 4 substituents selected from R⁷.
66. The compound of any one of embodiments 1-4 or 22-65, wherein R¹ and R⁴ are

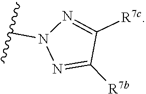

67. The compound of any one of embodiments 1-4 or 22-65, wherein R¹ and R⁴ are

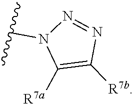

68. The compound of any one of embodiments 1-4 or 22-65, wherein R¹ and R⁴ are

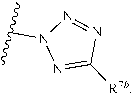

69. The compound of any one of embodiments 1-4 or 22-65, wherein R¹ and R⁴ are

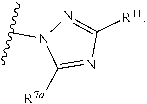

70. The compound of any one of embodiments 1-4 or 22-65, wherein R¹ and R⁴ are

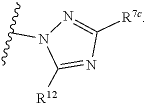

71. The compound of any one of embodiments 1-4 or 22-65, wherein R¹ and R⁴ are

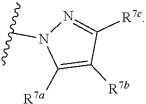

72. The compound of any one of embodiments 1-4 or 22-65, wherein R¹ and R⁴ are

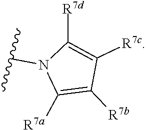

73. The compound of any one of embodiments 1-4 or 22-65, wherein R¹ and R⁴ are

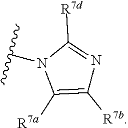

74. The compound of any one of embodiments 1-4 or 22-65, wherein R¹ and R⁴ are a bicyclic heteroaryl which is optionally substituted as allowed by valence with 1, 2, 3, or 4 substituents selected from R⁷.
75. The compound of any one of embodiments 1-4 or 22-65, wherein R⁴ is a heteroaryl group, where the bond to the sulfur atom is through the nitrogen present in the cycle, and each heteroaryl is optionally substituted as allowed by valence with 1, 2, 3, or 4 substituents selected from R⁷.
76. The compound of any one of embodiments 1-75, wherein R⁶ is independently selected at each instance from alkyl, haloalkyl, cycloalkyl, aryl, heterocycle, and heteroaryl; each of which is optionally substituted as allowed by valence with 1, 2, 3, or 4 substituents selected from R¹⁸.
77. The compound of any one of embodiments 1-75, wherein R⁶ is alkyl optionally substituted as allowed by valence with 1, 2, 3, or 4 substituents selected from R¹⁸.
78. The compound of any one of embodiments 1-75, wherein R⁶ is haloalkyl optionally substituted as allowed by valence with 1, 2, 3, or 4 substituents selected from R¹⁸.
79. The compound of any one of embodiments 1-75, wherein R⁶ is cycloalkyl optionally substituted as allowed by valence with 1, 2, 3, or 4 substituents selected from R¹⁸.
80. The compound of any one of embodiments 1-75, wherein R⁶ is aryl optionally substituted as allowed by valence with 1, 2, 3, or 4 substituents selected from R¹⁸.
81. The compound of any one of embodiments 1-75, wherein R⁶ is heterocycle optionally substituted as allowed by valence with 1, 2, 3, or 4 substituents selected from R¹⁸.
82. The compound of any one of embodiments 1-75, wherein R⁶ is heteroaryl optionally substituted as allowed by valence with 1, 2, 3, or 4 substituents selected from R¹⁸.
83. The compound of any one of embodiments 76-82, wherein R⁶ is not substituted with R¹⁸.
84. The compound of any one of embodiments 76-82, wherein R⁶ is substituted with 1 substituent selected from R¹⁸.
85. The compound of any one of embodiments 76-82, wherein R⁶ is substituted with 2 substituents independently selected from R¹⁸.
86. The compound of any one of embodiments 76-82, wherein R⁶ is substituted with 3 substituents independently selected from R¹⁸.
87. The compound of any one of embodiments 1-86, wherein R⁷, R^{7a}, R^{7b}, R^{7c}, and R^{7d} are independently selected at each instance from hydrogen, halogen, alkyl, haloalkyl, alkenyl, cycloalkyl, heterocycle, aryl, heteroaryl, cyano, and nitro, wherein each alkyl, haloalkyl, alkenyl, cycloalkyl, heterocycle, aryl, and heteroaryl is optionally substituted as allowed by valence with 1, 2, 3, or 4 substituents selected from R¹⁷.
88. The compound of any one of embodiments 1-87, wherein one of R⁷, R^{7a}, R^{7b}, R^{7c}, and R^{7d} is alkyl optionally substituted as allowed by valence with 1, 2, 3, or 4 substituents selected from R¹⁷.
89. The compound of any one of embodiments 1-87, wherein one of R⁷, R^{7a}, R^{7b}, R^{7c}, and R^{7d} is haloalkyl optionally substituted as allowed by valence with 1, 2, 3, or 4 substituents selected from R¹⁷.
90. The compound of any one of embodiments 1-87, wherein one of R⁷, R^{7a}, R^{7b}, R^{7c}, and R^{7d} is phenyl optionally substituted as allowed by valence with 1, 2, 3, or 4 substituents selected from R¹⁷.
91. The compound of any one of embodiments 1-87, wherein one of R⁷, R^{7a}, R^{7b}, R^{7c}, and R^{7d} is heteroaryl optionally substituted as allowed by valence with 1, 2, 3, or 4 substituents selected from R¹⁷.
92. The compound of any one of embodiments 1-87, wherein one of R⁷, R^{7a}, R^{7b}, R^{7c}, and R^{7d} is haloalkyl optionally substituted as allowed by valence with 1, 2, 3, or 4 substituents selected from R¹⁷.
93. The compound of any one of embodiments 1-87, wherein one of R⁷, R^{7a}, R^{7b}, R^{7c}, and R^{7d} is heterocycle optionally substituted as allowed by valence with 1, 2, 3, or 4 substituents selected from R¹⁷.
94. The compound of any one of embodiments 1-87, wherein one of R⁷, R^{7a}, R^{7b}, R^{7c}, and R^{7d} is cycloalkyl optionally substituted as allowed by valence with 1, 2, 3, or 4 substituents selected from R¹⁷.
95. The compound of any one of embodiments 87-94, wherein R⁷, R^{7a}, R^{7b}, R^{7c}, and R^{7d} are not substituted.
96. The compound of any one of embodiments 87-94, wherein R⁷, R^{7a}, R^{7b}, R^{7c}, and R^{7d} are optionally substituted with 1 or 2 substituents selected from R¹⁷.
97. The compound of any one of embodiments 1-87, wherein one of R⁷, R^{7a}, R^{7b}, R^{7c}, and R^{7d} is halogen.
98. The compound of any one of embodiments 1-87, wherein one of R⁷, R^{7a}, R^{7b}, R^{7c}, and R^{7d} is —OR⁶.
99. The compound of any one of embodiments 1-87, wherein one of R⁷, R^{7a}, R^{7b}, R^{7c}, and R^{7d} is ═O.
100. The compound of any one of embodiments 1-87, wherein one of R⁷, R^{7a}, R^{7b}, R^{7c}, and R^{7d} is cyano.
101. The compound of any one of embodiments 1-87, wherein one of R⁷, R^{7a}, R^{7b}, R^{7c}, and R^{7d} is nitro.
102. The compound of any one of embodiments 1-101, wherein R¹⁷ is selected in each instance from halogen, alkyl, haloalkyl, alkenyl, and cyano.
103. The compound of any one of embodiments 1-101, wherein R¹⁷ is selected in each instance from halogen, alkyl, and haloalkyl.
104. A compound selected from Table 1.
105. A pharmaceutical composition comprising a compound of any one of embodiments 1-104 or a pharmaceutically acceptable salt thereof and a pharmaceutically acceptable excipient.
106. The pharmaceutical composition of embodiment 105, wherein the composition is suitable for oral delivery.
107. The pharmaceutical composition of embodiment 105, wherein the composition is suitable for intravenous delivery.
108. The pharmaceutical composition of embodiment 105, wherein the composition is suitable for parental delivery.
109. A method of treating a disorder mediated by the Target Protein comprising administering an effective amount of a compound of any one of embodiments 1-104 or a pharmaceutically acceptable salt thereof or a pharmaceutical composition of any one of embodiments 105-108 to a patient in need thereof.
110. The method of embodiment 109, wherein the patient is a human.
111. The method of embodiment 109 or 110, wherein the disorder is a cancer.
112. The method of embodiment 111, wherein the cancer is a solid cancer.
113. The method of embodiment 111, wherein the cancer is a hematological cancer.
114. Use of a compound of any one of embodiments 1-104 or a pharmaceutically acceptable salt thereof or a pharmaceutical composition of any one of embodiments 105-108 to treat a disorder mediated by the Target Protein in a patient in need thereof.
115. Use of a compound of any one of embodiments 1-104 or a pharmaceutically acceptable salt thereof or a pharmaceutical composition of any one of embodiments 105-108 in the manufacture of a medicament to treat a disorder mediated by the Target Protein in a patient in need thereof.
116. The use of embodiment 114 or 115, wherein the patient is a human.
117. The use of any one of embodiments 114-116, wherein the disorder is a cancer.
118. The use of embodiment 117, wherein the cancer is a solid cancer.
119. The use of embodiment 117, wherein the cancer is a hematological cancer.
120. A compound of any one of embodiments 1-104 or a pharmaceutically acceptable salt thereof or a pharmaceutical composition of any one of embodiments 105-108 for use in the treatment of a disorder mediated by the Target Protein in a patient in need thereof.
121. The compound or pharmaceutical composition of embodiment 120, wherein the patient is a human.
122. The compound or pharmaceutical composition of embodiment 120 or 121, wherein the disorder is a cancer.
123. The compound or pharmaceutical composition of embodiment 122, wherein the cancer is a solid cancer.
124. The compound or pharmaceutical composition of embodiment 122, wherein the cancer is a hematological cancer.

### Terminology

As used herein, Anchor Bond is defined as the chemical bond between the Protein Recognition Moiety and the rest of the molecule for example a bond to R³, R⁹, or R¹⁶, as appropriate. Non-limiting examples of Anchor Bonds are shown in bold in the following structures:

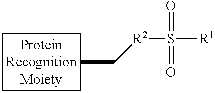

where R³ is methylene

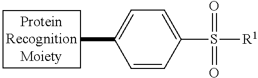

where R³ is bond and R² is phenyl,

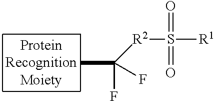

where R⁹ is difluoromethylene, and

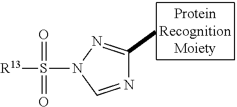

where R¹⁶ is 1,2,4-triazole.

Compounds are described using standard nomenclature. Unless defined otherwise, all technical and scientific terms used herein have the same meaning as is commonly understood by one of skill in the art to which this invention belongs.

The heteroaryl sulfonyl compounds in any of the Formulas described herein include enantiomers, mixtures of enantiomers, diastereomers, tautomers, racemates and other isomers, such as rotamers, as if each is specifically described, unless otherwise indicated or otherwise excluded by context.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

In certain embodiments the present invention includes heteroaryl sulfonyl compounds with at least one desired isotopic substitution of an atom, at an amount above the natural abundance of the isotope, i.e., enriched. In certain embodiments the present invention includes heteroaryl sulfonyl compounds that are not isotopically labeled.

Examples of isotopes that can be incorporated into heteroaryl sulfonyl compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁷O, ¹⁸O, ¹⁸F ³¹P, ³²p, ³⁵S, ³⁶Cl, and ¹²⁵I respectively. In one embodiment, isotopically labelled heteroaryl sulfonyl compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. For example, a ¹⁸F labeled heteroaryl sulfonyl compound may be desirable for PET or SPECT studies. Isotopically labeled heteroaryl sulfonyl compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (²H) and tritium (³H) may optionally be used anywhere in described structures that achieves the desired result. Alternatively, or in addition, isotopes of carbon, e.g., ¹³C and ¹⁴C, may be used. In one embodiment, the isotopic substitution is replacing hydrogen with a deuterium at one or more locations on the molecule to improve the performance of the drug, for example, the pharmacodynamics, pharmacokinetics, biodistribution, half-life, stability, AUC, Tmax, Cmax, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95 or 99% or more enriched in an isotope at any location of interest. In certain embodiments deuterium is 80, 85, 90, 95 or 99% enriched at a desired location. Unless otherwise stated, the enrichment at any point is above natural abundance, and in an embodiment is enough to alter a detectable property of the drug in a human.

In one embodiment, the substitution of a hydrogen atom for a deuterium atom occurs within any variable group. For example, when any variable group is, or contain for example through substitution, methyl, ethyl, or methoxy, the alkyl residue may be deuterated (in nonlimiting embodiments, CDH₂, CD₂H, CD₃, CD₂CD₃, CHDCH₂D, CH₂CD₃, CHDCHD₂, OCDH₂, OCD₂H, or OCD₃ etc.).

The heteroaryl sulfonyl compound of the present invention may form a solvate with solvents (including water). Therefore, in one embodiment, the invention includes a solvated form of the active heteroaryl sulfonyl compound. The term “solvate” refers to a molecular complex of a heteroaryl sulfonyl compound of the present invention (including a salt thereof) with one or more solvent molecules. Nonlimiting examples of solvents are water, ethanol, dimethyl sulfoxide, acetone and other common organic solvents. The term “hydrate” refers to a molecular complex comprising a heteroaryl sulfonyl compound of the invention and water. Pharmaceutically acceptable solvates in accordance with the invention include those wherein the solvent of crystallization may be isotopically substituted, e.g. D₂O, d₆-acetone, d₆-DMSO. A solvate can be in a liquid or solid form.

A dash (“—”) that is not between two letters or symbols is used to indicate a point of attachment for a substituent. For example, —(C═O)NH₂ is attached through carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example a pyridyl group substituted by oxo is a pyridone. Combinations of substituents and/or variables are permissible only if such combinations result in stable compounds or useful synthetic intermediates.

“Alkyl” is a branched, straight chain, or cyclic saturated aliphatic hydrocarbon group. In one embodiment, the alkyl contains from 1 to about 12 carbon atoms, more generally from 1 to about 6 carbon atoms, from 1 to about 4 carbon atoms, or from 1 to 3 carbon atoms. In one embodiment, the alkyl contains from 1 to about 8 carbon atoms. In certain embodiments, the alkyl is C₁-C₂, C₁-C₃, C₁-C₄, C₁-C₅ or C₁-C₆. The specified ranges as used herein indicate an alkyl group which is considered to explicitly disclose as individual species each member of the range described as a unique species. For example, the term C₁-C₆ alkyl as used herein indicates a straight or branched alkyl group having from 1, 2, 3, 4, 5, or 6 carbon atoms and also a carbocyclic alkyl group of 3, 4, 5, or 6 carbon atoms and is intended to mean that each of these is described as an independent species. For example, the term C₁-C₄alkyl as used herein indicates a straight or branched alkyl group having from 1, 2, 3, or 4 carbon atoms and is intended to mean that each of these is described as an independent species. When C₀-Cₙ alkyl is used herein in conjunction with another group, for example, (C₃-C₇cycloalkyl)C₀-C₄ alkyl, or —C₀-C₄alkyl(C₃-C₇cycloalkyl), the indicated group, in this case cycloalkyl, is either directly bound by a single covalent bond (C₀alkyl), or attached by an alkyl chain in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, 2,3-dimethylbutane, and hexyl.

When a term is used that includes “alk” it should be understood that “cycloalkyl” or “carbocyclic” can be considered part of the definition, unless unambiguously excluded by the context. For example and without limitation, the terms alkyl, alkenyl, alkynyl, alkoxy, alkanoyl, alkenloxy, haloalkyl, etc. can all be considered to include the cyclic forms of alkyl, unless unambiguously excluded by context.

“Alkenyl” is a branched or straight chain aliphatic hydrocarbon group having one or more carbon-carbon double bonds that may occur at a stable point along the chain. Nonlimiting examples are C₂-C₈alkenyl, C₂-C₇alkenyl, C₂-C₆alkenyl, C₂-C₅alkenyl and C₂-C₄alkenyl. The specified ranges as used herein indicate an alkenyl group having each member of the range described as an independent species, as described above for the alkyl moiety. Examples of alkenyl include, but are not limited to, ethenyl and propenyl.

“Alkynyl” is a branched or straight chain aliphatic hydrocarbon group having one or more carbon-carbon triple bonds that may occur at any stable point along the chain, for example, C₂-C₈alkynyl or C₂-C₆alkynyl. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

“Alkoxy” is an alkyl group as defined above covalently bound through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “alkylthio” or a“thioalkyl” group is an alkyl group as defined above with the indicated number of carbon atoms covalently bound through a sulfur bridge (—S—). In one embodiment, the alkoxy group is optionally substituted as described above.

“Haloalkyl” indicates both branched and straight-chain alkyl groups substituted with 1 or more halogen atoms, up to the maximum allowable number of halogen atoms. Examples of haloalkyl include, but are not limited to, trifluoromethyl, monofluoromethyl, difluoromethyl, 2-fluoroethyl, and penta-fluoroethyl.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. The term “aryl” includes groups where a saturated or partially unsaturated carbocycle group is fused with an aromatic ring. The term “aryl” also includes groups where a saturated or partially unsaturated heterocycle group is fused with an aromatic ring so long as the attachment point is the aromatic ring. Such compounds may include aryl rings fused to a 4 to 7 or a 5 to 7-membered saturated or partially unsaturated cyclic group that optionally contains 1, 2 or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group.

The term “heterocycle” refers to saturated and partially saturated heteroatom-containing ring radicals, where the heteroatoms may be selected from N, S, and O. The term “heterocycle” includes monocyclic 3-12 membered rings, as well as bicyclic 5-16 membered ring systems (which can include fused, bridged, or spiro, bicyclic ring systems). It does not include rings containing —O—O— or —S—S— portions. Examples of saturated heterocycle groups include saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4 to 6-membered monocyclic groups containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl. “Bicyclic heterocycle” includes groups wherein the heterocyclic radical is fused with an aryl radical wherein the point of attachment is the heterocycle ring. “Bicyclic heterocycle” also includes heterocyclic radicals that are fused or bridged with a carbocycle radical. For example partially unsaturated condensed heterocyclic group containing 1 to 5 nitrogen atoms, for example, indoline, isoindoline, partially unsaturated condensed heterocyclic group containing 1 to 2 oxygen atoms and 1 to 3 nitrogen atoms, partially unsaturated condensed heterocyclic group containing 1 to 2 sulfur atoms and 1 to 3 nitrogen atoms, and saturated condensed heterocyclic group containing 1 to 2 oxygen or sulfur atoms.

Non-limiting examples of bicyclic heterocycles include:

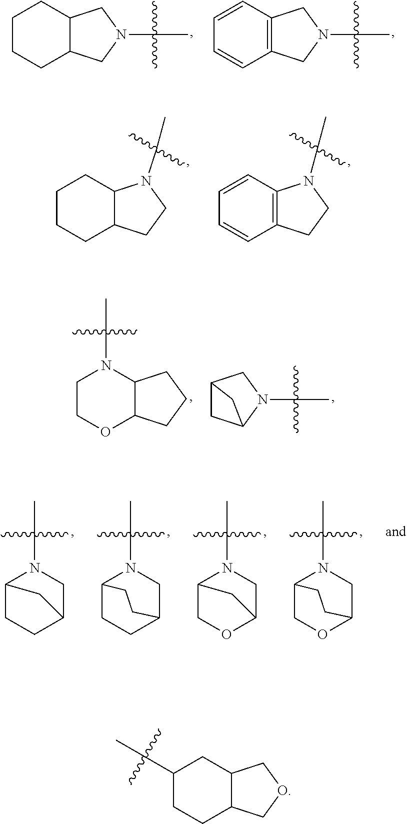

Unless otherwise drawn or clear from the context, the term “bicyclic heterocycle” includes cis and trans diastereomers. Non-limiting examples of chiral bicyclic heterocycles include:

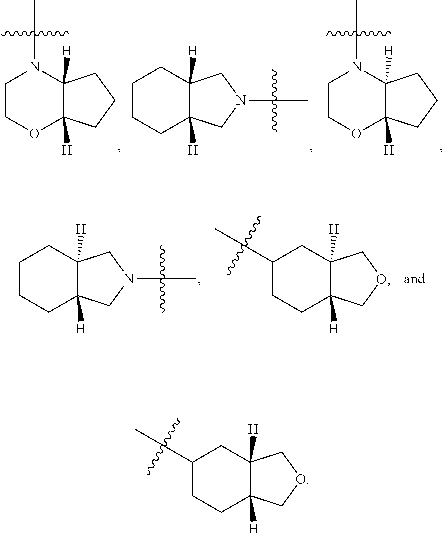

In certain alternative embodiments the term “heterocycle” refers to saturated and partially saturated heteroatom-containing ring radicals, where the heteroatoms may be selected from N, S, O, B, Si, and P.

The term “bicycle” refers to a ring system wherein two rings are fused together and each ring is independently selected from carbocycle, heterocycle, aryl, and heteroaryl. Non-limiting examples of bicycle groups include:

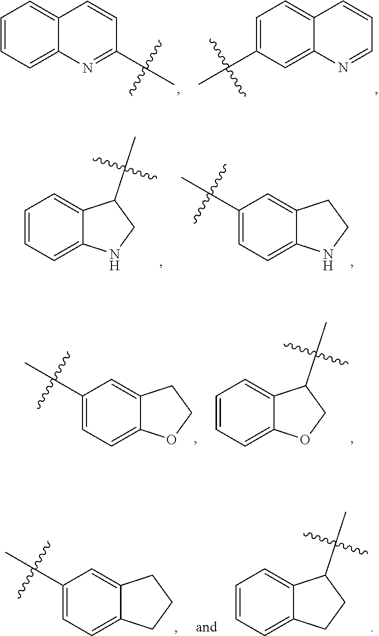

When the term “bicycle” is used in the context of a bivalent residue such as R², R³, or R⁵, the attachment points can be on separate rings or on the same ring. In certain embodiments both attachment points are on the same ring. In certain embodiments both attachment points are on different rings. Non-limiting examples of bivalent bicycle groups include:

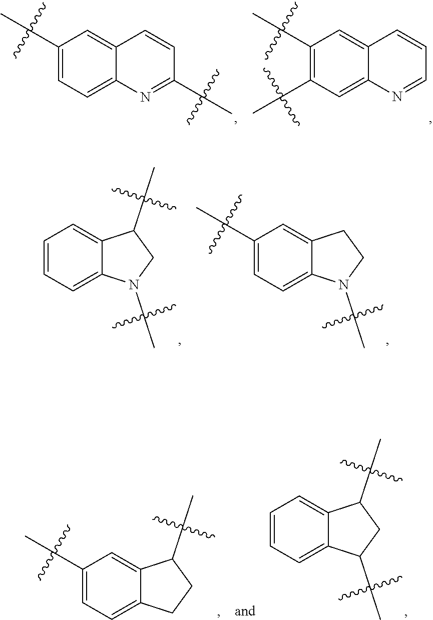 [SMILES: *c1ccc2nc(*)ccc2c1 ; *c1cc2cccnc2cc1* ; *C1CN(*)c2ccccc21 ; *c1ccc2c(c1)CCN2* ; *c1ccc2c(c1)C(*)CC2 ; *C1CC(*)c2ccccc21]

“Heteroaryl” refers to a stable monocyclic, bicyclic, or multicyclic aromatic ring which contains from 1 to 5, or in some embodiments from 1, 2, 3, 4, or 5 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B or P with remaining ring atoms being carbon. In one embodiment, the only heteroatom is nitrogen. In one embodiment, the only heteroatom is oxygen. In one embodiment, the only heteroatom is sulfur. Monocyclic heteroaryl groups typically have from 5 or 6 ring atoms. In some embodiments bicyclic heteroaryl groups are 8- to 10-membered heteroaryl groups, that is, groups containing 8 or 10 ring atoms in which one 5, 6, or 7-member aromatic ring is fused to a second aromatic or non-aromatic ring wherein the point of attachment is the aromatic ring. When the total number of S and O atoms in the heteroaryl group exceeds 1, these heteroatoms are not adjacent to one another. In one embodiment, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl. Heteroaryl groups are optionally substituted independently with one or more substituents described herein.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

“Pharmaceutical compositions” are compositions comprising at least one active agent, and at least one other substance, such as a carrier. The present invention includes pharmaceutical compositions of the described heteroaryl sulfonyl compounds.

“Pharmaceutical combinations” are combinations of at least two active agents which may be combined in a single dosage form or provided together in separate dosage forms with instructions that the active agents are to be used together to treat any disorder described herein.

A “pharmaceutically acceptable salt” is a derivative of the disclosed heteroaryl sulfonyl compound in which the parent heteroaryl sulfonyl compound is modified by making inorganic and organic, pharmaceutically acceptable, acid or base addition salts thereof. The salts of the present heteroaryl sulfonyl compounds can be synthesized from a parent heteroaryl sulfonyl compound that contains a basic or acidic moiety by conventional chemical methods. Generally, such salts can be prepared by reacting free acid forms of these heteroaryl sulfonyl compounds with a stoichiometric amount of the appropriate base (such as Na, Ca, Mg, or K hydroxide, carbonate, bicarbonate, or the like), or by reacting free base forms of these heteroaryl sulfonyl compounds with a stoichiometric amount of the appropriate acid. Such reactions are typically carried out in water or in an organic solvent, or in a mixture of the two. Salts of the present heteroaryl sulfonyl compounds further include solvates of the heteroaryl sulfonyl compounds and of the heteroaryl sulfonyl compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent heteroaryl sulfonyl compound formed, for example, from inorganic or organic acids. Examples, of such salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations of the invention refers to a diluent, excipient, or vehicle with which an active heteroaryl sulfonyl compound is provided.

A “pharmaceutically acceptable excipient” means an excipient that is useful in preparing a pharmaceutical composition/combination that is generally safe, acceptable for human consumption, and neither biologically nor otherwise inappropriate for administration to a host, typically a human. In one embodiment, an excipient is used that is acceptable for veterinary use.

A “patient” or “host” or “subject” is a human or non-human animal in need of treatment or prevention of any of the disorders as specifically described herein. Typically, the host is a human. A “patient” or “host” or “subject” also refers to for example, a mammal, primate (e.g., human), cow, sheep, goat, horse, dog, cat, rabbit, rat, mice, bird and the like.

A “therapeutically effective amount” of a heteroaryl sulfonyl compound, pharmaceutical composition, or combination of this invention means an amount effective, when administered to a host, provides a therapeutic benefit such as an amelioration of symptoms or reduction or diminution of the disease itself.

### Protein Recognition Moiety

Protein Recognition Moiety is typically a small molecule, peptide, protein, oligonucleotide, nucleotide, RNA, DNA, SiRNA, a biologic, an antibody or a fragment thereof, or other group which can bind to or otherwise interact with a Target Protein, either specifically or non-specifically, in order to bring the heteroaryl sulfonyl compound of the present invention in close spatial proximity to the Target Protein. In certain embodiments the Protein Recognition Moiety binds the Target Protein in a binding pocket for example a cleft, pocket, or cavity; catalytic site; allosteric site; or surface binding site for example a concave, convex or flat surface site.

The Protein Recognition Moiety is typically a ligand or a portion of a ligand that binds to the Target Protein. Non-limiting examples of Protein Recognition Moieties are provided in the Figures. The skilled artisan will recognize additional Protein Recognition Moieties that are known in the art and will know where to link the moiety to provide the desired effect. For example, the skilled artisan can look up the crystal structure for the protein that they want to covalently modify on https://www.rcsb.org/and then pull a list of ligands that bind that crystal structure. The skilled artisan can also determine where to attach the sulfur-heteroaryl group of the present invention based on the crystal structure provided which will allow identification of where in the binding pocket the sulfur-heteroaryl group can fit and which functional groups on the ligand are essential for activity.

In certain embodiments the Protein Recognition Moiety is a RNA that binds the Target Protein. The RNA can be a fragment, SiRNA, a sequence of naturally occurring RNA, a sequence of unnatural RNA, or a combination thereof. In certain embodiments the RNA binds a viral target (see, for example, the paper by Bader Alhatlani, “In silico identification of conserved cis-acting RNA elements in the SARS COV-2 genome” Future Virology 15(7) 409-417). In another embodiment the RNA binds a protein that mediates a non-viral disorder such as a cancer or a tumor (see, for example, the paper by Xiangping Liang, et. al., “RNA-based pharmacotherapy for tumors: From bench to clinic and back” Biomedicine and Pharmacotherapy Volume 125, 2020, 109997).

In certain embodiments the Protein Recognition Moiety is a DNA that binds the Target Protein. The DNA can be a fragment, a sequence of naturally occurring DNA, a sequence of unnatural DNA, or a combination thereof (see, for example, the paper by Siddhesh D Patil, et al. “DNA-based therapeutics and DNA delivery systems: a comprehensive review” AAPS J. 2005 8; 7(1)).

In certain embodiments the Protein Recognition Moiety binds a disease-related protein.

In certain embodiments the Protein Recognition Moiety binds a cancer-related protein.

In certain embodiments the Protein Recognition Moiety is a small molecule that binds a cancer-related protein.

In certain embodiments the Protein Recognition Moiety is a peptide that binds a cancer-related protein.

In certain embodiments the Protein Recognition Moiety is a ligand that binds at least one of the following proteins cancer mediating proteins: retinoid x receptor, dihydrofolate reductase, protein tyrosine phosphatase, aurora kinase, tyrosine kinase, heat shock protein 90, JAK2, ABL, anaplastic lymphoma kinase, MET kinase, mammalian target of rapamycin complex 1, mammalian target of rapamycin complex 2, mast/stem cell growth factor receptor or c-KIT, insulin-like growth factor 1 receptor, Mouse double minute 2 homolog, Bromodomain-containing protein 2, Bromodomain-containing protein 3, Bromodomain-containing protein 4, Bromodomain testis-specific protein, FKBP, A-RAF, B-RAF, c-RAF, Histone deacetylase, Androgen receptor, Estrogen receptor, Thyroid hormone receptor, AP1 and/or AP2, MCL1, IDH1, MERTK or MER, EGFR, FLT3, SMARCA2, CDK4, CDK6, CDK9, CDK12, CDK13, Glucocorticoid Receptor, HER3, BCL2, BCL3, BCL5, BCL6, BCL7A, BCL9, BCL10, BCL-XL, PPAR-gamma, BCR-ABL, ALK, LRRK2, PDGFRA, RET, Fatty Acid Binding Protein, 5-Lipoxygenase-activating protein, Lactoylglutathione Lyase, mPGES-1, KLK7, Cathepsin K, Cathepsin L2, Cathepsin S, MTH1, MDM4, MDMX, PARP1, PARP2, PARP3, PARP14, PARP15, PDZ domain, Phospholipase A2, S100A7, c-Src, JAK3, MEK1, KIT, KSR1, Beta-catenin, PAK1, PAK4, TNIK, MEN1, ERK1, CBP, IDO1, ASH1L, ATAD2, BAZ2A, BRD2, BRD4, BRDT, BRD9, TRIM24, BRPF1, CECR2, EP300, PCAF, PHIP, TAF1, TAF1L, HDAC2, HDAC4, HDAC6, HDAC7, HDAC8, KAT2A, HAT1, ATF2, KAT5, KDM1A, DOT1L, EHMT1, EHMT2, SMYD2, SETDB1, SETD8, SMYD3, SUV4-20H1, T877A mutant Androgen Receptor, W741L mutant Androgen Receptor, ErbB2, ErbB4, VEGFR1, VEGFR2, VEGFR3, PDGFRbeta, LYN, HCK, c-MET, TRKB, TIE2, AXL, ROS1, INSR, IGF1R, MST1R, FYN, EPHA2, HER2, BTK, FGFR1, FGFR2, FGFR3, DDR1, JAK1, EPHR, CDK8, CSF1R, MEK2, BRK, PI3Ka, EZH2, and Polycomb protein EED.

In certain embodiments, the Protein Recognition Moiety is myeloid leukemia factor 1 or a fragment thereof. In certain embodiments, the Protein Recognition Moiety has the sequence SEQ ID NO: 1 MIRSFSEPFGRDLL or is a 1, 2, 3, or 4 amino acid mutation thereof. In certain embodiments, the Protein Recognition Moiety has the sequence SEQ ID NO: 2 RRQRSAP or is a 1, 2, 3, or 4 amino acid mutation thereof. In certain embodiments, the Protein Recognition Moiety has the sequence SEQ ID NO: 3 SISR or is a 1, 2, 3, or 4 amino acid mutation thereof.

In certain embodiments the Protein Recognition Moiety is a ligand that binds 14-3-3 protein theta (YWHAQ). Non-limiting examples of crystal structures of YWHAQ with Protein Recognition Moieties include 6KZH, 6KZG, 2YEZ, 6BQT, 2BTP, 6BD1, 6BD2, 6BCR, 4DNK, and 5IQP. In certain embodiments tyrosine 48 of YWHAQ is covalently modified by a compound of the present invention with a YWHAQ Protein Recognition Moiety.

In certain embodiments, the Protein Recognition Moiety is a CIC pS173 peptide. In certain embodiments, the Protein Recognition Moiety has a SEQ ID NO: 4 RTQSLSAL or is a 1, 2, 3, or 4 amino acid mutation thereof. In certain embodiments, the Protein Recognition Moiety is a CIC S301 phosphorylated peptide. In certain embodiments, the Protein Recognition Moiety is ARG-SER-MET-SER-GLU-THR-GLY-THR. In certain embodiments, the Protein Recognition Moiety has a SEQ ID NO: 5 RSMSETGT or is a 1, 2, 3, or 4 amino acid mutation thereof. In certain embodiments, the Protein Recognition Moiety is a beta-2-microglobulin. In certain embodiments, the Protein Recognition Moiety has a SEQ ID NO: 6 TNPESKVFYL or is a 1, 2, 3, or 4 amino acid mutation thereof. In certain embodiments, the Protein Recognition Moiety is an insulin receptor substrate protein of 53 kDa, peptide (IRSp53). In certain embodiments, the Protein Recognition Moiety is a consensus peptide for 14-3-3 protein. In certain embodiments, the Protein Recognition Moiety has a SEQ ID NO: 7 RQRSAP or is a 1, 2, 3, or 4 amino acid mutation thereof.

In certain embodiments, the Protein Recognition Moiety is a Vacuolar protein sorting-associated protein 26B. In certain embodiments, the Protein Recognition Moiety is a Putative vacuolar protein sorting-associated protein. In certain embodiments, the Protein Recognition Moiety is a Vacuolar protein sorting-associated protein 29. In certain embodiments, the Protein Recognition Moiety is a Vacuolar protein sorting-associated protein 35. In certain embodiments, the Protein Recognition Moiety is a TBC1 domain family member 5. In certain embodiments, the Protein Recognition Moiety has a SEQ ID NO: 8 GQQDLMINNPLSQDEGSLWNKFFQDKE or is a 1, 2, 3, or 4 amino acid mutation thereof. In certain embodiments, the Protein Recognition Moiety is a Interaptin protein. In certain embodiments, the Protein Recognition Moiety is a Sorting nexin-3 protein.

In certain embodiments the Protein Recognition Moiety is a ligand that binds isocitrate dehydrogenase cytoplasmic protein (IDH1). Non-limiting examples of crystal structures of IDH1 with Protein Recognition Moieties include 5SUN, 5SVO, 5SVN, 5SVF, 4L03, 4L04, 4L06, 4KZO, 5DE1, 6B0Z, 5LGE, 6Q6F, 6ADG, 6ADI, 4I3K, 413L, 6BKX, 6BKZ, 6BL1, 6BL0, 6BL2, 5L58, 5L57, 3MAP, 3MAR, 3MAS, 5TQH, 4UMX, 4UMY, 4XRX, 4XS3, 6O2Y, 6U4J, 6I00, 3INM, 6VEI, 6VFZ, 6VGO, 602Z, 5YFM, 5K10, 5K11, 5YZI, 5YZH, 6PAY, 1TOL, 1T09, 4JA8, 5I95, and 5I96. In certain embodiments tyrosine 139 of IDH1 is covalently modified by a compound of the present invention with a IDH1 Protein Recognition Moiety.

In certain embodiments, the Protein Recognition Moiety is an axin peptide. In certain embodiments, the Protein Recognition Moiety has a SEQ ID NO: 9 VEPQKFAEELIHRLEAVQ or is a 1, 2, 3, or 4 amino acid mutation thereof.

In certain embodiments, the Protein Recognition Moiety is a ligand that binds phosphoglycerate mutase 1 (PGAM1). Non-limiting examples of crystal structures of PGAM1 with Protein Recognition Moieties include 5Y2U, 5Y2I, 6ISN, 5Y35, 5Y64, 5Y65, 5ZRM, and 5ZS8. In certain embodiments tyrosine 92 of PGAM1 is covalently modified by a compound of the present invention with a PGAM1 Protein Recognition Moiety.

In certain embodiments the Protein Recognition Moiety is a ligand that binds glutathione S-transferase P (GSTP1). In certain embodiments tyrosine 8 of GSTP1 is covalently modified by a compound of the present invention with a GSTP1 Protein Recognition Moiety. Non-limiting examples of crystal structures of GSTP1 with Protein Recognition Moieties include 2OA7, 2OAC, 2OAD, 1GLQ, 1GSY, 1GTI, 2GLR, 1GLP, 1PGT, 7GSS, 18GS, 12GS, 10GS, 19GS, 13GS, 20GS, 5X79, 1GSS, 1AQX, 1AQV, 3CSH, 3PGT, 2A2R, 6Y1E, and 2PGT.

In certain embodiments, the Protein Recognition Moiety is a ligand that binds nucleoside diphosphate kinase B (NME2). Non-limiting examples of crystal structures of NME2 with Protein Recognition Moieties include 3BBB, 3BBF, and INUE. In certain embodiments tyrosine 67 of NME2 is covalently modified by a compound of the present invention with a NME2 Protein Recognition Moiety.

In certain embodiments, the Protein Recognition Moiety is a ligand that binds Biliverdin reductase A (BLVRA). Non-limiting examples of crystal structures of BLVRA with Protein Recognition Moieties include 2H63 and ILC3. In certain embodiments tyrosine 98 of BLVRA is covalently modified by a compound of the present invention with a BLVRA Protein Recognition Moiety.

In certain embodiments, the Protein Recognition Moiety is a ligand that binds Ras-related C3 botulinum toxin substrate 3 (RAC3). Non-limiting examples of crystal structures of RAC3 with Protein Recognition Moieties include 2C2H. In certain embodiments tyrosine 139 of RAC3 is covalently modified by a compound of the present invention with a RAC3 Protein Recognition Moiety.

In certain embodiments, the Protein Recognition Moiety is a ligand that binds Thymidine kinase, cytosolic (TK1). Non-limiting examples of crystal structures of TK1 with Protein Recognition Moieties include 1XBT. In certain embodiments tyrosine 181 of TK1 is covalently modified by a compound of the present invention with a TK1 Protein Recognition Moiety.

In certain embodiments, the Protein Recognition Moiety is a ligand that binds Glutamine synthetase (GLUL or GS). Non-limiting examples of crystal structures of GS with Protein Recognition Moieties include 2QC8. In certain embodiments tyrosine 336 of GLUL is covalently modified by a compound of the present invention with a GLUL Protein Recognition Moiety.

In certain embodiments, the Protein Recognition Moiety is a ligand that binds Eukaryotic initiation factor 4A-III (EIF4A3). Non-limiting examples of crystal structures of EIF4A3 with Protein Recognition Moieties include 2JOS, 2JOQ and 2HYI. In certain embodiments tyrosine 207 of EIF4A3 is covalently modified by a compound of the present invention with a EIF4A3 Protein Recognition Moiety.

In certain embodiments, the Protein Recognition Moiety is a ligand that binds Hypoxanthine-guanine phosphoribosyltransferase (HPRT or HPRT1). Non-limiting examples of crystal structures of HPRT1 with Protein Recognition Moieties include 1BZY. In certain embodiments tyrosine 105 of HPRT1 is covalently modified by a compound of the present invention with a HPRT1 Protein Recognition Moiety.

In certain embodiments, the Protein Recognition Moiety is a ligand that binds Glycogen phosphorylase, brain form (PYGB). Non-limiting examples of crystal structures of PYGB with Protein Recognition Moieties include 5IKP. In certain embodiments tyrosine 197 of PYGB is covalently modified by a compound of the present invention with a PYGB Protein Recognition Moiety.

In certain embodiments, the Protein Recognition Moiety is a ligand that binds Vinculin (VCL). Non-limiting examples of crystal structures of VCL with Protein Recognition Moieties include 5L0C and 5L0D. In certain embodiments tyrosine 1133 of VCL is covalently modified by a compound of the present invention with a VCL Protein Recognition Moiety.

In certain embodiments, the Protein Recognition Moiety is a ligand that binds cytosolic Branched-chain-amino-acid aminotransferase (BCAT1). Non-limiting examples of crystal structures of BCAT1 with Protein Recognition Moieties include 2COJ, 2COI, 2COG, 2A1H, and 2ABJ. In certain embodiments tyrosine 161 of BCAT1 is covalently modified by a compound of the present invention with a BCAT1 Protein Recognition Moiety.

In certain embodiments, the Protein Recognition Moiety is a ligand that binds Nucleoside diphosphate kinase A (NME1). Non-limiting examples of crystal structures of NME1 with Protein Recognition Moieties include 2HVD, 2HVE and 5UI4. In certain embodiments tyrosine 52 of NME1 is covalently modified by a compound of the present invention with a NME1 Protein Recognition Moiety.

In certain embodiments, the Protein Recognition Moiety is a ligand that binds Adenylosuccinate lyase (ADSL). Non-limiting examples of crystal structures of ADSL with Protein Recognition Moieties include 2J91 and 2VD6. In certain embodiments tyrosine 21 of ADSL is covalently modified by a compound of the present invention with a ADSL Protein Recognition Moiety.

In certain embodiments, the Protein Recognition Moiety is a ligand that binds ADP-ribose pyrophosphatase, mitochondrial (NUDT9). Non-limiting examples of crystal structures of NUDT9 with Protein Recognition Moieties include 1QVJ and 1Q33. In certain embodiments tyrosine 321 of NUDT9 is covalently modified by a compound of the present invention with a NUDT9 Protein Recognition Moiety.

In certain embodiments, the Protein Recognition Moiety is a ligand that binds Peptidyl-prolyl cis-trans isomerase NIMA-interacting 1 (PIN1). Non-limiting examples of crystal structures of PIN1 with Protein Recognition Moieties include 2ITK and 3I6C. In certain embodiments, the Protein Recognition Moiety is D-peptide. In certain embodiments, the Protein Recognition Moiety has a sequence SEQ ID NO: 10 XFTXAQX or is a 1, 2, 3, or 4 amino acid mutation thereof. In certain embodiments tyrosine 23 of PIN1 is covalently modified by a compound of the present invention with a PIN1 Protein Recognition Moiety.

In certain embodiments, the Protein Recognition Moiety is a ligand that binds 14-3-3 protein beta/alpha (YWHAB). Non-limiting examples of crystal structures of YWHAB with Protein Recognition Moieties include 5N10, 6BYK and 6HEP. In certain embodiments, the Protein Recognition Moiety is both a nonavalent CFTR and the hexavalent LRRK2 protein. In certain embodiments, the Protein Recognition Moiety is a hexavalent LRRK2 protein. In certain embodiments, the Protein Recognition Moiety is a nonavalent CFTR. (Stevers et al., “A Thermodynamic Model for Multivalency in 14-3-3 Protein-Protein Interactions”, J Am Chem Soc., 2018, 140: 14498-14510) In certain embodiments tyrosine 130 or 50 of YWHAB is covalently modified by a compound of the present invention with a YWHAB Protein Recognition Moiety.

In certain embodiments, the Protein Recognition Moiety is a ligand that binds Bifunctional purine biosynthesis protein ATIC (ATIC). Non-limiting examples of crystal structures of ATIC with Protein Recognition Moieties include 1PLO, 5UZ0 and 5UY8. In certain embodiments tyrosine 208 of ATIC is covalently modified by a compound of the present invention with a ATIC Protein Recognition Moiety.

In certain embodiments, the Protein Recognition Moiety is a ligand that binds Glucose-6-phosphate 1-dehydrogenase (G6PD). Non-limiting examples of crystal structures of G6PD with Protein Recognition Moieties include 5UKW, 1QKI, 2BHL, 6JYU, and 2BH9. In certain embodiments tyrosine 202 of G6PD is covalently modified by a compound of the present invention with a G6PD Protein Recognition Moiety.

In certain embodiments, the Protein Recognition Moiety is a ligand that binds Glycogen phosphorylase, liver form (PYGL). Non-limiting examples of crystal structures of PYGL with Protein Recognition Moieties include 1FA9, 3DD1, 3DDS, 3DDW, 2QLL, 3CEH, and 3CEJ. In certain embodiments tyrosine 76 OR 574 of PYGL is covalently modified by a compound of the present invention with a PYGL Protein Recognition Moiety.

In certain embodiments, the Protein Recognition Moiety is a ligand that binds GDP-mannose 4,6 dehydratase (GMDS). Non-limiting examples of crystal structures of GMDS with Protein Recognition Moieties include 6GPK, 1T2A, and 6GPL. In certain embodiments tyrosine 323 of GMDS is covalently modified by a compound of the present invention with a GMDS Protein Recognition Moiety.

In certain embodiments, the Protein Recognition Moiety is a ligand that binds SR-related and CTD-associated factor 8 (SCAF8). Non-limiting examples of crystal structures of SCAF8 with Protein Recognition Moieties include 3D9K, 3D9M, 3D9N, and 3D90. In certain embodiments, the Protein Recognition Moiety is a RNA-binding protein 16. In certain embodiments, the Protein Recognition Moiety is a CTD-peptide. In certain embodiments, the Protein Recognition Moiety is a sequence SEQ ID NO: 11 YSPTSPSYSPTSPS or is a 1, 2, 3, or 4 amino acid mutation thereof. In certain embodiments tyrosine 64 of SCAF8 is covalently modified by a compound of the present invention with a SCAF8 Protein Recognition Moiety.

In certain embodiments, the Protein Recognition Moiety is a ligand that binds Peptidyl-prolyl cis-trans isomerase FKBPlA (FKBPlA). Non-limiting examples of crystal structures of FKBP1A with Protein Recognition Moieties include 1D7I, 1F40, 1FKD, 2FKE, 1FKF, 1FKJ, 1J4I, 1J4H, 1BL4, 1FKG, 1FKH, 1FKI, 1QPF, 3FAP, 2FAP, 6M4U, 1FAP, 1J4R, 1A7X, 1QPL, 1FKB, 2DG3, and 2DG9. In certain embodiments, the Protein Recognition Moiety is a FK506-binding protein. In certain embodiments tyrosine 83 of FKBPlA is covalently modified by a compound of the present invention with a FKBPlA Protein Recognition Moiety.

In certain embodiments the Protein Recognition Moiety is not a promiscuous protein binder, for example a promiscuous binder of enzymes or of kinases.

In certain embodiments the Protein Recognition Moiety is not a calcium channel blocker, ligand for N-RAS, ligand for a RXR protein, PPAR antagonist, serotonin 5-HT receptor antagonist, or TR4 nuclear receptor antagonist.

In certain embodiments the Protein Recognition Moiety is not flunarizine or a derivative or fragment thereof. For example in certain embodiments the Protein Recognition Moiety is not:

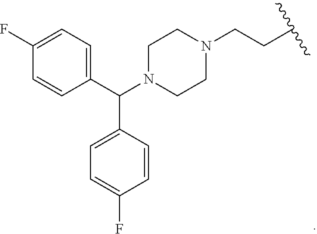 [SMILES: *CCN1CCN(C(c2ccc(F)cc2)c2ccc(F)cc2)CC1]

In certain embodiments the Protein Recognition Moiety is not ritanserin or a derivative or fragment thereof. For example, in certain embodiments the Protein Recognition Moiety is not:

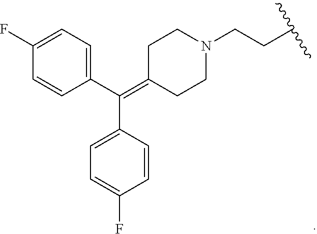 [SMILES: *CCN1CCC(=C(c2ccc(F)cc2)c2ccc(F)cc2)CC1]

In certain embodiments the Protein Recognition Moiety is not bexarotene, tonalide, or tamibarotene, or a derivative or fragment thereof. For example, in certain embodiments the Protein Recognition Moiety is not:

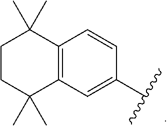 [SMILES: *c1ccc2c(c1)C(C)(C)CCC2(C)C]

In certain embodiments the Protein Recognition Moiety is not a promiscuous kinase ligand. For example, in certain embodiments the Protein Recognition Moiety is not:

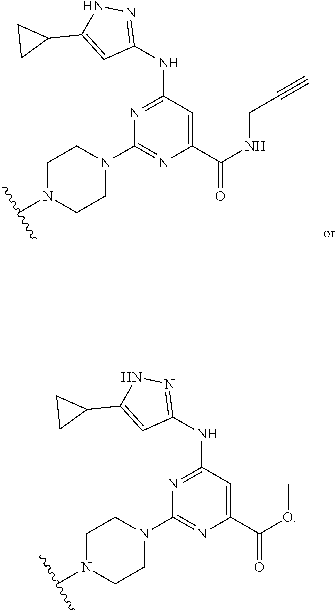 [SMILES: *N1CCN(c2nc(Nc3cc(C4CC4)[nH]n3)cc(C(=O)NCC#C)n2)CC1 ; *N1CCN(c2nc(Nc3cc(C4CC4)[nH]n3)cc(C(=O)OC)n2)CC1]

In certain embodiments the Protein Recognition Moiety binds fewer than 80, 70, 60, 50, 40, 30, 20, 15, 10, or 5 endogenous protein kinases with a KD50 of 10 μM or less.

In certain embodiments the Protein Recognition Moiety binds fewer than 80, 70, 60, 50, 40, 30, 20, 15, 10, or 5 endogenous protein kinases with a K_{d}50 of 5 μM or less.

In certain embodiments the Protein Recognition Moiety binds fewer than 80, 70, 60, 50, 40, 30, 20, 15, 10, or 5 endogenous protein kinases with a K_{d}50 of 2 μM or less.

In certain embodiments the Protein Recognition Moiety binds fewer than 80, 70, 60, 50, 40, 30, 20, 15, 10, or 5 endogenous protein kinases with a K_{d}50 of 1 μM or less.

In certain embodiments the Protein Recognition Moiety binds fewer than 80, 70, 60, 50, 40, 30, 20, 15, 10, or 5 endogenous protein kinases with a K_{d}50 of 0.5 μM or less.

### Target Protein

A Target Protein or Targeted Protein is typically a classically druggable or to date undruggable protein. Non-limiting examples of proteins that can be targeted by the present invention include enzymes, signaling proteins, structural proteins, surface proteins, intracellular proteins, and extracellular proteins.

In certain embodiments, the Target Protein is a cancer related fusion protein. In certain embodiments, the Target Protein is selected from VEGF, SOX7, c-MET, HGFR, PTTG, cyclin D1, KIF4A, ALK, ROS1, BRAF, C-KIT, EGFR, HER2, ERBB2, JAK2, PD-1, MAPK, PI3K, ERK, ROS proto-oncogene 1, ROS1, PD-L1, PD-L2, EGFRTK, COX-2, PKC, HRAS, RXR, CDK1, CDK4, CDK7, BCL-2, BCL-XL, CTLA-4, PARP, RAD51, ERB4, VEGFR, PDGFR, FLT-3, c-FMS, MEK, mTOR, CHK1, CHK2, CD28, NRAS, CTNNB1, PIK3CA, AKT, DDR2, LKB1, FGFR1, PTEN, SOX2, TP53, c-MYC, CCND1, Cyclin E, ERalpha, RB, BRCA1, BRCA2, IGF1R, HER1, HER3, CDK6, HSP90, FOXA1, COX-1, CXCL8, CCL2, CCR2, CCR5, CXCR4, CXCL12, PI, ZNF703, FLT3, HOXA9, HOXD13, HOXA9, HOXC, PRX1, PRX2, BCR, ABL1, SRC, ABCB1, ABCG2, NFkB, PML, RARalpha, PLZF, TRAIL, RAS, RB1, pRB, MYC, NEU, WNT-1, Cyclin D2, AML, NUP98, PDGFRbeta, STAT5, RAF, MAPK, CD30, BCL6, BTK, EZH2, BAFF, TGFbeta, SYK, PKCbeta, STAT3, mTORC1, mTORC2, RNA polymerase II, Aurora Kinase A, Aurora Kinase B, HDM2, BCL-W, BCL2A1, MCL-1, CDK5, IRF4, CD38, NAE1, DNMT1, DNMT3A, DNMT3B, PRMT5, HDAC2, HIF-1A, CD40, RANK, HDAC1, HDAC3, HDAC8, MMP-9, CD20, SlPRl, NFE2L2, AHR, cPLA2, CNR1, CERS2, KIR4.1, P2X1, P2X3, P2X7, TLR2, TLR4, TLR7, TLR9, NF-kB, IL-17, alpha-v beta-3, ANGPT1, TNFalpha, IL-6, CCL5, CXCL10, CXCL5, CXCL1, CXCL13, FLIP, SUMO-1, FAP, PBEF, STAT4, RF, ACPA, HLA-DRB1, PTPN22, TH-17, IL-21, IL-22, IL-23, GM-CSF, JAK1, JAK3, reverse transcriptase, aspartyl protease, integrase, matrix-2 protein, neuraminidase, viral RNA polymerase, viral DNA polymerase, NS2-3 protease, NS3-4A protease, NS5A, GP41, CCR5, CXCR4, CFTR, LCK, LYN, IL-1beta, ILK, S6K1, TIMP-1, alpha-SMA, MMP-2, CTGF, HGF, IL-1R1, IL-lbetaR, IFN-gammaR, IFN-alpha, ET-1 receptor, AT1 receptor, LPAR, PAR1, CB1, CB2, prostacyclin receptor, VIP receptor, CPB2, ELANE, relaxin receptor, SAP, integrin alpha5, TGM2, FAK1, INK, IKK, ROCK, 26S protease, caspase, PDE, cathepsin B, S100A9, procollagen-proline dioxygenase, PPAR, FXR, GR, ER, SMAD2, SMAD3, NOX1, NOX4, and EML4.

In certain embodiments, the Target Protein is a cancer related fusion protein.

In certain embodiments the Target Protein is BCAT1 and the BCAT1 mediated disorder is cancer for example gastric cancer, hepatocellular carcinoma, or brain cancer.

In certain embodiments the Target Protein is GLUL and the GLUL mediated disorder is an infectious disease.

In certain embodiments the Target Protein is FKBPlA and the FKBPlA mediated disorder is a dermatological disorder.

In certain embodiments the Target Protein is SCAF8 and the SCAF8 mediated disorder is cancer.

In certain embodiments the Target Protein is GMDS and the GMDS mediated disorder is cancer.

In certain embodiments the Target Protein is IDH1 and the IDH1 mediated disorder is cancer.

In certain embodiments the Target Protein is HPRT1 and the HPRT1 mediated disorder is cancer.

In certain embodiments the Target Protein is PYGL and the PYGL mediated disorder is cancer.

In certain embodiments the Target Protein is PYGB and the PYGB mediated disorder is cancer.

In certain embodiments the Target Protein is G6PD and the G6PD mediated disorder is a metabolic disorder, genetic disorder, dermatological disorder, immune disorder, cancer, or cardiovascular disorder.

In certain embodiments the Target Protein is NME1 and the NME1 mediated disorder is cancer.

In certain embodiments the Target Protein is PGAM1 and the PGAM1 mediated disorder is cancer.

In certain embodiments the Target Protein is YWHAQ and the YWHAQ mediated disorder is cancer or a neurodegenerative disease.

In certain embodiments the Target Protein is ADSL and the ADSL mediated disorder is cancer.

In certain embodiments the Target Protein is ATIC and the ATIC mediated disorder is cancer.

In certain embodiments the Target Protein is YWHAB and the YWHAB mediated disorder is cancer.

In certain embodiments the Target Protein is PIN1 and the PIN1 mediated disorder is cancer.

In certain embodiments the Target Protein is NUDT9 and the NUDT9 mediated disorder is cancer.

### 14-3-3 Protein Theta (YWHAQ)

In certain embodiments, the Target Protein is YWHAQ (Tyrosine 3-Monooxygenase/Tryptophan 5-Monooxygenase Activation Protein Theta). YWHAQ belongs to the 14-3-3 family of proteins which mediate signal transduction by binding to phosphoserine-containing proteins. Among its related pathways are Apoptosis Modulation and Signaling and Regulation of Wnt-mediated beta catenin signaling and target gene transcription.

In certain embodiments, the disease mediated by YWHAQ is an oncogene.

In certain embodiments, the disease mediated by YWHAQ is a neurodegenerative gene.

In certain embodiments, diseases associated with YWHAQ include, but are not limited to, amyotrophic lateral sclerosis, Creutzfeldt-Jakob Disease, cancer (for example, breast cancer, childhood acute lymphoblastic leukemia, colorectal cancer, human astrocytoma, osteosarcoma, prostate cancer, lung cancer, cervical cancer, thyroid cancer, stomach cancer, endometrial cancer, ovarian cancer, skin cancer, pancreatic cancer, esophageal cancer); neurodegenerative disease (for example, Alzheimer's disease, spinocerebellar ataxia type 1); cardiovascular disease, Diabetic kidney disease, type II diabetes and chronic degenerative diseases (for example, cardiovascular, metabolic, and neurodegenerative diseases). (Fan et al., “14-3-3 Proteins Are on the Crossroads of Cancer, Aging, and Age-Related Neurodegenerative Disease”, Int J Mol Sci., 2019 July; 20(14): 3518)

The Protein Data Bank website provides the crystal structure of YWHAQ searchable by 5IQP (Xiao et al., “Structure of a 14-3-3 protein and implications for coordination of multiple signalling pathways”, Nature, 1995, 376: 188-191); as well as the crystal structure of YWHAQ bound to various compounds searchable by 6KZH, 6KZG, 2YEZ, 6BQT, 2BTP, 6BD1, 6BD2, 6BCR, 4DNK, and 5IQP. In certain embodiments, YWHAQ binds to CIC pS173 peptide. In certain embodiments, YWHAQ binds to SEQ ID NO: 4 RTQSLSAL or is a 1, 2, 3, or 4 amino acid mutation thereof. In certain embodiments, YWHAQ binds to CIC S301 phosphorylated peptide.

### Isocitrate Dehydrogenase Cytoplasmic Protein (IDH1)

In certain embodiments, the Target Protein is IDH1. IDH1 is a Isocitrate dehydrogenases catalyze the oxidative decarboxylation of isocitrate to 2-oxoglutarate. These enzymes belong to two distinct subclasses, one of which utilizes NAD⁺ as the electron acceptor and the other NADP⁺.

In certain embodiments, the disease mediated by IDH1 is an oncogene.

In certain embodiments, the disease mediated by IDH1 is inflammation.

In certain embodiments, diseases associated with IDH1 include, but are not limited to, cancer (for example, glioma, for example, Diffuse midline glioma, pediatric diffuse gliomas; myeloid neoplasms, acute myeloid leukemia (AML), elapsed or Refractory Acute Myeloid Leukemia (AML), solid tumor, mutant tumors, Advanced Solid Tumor, chondrosarcoma, Myelodysplastic Syndromes, Recurrent Glioblastoma, Cholangiocarcinoma, Hepatocellular Carcinoma, Bile Duct Carcinoma, astrocytoma, Anaplastic Astrocytoma, malignant astrocytoma, colorectal cancer, breast cancer, prostate cancer, lung cancer, pancreatic cancer, ovarian cancer, anaplastic oligodendroglioma, non-small cell lung cancer (NSCLC), thyroid carcinoma, Glioblastoma, chronic myeloproliferative neoplasms, brain tumor, acute lymphoblastic leukemia, squamous cell carcinoma, sarcoma, conventional glioblastoma multiforme, inflammatory bowel disease-associated intestinal adenocarcinoma, Primary myelofibrosis), hematological disorders, autoimmune disorders (for example, vulvar lichen sclerosis (VLS)), Metaphyseal Enchondromatosis With D-2-Hydroxyglutaric Aciduria and Glioma Susceptibility 1, bone/skeletal disease (for example, Maffucci syndrome, Ollier disease), neurodegenerative disease (for example, Alzheimer's disease, Huntington disease); inflammatory disease (for example, inflammatory bowel disease, inflammatory bowel disease-associated intestinal adenocarcinoma) and cardiovascular disease (for example, coronary artery disease, cardiac dysfunction, cardiotoxicity). (Tommasini-Ghelfi et al., “Cancer-associated mutation and beyond: The emerging biology of isocitrate dehydrogenases in human disease”, Sci Adv., 2019 May; 5(5): eaaw4543; Krell D, et al., “IDH mutations in tumorigenesis and their potential role as novel therapeutic targets”, Future Oncol., 2013; 9(12):1923-1935; Prensner J R, Chinnaiyan A M. “Metabolism unhinged: IDH mutations in cancer”, Nat Med., 2011; 17(3):291-293; McKenney A S, Levine R L. “Isocitrate dehydrogenase mutations in leukemia”, J Clin Invest., 2013; 123(9):3672-3677”; Amary M F, et al., “Ollier disease and Maffucci syndrome are caused by somatic mosaic mutations of IDH1 and IDH2”, Nat Genet., 2011 Nov. 6; 43(12):1262-5)

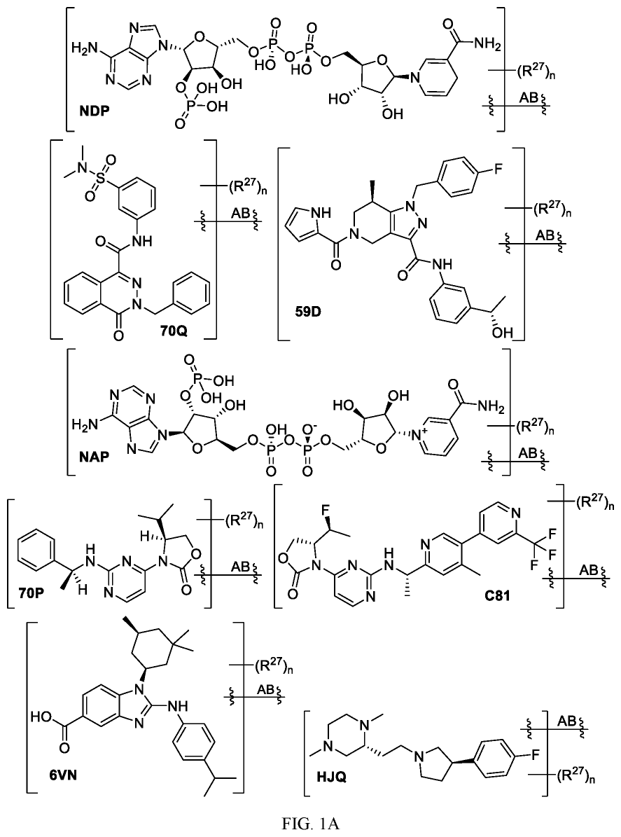

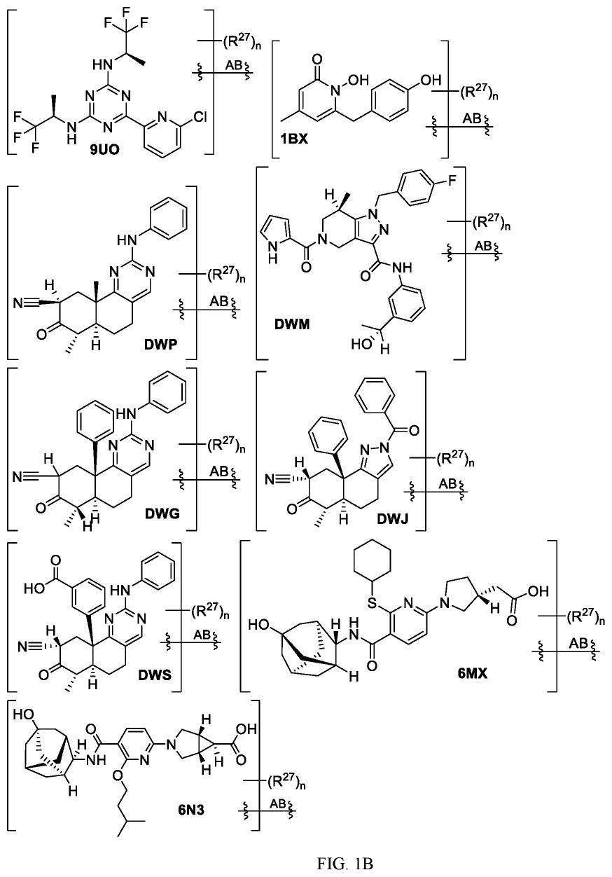

The Protein Data Bank website provides the crystal structure of IDH1 searchable by 3BLX (Taylor et al., “Allosteric Motions in Structures of Yeast NAD+-specific Isocitrate Dehydrogenase”, J Biol Chem., 2008, 283: 10872-10880); as well as the crystal structure of IDH1 bound to various compounds searchable by 5SUN, 5SVO, 5SVF, 4L03, 4L03, 4L04, 4L06, 4KZO, 5DE1, 6B0Z, 5LGE, 6Q6F, 6ADG, 4I3K, 6BKX, 6BKZ, 6BL1, 6BL0, 6BL2, 5L58, 5L57, 3MAP, 3MAR, 3MAS, 5TQH, 4UMX, 4UMY, 4XRX, 4XS3, 602Y, 602Z, 6I00, 3INM, 6VEI, 6VGO, 5YFM, 4JA8, 5195, and 5196. Representative IDH1 Targeting Ligands are provided in FIG. 1A, 1B and 1C.

### Phosphoglycerate Mutase 1 (PGAM1)

In certain embodiments, the Target Protein is PGAM1 is a protein coding gene. PGAM1 belongs to the phosphoglycerate mutase family, and is an enzyme that catalyzes the reversible conversion of 3-phosphoglycerate and 2-phosphoglycerate during the process of glycolysis.

In certain embodiments, the disease mediated by PGAM1 is an oncogene.

In certain embodiments, diseases associated with PGAM1 includes, but are not limited to, cancer (prostate cancer, renal cancer, head and neck cancer, pancreatic cancer, breast cancer, glioma, leukemia, lung cancer, non-small cell lung cancer (NSCLC), Hepatocellular carcinoma, Urothelial bladder cancer, glioblastoma, pancreatic ductal adenocarcinoma), neurological/neurodegenerative disorder (for example, Alzheimer's disease, Corticobasal Degeneration, cerebral ischemia, phenylketonuria, hypoxia, schizophrenia), genetic disorders (for example, Menkes disease), metabolic myopathy, and glycogen storage disease (for example, affecting skeletal muscle). (Li et al., “Phosphoglycerate Mutase 1: Its Glycolytic and Non-Glycolytic Roles in Tumor Malignant Behaviors and Potential Therapeutic Significance”, Onco Targets Ther. 2020; 13: 1787-1795.)

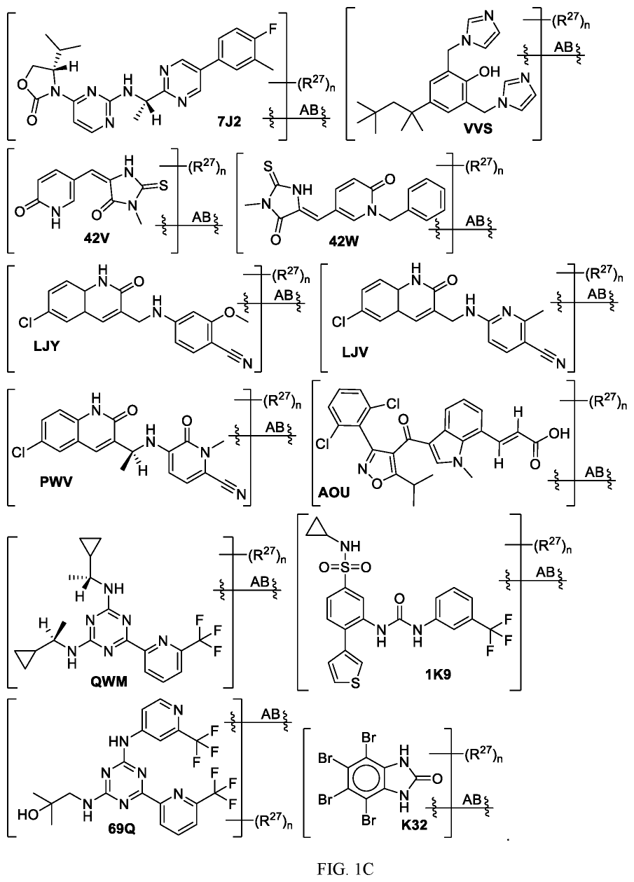

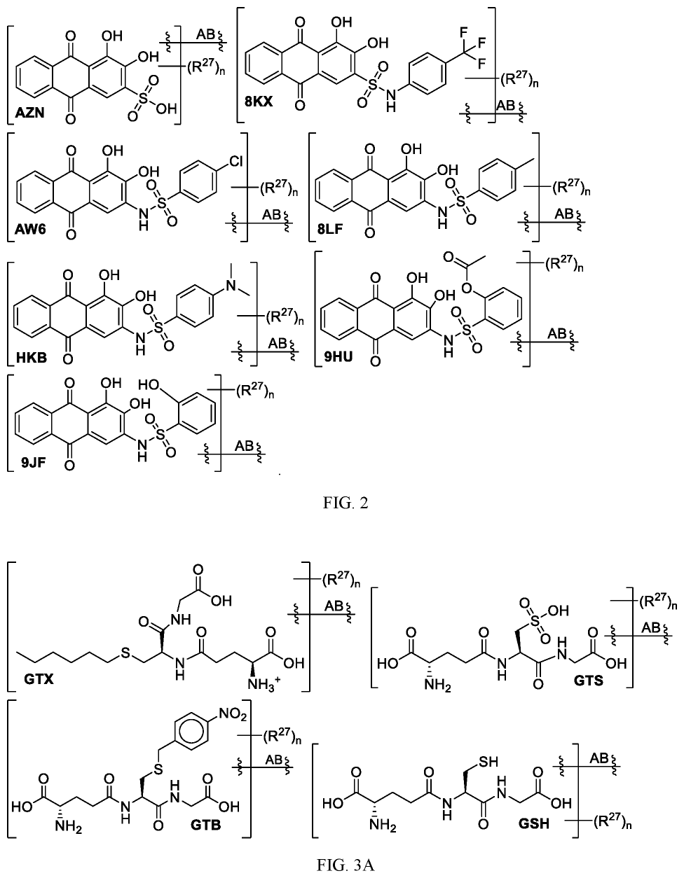

The Protein Data Bank website provides the crystal structure of PGAM1 searchable by 4GPZ and 4GPI (Hitosugi et al., “Tyr26 phosphorylation of PGAM1 provides a metabolic advantage to tumours by stabilizing the active conformation”, Nat Commun., 2013, 4: 1790-1790); as well as the crystal structure of PGAM1 bound to various compounds searchable by 5Y2U, 5Y2I, 6ISN, 5Y35, 5Y64, 5Y65, 5ZRM, and 5ZS8. Representative PGAM1 Targeting Ligands are provided in FIG. 2.

### Glutathione S-Transferase P (GSTP1)

In certain embodiments, the Target Protein is GSTP1 or GST pi, a protein coding gene and an enzyme involved in cell protection against toxic electrophiles and products of oxidative stress. GSTP1 is part of a family of enzymes that play an important role in detoxification by catalyzing the conjugation of many hydrophobic and electrophilic compounds with reduced glutathione.

In certain embodiments, the disease mediated by GSTP1 is a cancer.

In certain embodiments, the disease mediated by GSTP1 is non-small cell lung cancer, pancreatic cancer, colon cancer, and the like.

In certain embodiments, diseases associated with GSTP1 includes, but are not limited to, cancer (for example, prostate cancer, hepatocellular carcinoma, breast cancer, pancreatic cancer, squamous cell cancer, colorectal cancer, lung cancer, leukemia, acute leukemia, chronic myeloid leukemia, acute myeloid leukemia, acute lymphoblastic leukemia, bladder cancer, multiple myeloma, non-small cell lung cancer (NSCLC), gastric cancer, oral cancer, ovarian cancer, cervical cancer, glioma, Hodgkin's lymphoma, rectal cancer, melanoma, bladder cancer, skin cancer, esophageal cancer, head and neck cancer, endometrial carcinoma, osteosarcoma, brain tumor, lung squamous-cell carcinoma), respiratory disease/disorders (for example, asthma, Chronic obstructive pulmonary disease, antitrypsin deficiency), diabetes (for example, Type II diabetes, human obesity, nonalcoholic fatty liver disease), neurological/neurodegenerative disease (for example, Alzheimer's disease, Parkinson's disease, schizophrenia, Amyotrophic lateral sclerosis, epilepsy), inflammatory related disease (for example, heart failure); autistic disorder, and cardiovascular disease. (Allocati et al., “Glutathione transferases: substrates, inhibitors and pro-drugs in cancer and neurodegenerative diseases”, Oncogenesis, 2018, 7, 8; Shi et al., “Identification of Glutathione S-Transferase Pi as a Protein Involved in Parkinson Disease Progression”, Am J Pathol., 2009 July; 175(1): 54-65; Williams T A, et al. “Risk of autistic disorder in affected offspring of mothers with a glutathione s-transferase p1 haplotype”, Arch Pediatr Adolesc Med., 2007; 161(4):356-361)

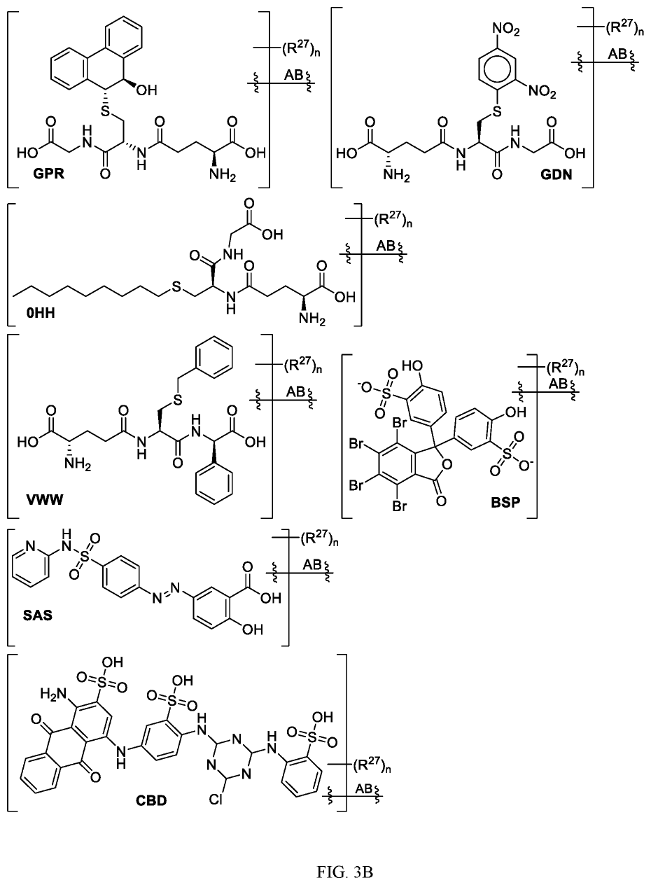

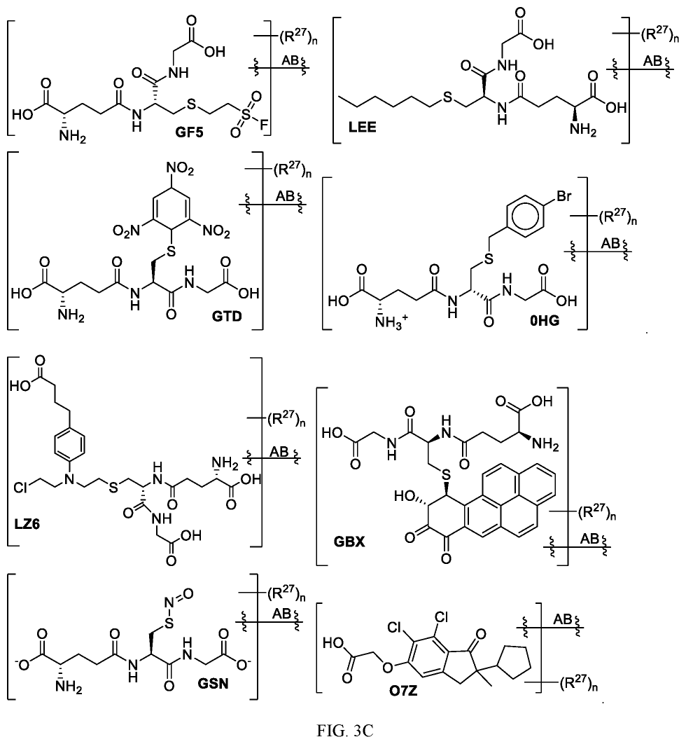

The Protein Data Bank website provides the crystal structure of GSTP1 searchable by 1EOG, 1EOH, and 17GS (Rossjohn et al., “Structures of thermolabile mutants of human glutathione transferase P1-1”, J Mol Biol., 2000, 302: 295-302; Oakley et al., “Glutathione S-transferase P1-1”, to be published); as well as the crystal structure of GSTP1 bound to various compounds searchable by 20A7, 2OAC, 2OAD, 1GLQ, 1GSY, 1GTI, 2GLR, 1GLP, 1PGT, 7GSS, 18GS, 12GS, 10GS, 19GS, 13GS, 20GS, 5X79, 1GSS, 1AQX, 1AQV, 3CSH, 3PGT, and 2A2R. Representative GSTP1 Targeting Ligands are provided in FIG. 3A, 3B, and 3C.

### Nucleoside Diphosphate Kinase B (NME2)

In certain embodiments, the Target Protein is nucleoside diphosphate kinase B (NME2). Nme2 (non metastasis protein 2, or non metastasis 2) is a nucleoside diphosphate kinase that plays multiple roles in signalling and metabolism. Nucleoside diphosphate kinase B is an enzyme that in humans is encoded by the NME2 gene, and is a non-metastatic cells gene. NME2 is identical to the beta subunit of human erythrocyte NDP kinase.

In certain embodiments, the disease mediated by NME2 is a cancer.

In certain embodiments, the disease mediated by NME2 is gastric cancer.

In certain embodiments, diseases associated with NME2 include, but are not limited to, Cancer (for example, endometrium carcinoma in situ, melanoma, breast carcinoma, breast cancer, prostate cancer, lung cancer, ovarian cancer, cervical cancer, pituitary cancer, ovarian carcinoma, colorectal neoplasm, skin neoplasm, leukemia, acute leukemia, myeloid leukemia, chronic myeloid leukemia (CML), acute myelocytic leukemia, neuroblastoma, urinary bladder cancer, teratoma, Germ Cell and Embryonal Neoplasms, mesothelioma, pleural mesothelioma, gastric cancer, osteosarcoma, glioma, neoplasm metastasis, malignant neoplasm, malignant paraganglionic neoplasm, pleural neoplasm, malignant tumor of the cervix, adenocarcinoma, neurofibromatosis 1), autoimmune disease (for example, Nemaline Myopathy 2, renal carcinoma), cardiovascular disease (for example, heart failure, congestive heart failure), bone disease (for example, arteriosclerosis, osteoarthritis), and neurodegenerative diseases (for example, Alzheimer's disease (AD) and Down syndrome (DS)). (Chang et al., “CARMA3 Represses Metastasis Suppressor NME2 to Promote Lung Cancer Stemness and Metastasis”, American Journal of Respiratory and Critical Care Medicine, 2015, 192(1), 64-75; Liu et al., “NME2 Reduces Proliferation, Migration and Invasion of Gastric Cancer Cells to Limit Metastasis”, Plos One, 2015, 10(2): e0115968; Li et al., “Nucleoside diphosphate kinase B promotes osteosarcoma proliferation through c-Myc”, Cancer Biology & Therapy, 2018, 19(7), 565-572; https://www.bosterbio.com/bosterbio-gene-info-cards/NME2. Kim et al., “Human brain nucleoside diphosphate kinase activity is decreased in Alzheimer's disease and Down syndrome”, Biochemical and Biophysical Research Communications, 2002, 296(4):970-5)

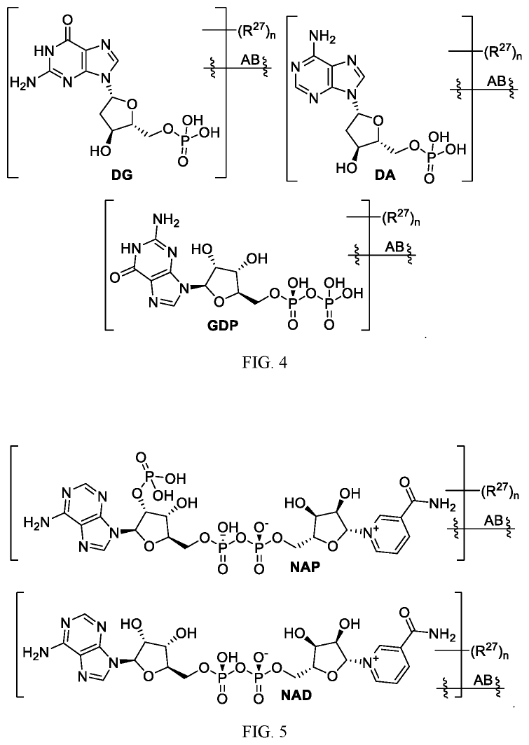

The Protein Data Bank website provides the crystal structure of NME2 searchable by INSK (Webb et al., “The crystal structure of a human nucleoside diphosphate kinase, NM23-H2”, J Mol Biol., 1995, 251: 574-587); as well as the crystal structure of NME2 bound to various compounds searchable by 3BBB, 3BBF, and 1NUE. Representative NME2 Targeting Ligands are provided in FIG. 4.

### Biliverdin Reductase A (BLVRA)

In certain embodiments, the Target Protein is Biliverdin reductase A (BLVRA or BVRA). BLVRA also called Biliverdin-IX-alpha reductase, reduces the gamma-methene bridge of the open tetrapyrrole, biliverdin IX alpha, to bilirubin with the concomitant oxidation of a NADH or NADPH cofactor. Biliverdin reductase-A is a pleiotropic enzyme involved not only in the reduction of biliverdin-IX-alpha into bilirubin-IX-alpha, but also in the regulation of glucose metabolism and cell growth secondary to its serine/threonine/tyrosine kinase activity.

In certain embodiments, the disease mediated by BLVRA is a cancer.

In certain embodiments, the disease mediated by BLVRA is an inflammatory disease.

In certain embodiments, diseases associated with BLVRA include, but are not limited to, cancer (for example, glioma, glioblastoma, thyroid cancer, head and neck cancer, pancreatic cancer, renal cancer, breast cancer, prostate cancer, cervical cancer, skin cancer, pancreatic cancer, urothelial cancer, endometrial cancer, melanoma, lymphoma, ovarian cancer, carcinoid, hepatocellular cancer, leukemia), viral infection (for example, Hepatitis C), Hyperbilirubinemia, cholestasis, multiple sclerosis, obesity, neurodegenerative/neurological disease (for example, Alzheimer's disease, mild cognitive impairment, Parkinson's disease, neonatal hemorrhagic stroke, hematoma), kidney disease, hepatic disease (for example, hepatic fibrosis, cirrhosis, nonalcoholic fatty liver disease, hepatic steatosis, hepatocellular carcinoma), coronary artery disease, retinopathy of prematurity, inflammatory disease (for example, inflammatory skin disease, inflammatory liver disease, immunity-mediated inflammation), and stress-mediated diseases. (Barone et al., “Biliverdin reductase—a protein levels and activity in the brains of subjects with Alzheimer disease and mild cognitive impairment”, Biochim Biophys Acta, 2011 April; 1812(4):480-7; Hu et al., “Biliverdin reductase A (BVRA) mediates macrophage expression of interleukin-10 in injured kidney”, International Journal of Molecular Sciences (2015), 16(9), 22621-22635)

The Protein Data Bank website provides the crystal structure of BLVRA searchable by 2H63 (Kavanagh et al., “Crystal Structure of Human Biliverdin Reductase A”, to be published); as well as the crystal structure of BLVRA bound to various compounds searchable by 2H63 and 1LC3. Representative BLVRA Targeting Ligands are provided in FIG. 5.

### Rac Family Small GTPase 3 (RAC3).

In certain embodiments, the Target Protein is Ras-related C3 botulinum toxin substrate 3 (RAC3). RAC3 is a GTPase which belongs to the RAS superfamily of small GTP-binding proteins. Members of this superfamily appear to regulate a diverse array of cellular events, including the control of cell growth, cytoskeletal reorganization, and the activation of protein kinases. Rac3 protein is co-expressed with Rac1 in developing neurons and in other cell types, with a pattern of expression more restricted compared to Rac1.

In certain embodiments, the disease mediated by RAC3 is breast cancer.

In certain embodiments, the disease mediated by RAC3 is n neurodegenerative disease.

In certain embodiments, diseases associated with RAC3 include, but are not limited to, cancer (for example, breast cancer, gastric cancer, colorectal cancer, hepatocellular carcinoma, gall bladder cancer, pancreatic cancer, prostate cancer, lymphoma, leukemia, lymphoblastic leukemia, chronic myeloid leukemia, myeloma, osteosarcoma, ovarian cancer, uterine endometrial cancer, lung carcinoma, hypopharyngeal squamous cell carcinoma, glioblastoma, lung adenocarcinoma, esophageal cancer, brain tumor), neurological/neurodevelopment disorders (for example, neurodevelopmental disorder with structural brain anomalies, dysmorphic facies, amyotrophic lateral sclerosis (ALS), Alzheimer's disease, Huntington's disease (HD), Parkinson's Disease, Charcot marie-tooth disease, spinal cord injury, Cerebral ischemia and reperfusion), deafness, autosomal recessive 104. (Curtis et al., “The Rac3 GTPase in Neuronal Development, Neurodevelopmental Disorders, and Cancer”, Cells, 2019 September; 8(9): 1063; Yan et al., “SRC-3/AIB1: transcriptional coactivator in oncogenesis”, Acta Pharmacologica Sinica (2006), 27(4), 387-394; Pai et al., “Rac GTPases in human diseases”, Disease Markers (2010), 29(3,4), 177-187; Usman et al., “Role and mechanism of autophagy-regulating factors in tumorigenesis and drug resistance”, Asia-Pacific journal of clinical oncology, 2020; Stankiewicz et al., “Rho family GTPases: key players in neuronal development, neuronal survival, and neurodegeneration”, Front Cell Neurosci. 2014; 8: 314)

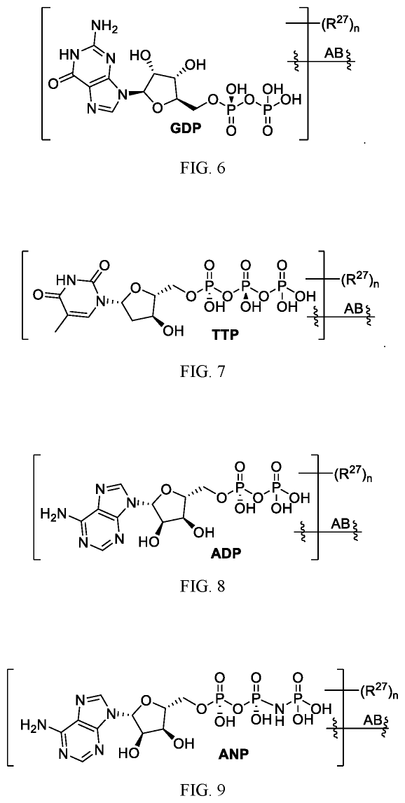

The Protein Data Bank website provides the crystal structure of RAC3 searchable by 2C2H (Kavanagh et al., “Crystal Structure of Human Biliverdin Reductase A”, to be published); as well as the crystal structure of RAC3 bound to various compounds searchable by 2C2H. Representative RAC3 Targeting Ligands are provided in FIG. 6.

### Thymidine Kinase, Cytosolic (TK1)

In certain embodiments, the Target Protein is Thymidine kinase, cytosolic (TK1).

TK1 is a cytosolic enzyme that catalyzes the addition of a gamma-phosphate group to thymidine. This creates dTMP and is the first step in the biosynthesis of dTTP, which is one component required for DNA replication. The encoded protein, whose levels fluctuate depending on the cell cycle stage, can act as a low activity dimer or a high activity tetramer. High levels of this protein have been used as a biomarker for diagnosing and categorizing many types of cancers (for example, a biomarker for CDK4/6 cancers). (McCartney A, et al., “Potential through simplicity: thymidine kinase-1 as a biomarker for CDK4/6 inhibitors”, Br J Cancer, 2020 July PMID 32382111)

In certain embodiments, the disease mediated by TK1 is cancer.

In certain embodiments, the disease mediated by TK1 is breast cancer, thyroid cancer, skin cancer, cervical cancer, lymphoma, liver cancer, pancreatic cancer, bladder cancer, or colon cancer.

In certain embodiments, diseases associated with TK1 include, but are not limited to, cancer (for example, breast cancer, metastatic breast cancer, ovarian cancer, cervical cancer, hepatocellular cancer, prostate cancer, non-small cell lung cancer, colorectal cancer, head and neck cancer, endometrial cancer, skin cancer, gastrointestinal cancer, lung cancer, pancreatic cancer, colon cancer, bladder cancer, small-cell lung cancer (SCLC), thyroid cancer, lung adenocarcinoma, malignant peripheral nerve sheath tumors (MPNST), malignant tumor, lymphoma, solid tumor, ovarian serous adenocarcinoma, brain tumor, leukemia, chronic lymphatic leukemia, glioma), inflammatory disease, autoimmune disease, Hypochondroplasia and Thanatophoric Dysplasia, Type I, thyroid disease. (Jagarlamudi et al., “Thymidine kinase 1 as a tumor biomarker: technical advances offer new potential to an old biomarker”, Biomarkers in Medicine (2018), 12(9), 1035-1048; Topolcan et al., “The role of thymidine kinase in cancer diseases”, Expert Opinion on Medical Diagnostics (2008), 2(2), 129-141; O-Neill et al., “Thymidine kinase: Diagnostic and prognostic potential”, Expert Review of Molecular Diagnostics (2001), 1(4), 428-433; Hallek et al., “Thymidine kinase: a tumor marker with prognostic value for non-Hodgkin's lymphoma and a broad range of potential clinical applications”, Annals of hematology (1992), 65(1), 1-5; Deng et al., “Application of thymidine kinase 1 in the cancer diseases”, Redai Yixue Zazhi (2009), 9(9), 1084-1087; Malvi P, et al., “Loss of thymidine kinase 1 inhibits lung cancer growth and metastatic attributes by reducing GDF15 expression”, 2019, PLoS Genet 15(10): e1008439; bitter et al., “Thymidine kinase 1 through the ages: a comprehensive review”, Cell Biosci., 2020, 10, 138)

The Protein Data Bank website provides the crystal structure of TK1 searchable by 1XBT (Welin et al., “Structures of thymidine kinase 1 of human and mycoplasmic origin”, Proc Natl Acad Sci USA., 2004, 101: 17970-17975); as well as the crystal structure of TK1 bound to various compounds searchable by 1XBT. Representative TK1 Targeting Ligands are provided in FIG. 7.

### Glutamine Synthetase (GS)

In certain embodiments, the Target Protein is Glutamine synthetase (GLUL or GS). GLUL or GS is an enzyme that plays an essential role in the metabolism of nitrogen by catalyzing the condensation of glutamate and ammonia to form glutamine:

In certain embodiments, the disease mediated by GS is a cancer. In certain embodiments, the cancer is gastric cancer, hepatocellular carcinoma, glioma, and the like.

In certain embodiments, the disease mediated by GS is an inflammatory disease.

In certain embodiments, the disease mediated by GS includes, but is not limited to, Cirrhosis or Urea Cycle Disorder.

The Protein Data Bank website provides the crystal structure of GS searchable by 5ZLI (Joo et al., “Structural Analysis of Glutamine Synthetase from Helicobacter pylori”, Sci Rep., 2018, 8: 11657-11657); as well as the crystal structure of GS bound to various compounds searchable by 2QC8. Representative GS Targeting Ligands are provided in FIG. 8.

### Eukaryotic Initiation Factor 4A-III (EIF4A3)

In certain embodiments, the Target Protein is Eukaryotic initiation factor 4A-III (EIF4A3). EIF4A3 is a protein that in humans is encoded by the EIF4A3 gene. This gene encodes a member of the DEAD box protein family. DEAD box proteins, characterized by the conserved motif Asp-Glu-Ala-Asp (DEAD), are putative RNA helicases.

In certain embodiments, the disease mediated by EIF4A3 is a cancer.

In certain embodiments, the disease associated with EIF4A3 include, but are not limited to, Robin Sequence with Cleft Mandible and Limb Anomalies and Schopf-Schulz-Passarge Syndrome; cancer (for example, breast cancer, glioblastoma. Chronic lymphocytic leukemia, neoplasm, ovarian cancer, non-small cell lung cancer, colorectal cancer), craniofacial disorder (for example, Richieri-Costa-Pereira syndrome).

The Protein Data Bank website provides the crystal structure of EIF4A3 searchable by 2J0U and 2HXY (Bono et al., “The Crystal Structure of the Exon Junction Complex Reveals How It Mantains a Stable Grip on Mrna”, Cell, 2006, 126: 713; Anderson et al., “Structure of the exon junction core complex with a trapped DEAD-box ATPase bound to RNA”, Science, 2006, 313: 1968-1972); as well as the crystal structure of EIF4A3 bound to various compounds searchable by 2JOS, 2JOQ, and 2HYI. Representative EIF4A3 Targeting Ligands are provided in FIG. 9.

### Hypoxanthine-Guanine Phosphoribosyltransferase (HPRT or HPRT1)

In certain embodiments, the Target Protein is Hypoxanthine-guanine phosphoribosyltransferase (HPRT or HPRT1). The protein encoded by this gene is a transferase, which catalyzes conversion of hypoxanthine to inosine monophosphate and guanine to guanosine monophosphate via transfer of the 5-phosphoribosyl group from 5-phosphoribosyl 1-pyrophosphate. This enzyme plays a central role in the generation of purine nucleotides through the purine salvage pathway.

In certain embodiments, the disease mediated by HPRT1 is a cancer.

In certain embodiments, the disease mediated by HPRT1 include, but is not limited to Lesch-Nyhan syndrome, gout, or Kelley-Seegmiller Syndrome.

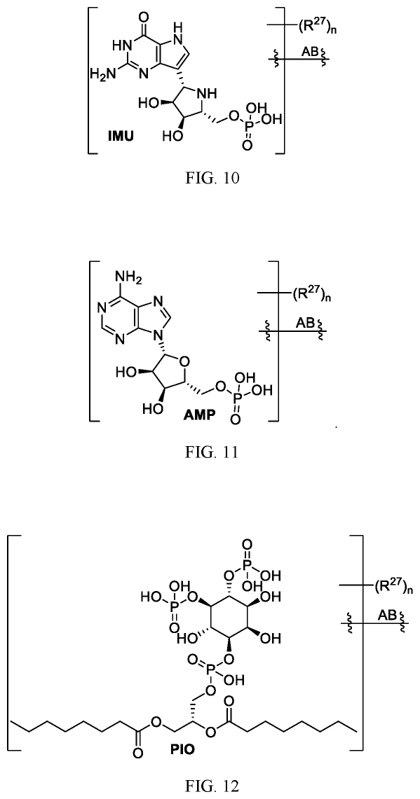

The Protein Data Bank website provides the crystal structure of HPRT1 searchable by 1Z7G (Keough et al., “The Crystal Structure of Free Human Hypoxanthine-guanine Phosphoribosyltransferase Reveals Extensive Conformational Plasticity Throughout the Catalytic Cycle”, J Mol Biol., 2005, 351: 170-181); as well as the crystal structure of HPRT1 bound to various compounds searchable by 1BZY. Representative HPRT1 Targeting Ligands are provided in FIG. 10.

### Glycogen Phosphorylase, Brain Form (PYGB)

In certain embodiments, the Target Protein is Glycogen phosphorylase, brain form (PYGB). PYGB is encoded by this gene is a glycogen phosphorylase found predominantly in the brain. The encoded protein forms homodimers which can associate into homotetramers, the enzymatically active form of glycogen phosphorylase.

In certain embodiments, the disease mediated by PYGB is a cancer.

In certain embodiments, the disease mediated by PYGB is a neurodegenerative disease or disorder.

In certain embodiments, diseases associated with PYGB include, but are not limited to, Glycogen Storage Disease Viii and Glycogen Storage Disease Iii.

The Protein Data Bank website provides the crystal structure of PYGB searchable by 5IKO (Mathieu et al., “Insights into brain glycogen metabolism: the structure of human brain glycogen phosphorylase”, J Biol Chem., 2016, 291: 18072-18083); as well as the crystal structure of PYGB bound to various compounds searchable by 5IKP. Representative PYGB Targeting Ligands are provided in FIG. 11.

### Vinculin (VCL)

In certain embodiments, the Target Protein is Vinculin (VCL). VCL is a cytoskeletal protein associated with cell-cell and cell-matrix junctions, where it is thought to function as one of several interacting proteins involved in anchoring F-actin to the membrane.

In certain embodiments, the disease mediated by VCL is a cancer. In certain embodiments, the cancer is thyroid cancer, lung cancer, Urothelial cancer, colorectal cancer, head and neck cancer, stomach cancer, liver cancer, pancreatic cancer, testicular cancer, breast cancer, cervical cancer, endometrial cancer, ovarian cancer, skin cancer, glioma, or melanoma.

In certain embodiments, the disease mediated by VCL is cardiovascular disease.

In certain embodiments, the disease mediated by VCL is immunological disease from pathogens causing gastroenteritis.

In certain embodiments, diseases associated with VCL include, but are not limited to, Cardiomyopathy, Dilated, 1W and Cardiomyopathy, Familial Hypertrophic, 15, Acute gastroenteritis.

The Protein Data Bank website provides the crystal structure of VCL searchable by 1QKR and 5L0J (Bakolitsa et al., “Crystal Structure of the Vinculin Tail and a Pathway for Activation”, Cell., 1999, 99: 603; Chinthalapudi et al., “Differential lipid binding of vinculin isoforms promotes quasi-equivalent dimerization”, Proc Natl Acad Sci USA., 2016, 113: 9539-9544); as well as the crystal structure of VCL bound to various compounds searchable by 5L0C and 5L0D. Representative VCL Targeting Ligands are provided in FIG. 12.

### Branched-Chain-Amino-Acid Aminotransferase, Cytosolic (BCAT1)

In certain embodiments, the Target Protein is cytosolic Branched-chain-amino-acid aminotransferase (BCAT1). BCAT1 gene encodes the cytosolic form of the enzyme branched-chain amino acid transaminase. This enzyme catalyzes the reversible transamination of branched-chain alpha-keto acids to branched-chain L-amino acids essential for cell growth.

In certain embodiments, the disease mediated by BCAT1 is a cancer. In certain embodiments, the cancer is gastric cancer, hepatocellulcar cancer, thyroid cancer, endometrial cancer, skin cancer, melanoma, testicular cancer, or glioma.

In certain embodiments, diseases associated with BCAT1 include, but are not limited to, Hyperleucine-Isoleucinemia and Hypervalinemia and Hyperleucine-Isoleucinemia.

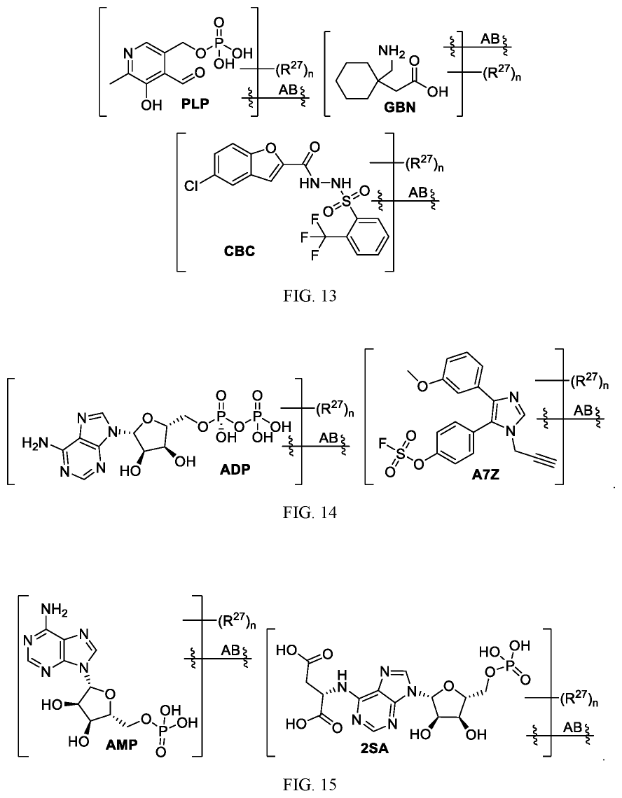

The Protein Data Bank website provides the crystal structure of BCAT1 searchable by 6NST and 1A3G (Chang et al., “Crystal structure of branched chain amino acid aminotransferase from Pseudomonas aeruginosa”, to be published; Okada et al., “Three-dimensional structure of Escherichia coli branched-chain amino acid aminotransferase at 2.5 A resolution”, J Biochem., 1997, 121: 637-641); as well as the crystal structure of BCAT1 bound to various compounds searchable by 2COJ, 2COI, 2COG, 2A1H, and 2ABJ. Representative BCAT1 Targeting Ligands are provided in FIG. 13.

### Nucleoside Diphosphate Kinase a (NME1)

In certain embodiments, the Target Protein is Nucleoside diphosphate kinase A (NME1). NME1 gene was identified because of its reduced mRNA transcript levels in highly metastatic cells. Nucleoside diphosphate kinase (NDK) exists as a hexamer composed of ′A.

In certain embodiments, the disease mediated by NME1 is cancer. In certain embodiments, the cancer is thyroid cancer, colorectal cancer, head and neck cancer, stomach cancer, pancreatic cancer, prostate cancer, Urothelial cancer, testicular cancer, breast cancer, endometrial cancer, ovarian cancer, melanoma, skin cancer, lymphoma, liver cancer, glioma, Anal Canal Carcinoma, neuroblastoma, or Larynx Cancer, and the like.

The Protein Data Bank website provides the crystal structure of NME1 searchable by 1BHN (Ladner et al., “The three-dimensional structures of two isoforms of nucleoside diphosphate kinase from bovine retina”, Acta Crystallogr D Biol Crystallogr., 1999, 55: 1127-1135); as well as the crystal structure of NME1 bound to various compounds searchable by 2HVD, 2HVE and 5UI4. Representative NME1 Targeting Ligands are provided in FIG. 14.

### Adenylosuccinate Lyase (ADSL)

In certain embodiments, the Target Protein is Adenylosuccinate lyase (ADSL). The protein encoded by this gene belongs to the lyase 1 family. It is an essential enzyme involved in purine metabolism, and catalyzes two non-sequential reactions in the de novo purine biosynthetic pathway: the conversion of succinylaminoimidazole carboxamide ribotide (SAICAR) to aminoimidazole carboxamide ribotide (AICAR) and the conversion of adenylosuccinate (S-AMP) to adenosine monophosphate (AMP).

In certain embodiments, the disease mediated by ADSL is a cancer. In certain embodiments, the cancer is thyroid cancer, lung cancer, colorectal cancer, head and neck cancer, stomach cancer, liver cancer, pancreatic cancer, renal cancer, Urothelial cancer, prostate cancer, testicular cancer, breast cancer, cervical cancer, endometrial cancer, ovarian cancer, skin cancer, glioma, lymphoma, or melanoma.

In certain embodiments, diseases associated with ADSL include, but are not limited to, Adenylosuccinase Deficiency and Histidinemia.

The Protein Data Bank website provides the crystal structure of ADSL searchable by 1P9B and 1ADI (Eaazhisai et al., “Crystal Structure of Fully Ligated Adenylosuccinate Synthetase from Plasmodium falciparum”, J Mol Biol., 2004, 335: 1251-1264; Silva et al., “Refined crystal structures of unligated adenylosuccinate synthetase from Escherichia coli”, J Mol Biol., 1995, 254: 431-446); as well as the crystal structure of ADSL bound to various compounds searchable by 2J91 and 2VD6. Representative ADSL Targeting Ligands are provided in FIG. 15.

### ADP-Ribose Pyrophosphatase, Mitochondrial (NUDT9)

In certain embodiments, the Target Protein is ADP-ribose pyrophosphatase, mitochondrial (NUDT9). The protein encoded by this gene belongs to the Nudix hydrolase family. Nudix boxes are found in a family of diverse enzymes that catalyze the hydrolysis of nucleoside diphosphate derivatives. This enzyme is an ADP-ribose pyrophosphatase that catalyzes the hydrolysis of ADP-ribose to AMP and ribose-5-P.

In certain embodiments, the disease mediated by NUDT9 is a cancer. In certain embodiments, the cancer is thyroid cancer, lung cancer, colorectal cancer, head and neck cancer, stomach cancer, liver cancer, pancreatic cancer, renal cancer, Urothelial cancer, prostate cancer, testicular cancer, breast cancer, cervical cancer, endometrial cancer, ovarian cancer, skin cancer, glioma, lymphoma, or melanoma.

In certain embodiments, diseases associated with NUDT9 include, but are not limited to, Psoriasis 11 and Type 1 Diabetes Mellitus 10.

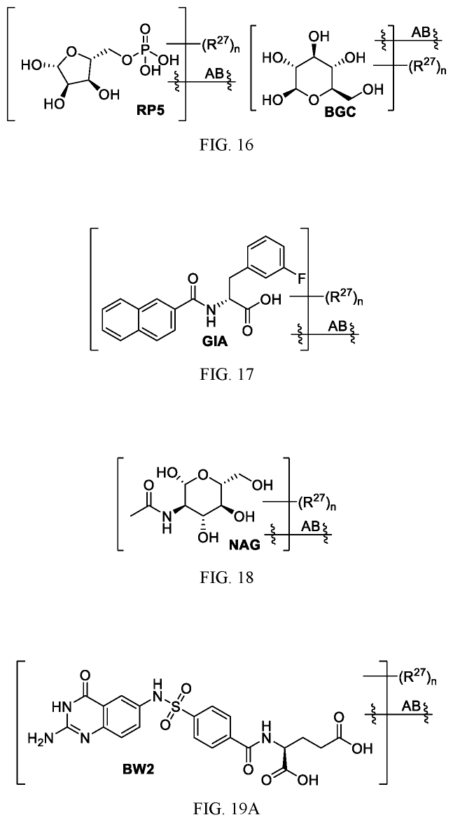

The Protein Data Bank website provides the crystal structure of NUDT9 searchable by 1Q33 (Shen et al., “The crystal structure and mutational analysis of human NUDT9”, J Mol Biol., 2003, 332: 385-398); as well as the crystal structure of NUDT9 bound to various compounds searchable by 1QVJ and 1Q33. Representative NUDT9 Targeting Ligands are provided in FIG. 16.

### Peptidyl-Prolyl Cis-Trans Isomerase NIMA-Interacting 1 (PIN1)

In certain embodiments, the Target Protein is Peptidyl-prolyl cis-trans isomerase NIMA-interacting 1 (PIN1). Peptidyl-prolyl cis/trans isomerases (PPIases) catalyze the cis/trans isomerization of peptidyl-prolyl peptide bonds. This gene encodes one of the PPIases, which specifically binds to phosphorylated ser/thr-pro motifs to catalytically regulate the post-phosphorylation conformation of its substrates.

In certain embodiments, the disease mediated by PIN1 is a cancer. In certain embodiments, the cancer is Lower Gum Cancer, glioma, carcinoid, prostate cancer, breast cancer, Gastric or Liposarcoma.

In certain embodiments, the disease mediated by PIN1 is a neurodegenerative disease.

In certain embodiments, the disease mediated by PIN1 is an infectious disease.

In certain embodiments, diseases associated with PIN1 include, but are not limited to, asthma.

The Protein Data Bank website provides the crystal structure of PIN1 searchable by 1F8A (Verdecia et al., “Structural basis for phosphoserine-proline recognition by group IV WW domains”, Nat Struct Biol., 2000, 7: 639-643); as well as the crystal structure of PIN1 bound to various compounds searchable by 2ITK and 3I6C. Representative PIN1 Targeting Ligands are provided in FIG. 17.

### 14-3-3 Protein Beta/Alpha (YWHAB)

In certain embodiments, the Target Protein is 14-3-3 protein beta/alpha (YWHAB). This gene encodes a protein belonging to the 14-3-3 family of proteins, members of which mediate signal transduction by binding to phosphoserine-containing proteins.

In certain embodiments, the disease mediated by YWHAB is a cancer. In certain embodiments, the cancer is thyroid cancer, lung cancer, colorectal cancer, head and neck cancer, stomach cancer, liver cancer, pancreatic cancer, renal cancer, Urothelial cancer, prostate cancer, testicular cancer, breast cancer, cervical cancer, endometrial cancer, ovarian cancer, skin cancer, glioma, lymphoma, or melanoma.

In certain embodiments, the disease mediated by YWHAB is a neurodegenerative disease.

In certain embodiments, the disease mediated by YWHAB is an infection.

In certain embodiments, diseases associated with YWHAB include, but are not limited to, Chlamydia and Eosinophilic Meningitis.

The Protein Data Bank website provides the crystal structure of YWHAB searchable by 4DNK and 2BQ0 (Joint Center for Structural Genomics (JCSG), Partnership for T-Cell Biology (TCELL) “Crystal structure of a tyrosine 3-monooxygenase/tryptophan 5-monooxygenase activation protein, beta polypeptide (YWHAB) from Homo sapiens at 2.20 A resolution”, to be published; Yang et al., “Structural Basis for Protein-Protein Interactions in the 14-3-3 Protein Family”, Proc Natl Acad Sci USA., 2006, 103: 17237); as well as the crystal structure of YWHAB bound to various compounds searchable by 5N10, 6BYK and 6HEP. Representative YWHAB Targeting Ligands are provided in FIG. 18.

### Bifunctional Purine Biosynthesis Protein ATIC (ATIC)

In certain embodiments, the Target Protein is Bifunctional purine biosynthesis protein ATIC (ATIC). This gene encodes a bifunctional protein that catalyzes the last two steps of the de novo purine biosynthetic pathway. The N-terminal domain has phosphoribosylaminoimidazole carboxamide formyltransferase activity, and the C-terminal domain has IMP cyclohydrolase activity.

In certain embodiments, the disease mediated by ATIC is a cancer. In certain embodiments, the cancer is thyroid cancer, lung cancer, colorectal cancer, head and neck cancer, stomach cancer, liver cancer, pancreatic cancer, renal cancer, Urothelial cancer, prostate cancer, testicular cancer, breast cancer, cervical cancer, endometrial cancer, ovarian cancer, skin cancer, glioma, lymphoma, or melanoma.

In certain embodiments, diseases associated with ATIC include, but are not limited to, Chronic Kidney Disease, Aicar Transformylase/Imp Cyclohydrolase Deficiency and Pediatric Osteosarcoma.

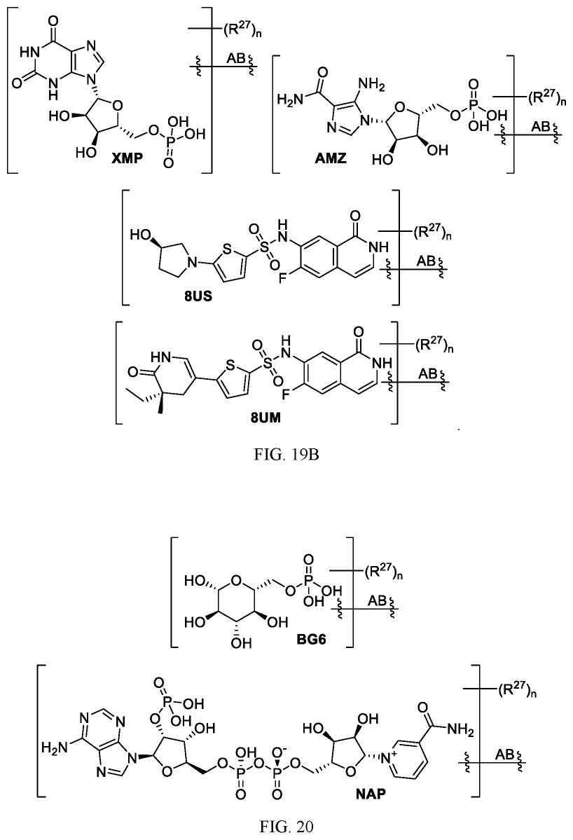

The Protein Data Bank website provides the crystal structure of ATIC bound to various compounds searchable by 1PLO, 5UZ0 and 5UY8. Representative ATIC Targeting Ligands are provided in FIGS. 19A and 19B.

### Glucose-6-Phosphate 1-Dehydrogenase (G6PD)

In certain embodiments, the Target Protein is Glucose-6-phosphate 1-dehydrogenase (G6PD). This gene encodes glucose-6-phosphate dehydrogenase. This protein is a cytosolic enzyme encoded by a housekeeping X-linked gene whose main function is to produce NADPH, a key electron donor in the defense against oxidizing agents and in reductive biosynthetic reactions.

In certain embodiments, the disease mediated by G6PD is a cancer.

In certain embodiments, the disease mediated by G6PD is a metabolic disorder.

In certain embodiments, the disease mediated by G6PD is a genetic disorder.

In certain embodiments, the disease mediated by G6PD is a dermatological disease.

In certain embodiments, the disease mediated by G6PD is an immunological disorder.

In certain embodiments, the disease mediated by G6PD is a cardiovascular disease.

In certain embodiments, the disease mediated by G6PD is a parasitic infection.

In certain embodiments, diseases associated with G6PD include, but are not limited to, Glucose-6-Phosphate Dehydrogenase Deficiency, Malaria, Vivax G6PD Deficiency, stroke, Favism, Acne Vulgaris, Neonatal Hyperbilirubinemia, diabetes, obesity, Hyperbilirubinemia, Hemolytic Disease, Hemolytic Disorders, Anemia, or Nonspherocytic Hemolytic.

The Protein Data Bank website provides the crystal structure of G6PD bound to various compounds searchable by 5UKW, 1QKI, 2BHL, 6JYU, and 2BH9. Representative G6PD Targeting Ligands are provided in FIG. 20.

### Glycogen Phosphorylase, Liver Form (PYGL)

In certain embodiments, the Target Protein is Glycogen phosphorylase, liver form (PYGL). This gene encodes a homodimeric protein that catalyzes the cleavage of alpha-1,4-glucosidic bonds to release glucose-1-phosphate from liver glycogen stores. This protein switches from inactive phosphorylase B to active phosphorylase A by phosphorylation of serine residue 15.

In certain embodiments, the disease mediated by PYGL is a cancer. In certain embodiments the cancer is liver cancer, renal cancer, Urothelial cancer, testicular cancer, melanoma.

In certain embodiments, the disease mediated by PYGL is a neurodegenerative disease.

In certain embodiments, diseases associated with PYGL include, but are not limited to, Glycogen Storage Disease Vi and Glycogen Storage Disease.

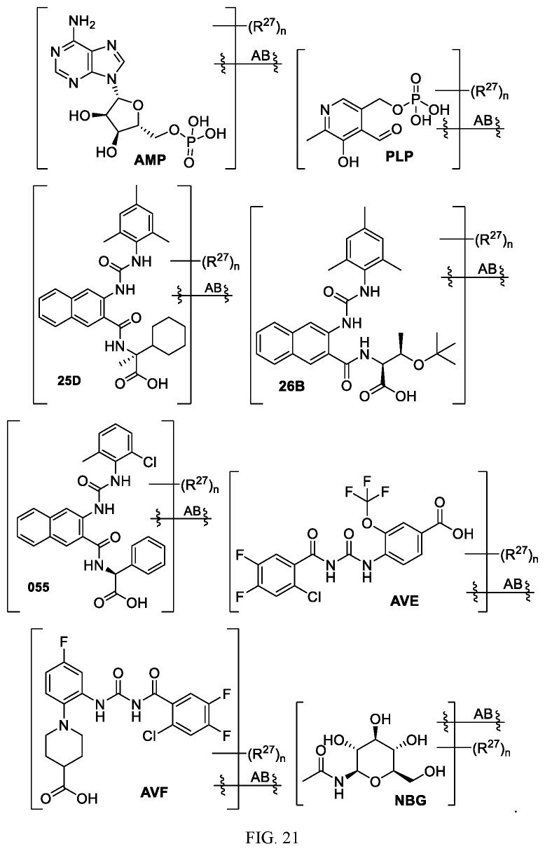

The Protein Data Bank website provides the crystal structure of PYGL bound to various compounds searchable by 1FA9, 3DD1, 3DDS, 3DDW, 2QLL, 3CEH, and 3CEJ. Representative PGYL Targeting Ligands are provided in FIG. 21.

### GDP-Mannose 4,6 Dehydratase (GMDS)

In certain embodiments, the Target Protein is GDP-mannose 4,6 dehydratase (GMDS). GDP-mannose 4,6-dehydratase catalyzes the conversion of GDP-mannose to GDP-4-keto-6-deoxymannose.

In certain embodiments, the disease mediated by GMDS is a cancer. In certain embodiments, the cancer, is glioma, thyroid cancer, colorectal cancer, head and neck cancer, stomach cancer, liver cancer, carcinoid, pancreatic cancer, renal cancer, prostate cancer, testicular cancer, Urothelial cancer, breast cancer, lymphoma, ovarian cancer.

In certain embodiments, diseases associated with GMDS include, but are not limited to, Congenital Disorder of Glycosylation, Type Iic or Phacolytic Glaucoma.

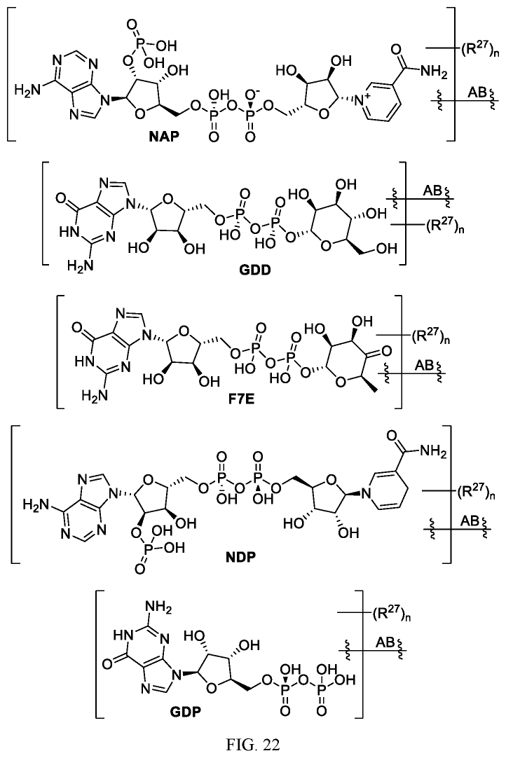

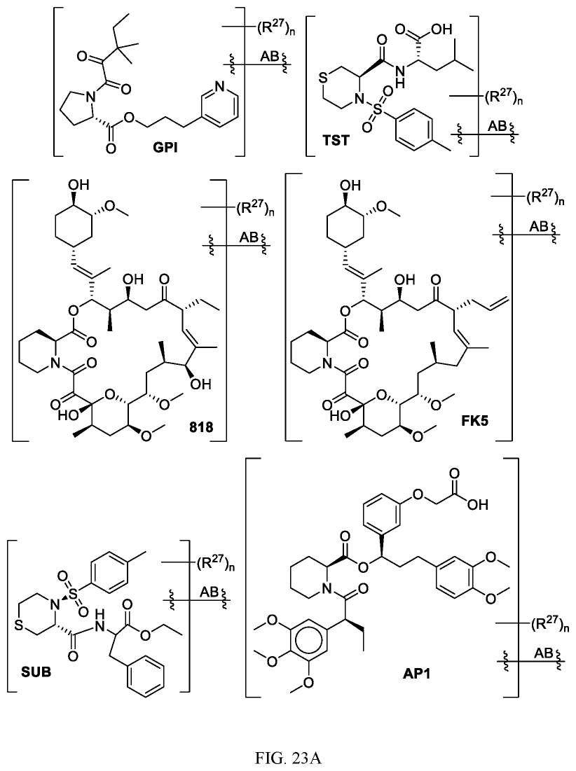

The Protein Data Bank website provides the crystal structure of GMDS searchable by 1T2A (Vedadi et al., “Crystal Structure and Biophysical Characterization of Human GDP-D-mannose 4,6-dehydratase”, to be published); as well as the crystal structure of GMDS bound to various compounds searchable by 6GPK, 1T2A, and 6GPL. Representative GMDS Targeting Ligands are provided in FIG. 22.

### SR-Related and CTD-Associated Factor 8 (SCAF8)

In certain embodiments, the Target Protein is SR-related and CTD-associated factor 8 (SCAF8). SCAF8 is an anti-terminator protein required to prevent early mRNA termination during transcription. Together with SCAF4, acts by suppressing the use of early, alternative poly(A) sites, thereby preventing the accumulation of non-functional truncated proteins.

In certain embodiments, the disease mediated by SCAF8 is a cancer. In certain embodiments, the cancer is thyroid cancer, lung cancer, colorectal cancer, head and neck cancer, stomach cancer, liver cancer, pancreatic cancer, renal cancer, Urothelial cancer, prostate cancer, testicular cancer, breast cancer, cervical cancer, endometrial cancer, ovarian cancer, skin cancer, glioma, lymphoma, or melanoma.

In certain embodiments, diseases associated with SCAF8 include, but are not limited to, Acrocallosal Syndrome.

The Protein Data Bank website provides the crystal structure of SCAF8 searchable by 2DIW (Dang et al., “Solution structure of the RPR domain of Putative RNA-binding protein 16”, to be published); as well as the crystal structure of SCAF8 bound to various compounds searchable by 3D9K, 3D9M, 3D9N, and 3D90. (Becker et al., “Snapshots of the RNA Processing Factor SCAF8 Bound to Different Phosphorylated Forms of the Carboxyl-terminal Domain of RNA Polymerase II”, J Biol Chem., 2008, 283: 22659-22669).

### Peptidyl-Prolyl Cis-Trans Isomerase FKBP1A (FKBP1A)

In certain embodiments, the Target Protein is Peptidyl-prolyl cis-trans isomerase FKBPlA (FKBPlA). The protein encoded by this gene is a member of the immunophilin protein family, which play a role in immunoregulation and basic cellular processes involving protein folding and trafficking.

In certain embodiments, the disease mediated by FKBPlA is a dermatological disease.

In certain embodiments, the disease mediated by FKBPlA is an immunological disorder

In certain embodiments, the disease mediated by FKBPlA is a cancer. In certain embodiments, the cancer is thyroid cancer, lung cancer, colorectal cancer, head and neck cancer, liver cancer, Urothelial cancer, endometrial cancer, ovarian cancer, melanoma.

In certain embodiments, the disease mediated by FKBPlA is a connective tissue disorder.

In certain embodiments, diseases associated with FKBPIA include, but are not limited to, Fibrodysplasia Ossificans Progressiva and Subependymal Glioma.

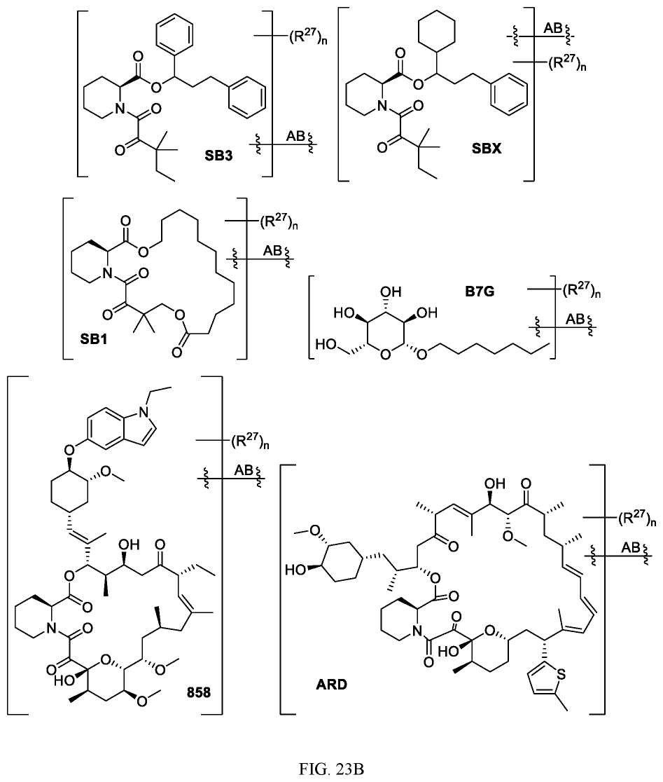

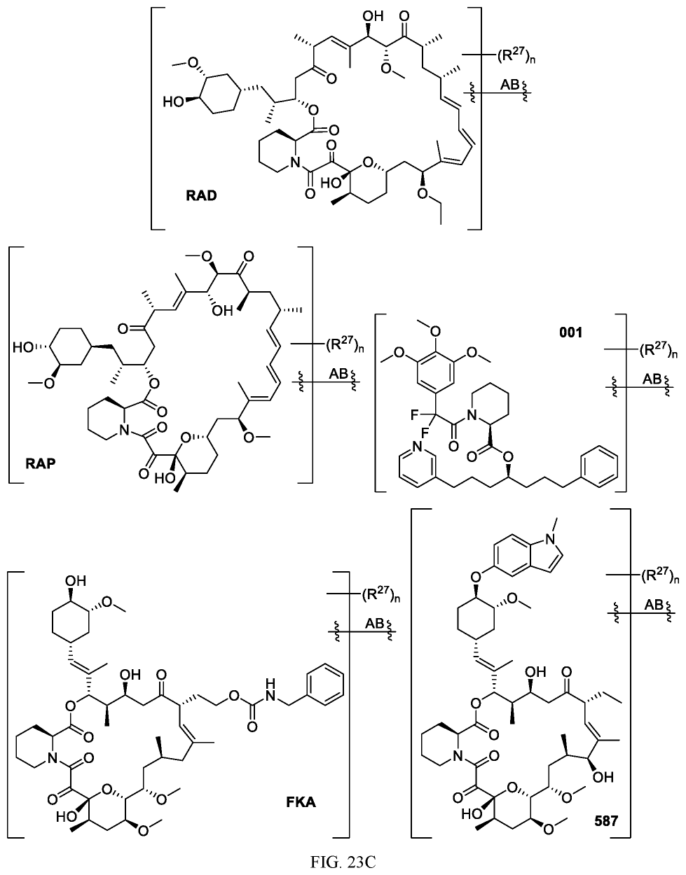

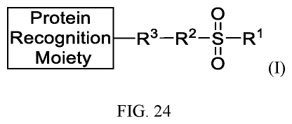

The Protein Data Bank website provides the crystal structure of FKBPIA bound to various compounds searchable by include 1D71, 1F40, 1FKD, 2FKE, 1FKF, 1FKJ, 1J4I, 1BL4, 1FKG, 1FKH, 1QPF, 1J4H, 3FAP, 6M4U, 1FAP, 1J4R, 1FKI, 1A7X, 1QPL, 1FKB, 2DG3, 2FAP, and 2DG9. Representative FKBPIA Targeting Ligands are provided in FIGS. 23A, 23B, and 23C.

In certain embodiments, the Target Protein is a cancer related protein selected from VEGF, SOX7, c-MET, HGFR, PTTG, cyclin D1, KIF4A, ALK, ROS1, BRAF, C-KIT, EGFR, HER2, ERBB2, JAK2, PD-1, MAPK, PI3K, ERK, ROS proto-oncogene 1, ROS1, PD-L1, PD-L2, EGFRTK, COX-2, PKC, HRAS, RXR, CDK1, CDK4, CDK7, BCL-2, BCL-XL, CTLA-4, PARP, RAD51, ERB4, VEGFR, PDGFR, FLT-3, c-FMS, MEK, mTOR, CHK1, CHK2, CD28, NRAS, CTNNB1, PIK3CA, AKT, DDR2, LKB1, FGFR1, PTEN, SOX2, TP53, c-MYC, CCND1, Cyclin E, ERalpha, RB, BRCA1, BRCA2, IGF1R, HER1, HER3, CDK6, HSP90, FOXA1, COX-1, CXCL8, CCL2, CCR2, CCR5, CXCR4, CXCL12, PI, ZNF703, FLT3, HOXA9, HOXD13, HOXA9, HOXC, PRX1, PRX2, BCR, ABL1, SRC, ABCB1, ABCG2, NFkB, PML, RARalpha, PLZF, TRAIL, RAS, RB1, pRB, MYC, NEU, WNT-1, Cyclin D2, AML, NUP98, PDGFRbeta, STAT5, RAF, MAPK, CD30, BCL6, BTK, EZH2, BAFF, TGFbeta, SYK, PKCbeta, STAT3, mTORC1, mTORC2, RNA polymerase II, Aurora Kinase A, Aurora Kinase B, HDM2, BCL-W, BCL2A1, MCL-1, CDK5, IRF4, CD38, NAE1, DNMT1, DNMT3A, DNMT3B, PRMT5, HDAC2, HIF-IA, CD40, RANK, HDAC1, HDAC3, HDAC8, and EML4.

In certain embodiments, the Target Protein is a breast cancer related protein selected from HER2, c-MYC, HRAS, CCND1, Cyclin E, ERalpha, RB, TP53, BRCA1, BRCA2, ERBB2, PI3K, AKT, FGFR, mTOR, IGF1R, PTEN, HER1, HER3, ERK, PARP, BRCA, B-RAF, VEGF, CDK4, CDK6, MAPK, HSP90, EGFR, FOXA1, cyclin D1, COX-1, COX-2, CXCL8, CCL2, CCR2, CCR5, CXCR4, CXCL12, MEK, PI, PIK3CA and ZNF703.

In certain embodiments, the Target Protein is a lung cancer related protein selected from VEGF, SOX7, c-MET, HGFR, PTTG, cyclin D1, KIF4A, ALK, ROS1, BRAF, C-KIT, EGFR, HER2, ERBB2, JAK2, PD-1, MAPK, PI3K, ERK, ROS proto-oncogene 1, ROS1, PD-L1, PD-L2, EGFRTK, COX-2, PKC, HRAS, RXR, CDK1, CDK4, CDK7, BCL-2, BCL-XL, CTLA-4, PARP, RAD51, ERB4, VEGFR, PDGFR, FLT-3, c-FMS, MEK, mTOR, CHK1, CHK2, CD28, NRAS, CTNNB1, PIK3CA, AKT, DDR2, LKB1, FGFR1, PTEN, SOX2, TP53, and EML4.

In certain embodiments, the Target Protein is a leukemia related protein selected from FLT3, mTOR, HOXA9, PRX1, PRX2, BCR, ABL1, SRC, ABCB1, ABCG2, NFkB, BCL-2, PML, RARalpha, PLZF, TRAIL, RAS, RB1, pRB, MYC, NEU, WNT-1, Cyclin D1, Cyclin D2, AML, NUP98, PDGFRbeta, HOXD13, HOXA9, HOXC, PI3K, RAF, MAPK, and STAT5.

In certain embodiments, the Target Protein is a lymphoma related protein selected from PI3K, AKT, mTOR, CD30, BCL6, BTK, EZH2, HSP90, cyclin D1, BAFF, CDK4, CDK6, WNT, TGFbeta, BCR, SYK, PKCbeta, STAT3, STAT5, JAK-2, MEK, mTORC1, mTORC2, RNA polymerase II, Aurora Kinase A, Aurora Kinase B, HDM2, PARP, CDK1, BCL-2, BCL-XL, BCL-W, BCL2A1, MCL-1, CDK5, IRF4, CD38, NAE1, DNMT1, DNMT3A, DNMT3B, PRMT5, HDAC2, HIF-1A, CD40, RANK, HDAC1, HDAC3, and HDAC8.

In certain embodiments, the Target Protein is involved in an autoimmune disorder, for example NF-kB, MMP-9, CD20, S1PR1, NFE2L2, AHR, cPLA2, CNR1, CERS2, KIR4.1, P2X1, P2X3, P2X7, TLR2, TLR4, TLR7, TLR9, IL-17, alpha-v beta-3, ANGPT1, SYK, CTLA4, TNFalpha, IL-6, CXCL8, CCL2, CCL5, CXCL10, CXCL5, CXCL1, CXCL12, CXCL13, CCL21, FLIP, SUMO-1, RAS, MYC, MAPK, PDGFR, C-FMS, C-KIT, FAP, PBEF, STAT4, RF, ACPA, HLA-DRB1, PTPN22, TH-17, IL-21, IL-22, IL-23, GM-CSF, JAK1, JAK2, and JAK3.

In certain embodiments, the Target Protein is involved in diseases caused by retroviruses, for example reverse transcriptase, aspartyl protease, integrase, matrix-2 protein, neuraminidase, viral RNA polymerase, viral DNA polymerase, NS2-3 protease, NS3-4A protease, NS5A, GP41, CCR5, and CXCR4.

In certain embodiments, the Target Protein plays a role in fibrotic disorders, for example CFTR, LCK, LYN, SRC, PDGFR, FGFR, VEGFR, FLT3, TGF-beta, TNF-alpha, IL-1beta, ILK, PDGF, IL-13, IL-4, LGALS3, LOXL2, ACTA2, IL-6, STAT3, MAPK, WNT, S6K1, TIMP-1, alpha-SMA, MMP-2, CTGF, HGF, IL-1R1, IL-lbetaR, CCL2, CCR5, CCR2, IFN-gammaR, IFN-alpha, MMP-9, ET-1 receptor, AT1 receptor, LPAR, PAR1, CB1, CB2, prostacyclin receptor, VIP receptor, CPB2, ELANE, relaxin receptor, SAP, integrin alpha5, TGM2, mTORC1, mTORC2, JAK1, JAK2, AKT, FAK1, INK, IKK, NF-kB, ROCK, 26S protease, caspase, PDE, cathepsin B, S100A9, procollagen-proline dioxygenase, PPAR, FXR, GR, ER, SMAD2, SMAD3, NOX1, NOX4, and ROS.

In certain embodiments the Target Protein is selected from A1BG, A1CF, A2M, A2ML1, A3GALT2, A4GALT, A4GNT, AAAS, AACS, AADAC, AADACL2, AADACL3, AADACL4, AADAT, AAED1, AAGAB, AAK1, AAMDC, AAMP, AANAT, AAR₂, AARD, AARS, AARS2, AARSD1, AASDH, AASDHPPT, AASS, AATF, AATK, AATK-AS1, ABAT, ABCA1, ABCA10, ABCA12, ABCA13, ABCA2, ABCA3, ABCA4, ABCA5, ABCA6, ABCA7, ABCA8, ABCA9, ABCB1, ABCB10, ABCB11, ABCB4, ABCB5, ABCB6, ABCB7, ABCB8, ABCB9, ABCC1, ABCC10, ABCC11, ABCC12, ABCC2, ABCC3, ABCC4, ABCC5, ABCC6, ABCC8, ABCC9, ABCD1, ABCD2, ABCD3, ABCD4, ABCE1, ABCF1, ABCF2, ABCF3, ABCG1, ABCG2, ABCG4, ABCG5, ABCG8, ABHD1, ABHD10, ABHD11, ABHD12, ABHD12B, ABHD13, ABHD14A, ABHD14A-ACY1, ABHD14B, ABHD15, ABHD16A, ABHD16B, ABHD17A, ABHD17B, ABHD17C, ABHD18, ABHD2, ABHD3, ABHD4, ABHD5, ABHD6, ABHD8, ABI1, ABI2, ABI3, ABI3BP, ABL1, ABL2, ABLIM1, ABLIM2, ABLIM3, ABO, ABR, ABRA, ABRACL, ABRAXAS1, ABRAXAS2, ABT1, ABTB1, ABTB2, AC001226.2, AC002094.3, AC002115.2, AC002310.4, AC002310.5, AC002429.2, AC002985.1, AC002996.1, AC003002.1, AC003002.2, AC003002.3, AC003002.4, AC003005.1, AC003006.1, AC003688.1, AC004076.1, AC004080.3, AC004223.3, AC004233.2, AC004556.1, AC004691.2, AC004706.4, AC004754.1, AC004805.1, AC004832.3, AC004922.1, AC004997.1, AC005020.2, AC005041.1, AC005154.6, AC005258.1, AC005324.3, AC005324.4, AC005520.1, AC005551.1, AC005670.2, AC005697.1, AC005702.2, AC005726.2, AC005779.2, AC005832.4, AC005833.1, AC005833.3, AC005837.2, AC005841.2, AC005885.1, AC005943.1, AC006030.1, AC006254.1, AC006269.1, AC006449.4, AC006486.1, AC006538.2, AC006978.2, AC007040.2, AC007192.1, AC007240.1, AC007325.1, AC007325.2, AC007325.4, AC007326.4, AC007375.2, AC007383.6, AC007537.5, AC007731.5, AC007906.2, AC007998.2, AC008073.3, AC008162.2, AC008393.2, AC008403.1, AC008481.3, AC008537.1, AC008560.1, AC008575.1, AC008575.2, AC008687.1, AC008687.4, AC008687.8, AC008695.1, AC008735.6, AC008750.8, AC008758.1, AC008758.4, AC008758.5, AC008758.6, AC008763.2, AC008763.3, AC008764.1, AC008764.4, AC008770.2, AC008770.3, AC008878.1, AC008878.2, AC008878.3, AC008982.1, AC008982.3, AC009014.1, AC009086.2, AC009119.2, AC009122.1, AC009133.6, AC009163.2, AC009163.4, AC009286.3, AC009336.2, AC009477.2, AC009690.1, AC009690.3, AC009779.3, AC010132.3, AC010255.3, AC010319.2, AC010323.1, AC010325.1, AC010326.2, AC010327.1, AC010422.3, AC010422.5, AC010422.6, AC010463.1, AC010487.3, AC010522.1, AC010531.1, AC010542.3, AC010547.4, AC010547.5, AC010615.4, AC010616.1, AC010619.1, AC010646.1, AC010724.2, AC011005.1, AC011043.1, AC011043.2, AC011195.2, AC011295.1, AC011346.1, AC011448.1, AC011452.1, AC011455.3, AC011455.4, AC011462.1, AC011473.4, AC011479.1, AC011498.4, AC011499.1, AC011511.1, AC011511.4, AC011530.1, AC011604.2, AC011841.1, AC012184.2, AC012254.2, AC012309.1, AC012314.1, AC012314.10, AC012314.11, AC012314.12, AC012314.4, AC012314.5, AC012314.6, AC012314.8, AC012531.3, AC012651.1, AC013269.1, AC013271.1, AC013394.1, AC013470.2, AC015688.5, AC015802.6, AC015813.2, AC017081.3, AC017081.4, AC017081.5, AC017083.4, AC018512.1, AC018523.2, AC018554.3, AC018630.6, AC018709.1, AC018755.2, AC018793.1, AC018793.2, AC018793.3, AC018793.4, AC018793.5, AC019117.3, AC020636.2, AC020909.1, AC020914.1, AC020915.1, AC020915.2, AC020915.6, AC020922.1, AC020934.3, AC021072.1, AC022016.2, AC022167.5, AC022335.1, AC022384.1, AC022400.6, AC022826.2, AC023055.1, AC023491.2, AC023509.3, AC024592.3, AC024940.1, AC024940.6, AC025165.3, AC025263.2, AC025283.2, AC025287.4, AC025594.2, AC026369.8, AC026398.1, AC026461.4, AC026464.1, AC026464.3, AC026464.4, AC026786.1, AC026954.2, AC027796.3, AC034102.2, AC036214.3, AC037459.1, AC037482.2, AC037482.3, AC040162.1, AC040162.4, AC044810.8, AC046185.1, AC048338.1, AC051649.2, AC053481.5, AC055811.2, AC058822.1, AC064853.2, AC064853.3, AC064853.4, AC064853.5, AC064853.6, AC067968.1, AC068234.1, AC068533.4, AC068547.1, AC068580.4, AC068631.2, AC068775.1, AC068775.2, AC068790.8, AC068896.1, AC068946.1, AC068987.5, AC069257.3, AC069368.1, AC069503.2, AC069544.2, AC072022.1, AC073082.1, AC073111.3, AC073111.5, AC073264.3, AC073508.2, AC073610.2, AC073610.3, AC073612.1, AC073896.1, AC074143.1, AC078927.1, AC079325.2, AC079447.1, AC079594.2, AC083800.1, AC083902.2, AC084337.2, AC087289.3, AC087498.1, AC087632.1, AC090004.1, AC090227.1, AC090360.1, AC090527.2, AC090958.3, AC091167.3, AC091167.7, AC091167.8, AC091304.7, AC091491.1, AC091551.1, AC091959.3, AC091980.2, AC092017.3, AC092042.3, AC092073.1, AC092111.3, AC092143.1, AC092329.3, AC092442.1, AC092587.1, AC092647.5, AC092718.3, AC092718.8, AC092821.1, AC092824.3, AC092835.1, AC093155.3, AC093227.3, AC093423.3, AC093525.1, AC093525.2, AC093668.1, AC093762.1, AC093762.2, AC093762.3, AC093899.2, AC096582.3, AC096887.1, AC097372.1, AC097495.1, AC097637.1, AC097662.2, AC098484.3, AC098650.1, AC098850.4, AC099329.3, AC099489.1, AC099518.3, AC099811.2, AC099850.2, AC100868.1, AC104109.3, AC104151.1, AC104304.1, AC104452.1, AC104532.1, AC104534.3, AC104581.1, AC104581.3, AC104662.2, AC104836.1, AC105001.2, AC105052.1, AC106774.10, AC106774.5, AC106774.6, AC106774.7, AC106774.8, AC106774.9, AC106782.1, AC106886.5, AC107871.1, AC108488.2, AC108750.1, AC108941.2, AC109583.3, AC110275.1, AC112229.3, AC112484.1, AC113189.6, AC113189.9, AC113331.2, AC113554.2, AC114296.1, AC114490.2, AC115220.1, AC116366.3, AC116565.1, AC117457.1, AC118470.1, AC118553.2, AC119396.1, AC119674.2, AC120057.3, AC120114.5, AC124312.1, AC126755.2, AC127537.5, AC127537.6, AC127537.8, AC129492.3, AC131097.2, AC131160.1, AC133551.1, AC133555.3, AC134669.2, AC134772.2, AC135050.2, AC135068.1, AC135068.2, AC135068.3, AC135068.8, AC135178.2, AC135586.2, AC136352.3, AC136352.4, AC136428.1, AC136612.1, AC136616.1, AC136616.2, AC136616.3, AC137834.1, AC138517.2, AC138647.1, AC138696.1, AC138811.2, AC138894.1, AC138969.1, AC139530.2, AC139677.1, AC139677.2, AC140504.1, AC141272.1, AC142391.1, AC142525.4, AC145029.2, AC145212.1, AC145212.2, AC171558.1, AC171558.3, AC171558.5, AC171558.6, AC187653.1, AC207056.1, AC209232.1, AC209539.2, AC210544.1, AC213203.1, AC229888.1, AC229888.10, AC229888.2, AC229888.3, AC229888.4, AC229888.5, AC229888.6, AC229888.7, AC229888.8, AC229888.9, AC233282.1, AC233282.2, AC233723.1, AC233724.12, AC233724.16, AC233724.17, AC233724.18, AC233724.19, AC233724.20, AC233724.21, AC233724.6, AC233755.1, AC233755.2, AC233992.2, AC234301.1, AC234301.3, AC234635.1, AC234635.3, AC234635.4, AC234635.5, AC236040.1, AC239612.1, AC239618.1, AC239618.2, AC239618.3, AC239618.4, AC239618.5, AC239618.6, AC239618.7, AC239618.9, AC239799.1, AC240274.1, AC241401.1, AC241409.2, AC241410.1, AC241556.3, AC241556.4, AC241640.1, AC241640.2, AC241640.4, AC242528.1, AC242528.2, AC243547.3, AC243733.1, AC243734.1, AC243756.1, AC243790.1, AC243967.1, AC244196.1, AC244196.2, AC244196.3, AC244196.4, AC244196.5, AC244197.3, AC244216.4, AC244216.5, AC244226.1, AC244226.2, AC244472.1, AC244472.2, AC244472.3, AC244472.4, AC244472.5, AC244489.1, AC244489.2, AC244517.10, AC244517.6, AC245033.1, AC245034.2, AC245078.1, AC245088.2, AC245088.3, AC245369.1, AC245369.2, AC245369.3, AC245369.4, AC245369.6, AC245427.1, AC245427.3, AC245427.4, AC245427.5, AC245427.6, AC245427.7, AC245427.8, AC245427.9, AC245748.1, AC247036.3, AC247036.4, AC247036.5, AC247036.6, AC254560.1, AC254788.1, AC254788.2, AC254952.1, AC255093.3, AC255093.5, AC256236.1, AC256236.2, AC256236.3, AC256300.2, AC256309.2, AC270107.1, AC270107.10, AC270107.12, AC270107.2, AC270107.3, AC270107.4, AC270107.5, AC270107.7, AC270107.8, AC270107.9, AC270227.1, AC270306.4, AC275455.2, ACAA1, ACAA2, ACACA, ACACB, ACAD10, ACAD11, ACAD8, ACAD9, ACADL, ACADM, ACADS, ACADSB, ACADVL, ACAN, ACAP1, ACAP2, ACAP3, ACAT1, ACAT2, ACBD3, ACBD4, ACBD5, ACBD6, ACBD7, ACCS, ACCSL, ACD, ACE, ACE2, ACER1, ACER2, ACER3, ACHE, ACIN1, ACKR1, ACKR2, ACKR3, ACKR4, ACLY, ACMSD, ACO1, ACO2, ACOD1, ACOT1, ACOT11, ACOT12, ACOT13, ACOT2, ACOT4, ACOT6, ACOT7, ACOT8, ACOT9, ACOX1, ACOX2, ACOX3, ACOXL, ACP1, ACP2, ACP4, ACP5, ACP6, ACP7, ACPP, ACR, ACRBP, ACRV1, ACSBG1, ACSBG2, ACSF2, ACSF3, ACSL1, ACSL3, ACSL4, ACSL5, ACSL6, ACSM1, ACSM2A, ACSM2B, ACSM3, ACSM4, ACSM5, ACSM6, ACSS1, ACSS2, ACSS3, ACTA1, ACTA2, ACTB, ACTBL2, ACTC1, ACTG1, ACTG2, ACTL10, ACTL6A, ACTL6B, ACTL7A, ACTL7B, ACTL8, ACTL9, ACTN1, ACTN2, ACTN3, ACTN4, ACTR10, ACTRIA, ACTRIB, ACTR2, ACTR3, ACTR3B, ACTR3C, ACTR5, ACTR6, ACTR8, ACTRT1, ACTRT2, ACTRT3, ACVR1, ACVR1B, ACVR1C, ACVR2A, ACVR2B, ACVRL1, ACY1, ACY3, ACYP1, ACYP2, AD000671.1, AD000671.2, ADA, ADA2, ADAD1, ADAD2, ADAL, ADAM10, ADAM11, ADAM12, ADAM15, ADAM17, ADAM18, ADAM19, ADAM2, ADAM20, ADAM21, ADAM22, ADAM23, ADAM28, ADAM29, ADAM30, ADAM32, ADAM33, ADAM7, ADAM8, ADAM9, ADAMDEC1, ADAMTS1, ADAMTS10, ADAMTS12, ADAMTS13, ADAMTS14, ADAMTS15, ADAMTS16, ADAMTS17, ADAMTS18, ADAMTS19, ADAMTS2, ADAMTS20, ADAMTS3, ADAMTS4, ADAMTS5, ADAMTS6, ADAMTS7, ADAMTS8, ADAMTS9, ADAMTSL1, ADAMTSL2, ADAMTSL3, ADAMTSL4, ADAMTSL5, ADAP1, ADAP2, ADAR, ADARB1, ADARB2, ADAT1, ADAT2, ADAT3, ADCK1, ADCK2, ADCK5, ADCY1, ADCY10, ADCY2, ADCY3, ADCY4, ADCY5, ADCY6, ADCY7, ADCY8, ADCY9, ADCYAP1, ADCYAP1R1, ADD1, ADD2, ADD3, ADGB, ADGRA1, ADGRA2, ADGRA3, ADGRB1, ADGRB2, ADGRB3, ADGRD1, ADGRD2, ADGRE1, ADGRE2, ADGRE3, ADGRE5, ADGRF1, ADGRF2, ADGRF3, ADGRF4, ADGRF5, ADGRG1, ADGRG2, ADGRG3, ADGRG4, ADGRG5, ADGRG6, ADGRG7, ADGRL1, ADGRL2, ADGRL3, ADGRL4, ADGRV1, ADH1A, ADH1B, ADH1C, ADH4, ADH5, ADH6, ADH7, ADHFEl, ADIl, ADIG, ADIPOQ, ADIPOR1, ADIPOR₂, ADIRF, ADK, ADM, ADM2, ADM5, ADNP, ADNP2, ADO, ADORA1, ADORA2A, ADORA2B, ADORA3, ADPGK, ADPRH, ADPRHL1, ADPRHL2, ADPRM, ADRA1A, ADRAIB, ADRA1D, ADRA2A, ADRA2B, ADRA2C, ADRB1, ADRB2, ADRB3, ADRM1, ADSL, ADSS, ADSSL1, ADTRP, AEBP1, AEBP2, AEN, AES, AF130351.1, AF241726.2, AFAP1, AFAPIL1, AFAP1L2, AFDN, AFF1, AFF2, AFF3, AFF4, AFG1L, AFG3L2, AFM, AFMID, AFP, AFTPH, AGA, AGAP1, AGAP2, AGAP3, AGAP4, AGAP5, AGAP6, AGAP9, AGBL1, AGBL2, AGBL3, AGBL4, AGBL5, AGER, AGFG1, AGFG2, AGGF1, AGK, AGL, AGMAT, AGMO, AGO1, AGO2, AGO3, AGO4, AGPAT1, AGPAT2, AGPAT3, AGPAT4, AGPAT5, AGPS, AGR2, AGR3, AGRN, AGRP, AGT, AGTPBP1, AGTR1, AGTR2, AGTRAP, AGXT, AGXT2, AHCTF1, AHCY, AHCYL1, AHCYL2, AHDC1, AHIl, AHNAK, AHNAK2, AHR, AHRR, AHSA1, AHSA2, AHSG, AHSP, AICDA, AIDA, AIFI, AIFIL, AIFM1, AIFM2, AIFM3, AIGI, AIM2, AIMPI, AIMP2, AIP, AIPL1, AIRE, AJAP1, AJUBA, AK1, AK2, AK3, AK4, AK5, AK6, AK7, AK8, AK9, AKAIN1, AKAP1, AKAP10, AKAP11, AKAP12, AKAP13, AKAP14, AKAP17A, AKAP2, AKAP3, AKAP4, AKAP5, AKAP6, AKAP7, AKAP8, AKAP8L, AKAP9, AKIP1, AKIRIN1, AKIRIN2, AKNA, AKNAD1, AKR1A1, AKR1B1, AKR1B10, AKR1B15, AKR1C1, AKR1C2, AKR1C3, AKR1C4, AKR1D1, AKR1E2, AKR7A2, AKR7A3, AKR7L, AKT1, AKT1S1, AKT2, AKT3, AKTIP, AL020996.2, AL021154.3, AL021546.1, AL021997.3, AL022238.4, AL022318.4, AL024498.2, AL031708.1, AL032819.3, AL033529.1, AL035425.2, AL035460.1, AL049634.2, AL049650.1, AL049697.1, AL049779.1, AL049839.2, AL049844.1, AL049844.3, AL080251.1, AL096814.1, AL096870.1, AL109810.2, AL109811.4, AL109827.1, AL109936.3, AL109936.4, AL110118.2, AL110118.4, AL117258.1, AL117339.5, AL117348.2, AL121581.1, AL121594.3, AL121722.1, AL121753.1, AL121758.1, AL121845.2, AL121845.3, AL132671.2, AL132780.3, AL133352.1, AL133414.1, AL133414.2, AL136295.1, AL136295.3, AL136295.4, AL136295.5, AL136373.1, AL136531.2, AL138694.1, AL138752.2, AL138826.1, AL139011.2, AL139260.3, AL139300.1, AL139353.1, AL157392.5, AL159163.1, AL160275.1, AL160276.1, AL160396.2, AL161669.4, AL161911.1, AL162231.1, AL162231.3, AL163195.3, AL163636.2, AL353572.3, AL353588.1, AL354761.2, AL354822.1, AL355102.2, AL355315.1, AL355860.1, AL355916.3, AL355987.1, AL355987.3, AL356585.9, AL357673.1, AL358075.4, AL359736.1, AL359736.3, AL359922.1, AL360181.3, AL360181.5, AL365205.1, AL365214.3, AL365232.1, AL365273.2, AL391650.1, AL449266.1, AL451007.3, AL512428.1, AL512506.3, AL512785.2, AL513165.2, AL513523.10, AL513523.9, AL583836.1, AL589666.1, AL590132.1, AL590560.1, AL591806.3, AL592183.1, AL592490.1, AL593848.2, AL603832.3, AL645922.1, AL645941.2, AL662828.1, AL662852.6, AL662899.1, AL662899.2, AL662899.3, AL669918.1, AL672043.1, AL672142.1, AL691442.1, AL713999.1, AL772284.2, AL807752.6, AL807752.7, AL844853.2, AL845331.2, AL845464.1, AL928654.4, AL929554.1, AL929561.7, ALAD, ALAS1, ALAS2, ALB, ALCAM, ALDH16A1, ALDH18A1, ALDH1A1, ALDH1A2, ALDH1A3, ALDH1B1, ALDH1L1, ALDH1L2, ALDH2, ALDH3A1, ALDH3A2, ALDH3B1, ALDH3B2, ALDH4A1, ALDH5A1, ALDH6A1, ALDH7A1, ALDH8A1, ALDH9A1, ALDOA, ALDOB, ALDOC, ALG1, ALG10, ALG10B, ALG11, ALG12, ALG13, ALG14, ALG1L, ALG1L2, ALG2, ALG3, ALG5, ALG6, ALG8, ALG9, ALK, ALKAL1, ALKAL2, ALKBH1, ALKBH2, ALKBH3, ALKBH4, ALKBH5, ALKBH6, ALKBH7, ALKBH8, ALLC, ALMS1, ALOX12, ALOX12B, ALOX15, ALOX15B, ALOX5, ALOX5AP, ALOXE3, ALPI, ALPK1, ALPK2, ALPK3, ALPL, ALPP, ALPPL2, ALS2, ALS2CL, ALS2CR12, ALX1, ALX3, ALX4, ALYREF, AMACR, AMBN, AMBP, AMBRA1, AMD1, AMDHD1, AMDHD2, AMELX, AMELY, AMER1, AMER2, AMER3, AMFR, AMH, AMHR2, AMIGO1, AMIGO2, AMIGO3, AMMECRI, AMMECRIL, AMN, AMN1, AMOT, AMOTL1, AMOTL2, AMPD1, AMPD2, AMPD3, AMPH, AMT, AMTN, AMY1A, AMYlB, AMYlC, AMY2A, AMY2B, AMZ1, AMZ2, ANAPC1, ANAPC10, ANAPC11, ANAPC13, ANAPC15, ANAPC16, ANAPC2, ANAPC4, ANAPC5, ANAPC7, ANG, ANGEL1, ANGEL2, ANGPT1, ANGPT2, ANGPT4, ANGPTL1, ANGPTL2, ANGPTL3, ANGPTL4, ANGPTL5, ANGPTL6, ANGPTL7, ANGPTL8, ANHX, ANK1, ANK2, ANK3, ANKAR, ANKDD1A, ANKDD1B, ANKEF1, ANKFN1, ANKFYl, ANKH, ANKHD1, ANKHD1-EIF4EBP3, ANKIBI, ANKK1, ANKLE1, ANKLE2, ANKMY1, ANKMY2, ANKRA2, ANKRD1, ANKRD10, ANKRD11, ANKRD12, ANKRD13A, ANKRD13B, ANKRD13C, ANKRD13D, ANKRD16, ANKRD17, ANKRD18A, ANKRD18B, ANKRD2, ANKRD20A1, ANKRD20A2, ANKRD20A3, ANKRD20A4, ANKRD20A8P, ANKRD22, ANKRD23, ANKRD24, ANKRD26, ANKRD27, ANKRD28, ANKRD29, ANKRD30A, ANKRD30B, ANKRD30BL, ANKRD31, ANKRD33, ANKRD33B, ANKRD34A, ANKRD34B, ANKRD34C, ANKRD35, ANKRD36, ANKRD36B, ANKRD36C, ANKRD37, ANKRD39, ANKRD40, ANKRD42, ANKRD44, ANKRD45, ANKRD46, ANKRD49, ANKRD50, ANKRD52, ANKRD53, ANKRD54, ANKRD55, ANKRD6, ANKRD60, ANKRD61, ANKRD62, ANKRD63, ANKRD65, ANKRD66, ANKRD7, ANKRD9, ANKS1A, ANKS1B, ANKS3, ANKS4B, ANKS6, ANKUB1, ANKZF1, ANLN, ANO1, ANO10, ANO2, ANO3, ANO4, ANO5, ANO6, ANO7, ANO8, ANO9, ANOS1, ANP32A, ANP32B, ANP32D, ANP32E, ANPEP, ANTXR1, ANTXR2, ANTXRL, ANXA1, ANXA10, ANXA11, ANXA13, ANXA2, ANXA2R, ANXA3, ANXA4, ANXA5, ANXA6, ANXA7, ANXA8, ANXA8L1, ANXA9, AOAH, AOC1, AOC2, AOC3, AOX1, AP000275.2, AP000295.1, AP000311.1, AP000322.1, AP000349.1, AP000350.12, AP000350.4, AP000351.3, AP000351.7, AP000721.1, AP000781.2, AP001160.5, AP001273.2, AP001458.2, AP001781.3, AP001931.1, AP002360.1, AP002373.1, AP002495.1, AP002512.3, AP002512.4, AP002748.4, AP002990.1, AP003071.5, AP003108.2, AP003419.2, AP004243.1, AP006285.3, AP1AR, AP1B1, AP1G1, AP1G2, AP1M1, AP1M2, AP1S1, AP1S2, AP1S3, AP2A1, AP2A2, AP2B1, AP2M1, AP2S1, AP3B1, AP3B2, AP3D1, AP3M1, AP3M2, AP3S1, AP3S2, AP4B1, AP4E1, AP4M1, AP4S1, AP5B1, AP5M1, AP5S1, AP5Z1, APAF1, APBA1, APBA2, APBA3, APBB1, APBBlIP, APBB2, APBB3, APC, APC2, APCDD1, APCDD1L, APCS, APEH, APELA, APEX1, APEX2, APH1A, APH1B, API5, APIP, APLF, APLN, APLNR, APLP1, APLP2, APMAP, APOA1, APOA2, APOA4, APOA5, APOB, APOBEC1, APOBEC2, APOBEC3A, APOBEC3B, APOBEC3C, APOBEC3D, APOBEC3F, APOBEC3G, APOBEC3H, APOBEC4, APOBR, APOC1, APOC2, APOC3, APOC4, APOC4-APOC2, APOD, APOE, APOF, APOH, APOL1, APOL2, APOL3, APOL4, APOL5, APOL6, APOLD1, APOM, APOO, APOOL, APOPT1, APP, APPBP2, APPL1, APPL2, APRT, APTX, AQP1, AQP10, AQP11, AQP12A, AQP12B, AQP2, AQP3, AQP4, AQP5, AQP6, AQP7, AQP8, AQP9, AQR, AR, ARAF, ARAP1, ARAP2, ARAP3, ARC, ARCN1, AREG, AREL1, ARF1, ARF3, ARF4, ARF5, ARF6, ARFGAP1, ARFGAP2, ARFGAP3, ARFGEF1, ARFGEF2, ARFGEF3, ARFIP1, ARFIP2, ARFRP1, ARG1, ARG2, ARGFX, ARGLU1, ARHGAP1, ARHGAP10, ARHGAP11 A, ARHGAP11B, ARHGAP12, ARHGAP15, ARHGAP17, ARHGAP18, ARHGAP19, ARHGAP19-SLIT1, ARHGAP20, ARHGAP21, ARHGAP22, ARHGAP23, ARHGAP24, ARHGAP25, ARHGAP26, ARHGAP27, ARHGAP28, ARHGAP29, ARHGAP30, ARHGAP31, ARHGAP32, ARHGAP33, ARHGAP35, ARHGAP36, ARHGAP39, ARHGAP4, ARHGAP40, ARHGAP42, ARHGAP44, ARHGAP45, ARHGAP5, ARHGAP6, ARHGAP8, ARHGAP9, ARHGDIA, ARHGDIB, ARHGDIG, ARHGEF1, ARHGEF10, ARHGEF10L, ARHGEF11, ARHGEF12, ARHGEF15, ARHGEF16, ARHGEF17, ARHGEF18, ARHGEF19, ARHGEF2, ARHGEF25, ARHGEF26, ARHGEF28, ARHGEF3, ARHGEF33, ARHGEF35, ARHGEF37, ARHGEF38, ARHGEF39, ARHGEF4, ARHGEF40, ARHGEF5, ARHGEF6, ARHGEF7, ARHGEF9, ARID1A, ARIDIB, ARID2, ARID3A, ARID3B, ARID3C, ARID4A, ARID4B, ARID5A, ARID5B, ARIH1, ARIH2, ARIH2OS, ARL1, ARL10, ARL11, ARL13A, ARL13B, ARL14, ARL14EP, ARL14EPL, ARL15, ARL16, ARL17A, ARL17B, ARL2, ARL2BP, ARL2-SNX15, ARL3, ARL4A, ARL4C, ARL4D, ARL5A, ARL5B, ARL5C, ARL6, ARL6IP1, ARL6IP4, ARL6IP5, ARL6IP6, ARL8A, ARL8B, ARL9, ARMC1, ARMC10, ARMC12, ARMC2, ARMC3, ARMC4, ARMC5, ARMC6, ARMC7, ARMC8, ARMC9, ARMCX1, ARMCX2, ARMCX3, ARMCX4, ARMCX5, ARMCX6, ARMS2, ARMT1, ARNT, ARNT2, ARNTL, ARNTL2, ARPC1A, ARPCIB, ARPC2, ARPC3, ARPC4, ARPC4-TTLL3, ARPC5, ARPC5L, ARPIN, ARPP19, ARPP21, ARR3, ARRB1, ARRB2, ARRDC1, ARRDC2, ARRDC3, ARRDC4, ARRDC5, ARSA, ARSB, ARSD, ARSE, ARSF, ARSG, ARSH, ARSI, ARSJ, ARSK, ART1, ART3, ART4, ART5, ARTN, ARV1, ARVCF, ARX, AS3MT, ASAH1, ASAH2, ASAH2B, ASAP1, ASAP2, ASAP3, ASB1, ASB10, ASB11, ASB12, ASB13, ASB14, ASB15, ASB16, ASB17, ASB18, ASB2, ASB3, ASB4, ASB5, ASB6, ASB7, ASB8, ASB9, ASCC1, ASCC2, ASCC3, ASCL1, ASCL2, ASCL3, ASCL4, ASCL5, ASF1A, ASF1B, ASGR1, ASGR2, ASH1L, ASH2L, ASIC1, ASIC2, ASIC3, ASIC4, ASIC5, ASIP, ASL, ASMT, ASMTL, ASNA1, ASNS, ASNSD1, ASPA, ASPDH, ASPG, ASPH, ASPHD1, ASPHD2, ASPM, ASPN, ASPRV1, ASPSCR1, ASRGL1, ASS1, ASTE1, ASTL, ASTN1, ASTN2, ASXL1, ASXL2, ASXL3, ASZ1, ATAD1, ATAD2, ATAD2B, ATAD3A, ATAD3B, ATAD3C, ATAD5, ATAT1, ATCAY, ATE1, ATF1, ATF2, ATF3, ATF4, ATF5, ATF6, ATF6B, ATF7, ATF7IP, ATF7IP2, ATG10, ATG101, ATG12, ATG13, ATG14, ATG16L1, ATG16L2, ATG2A, ATG2B, ATG3, ATG4A, ATG4B, ATG4C, ATG4D, ATG5, ATG7, ATG9A, ATG9B, ATIC, ATL1, ATL2, ATL3, ATM, ATMIN, ATN1, ATOH1, ATOH7, ATOH8, ATOX1, ATP10A, ATP10B, ATP10D, ATP11A, ATP11B, ATP11C, ATP12A, ATP13A1, ATP13A2, ATP13A3, ATP13A4, ATP13A5, ATP1A1, ATP1A2, ATP1A3, ATP1A4, ATP1B1, ATP1B2, ATP1B3, ATP1B4, ATP23, ATP2A1, ATP2A2, ATP2A3, ATP2B1, ATP2B2, ATP2B3, ATP2B4, ATP2C1, ATP2C2, ATP4A, ATP4B, ATP5A1, ATP5B, ATP5C1, ATP5D, ATP5E, ATP5EP2, ATP5F1, ATP5G1, ATP5G2, ATP5G3, ATP5H, ATP5I, ATP5J, ATP5J2, ATP5J2-PTCD1, ATP5L, ATP5L2, ATP50, ATP5S, ATP6AP1, ATP6AP1L, ATP6AP2, ATP6VOA1, ATP6VOA2, ATP6VOA4, ATP6VOB, ATP6VOC, ATP6VOD1, ATP6VOD2, ATP6V0E1, ATP6VOE2, ATP6V1A, ATP6V1B1, ATP6V1B2, ATP6V1C1, ATP6V1C2, ATP6V1D, ATP6V1E1, ATP6V1E2, ATP6V1F, ATP6V1G1, ATP6V1G2, ATP6V1G2-DDX39B, ATP6V1G3, ATP6V1H, ATP7A, ATP7B, ATP8A1, ATP8A2, ATP8B1, ATP8B2, ATP8B3, ATP8B4, ATP9A, ATP9B, ATPAF1, ATPAF2, ATPIF1, ATR, ATRAID, ATRIP, ATRN, ATRNL1, ATRX, ATXN1, ATXN10, ATXN1L, ATXN2, ATXN2L, ATXN3, ATXN3L, ATXN7, ATXN7L1, ATXN7L2, ATXN7L3, ATXN7L3B, AUH, AUNIP, AUP1, AURKA, AURKAIP1, AURKB, AURKC, AUTS2, AVEN, AVIL, AVL9, AVP, AVPI1, AVPR1A, AVPR1B, AVPR2, AWAT1, AWAT2, AXDND1, AXIN1, AXIN2, AXL, AZGP1, AZI2, AZIN1, AZIN2, AZUl, B2M, B3GALNT1, B3GALNT2, B3GALT1, B3GALT2, B3GALT4, B3GALT5, B3GALT6, B3GAT1, B3GAT2, B3GAT3, B3GLCT, B3GNT2, B3GNT3, B3GNT4, B3GNT5, B3GNT6, B3GNT7, B3GNT8, B3GNT9, B3GNTL1, B4GALNT1, B4GALNT2, B4GALNT3, B4GALNT4, B4GALT1, B4GALT2, B4GALT3, B4GALT4, B4GALT5, B4GALT6, B4GALT7, B4GAT1, B9D1, B9D2, BAALC, BAAT, BABAM1, BABAM2, BACE1, BACE2, BACH1, BACH2, BAD, BAG1, BAG2, BAG3, BAG4, BAG5, BAG6, BAGE3, BAHCC1, BAHD1, BAIAP2, BAIAP2L1, BAIAP2L2, BAIAP3, BAK1, BAMBI, BANF1, BANF2, BANK1, BANP, BAP1, BARD1, BARHL1, BARHL2, BARX1, BARX2, BASP1, BATF, BATF2, BATF3, BAX, BAZ1A, BAZ1B, BAZ2A, BAZ2B, BBC3, BBIP1, BBOF1, BBOX1, BBS1, BBS10, BBS12, BBS2, BBS4, BBS5, BBS7, BBS9, BBX, BCAM, BCAN, BCAP29, BCAP31, BCAR1, BCAR3, BCAS1, BCAS2, BCAS3, BCAS4, BCAT1, BCAT2, BCCIP, BCDIN3D, BCHE, BCKDHA, BCKDHB, BCKDK, BCL10, BCL11A, BCL11B, BCL2, BCL2A1, BCL2L1, BCL2L10, BCL2L11, BCL2L12, BCL2L13, BCL2L14, BCL2L15, BCL2L2, BCL2L2-PABPN1, BCL3, BCL6, BCL6B, BCL7A, BCL7B, BCL7C, BCL9, BCL9L, BCLAF1, BCLAF3, BCO1, BCO2, BCOR, BCORL1, BCR, BCS1L, BDH1, BDH2, BDKRB1, BDKRB2, BDNF, BDP1, BEAN1, BECN1, BECN2, BEGAIN, BEND2, BEND3, BEND4, BEND5, BEND6, BEND7, BEST1, BEST2, BEST3, BEST4, BET1, BET1L, BEX1, BEX2, BEX3, BEX4, BEX5, BFAR, BFSP1, BFSP2, BGLAP, BGN, BHLHA15, BHLHA9, BHLHB9, BHLHE22, BHLHE23, BHLHE40, BHLHE41, BHMG1, BHMT, BHMT2, BICC1, BICD1, BICD2, BICDL1, BICDL2, BICRA, BICRAL, BID, BIK, BIN1, BIN2, BIN3, BIRC2, BIRC3, BIRC5, BIRC6, BIRC7, BIRC8, BIVM, BIVM-ERCC5, BLACE, BLCAP, BLID, BLK, BLM, BLMH, BLNK, BLOC1S1, BLOC1S2, BLOC1S3, BLOC1S4, BLOC1S5, BLOC1S5-TXNDC5, BLOC1S6, BLVRA, BLVRB, BLZF1, BMF, BMI1, BMP1, BMP10, BMP15, BMP2, BMP2K, BMP3, BMP4, BMP5, BMP6, BMP7, BMP8A, BMP8B, BMPER, BMPR1A, BMPR1B, BMPR2, BMS1, BMT2, BMX, BNC1, BNC2, BNIP1, BNIP2, BNIP3, BNIP3L, BNIPL, BOC, BOD1, BOD1L1, BOD1L2, BOK, BOLA1, BOLA2, BOLA2B, BOLA2-SMG1P6, BOLA3, BOLL, BOP1, BORA, BORCS5, BORCS6, BORCS7, BORCS7-ASMT, BORCS8, BORCS8-MEF2B, BPGM, BPHL, BPI, BPIFA1, BPIFA2, BPIFA3, BPIFB1, BPIFB2, BPIFB3, BPIFB4, BPIFB6, BPIFC, BPNT1, BPTF, BPY2, BPY2B, BPY2C, BRAF, BRAP, BRAT1, BRCA1, BRCA2, BRCC3, BRD1, BRD2, BRD3, BRD4, BRD7, BRD8, BRD9, BRDT, BRF1, BRF2, BRI3, BRI3BP, BRICD5, BRINP1, BRINP2, BRINP3, BRIP1, BRIX1, BRK1, BRMS1, BRMS1L, BROX, BRPF1, BRPF3, BRS3, BRSK1, BRSK2, BRWD1, BRWD3, BSCL2, BSDC1, BSG, BSN, BSND, BSPH1, BSPRY, BST1, BST2, BSX, BTAF1, BTBD1, BTBD10, BTBD11, BTBD16, BTBD17, BTBD18, BTBD19, BTBD2, BTBD3, BTBD6, BTBD7, BTBD8, BTBD9, BTC, BTD, BTF3, BTF3L4, BTG1, BTG2, BTG3, BTG4, BTK, BTLA, BTN1A1, BTN2A1, BTN2A2, BTN3A1, BTN3A2, BTN3A3, BTNL2, BTNL3, BTNL8, BTNL9, BTRC, BUB1, BUB1B, BUB1B-PAK6, BUB3, BUD13, BUD23, BUD31, BVES, BX004987.1, BX072566.1, BX088645.1, BX248244.1, BX248413.4, BX248415.1, BX248516.1, BX276092.9, BYSL, BZW1, BZW2, C10orf10, C10orf105, C10orf107, C10orf113, C10orf120, C10orf126, C10orf128, C10orf142, C10orf35, C10orf53, C10orf55, C10orf62, C10orf67, C10orf71, C10orf76, C10orf82, C10orf88, C10orf90, C10orf95, C10orf99, C11orf1, C11orf16, C11orf2l, C11orf24, C11orf40, C11orf42, C11orf45, C11orf49, C11orf52, C11orf53, C11orf54, C11orf57, C11orf58, C11orf63, C11orf65, C11orf68, C11orf70, C11orf71, C11orf74, C11orf80, C11orf84, C11orf86, C11orf87, C11orf88, C11orf91, C11orf94, C11orf95, C11orf96, C11orf97, C11orf98, C12orf10, C12orf29, C12orf4, C12orf40, C12orf42, C12orf43, C12orf45, C12orf49, C12orf50, C12orf54, C12orf56, C12orf57, C12orf60, C12orf65, C12orf66, C12orf71, C12orf73, C12orf74, C12orf75, C12orf76, C13orf42, C14orf105, C14orf119, C14orf132, C14orf159, C14orf166, C14orf177, C14orf178, C14orf180, C14orf2, C14orf28, C14orf37, C14orf39, C14orf79, C14orf80, C14orf93, C15orf38-AP3S2, C15orf39, C15orf40, C15orf41, C15orf48, C15orf52, C15orf53, C15orf59, C15orf61, C15orf62, C15orf65, C16orf45, C16orf46, C16orf52, C16orf54, C16orf58, C16orf59, C16orf62, C16orf70, C16orf71, C16orf72, C16orf74, C16orf78, C16orf82, C16orf86, C16orf87, C16orf89, C16orf90, C16orf91, C16orf92, C16orf95, C16orf96, C17orf100, C17orf105, C17orf107, C17orf113, C17orf47, C17orf49, C17orf50, C17orf51, C17orf53, C17orf58, C17orf62, C17orf64, C17orf67, C17orf74, C17orf75, C17orf78, C17orf80, C17orf97, C17orf98, C17orf99, C18orf21, C18orf25, C18orf32, C18orf54, C18orf63, C18orf8, C19orf12, C19orf18, C19orf24, C19orf25, C19orf33, C19orf35, C19orf38, C19orf44, C19orf47, C19orf48, C19orf53, C19orf54, C19orf57, C19orf60, C19orf66, C19orf67, C19orf68, C19orf70, C19orf71, C19orf73, C19orf81, C19orf84, C1D, C1GALT1, C1GALT1C1, C1GALT1C1L, C1orf100, C1orf105, C1orf109, C1orf112, C1orf115, C1orf116, C1orf122, C1orf123, C1orf127, C1orf131, C1orf141, C1orf146, C1orf158, C1orf159, C1orf162, C1orf167, C1orf174, C1orf185, C1orf186, C1orf189, C1orf194, C1orf198, C1orf21, C1orf210, C1orf216, C1orf226, C1orf228, C1orf232, C1orf27, C1orf35, C1orf43, C1orf50, C1orf52, C1orf53, C1orf54, C1orf56, C1orf6l, C1orf64, C1orf68, C1orf74, C1orf87, C1orf94, C1QA, C1QB, C1QBP, C1QC, C1QL1, C1QL2, C1QL3, C1QL4, C1QTNF1, C1QTNF12, C1QTNF2, C1QTNF3, C1QTNF3-AMACR, ClQTNF4, C1QTNF5, C1QTNF6, C1QTNF7, C1QTNF8, C1QTNF9, C1QTNF9B, ClR, C1RL, CIS, C2, C20orf141, C20orf144, C20orf173, C20orf194, C20orf196, C20orf202, C20orf204, C20orf24, C20orf27, C20orf85, C20orf96, C21orf140, C21orf2, C21orf33, C21orf58, C21orf59, C21orf62, C21orf91, C22orf15, C22orf23, C22orf31, C22orf39, C22orf42, C22orf46, C2CD2, C2CD2L, C2CD3, C2CD4A, C2CD4B, C2CD4C, C2CD4D, C2CD5, C2CD6, C2orf15, C2orf16, C2orf40, C2orf42, C2orf49, C2orf50, C2orf54, C2orf66, C2orf68, C2orf69, C2orf70, C2orf71, C2orf72, C2orf73, C2orf74, C2orf76, C2orf78, C2orf80, C2orf81, C2orf82, C2orf83, C2orf88, C2orf91, C3, C3AR1, C3orf14, C3orf18, C3orf20, C3orf22, C3orf30, C3orf33, C3orf35, C3orf36, C3orf38, C3orf49, C3orf52, C3orf56, C3orf58, C3orf62, C3orf67, C3orf70, C3orf80, C3orf84, C3orf85, C4A, C4B, C4B_2, C4BPA, C4BPB, C4orf17, C4orf19, C4orf22, C4orf26, C4orf3, C4orf32, C4orf33, C4orf36, C4orf45, C4orf46, C4orf47, C4orf48, C4orf50, C4orf51, C5, C5AR1, C5AR2, C5orf15, C5orf22, C5orf24, C5orf30, C5orf34, C5orf38, C5orf42, C5orf46, C5orf47, C5orf49, C5orf51, C5orf52, C5orf56, C5orf58, C5orf60, C5orf63, C5orf67, C6, C6orf10, C6orf106, C6orf118, C6orf120, C6orf132, C6orf136, C6orf141, C6orf15, C6orf163, C6orf201, C6orf203, C6orf222, C6orf223, C6orf226, C6orf229, C6orf47, C6orf48, C6orf52, C6orf58, C6orf62, C6orf89, C7, C7orf25, C7orf26, C7orf31, C7orf33, C7orf34, C7orf43, C7orf49, C7orf50, C7orf55-LUC7L2, C7orf57, C7orf61, C7orf72, C7orf73, C7orf77, C8A, C8B, C8G, C8orf22, C8orf33, C8orf34, C8orf37, C8orf4, C8orf44, C8orf44-SGK3, C8orf46, C8orf48, C8orf58, C8orf59, C8orf74, C8orf76, C8orf82, C8orf86, C8orf88, C8orf89, C9, C9orf116, C9orf129, C9orf131, C9orf135, C9orf152, C9orf153, C9orf16, C9orf172, C9orf24, C9orf3, C9orf40, C9orf43, C9orf47, C9orf50, C9orf57, C9orf64, C9orf66, C9orf72, C9orf78, C9orf84, C9orf85, C9orf92, CA1, CA10, CA11, CA12, CA13, CA14, CA2, CA3, CA4, CA5A, CA5B, CA6, CA7, CA8, CA9, CAAP1, CAB39, CAB39L, CABIN1, CABLES1, CABLES2, CABP1, CABP2, CABP4, CABP5, CABP7, CABS1, CABYR, CACFD1, CACHDI, CACNA1A, CACNAlB, CACNA1C, CACNA1D, CACNA1E, CACNA1F, CACNA1G, CACNA1H, CACNA1I, CACNAlS, CACNA2D1, CACNA2D2, CACNA2D3, CACNA2D4, CACNB1, CACNB2, CACNB3, CACNB4, CACNG1, CACNG2, CACNG3, CACNG4, CACNG5, CACNG6, CACNG7, CACNG8, CACTIN, CACULl, CACYBP, CAD, CADM1, CADM2, CADM3, CADM4, CADPS, CADPS2, CAGE1, CALB1, CALB2, CALCA, CALCB, CALCOCO1, CALCOCO2, CALCR, CALCRL, CALD1, CALHM1, CALHM2, CALHM3, CALM1, CALM2, CALM3, CALML3, CALML4, CALML5, CALML6, CALN1, CALR, CALR3, CALU, CALY, CAMK1, CAMKlD, CAMK1G, CAMK2A, CAMK2B, CAMK2D, CAMK2G, CAMK2N1, CAMK2N2, CAMK4, CAMKK1, CAMKK2, CAMKMT, CAMKV, CAMLG, CAMP, CAMSAP1, CAMSAP2, CAMSAP3, CAMTA1, CAMTA2, CAND1, CAND2, CANT1, CANX, CAP1, CAP2, CAPG, CAPN1, CAPN10, CAPN11, CAPN12, CAPN13, CAPN14, CAPN15, CAPN2, CAPN3, CAPN5, CAPN6, CAPN7, CAPN8, CAPN9, CAPNS1, CAPNS2, CAPRIN1, CAPRIN2, CAPS, CAPS2, CAPSL, CAPZA1, CAPZA2, CAPZA3, CAPZB, CARD10, CARD11, CARD14, CARD16, CARD17, CARD18, CARD19, CARD6, CARD8, CARD9, CARF, CARHSP1, CARM1, CARMIL1, CARMIL2, CARMIL3, CARNMT1, CARNS1, CARS, CARS2, CARTPT, CASC1, CASC10, CASC3, CASC4, CASD1, CASK, CASKIN1, CASKIN2, CASP1, CASP10, CASP12, CASP14, CASP2, CASP3, CASP4, CASP5, CASP6, CASP7, CASP8, CASP8AP2, CASP9, CASQ1, CASQ2, CASR, CASS4, CAST, CASTOR1, CASTOR₂, CASZ1, CAT, CATIP, CATSPER1, CATSPER2, CATSPER3, CATSPER4, CATSPERB, CATSPERD, CATSPERE, CATSPERG, CATSPERZ, CAV1, CAV2, CAV3, CAVIN1, CAVIN2, CAVIN3, CAVIN4, CBARP, CBFA2T2, CBFA2T3, CBFB, CBL, CBLB, CBLC, CBLL1, CBLN1, CBLN2, CBLN3, CBLN4, CBR1, CBR3, CBR4, CBS, CBSL, CBWD1, CBWD2, CBWD3, CBWD5, CBWD6, CBX1, CBX2, CBX3, CBX4, CBX5, CBX6, CBX7, CBX8, CBY1, CBY3, CC2D1A, CC2D1B, CC2D2A, CC2D2B, CCAR1, CCAR2, CCBE1, CCDC102A, CCDC102B, CCDC103, CCDC105, CCDC106, CCDC107, CCDC110, CCDC112, CCDC113, CCDC114, CCDC115, CCDC116, CCDC117, CCDC12, CCDC120, CCDC121, CCDC122, CCDC124, CCDC125, CCDC126, CCDC127, CCDC129, CCDC13, CCDC130, CCDC134, CCDC136, CCDC137, CCDC138, CCDC14, CCDC140, CCDC141, CCDC142, CCDC144A, CCDC144NL, CCDC146, CCDC148, CCDC149, CCDC15, CCDC150, CCDC151, CCDC152, CCDC153, CCDC154, CCDC155, CCDC157, CCDC158, CCDC159, CCDC160, CCDC163, CCDC166, CCDC167, CCDC168, CCDC169, CCDC169-SOHLH2, CCDC17, CCDC170, CCDC171, CCDC172, CCDC173, CCDC174, CCDC175, CCDC177, CCDC178, CCDC179, CCDC18, CCDC180, CCDC181, CCDC182, CCDC183, CCDC184, CCDC185, CCDC186, CCDC187, CCDC188, CCDC189, CCDC190, CCDC191, CCDC192, CCDC194, CCDC195, CCDC196, CCDC197, CCDC22, CCDC24, CCDC25, CCDC27, CCDC28A, CCDC28B, CCDC3, CCDC30, CCDC32, CCDC33, CCDC34, CCDC36, CCDC38, CCDC39, CCDC40, CCDC42, CCDC43, CCDC47, CCDC50, CCDC51, CCDC54, CCDC57, CCDC58, CCDC59, CCDC6, CCDC60, CCDC61, CCDC62, CCDC63, CCDC65, CCDC66, CCDC68, CCDC69, CCDC7, CCDC70, CCDC71, CCDC71L, CCDC73, CCDC74A, CCDC74B, CCDC77, CCDC78, CCDC8, CCDC80, CCDC81, CCDC82, CCDC83, CCDC84, CCDC85A, CCDC85B, CCDC85C, CCDC86, CCDC87, CCDC88A, CCDC88B, CCDC88C, CCDC89, CCDC9, CCDC90B, CCDC91, CCDC92, CCDC93, CCDC94, CCDC96, CCDC97, CCER1, CCER2, CCHCR1, CCIN, CCK, CCKAR, CCKBR, CCL1, CCL11, CCL13, CCL14, CCL15, CCL15-CCL14, CCL16, CCL17, CCL18, CCL19, CCL2, CCL20, CCL21, CCL22, CCL23, CCL24, CCL25, CCL26, CCL27, CCL28, CCL3, CCL3L1, CCL3L3, CCL4, CCL4L2, CCL5, CCL7, CCL8, CCM2, CCM2L, CCNA1, CCNA2, CCNB1, CCNB1IP1, CCNB2, CCNB3, CCNC, CCND1, CCND2, CCND3, CCNDBP1, CCNE1, CCNE2, CCNF, CCNG1, CCNG2, CCNH, CCNI, CCNI2, CCNJ, CCNJL, CCNK, CCNL1, CCNL2, CCNO, CCNT1, CCNT2, CCNY, CCNYL1, CCP110, CCPG1, CCR1, CCR10, CCR2, CCR3, CCR4, CCR5, CCR6, CCR7, CCR8, CCR9, CCRL2, CCS, CCSAP, CCSER1, CCSER2, CCT2, CCT3, CCT4, CCT5, CCT6A, CCT6B, CCT7, CCT8, CCT8L2, CCZ1, CCZ1B, CD101, CD109, CD14, CD151, CD160, CD163, CD163L1, CD164, CD164L2, CD177, CD180, CD19, CD1A, CD1B, CD1C, CD1D, CD1E, CD2, CD200, CD200R1, CD200R1L, CD207, CD209, CD22, CD226, CD24, CD244, CD247, CD248, CD27, CD274, CD276, CD28, CD2AP, CD2BP2, CD300A, CD300C, CD300E, CD300LB, CD300LD, CD300LF, CD300LG, CD302, CD320, CD33, CD34, CD36, CD37, CD38, CD3D, CD3E, CD3EAP, CD3G, CD4, CD40, CD40LG, CD44, CD46, CD47, CD48, CD5, CD52, CD53, CD55, CD58, CD59, CD5L, CD6, CD63, CD68, CD69, CD7, CD70, CD72, CD74, CD79A, CD79B, CD80, CD81, CD82, CD83, CD84, CD86, CD8A, CD8B, CD9, CD93, CD96, CD99, CD99L2, CDA, CDADC1, CDAN1, CDC123, CDC14A, CDC14B, CDC16, CDC20, CDC20B, CDC23, CDC25A, CDC25B, CDC25C, CDC26, CDC27, CDC34, CDC37, CDC37L1, CDC40, CDC42, CDC42BPA, CDC42BPB, CDC42BPG, CDC42EP1, CDC42EP2, CDC42EP3, CDC42EP4, CDC42EP5, CDC42SE1, CDC42SE2, CDC45, CDC5L, CDC6, CDC7, CDC73, CDCA2, CDCA3, CDCA4, CDCA5, CDCA7, CDCA7L, CDCA8, CDCP1, CDCP2, CDH1, CDH10, CDH11, CDH12, CDH13, CDH15, CDH16, CDH17, CDH18, CDH19, CDH2, CDH20, CDH22, CDH23, CDH24, CDH26, CDH3, CDH4, CDH5, CDH6, CDH7, CDH8, CDH9, CDHR1, CDHR2, CDHR3, CDHR4, CDHR5, CDIP1, CDIPT, CDK1, CDK10, CDK11 A, CDK11B, CDK12, CDK13, CDK14, CDK15, CDK16, CDK17, CDK18, CDK19, CDK20, CDK2AP1, CDK2AP2, CDK3, CDK4, CDK5, CDK5R1, CDK5R2, CDK5RAP1, CDK5RAP2, CDK5RAP3, CDK6, CDK7, CDK8, CDK9, CDKAL1, CDKL1, CDKL2, CDKL3, CDKL4, CDKL5, CDKN1A, CDKN1B, CDKN1C, CDKN2A, CDKN2AIP, CDKN2AIPNL, CDKN2B, CDKN2C, CDKN2D, CDKN3, CDNF, CDO1, CDON, CDPF1, CDR1, CDR2, CDR2L, CDRT1, CDRT15, CDRT15L2, CDRT4, CDS1, CDS2, CDSN, CDT1, CDV3, CDX1, CDX2, CDX4, CDY1, CDY1B, CDY2A, CDY2B, CDYL, CDYL2, CEACAMI, CEACAM16, CEACAM19, CEACAM20, CEACAM21, CEACAM3, CEACAM4, CEACAM5, CEACAM6, CEACAM7, CEACAM8, CEBPA, CEBPB, CEBPD, CEBPE, CEBPG, CEBPZ, CEBPZOS, CECR2, CEL, CELA1, CELA2A, CELA2B, CELA3A, CELA3B, CELF1, CELF2, CELF3, CELF4, CELF5, CELF6, CELSR1, CELSR2, CELSR3, CEMIP, CEMP1, CEND1, CENPA, CENPB, CENPBD1, CENPC, CENPE, CENPF, CENPH, CENPI, CENPJ, CENPK, CENPL, CENPM, CENPN, CENPO, CENPP, CENPQ, CENPS, CENPS-CORT, CENPT, CENPU, CENPV, CENPVL1, CENPVL2, CENPVL3, CENPW, CENPX, CEP104, CEP112, CEP120, CEP126, CEP128, CEP131, CEP135, CEP152, CEP162, CEP164, CEP170, CEP170B, CEP19, CEP192, CEP250, CEP290, CEP295, CEP295NL, CEP350, CEP41, CEP44, CEP55, CEP57, CEP57L1, CEP63, CEP68, CEP70, CEP72, CEP76, CEP78, CEP83, CEP85, CEP85L, CEP89, CEP95, CEP97, CEPT1, CER1, CERCAM, CERK, CERKL, CERS1, CERS2, CERS3, CERS4, CERS5, CERS6, CES1, CES2, CES3, CES4A, CESSA, CETN1, CETN2, CETN3, CETP, CFAP100, CFAP126, CFAP157, CFAP161, CFAP20, CFAP206, CFAP221, CFAP36, CFAP43, CFAP44, CFAP45, CFAP46, CFAP47, CFAP52, CFAP53, CFAP54, CFAP57, CFAP58, CFAP61, CFAP65, CFAP69, CFAP70, CFAP73, CFAP74, CFAP77, CFAP97, CFAP99, CFB, CFC1, CFC1B, CFD, CFDP1, CFH, CFHR1, CFHR2, CFHR3, CFHR4, CFHR5, CFI, CFL1, CFL2, CFLAR, CFP, CFTR, CGA, CGB1, CGB2, CGB3, CGB5, CGB7, CGB8, CGGBP1, CGN, CGNL1, CGREF1, CGRRF1, CH25H, CHAC1, CHAC2, CHAD, CHADL, CHAF1A, CHAFlB, CHAMP1, CHAT, CHCHD1, CHCHD10, CHCHD2, CHCHD3, CHCHD4, CHCHD5, CHCHD6, CHCHD7, CHD1, CHD1L, CHD2, CHD3, CHD4, CHD5, CHD6, CHD7, CHD8, CHD9, CHDH, CHEK1, CHEK2, CHERP, CHFR, CHGA, CHGB, CHI3L1, CHI3L2, CHIA, CHIC1, CHIC2, CHID1, CHIT1, CHKA, CHKB, CHKB-CPT1B, CHL1, CHM, CHML, CHMPlA, CHMPIB, CHMP2A, CHMP2B, CHMP3, CHMP4A, CHMP4B, CHMP4C, CHMP5, CHMP6, CHMP7, CHN1, CHN2, CHODL, CHORDC1, CHP1, CHP2, CHPF, CHPF2, CHPT1, CHRAC1, CHRD, CHRDL1, CHRDL2, CHRFAM7A, CHRM1, CHRM2, CHRM3, CHRM4, CHRM5, CHRNA1, CHRNA10, CHRNA2, CHRNA3, CHRNA4, CHRNA5, CHRNA6, CHRNA7, CHRNA9, CHRNB1, CHRNB2, CHRNB3, CHRNB4, CHRND, CHRNE, CHRNG, CHST1, CHST10, CHST11, CHST12, CHST13, CHST14, CHST15, CHST2, CHST3, CHST4, CHST5, CHST6, CHST7, CHST8, CHST9, CHSY1, CHSY3, CHTF18, CHTF8, CHTOP, CHUK, CHURC1, CHURC1-FNTB, CIAO1, CIAPIN1, CIART, CIB1, CIB2, CIB3, CIB4, CIC, CIDEA, CIDEB, CIDEC, CIITA, CILP, CILP2, CINP, CIPC, CIR1, CIRBP, CISD1, CISD2, CISD3, CISH, CIT, CITED1, CITED2, CITED4, CIZ1, CKAP2, CKAP2L, CKAP4, CKAP5, CKB, CKLF, CKLF-CMTM1, CKM, CKMT1A, CKMTlB, CKMT2, CKS1B, CKS2, CLASP1, CLASP2, CLASRP, CLC, CLCA1, CLCA2, CLCA4, CLCC1, CLCF1, CLCN1, CLCN2, CLCN3, CLCN4, CLCN5, CLCN6, CLCN7, CLCNKA, CLCNKB, CLDN1, CLDN10, CLDN11, CLDN12, CLDN14, CLDN15, CLDN16, CLDN17, CLDN18, CLDN19, CLDN2, CLDN20, CLDN22, CLDN23, CLDN24, CLDN25, CLDN3, CLDN34, CLDN4, CLDN5, CLDN6, CLDN7, CLDN8, CLDN9, CLDND1, CLDND2, CLEC10A, CLEC11A, CLEC12A, CLEC12B, CLEC14A, CLEC16A, CLEC17A, CLEC18A, CLEC18B, CLEC18C, CLEC19A, CLEC1A, CLEClB, CLEC20A, CLEC2A, CLEC2B, CLEC2D, CLEC2L, CLEC3A, CLEC3B, CLEC4A, CLEC4C, CLEC4D, CLEC4E, CLEC4F, CLEC4G, CLEC4M, CLEC5A, CLEC6A, CLEC7A, CLEC9A, CLECLI, CLGN, CLHC1, CLIC1, CLIC2, CLIC3, CLIC4, CLIC5, CLIC6, CLINT1, CLIP1, CLIP2, CLIP3, CLIP4, CLK1, CLK2, CLK3, CLK4, CLLU1, CLLU1OS, CLMN, CLMP, CLN3, CLN5, CLN6, CLN8, CLNK, CLNS1A, CLOCK, CLP1, CLPB, CLPP, CLPS, CLPSL1, CLPSL2, CLPTM1, CLPTM1L, CLPX, CLRN1, CLRN2, CLRN3, CLSPN, CLSTN1, CLSTN2, CLSTN3, CLTA, CLTB, CLTC, CLTCL1, CLU, CLUAP1, CLUH, CLUL1, CLVS1, CLVS2, CLYBL, CMA1, CMAS, CMBL, CMC1, CMC2, CMC4, CMIP, CMKLR1, CMPK1, CMPK2, CMSS1, CMTM1, CMTM2, CMTM3, CMTM4, CMTM5, CMTM6, CMTM7, CMTM8, CMTR1, CMTR2, CMYA5, CNBD1, CNBD2, CNBP, CNDP1, CNDP2, CNEP1R1, CNFN, CNGA1, CNGA2, CNGA3, CNGA4, CNGB1, CNGB3, CNIH1, CNIH2, CNIH3, CNIH4, CNKSR1, CNKSR2, CNKSR3, CNMD, CNN1, CNN2, CNN3, CNNM1, CNNM2, CNNM3, CNNM4, CNOT1, CNOT10, CNOT11, CNOT2, CNOT3, CNOT4, CNOT6, CNOT6L, CNOT7, CNOT8, CNOT9, CNP, CNPPD1, CNPY1, CNPY2, CNPY3, CNPY4, CNR1, CNR2, CNRIP1, CNST, CNTD1, CNTD2, CNTF, CNTFR, CNTLN, CNTN1, CNTN2, CNTN3, CNTN4, CNTN5, CNTN6, CNTNAP1, CNTNAP2, CNTNAP3, CNTNAP3B, CNTNAP4, CNTNAP5, CNTRL, CNTROB, COAl, COA3, COA4, COA5, COA6, COA7, COASY, COBL, COBLL1, COCH, COG1, COG2, COG3, COG4, COG5, COG6, COG7, COG8, COIL, COL10A1, COL11A1, COL11A2, COL12A1, COL13A1, COL14A1, COL15A1, COL16A1, COL17A1, COL18A1, COL19A1, COL1A1, COL1A2, COL20A1, COL21A1, COL22A1, COL23A1, COL24A1, COL25A1, COL26A1, COL27A1, COL28A1, COL2A1, COL3A1, COL4A1, COL4A2, COL4A3, COL4A3BP, COL4A4, COL4A5, COL4A6, COL5A1, COL5A2, COL5A3, COL6A1, COL6A2, COL6A3, COL6A5, COL6A6, COL7A1, COL8A1, COL8A2, COL9A1, COL9A2, COL9A3, COLCA2, COLEC10, COLEC11, COLEC12, COLGALT1, COLGALT2, COLQ, COMMD1, COMMD10, COMMD2, COMMD3, COMMD3-BMI1, COMMD4, COMMD5, COMMD6, COMMD7, COMMD8, COMMD9, COMP, COMT, COMTD1, COPA, COPB1, COPB2, COPE, COPG1, COPG2, COPRS, COPS2, COPS3, COPS4, COPS5, COPS6, COPS7A, COPS7B, COPS8, COPS9, COPZ1, COPZ2, COQ10A, COQ10B, COQ2, COQ3, COQ4, COQ5, COQ6, COQ7, COQ8A, COQ8B, COQ9, CORIN, CORO1A, COROIB, COROIC, CORO2A, CORO2B, CORO6, CORO7, CORO7-PAM16, CORT, COTL1, COX10, COX11, COX14, COX15, COX16, COX17, COX18, COX19, COX20, COX4I1, COX4I2, COX5A, COX5B, COX6A1, COX6A2, COX6B1, COX6B2, COX6C, COX7A1, COX7A2, COX7A2L, COX7B, COX7B2, COX7C, COX8A, COX8C, CP, CPA1, CPA2, CPA3, CPA4, CPA5, CPA6, CPAMD8, CPB1, CPB2, CPD, CPE, CPEB1, CPEB2, CPEB3, CPEB4, CPED1, CPLX1, CPLX2, CPLX3, CPLX4, CPM, CPN1, CPN2, CPNE1, CPNE2, CPNE3, CPNE4, CPNE5, CPNE6, CPNE7, CPNE8, CPNE9, CPO, CPOX, CPPED1, CPQ, CPS1, CPSF1, CPSF2, CPSF3, CPSF4, CPSF4L, CPSF6, CPSF7, CPT1A, CPT1B, CPT1C, CPT2, CPTP, CPVL, CPXCR1, CPXM1, CPXM2, CPZ, CR1, CR1L, CR2, CR354443.1, CR354443.2, CR388407.3, CR547123.3, CR753842.1, CR753845.2, CR759815.2, CR788250.1, CR847794.2, CR854858.1, CR933783.3, CR936239.1, CRABP1, CRABP2, CRACR2A, CRACR2B, CRADD, CRAMP1, CRAT, CRB1, CRB2, CRB3, CRBN, CRCP, CRCT1, CREB1, CREB3, CREB3L1, CREB3L2, CREB3L3, CREB3L4, CREB5, CREBBP, CREBL2, CREBRF, CREBZF, CREG1, CREG2, CRELD1, CRELD2, CREM, CRH, CRHBP, CRHR1, CRHR2, CRIM1, CRIP1, CRIP2, CRIP3, CRIPT, CRISP1, CRISP2, CRISP3, CRISPLD1, CRISPLD2, CRK, CRKL, CRLF1, CRLF2, CRLF3, CRLS1, CRMP1, CRNKL1, CRNN, CROCC, CROCC2, CROT, CRP, CRTAC1, CRTAM, CRTAP, CRTC1, CRTC2, CRTC3, CRX, CRY1, CRY2, CRYAA, CRYAB, CRYBA1, CRYBA2, CRYBA4, CRYBB1, CRYBB2, CRYBB3, CRYBG1, CRYBG2, CRYBG3, CRYGA, CRYGB, CRYGC, CRYGD, CRYGN, CRYGS, CRYL1, CRYM, CRYZ, CRYZL1, CS, CSAD, CSAG1, CSAG2, CSAG3, CSDC2, CSDE1, CSE1L, CSF1, CSF1R, CSF2, CSF2RA, CSF2RB, CSF3, CSF3R, CSGALNACT1, CSGALNACT2, CSH1, CSH2, CSHL1, CSK, CSMD1, CSMD2, CSMD3, CSN1S1, CSN2, CSN3, CSNK1A1, CSNK1A1L, CSNK1D, CSNK1E, CSNK1G1, CSNK1G2, CSNK1G3, CSNK2A1, CSNK2A2, CSNK2A3, CSNK2B, CSPG4, CSPG5, CSPP1, CSRNP1, CSRNP2, CSRNP3, CSRP1, CSRP2, CSRP3, CST1, CST11, CST2, CST3, CST4, CST5, CST6, CST7, CST8, CST9, CST9L, CSTA, CSTB, CSTF1, CSTF2, CSTF2T, CSTF3, CSTL1, CT45A1, CT45A10, CT45A2, CT45A3, CT45A5, CT45A6, CT45A7, CT45A8, CT45A9, CT476828.1, CT476828.10, CT476828.11, CT476828.12, CT476828.13, CT476828.14, CT476828.15, CT476828.16, CT476828.17, CT476828.18, CT476828.19, CT476828.2, CT476828.20, CT476828.21, CT476828.22, CT476828.3, CT476828.4, CT476828.5, CT476828.6, CT476828.7, CT476828.8, CT476828.9, CT47A1, CT47A10, CT47A11, CT47A12, CT47A2, CT47A3, CT47A4, CT47A5, CT47A6, CT47A7, CT47A8, CT47A9, CT47B1, CT55, CT62, CT83, CTAG1A, CTAG1B, CTAG2, CTAGEl, CTAGE15, CTAGE4, CTAGE5, CTAGE6, CTAGE8, CTAGE9, CTBP1, CTBP2, CTBS, CTC1, CTCF, CTCFL, CTDNEP1, CTDP1, CTDSP1, CTDSP2, CTDSPL, CTDSPL2, CTF1, CTGF, CTH, CTHRC1, CTIF, CTLA4, CTNNA1, CTNNA2, CTNNA3, CTNNAL1, CTNNB1, CTNNBIP1, CTNNBL1, CTNND1, CTNND2, CTNS, CTPS1, CTPS2, CTR9, CTRB1, CTRB2, CTRC, CTRL, CTSA, CTSB, CTSC, CTSD, CTSE, CTSF, CTSG, CTSH, CTSK, CTSL, CTSO, CTSS, CTSV, CTSW, CTSZ, CTTN, CTTNBP2, CTTNBP2NL, CTU1, CTU2, CTXN1, CTXN2, CTXN3, CTXND1, CU464060.1, CU633846.1, CU633980.1, CU633980.2, CU639417.1, CU639417.2, CUBN, CUEDC1, CUEDC2, CUL1, CUL2, CUL3, CUL4A, CUL4B, CUL5, CUL7, CUL9, CUTA, CUTC, CUX1, CUX2, CUZD1, CWC15, CWC22, CWC25, CWC27, CWF19L1, CWF19L2, CWH43, CX3CL1, CX3CR1, CXADR, CXCL1, CXCL10, CXCL11, CXCL12, CXCL13, CXCL14, CXCL16, CXCL17, CXCL2, CXCL3, CXCL5, CXCL6, CXCL8, CXCL9, CXCR1, CXCR2, CXCR3, CXCR4, CXCR5, CXCR6, CXorf21, CXorf36, CXorf38, CXorf40A, CXorf40B, CXorf49, CXorf49B, CXorf51A, CXorf51B, CXorf56, CXorf57, CXorf58, CXorf65, CXorf66, CXorf67, CXXC1, CXXC4, CXXC5, CYB561, CYB561A3, CYB561D1, CYB561D2, CYBSA, CYB5B, CYB5D1, CYB5D2, CYB5R1, CYB5R2, CYB5R3, CYB5R4, CYB5RL, CYBA, CYBB, CYBRD1, CYC1, CYCS, CYFIP1, CYFIP2, CYGB, CYHR1, CYLC1, CYLC2, CYLD, CYP11A1, CYP11B1, CYP11B2, CYP17A1, CYP19A1, CYP1A1, CYP1A2, CYP1B1, CYP20A1, CYP21A2, CYP24A1, CYP26A1, CYP26B1, CYP26C1, CYP27A1, CYP27B1, CYP27C1, CYP2A13, CYP2A6, CYP2A7, CYP2B6, CYP2C18, CYP2C19, CYP2C8, CYP2C9, CYP2D6, CYP2D7, CYP2E1, CYP2F1, CYP2J2, CYP2R1, CYP2S1, CYP2U1, CYP2W1, CYP39A1, CYP3A4, CYP3A43, CYP3A5, CYP3A7, CYP3A7-CYP3A51P, CYP46A1, CYP4A11, CYP4A22, CYP4B1, CYP4F11, CYP4F12, CYP4F2, CYP4F22, CYP4F3, CYP4F8, CYP4V2, CYP4X1, CYP4Z1, CYP51A1, CYP7A1, CYP7B1, CYP8B1, CYR61, CYS1, CYSLTR1, CYSLTR2, CYSRT1, CYSTM1, CYTH1, CYTH2, CYTH3, CYTH4, CYTIP, CYTL1, CYYR1, D2HGDH, DAAM1, DAAM2, DAB1, DAB2, DAB2IP, DACH1, DACH2, DACT1, DACT2, DACT3, DAD1, DAG1, DAGLA, DAGLB, DALRD3, DAND5, DAO, DAOA, DAP, DAP3, DAPK1, DAPK2, DAPK3, DAPL1, DAPP1, DARS, DARS2, DAW1, DAXX, DAZ1, DAZ2, DAZ3, DAZ4, DAZAP1, DAZAP2, DAZL, DBF4, DBF4B, DBH, DBI, DBN1, DBNDD1, DBNDD2, DBNL, DBP, DBR1, DBT, DBX1, DBX2, DCAF1, DCAF10, DCAF11, DCAF12, DCAF12L1, DCAF12L2, DCAF13, DCAF15, DCAF16, DCAF17, DCAF4, DCAF4L1, DCAF4L2, DCAF5, DCAF6, DCAF7, DCAF8, DCAF8L1, DCAF8L2, DCAKD, DCANP1, DCBLD1, DCBLD2, DCC, DCD, DCDC1, DCDC2, DCDC2B, DCDC2C, DCHS1, DCHS2, DCK, DCLK1, DCLK2, DCLK3, DCLRElA, DCLRElB, DCLRElC, DCN, DCP1A, DCP1B, DCP2, DCPS, DCST1, DCST2, DCSTAMP, DCT, DCTD, DCTN1, DCTN2, DCTN3, DCTN4, DCTN5, DCTN6, DCTPP1, DCUN1D1, DCUN1D2, DCUN1D3, DCUN1D4, DCUN1D5, DCX, DCXR, DDA1, DDAH1, DDAH2, DDB1, DDB2, DDC, DDHD1, DDHD2, DDI1, DDI2, DDIAS, DDIT3, DDIT4, DDIT4L, DDN, DDO, DDOST, DDR1, DDR2, DDRGK1, DDT, DDTL, DDX1, DDX10, DDX11, DDX17, DDX18, DDX19A, DDX19B, DDX20, DDX21, DDX23, DDX24, DDX25, DDX27, DDX28, DDX31, DDX39A, DDX39B, DDX3X, DDX3Y, DDX4, DDX41, DDX42, DDX43, DDX46, DDX47, DDX49, DDX5, DDX50, DDX51, DDX52, DDX53, DDX54, DDX55, DDX56, DDX58, DDX59, DDX6, DDX60, DDX60L, DEAF1, DECl, DECR1, DECR2, DEDD, DEDD2, DEF6, DEF8, DEFA1, DEFAIB, DEFA3, DEFA4, DEFA5, DEFA6, DEFB1, DEFB103A, DEFB103B, DEFB104A, DEFB104B, DEFB105A, DEFB105B, DEFB106A, DEFB106B, DEFB107A, DEFB107B, DEFB108B, DEFB110, DEFB112, DEFB113, DEFB114, DEFB115, DEFB116, DEFB118, DEFB119, DEFB121, DEFB123, DEFB124, DEFB125, DEFB126, DEFB127, DEFB128, DEFB129, DEFB130A, DEFB130B, DEFB131A, DEFB131B, DEFB132, DEFB133, DEFB134, DEFB135, DEFB136, DEFB4A, DEFB4B, DEGS1, DEGS2, DEK, DENND1A, DENND1B, DENND1C, DENND2A, DENND2C, DENND2D, DENND3, DENND4A, DENND4B, DENND4C, DENND5A, DENND5B, DENND6A, DENND6B, DENR, DEPDC1, DEPDClB, DEPDC4, DEPDC5, DEPDC7, DEPTOR, DERA, DERL1, DERL2, DERL3, DES, DESI1, DESI2, DET1, DEUP1, DEXI, DFFA, DFFB, DFNA5, DFNB59, DGAT1, DGAT2, DGAT2L6, DGCR2, DGCR6, DGCR6L, DGCR8, DGKA, DGKB, DGKD, DGKE, DGKG, DGKH, DGKI, DGKK, DGKQ, DGKZ, DGUOK, DHCR24, DHCR7, DHDDS, DHDH, DHFR, DHFR2, DHH, DHODH, DHPS, DHRS1, DHRS11, DHRS12, DHRS13, DHRS2, DHRS3, DHRS4, DHRS4L2, DHRS7, DHRS7B, DHRS7C, DHRS9, DHRSX, DHTKD1, DHX15, DHX16, DHX29, DHX30, DHX32, DHX33, DHX34, DHX35, DHX36, DHX37, DHX38, DHX40, DHX57, DHX58, DHX8, DHX9, DIABLO, DIAPHI, DIAPH2, DIAPH3, DICERI, DIDO1, DIEXF, DIMT1, DIO1, DIO2, DIO3, DIP2A, DIP2B, DIP2C, DIRAS1, DIRAS2, DIRAS3, DIRC1, DIRC2, DIRC3, DIS3, DIS3L, DIS3L2, DISCI, DISP1, DISP2, DISP3, DIXDC1, DKC1, DKK1, DKK2, DKK3, DKK4, DKKL1, DLAT, DLC1, DLD, DLEC1, DLEU7, DLG1, DLG2, DLG3, DLG4, DLG5, DLGAP1, DLGAP2, DLGAP3, DLGAP4, DLGAP5, DLK1, DLK2, DLL1, DLL3, DLL4, DLST, DLX1, DLX2, DLX3, DLX4, DLX5, DLX6, DMAC1, DMAC2, DMAP1, DMBT1, DMBX1, DMC1, DMD, DMGDH, DMKN, DMP1, DMPK, DMRT1, DMRT2, DMRT3, DMRTA1, DMRTA2, DMRTB1, DMRTC1, DMRTC1B, DMRTC2, DMTF1, DMTN, DMWD, DMXL1, DMXL2, DNA2, DNAAF1, DNAAF2, DNAAF3, DNAAF4, DNAAF5, DNAH1, DNAH10, DNAH10OS, DNAH11, DNAH12, DNAH14, DNAH17, DNAH2, DNAH3, DNAH5, DNAH6, DNAH7, DNAH8, DNAH9, DNAI1, DNAI2, DNAJA1, DNAJA2, DNAJA3, DNAJA4, DNAJB1, DNAJB11, DNAJB12, DNAJB13, DNAJB14, DNAJB2, DNAJB4, DNAJB5, DNAJB6, DNAJB7, DNAJB8, DNAJB9, DNAJC1, DNAJC10, DNAJC11, DNAJC12, DNAJC13, DNAJC14, DNAJC15, DNAJC16, DNAJC17, DNAJC18, DNAJC19, DNAJC2, DNAJC21, DNAJC22, DNAJC24, DNAJC25, DNAJC25-GNG10, DNAJC27, DNAJC28, DNAJC3, DNAJC30, DNAJC4, DNAJC5, DNAJC5B, DNAJC5G, DNAJC6, DNAJC7, DNAJC8, DNAJC9, DNAL1, DNAL4, DNALIl, DNASE1, DNASE1L1, DNASE1L2, DNASE1L3, DNASE2, DNASE2B, DND1, DNER, DNHD1, DNLZ, DNM1, DNM1L, DNM2, DNM3, DNMBP, DNMT1, DNMT3A, DNMT3B, DNMT3L, DNPEP, DNPH1, DNTT, DNTTIP1, DNTTIP2, DOC2A, DOC2B, DOCK1, DOCK10, DOCK11, DOCK2, DOCK3, DOCK4, DOCK5, DOCK6, DOCK7, DOCK8, DOCK9, DOHH, DOK1, DOK2, DOK3, DOK4, DOK5, DOK6, DOK7, DOLK, DOLPP1, DONSON, DOPEY1, DOPEY2, DOT1L, DPAGT1, DPCD, DPCR1, DPEP1, DPEP2, DPEP3, DPF1, DPF2, DPF3, DPH1, DPH2, DPH3, DPH5, DPH6, DPH7, DPM1, DPM2, DPM3, DPP10, DPP3, DPP4, DPP6, DPP7, DPP8, DPP9, DPPA2, DPPA3, DPPA4, DPPA5, DPRX, DPT, DPY19L1, DPY19L2, DPY19L3, DPY19L4, DPY30, DPYD, DPYS, DPYSL2, DPYSL3, DPYSL4, DPYSL5, DQX1, DR1, DRAM1, DRAM2, DRAP1, DRAXIN, DRC1, DRC3, DRC7, DRD1, DRD2, DRD3, DRD4, DRD5, DRG1, DRG2, DRGX, DRICHI, DROSHA, DRP2, DSC1, DSC2, DSC3, DSCAM, DSCAML1, DSCC1, DSCR3, DSCR4, DSCR8, DSE, DSEL, DSG1, DSG2, DSG3, DSG4, DSN1, DSP, DSPP, DST, DSTN, DSTYK, DTD1, DTD2, DTHD1, DTL, DTNA, DTNB, DTNBP1, DTWD1, DTWD2, DTX1, DTX2, DTX3, DTX3L, DTX4, DTYMK, DUOX1, DUOX2, DUOXA1, DUOXA2, DUPD1, DUS1L, DUS2, DUS3L, DUS4L, DUSP1, DUSP10, DUSP11, DUSP12, DUSP13, DUSP14, DUSP15, DUSP16, DUSP18, DUSP19, DUSP2, DUSP21, DUSP22, DUSP23, DUSP26, DUSP27, DUSP28, DUSP3, DUSP4, DUSP5, DUSP6, DUSP7, DUSP8, DUSP9, DUT, DUX4, DUXA, DUXB, DVL1, DVL2, DVL3, DWORF, DXO, DYDC1, DYDC2, DYM, DYNAP, DYNCIHI, DYNC111, DYNC112, DYNCILIl, DYNC1LI2, DYNC2H1, DYNC2LI1, DYNLL1, DYNLL2, DYNLRB1, DYNLRB2, DYNLT1, DYNLT3, DYRKIA, DYRKIB, DYRK2, DYRK3, DYRK4, DYSF, DYTN, DZANK1, DZIP1, DZIP1L, DZIP3, E2F1, E2F2, E2F3, E2F4, E2F5, E2F6, E2F7, E2F8, E4F1, EAF1, EAF2, EAPP, EARS2, EBAG9, EBF1, EBF2, EBF3, EBF4, EBI3, EBLN1, EBLN2, EBNA1BP2, EBP, EBPL, ECD, ECE1, ECE2, ECEL1, ECH1, ECHDC1, ECHDC2, ECHDC3, ECHS1, ECI1, ECI2, ECM1, ECM2, ECSCR, ECSIT, ECT2, ECT2L, EDA, EDA2R, EDAR, EDARADD, EDC3, EDC4, EDDM13, EDDM3A, EDDM3B, EDEM1, EDEM2, EDEM3, EDF1, EDIL3, EDN1, EDN2, EDN3, EDNRA, EDNRB, EDRF1, EEA1, EED, EEF1A1, EEF1A2, EEF1AKMT1, EEF1AKMT2, EEF1AKMT3, EEF1B2, EEFID, EEF1E1, EEF1E1-BLOC1S5, EEF1G, EEF2, EEF2K, EEF2KMT, EEFSEC, EEPD1, EFCAB1, EFCAB10, EFCAB11, EFCAB12, EFCAB13, EFCAB14, EFCAB2, EFCAB3, EFCAB5, EFCAB6, EFCAB7, EFCAB8, EFCAB9, EFCC1, EFEMP1, EFEMP2, EFHB, EFHC1, EFHC2, EFHD1, EFHD2, EFL1, EFNA1, EFNA2, EFNA3, EFNA4, EFNA5, EFNB1, EFNB2, EFNB3, EFR3A, EFR3B, EFS, EFTUD2, EGF, EGFL6, EGFL7, EGFL8, EGFLAM, EGFR, EGLN1, EGLN2, EGLN3, EGR1, EGR2, EGR3, EGR4, EHBP1, EHBP1L1, EHD1, EHD2, EHD3, EHD4, EHF, EHHADH, EHMT1, EHMT2, E124, EID1, EID2, EID2B, EID3, EIF1, EIF1AD, EIF1AX, EIF1AY, EIF1B, EIF2A, EIF2AK1, EIF2AK2, EIF2AK3, EIF2AK4, EIF2B1, EIF2B2, EIF2B3, EIF2B4, EIF2B5, EIF2D, EIF2S1, EIF2S2, EIF2S3, EIF3A, EIF3B, EIF3C, EIF3CL, EIF3D, EIF3E, EIF3F, EIF3G, EIF3H, EIF3I, EIF3J, EIF3K, EIF3L, EIF3M, EIF4A1, EIF4A2, EIF4A3, EIF4B, EIF4E, EIF4ElB, EIF4E2, EIF4E3, EIF4EBP1, EIF4EBP2, EIF4EBP3, EIF4ENIF1, EIF4G1, EIF4G2, EIF4G3, EIF4H, EIF5, EIF5A, EIF5A2, EIF5AL1, EIF5B, EIF6, EIPR1, ELAC1, ELAC2, ELANE, ELAVL1, ELAVL2, ELAVL3, ELAVL4, ELF1, ELF2, ELF3, ELF4, ELF5, ELFN1, ELFN2, ELK1, ELK3, ELK4, ELL, ELL2, ELL3, ELMO1, ELMO2, ELMO3, ELMOD1, ELMOD2, ELMOD3, ELMSAN1, ELN, ELOA, ELOA2, ELOA3, ELOA3B, ELOA3C, ELOA3D, ELOB, ELOC, ELOF1, ELOVL1, ELOVL2, ELOVL3, ELOVL4, ELOVL5, ELOVL6, ELOVL7, ELP1, ELP2, ELP3, ELP4, ELP5, ELP6, ELSPBP1, EMB, EMC1, EMC10, EMC2, EMC3, EMC4, EMC6, EMC7, EMC8, EMC9, EMCN, EMD, EME1, EME2, EMG1, EMID1, EMILIN1, EMILIN2, EMILIN3, EML1, EML2, EML3, EML4, EML5, EML6, EMP1, EMP2, EMP3, EMSY, EMX1, EMX2, EN1, EN2, ENAH, ENAM, ENC1, ENDODI, ENDOG, ENDOU, ENDOV, ENG, ENGASE, ENHO, ENKD1, ENKUR, ENO1, ENO2, ENO3, ENO4, ENOPHI, ENOSFI, ENOX1, ENOX2, ENPEP, ENPP1, ENPP2, ENPP3, ENPP4, ENPP5, ENPP6, ENPP7, ENSA, ENTHDI, ENTPD1, ENTPD2, ENTPD3, ENTPD4, ENTPD5, ENTPD6, ENTPD7, ENTPD8, ENY2, EOGT, EOMES, EP300, EP400, EPAS1, EPB41, EPB41L1, EPB41L2, EPB41L3, EPB41L4A, EPB41L4B, EPB41L5, EPB42, EPC1, EPC2, EPCAM, EPDR1, EPG5, EPGN, EPHA1, EPHA10, EPHA2, EPHA3, EPHA4, EPHA5, EPHA6, EPHA7, EPHA8, EPHB1, EPHB2, EPHB3, EPHB4, EPHB6, EPHX1, EPHX2, EPHX3, EPHX4, EPM2A, EPM2AIP1, EPN1, EPN2, EPN3, EPO, EPOP, EPOR, EPPIN, EPPIN-WFDC6, EPPK1, EPRS, EPS15, EPS15L1, EPS8, EPS8L1, EPS8L2, EPS8L3, EPSTIl, EPX, EPYC, EQTN, ERAL1, ERAP1, ERAP2, ERAS, ERBB2, ERBB3, ERBB4, ERBIN, ERC1, ERC2, ERCC1, ERCC2, ERCC3, ERCC4, ERCC5, ERCC6, ERCC6L, ERCC6L2, ERCC8, EREG, ERF, ERFE, ERG, ERG28, ERGIC1, ERGIC2, ERGIC3, ERH, ERI1, ERI2, ERI3, ERICH1, ERICH2, ERICH3, ERICH4, ERICH5, ERICH6, ERICH6B, ERLEC1, ERLIN1, ERLIN2, ERMAP, ERMARD, ERMN, ERMP1, ERN1, ERN2, ERO1A, ERO1B, ERP27, ERP29, ERP44, ERRFI1, ERV3-1, ERVFRD-1, ERVMER34-1, ERVV-1, ERVV-2, ERVW-1, ESAM, ESCO1, ESCO2, ESD, ESF1, ESM1, ESPL1, ESPN, ESPNL, ESR1, ESR2, ESRP1, ESRP2, ESRRA, ESRRB, ESRRG, ESS2, ESX1, ESYT1, ESYT2, ESYT3, ETAA1, ETDA, ETDB, ETDC, ETF1, ETFA, ETFB, ETFBKMT, ETFDH, ETFRF1, ETHEl, ETNK1, ETNK2, ETNPPL, ETS1, ETS2, ETV1, ETV2, ETV3, ETV3L, ETV4, ETV5, ETV6, ETV7, EVA1A, EVA1B, EVA1C, EVC, EVC2, EVI2A, EVI2B, EVI5, EVI5L, EVL, EVPL, EVPLL, EVX1, EVX2, EWSR1, EXD1, EXD2, EXD3, EXO1, EX05, EXOC1, EXOCIL, EXOC2, EXOC3, EXOC3L1, EXOC3L2, EXOC3L4, EXOC4, EXOC5, EXOC6, EXOC6B, EXOC7, EXOC8, EXOG, EXOSC1, EXOSC10, EXOSC2, EXOSC3, EXOSC4, EXOSC5, EXOSC6, EXOSC7, EXOSC8, EXOSC9, EXPH5, EXT1, EXT2, EXTL1, EXTL2, EXTL3, EYA1, EYA2, EYA3, EYA4, EYS, EZH1, EZH2, EZR, F10, F11, F1R, F12, F13A1, F13B, F2, F2R, F2RL1, F2RL2, F2RL3, F3, F5, F7, F8, F8A1, F8A2, F8A3, F9, FA2H, FAAH, FAAH2, FAAP100, FAAP20, FAAP24, FABP1, FABP12, FABP2, FABP3, FABP4, FABP5, FABP6, FABP7, FABP9, FADD, FADS1, FADS2, FADS3, FADS6, FAF1, FAF2, FAH, FAHD1, FAHD2A, FAHD2B, FAIM, FAIM2, FAM102A, FAM102B, FAM103A1, FAM104A, FAM104B, FAM105A, FAM106A, FAM107A, FAM107B, FAM109A, FAM109B, FAM110A, FAM110B, FAM110C, FAM110D, FAM111A, FAM111B, FAM114A1, FAM114A2, FAM117A, FAM117B, FAM118A, FAM118B, FAM120A, FAM120AOS, FAM120B, FAM120C, FAM122A, FAM122B, FAM122C, FAM124A, FAM124B, FAM126A, FAM126B, FAM129A, FAM129B, FAM129C, FAM131A, FAM131B, FAM131C, FAM133A, FAM133B, FAM135A, FAM135B, FAM136A, FAM13A, FAM13B, FAM13C, FAM149A, FAM149B1, FAM151A, FAM151B, FAM153A, FAM153B, FAM153C, FAM155A, FAM155B, FAM156A, FAM156B, FAM159A, FAM159B, FAM160A1, FAM160A2, FAM160B1, FAM160B2, FAM161A, FAM161B, FAM162A, FAM162B, FAM163A, FAM163B, FAM166A, FAM166B, FAM167A, FAM167B, FAM168A, FAM168B, FAM169A, FAM169B, FAM170A, FAM170B, FAM171A1, FAM171A2, FAM171B, FAM172A, FAM173A, FAM173B, FAM174A, FAM174B, FAM177A1, FAM177B, FAM178B, FAM180A, FAM180B, FAM181A, FAM181B, FAM182B, FAM183A, FAM184A, FAM184B, FAM185A, FAM186A, FAM186B, FAM187A, FAM187B, FAM189A1, FAM189A2, FAM189B, FAM192A, FAM193A, FAM193B, FAM196A, FAM196B, FAM198A, FAM198B, FAM199X, FAM19A1, FAM19A2, FAM19A3, FAM19A4, FAM19A5, FAM200A, FAM200B, FAM204A, FAM205A, FAM205C, FAM206A, FAM207A, FAM208A, FAM208B, FAM209A, FAM209B, FAM20A, FAM20B, FAM20C, FAM210A, FAM210B, FAM212A, FAM212B, FAM213A, FAM213B, FAM214A, FAM214B, FAM216A, FAM216B, FAM217A, FAM217B, FAM218A, FAM219A, FAM219B, FAM220A, FAM221A, FAM221B, FAM222A, FAM222B, FAM227A, FAM227B, FAM228A, FAM228B, FAM229A, FAM229B, FAM230A, FAM231A, FAM231B, FAM231C, FAM231D, FAM234A, FAM234B, FAM236A, FAM236B, FAM236C, FAM236D, FAM237A, FAM237B, FAM240A, FAM240B, FAM24A, FAM24B, FAM25A, FAM25C, FAM25G, FAM26D, FAM26E, FAM26F, FAM32A, FAM35A, FAM3A, FAM3B, FAM3C, FAM3D, FAM43A, FAM43B, FAM45A, FAM46A, FAM46B, FAM46C, FAM46D, FAM47A, FAM47B, FAM47C, FAM47E, FAM47E-STBD1, FAM49A, FAM49B, FAM50A, FAM50B, FAM53A, FAM53B, FAM53C, FAM57A, FAM57B, FAM58A, FAM60A, FAM69A, FAM69B, FAM69C, FAM71A, FAM71B, FAM71C, FAM71D, FAM71E1, FAM71E2, FAM71F1, FAM71F2, FAM72A, FAM72B, FAM72C, FAM72D, FAM76A, FAM76B, FAM78A, FAM78B, FAM81A, FAM81B, FAM83A, FAM83B, FAM83C, FAM83D, FAM83E, FAM83F, FAM83G, FAM83H, FAM84A, FAM84B, FAM86B1, FAM86B2, FAM86C1, FAM89A, FAM89B, FAM8A1, FAM90A1, FAM90A26, FAM91A1, FAM92A, FAM92B, FAM95C, FAM96A, FAM96B, FAM98A, FAM98B, FAM98C, FAM9A, FAM9B, FAM9C, FAN1, FANCA, FANCB, FANCC, FANCD2, FANCD2OS, FANCE, FANCF, FANCG, FANCI, FANCL, FANCM, FANK1, FAP, FAR1, FAR2, FARP1, FARP2, FARS2, FARSA, FARSB, FAS, FASLG, FASN, FASTK, FASTKD1, FASTKD2, FASTKD3, FASTKD5, FAT1, FAT2, FAT3, FAT4, FATE1, FAU, FAXC, FAXDC2, FBF1, FBL, FBLIM1, FBLL1, FBLN1, FBLN2, FBLN5, FBLN7, FBN1, FBN2, FBN3, FBP1, FBP2, FBRS, FBRSL1, FBXL12, FBXL13, FBXL14, FBXL15, FBXL16, FBXL17, FBXL18, FBXL19, FBXL2, FBXL20, FBXL22, FBXL3, FBXL4, FBXL5, FBXL6, FBXL7, FBXL8, FBXO10, FBXO11, FBX015, FBX016, FBX017, FBX018, FBXO2, FBXO21, FBXO22, FBXO24, FBXO25, FBXO27, FBXO28, FBXO3, FBXO30, FBXO31, FBXO32, FBXO33, FBXO34, FBXO36, FBXO38, FBXO39, FBXO4, FBXO40, FBXO41, FBXO42, FBXO43, FBXO44, FBXO45, FBXO46, FBXO47, FBXO48, FBX05, FBXO6, FBXO7, FBXO8, FBXO9, FBXW10, FBXW11, FBXW12, FBXW2, FBXW4, FBXW5, FBXW7, FBXW8, FBXW9, FCAMR, FCAR, FCER1A, FCER1G, FCER2, FCF1, FCGBP, FCGR1A, FCGR1B, FCGR2A, FCGR2B, FCGR2C, FCGR3A, FCGR3B, FCGRT, FCHO1, FCHO2, FCHSD1, FCHSD2, FCMR, FCN1, FCN2, FCN3, FCRL1, FCRL2, FCRL3, FCRL4, FCRL5, FCRL6, FCRLA, FCRLB, FDCSP, FDFT1, FDPS, FDX1, FDX2, FDXACB1, FDXR, FECH, FEM1A, FEM1B, FEM1C, FEN1, FER, FER1L5, FER1L6, FERD3L, FERMT1, FERMT2, FERMT3, FES, FETUB, FEV, FEZ1, FEZ2, FEZF1, FEZF2, FFAR1, FFAR2, FFAR3, FFAR4, FGA, FGB, FGD1, FGD2, FGD3, FGD4, FGD5, FGD6, FGF1, FGF10, FGF11, FGF12, FGF13, FGF14, FGF16, FGF17, FGF18, FGF19, FGF2, FGF20, FGF21, FGF22, FGF23, FGF3, FGF4, FGF5, FGF6, FGF7, FGF8, FGF9, FGFBP1, FGFBP2, FGFBP3, FGFR1, FGFR1OP, FGFR1OP2, FGFR2, FGFR3, FGFR4, FGFRL1, FGG, FGGY, FGL1, FGL2, FGR, FH, FHAD1, FHDC1, FHIT, FHL1, FHL2, FHL3, FHL5, FHOD1, FHOD3, FIBCD1, FIBIN, FIBP, FICD, FIG. 4, FIGLA, FIGN, FIGNL1, FIGNL2, FILIP1, FILIP1L, FIP1L1, FIS1, FITM1, FITM2, FIZ1, FJX1, FKBP10, FKBP11, FKBP14, FKBP15, FKBPlA, FKBP1B, FKBP1C, FKBP2, FKBP3, FKBP4, FKBP5, FKBP6, FKBP7, FKBP8, FKBP9, FKBPL, FKRP, FKTN, FLAD1, FLCN, FLG, FLG2, FLI1, FLII, FLNA, FLNB, FLNC, FLOT1, FLOT2, FLRT1, FLRT2, FLRT3, FLT1, FLT3, FLT3LG, FLT4, FLVCR1, FLVCR2, FLYWCH1, FLYWCH2, FMC1, FMN1, FMN2, FMNL1, FMNL2, FMNL3, FMO1, FMO2, FMO3, FMO4, FMO5, FMOD, FMR1, FMR1NB, FN1, FN3K, FN3KRP, FNBP1, FNBP1L, FNBP4, FNDC1, FNDC10, FNDC11, FNDC3A, FNDC3B, FNDC4, FNDC5, FNDC7, FNDC8, FNDC9, FNIP1, FNIP2, FNTA, FNTB, F0681492.1, F0681542.1, FOCAD, FOLH1, FOLR1, FOLR2, FOLR3, FOPNL, FOS, FOSB, FOSL1, FOSL2, FOXA1, FOXA2, FOXA3, FOXB1, FOXB2, FOXC1, FOXC2, FOXD1, FOXD2, FOXD3, FOXD4, FOXD4L1, FOXD4L3, FOXD4L4, FOXD4L5, FOXD4L6, FOXE1, FOXE3, FOXF1, FOXF2, FOXG1, FOXH1, FOXI1, FOXI2, FOXI3, FOXJ1, FOXJ2, FOXJ3, FOXK1, FOXK2, FOXL1, FOXL2, FOXL2NB, FOXM1, FOXN1, FOXN2, FOXN3, FOXN4, FOXO1, FOXO3, FOXO4, FOXO6, FOXP1, FOXP2, FOXP3, FOXP4, FOXQ1, FOXR1, FOXR2, FOXRED1, FOXRED2, FOXS1, FP236240.1, FP565260.1, FP565260.2, FP565260.3, FP565260.4, FP565260.6, FP565260.7, FP565324.1, FP565324.2, FPGS, FPGT, FPGT-TNNI3K, FPR1, FPR2, FPR3, FRA10AC1, FRAS1, FRAT1, FRAT2, FREM1, FREM2, FREM3, FRG1, FRG2, FRG2B, FRG2C, FRK, FRMD1, FRMD3, FRMD4A, FRMD4B, FRMD5, FRMD6, FRMD7, FRMD8, FRMPD1, FRMPD2, FRMPD3, FRMPD4, FRRS1, FRRS1L, FRS2, FRS3, FRY, FRYL, FRZB, FSBP, FSCB, FSCN1, FSCN2, FSCN3, FSD1, FSD1L, FSD2, FSHB, FSHR, FSIP1, FSIP2, FST, FSTL1, FSTL3, FSTL4, FSTL5, FTCD, FTCDNL1, FTH1, FTHL17, FTL, FTMT, FTO, FTSJ1, FTSJ3, FUBP1, FUBP3, FUCA1, FUCA2, FUK, FUNDC1, FUNDC2, FUOM, FURIN, FUS, FUT1, FUT10, FUT11, FUT2, FUT3, FUT4, FUT5, FUT6, FUT7, FUT8, FUT9, FUZ, FXN, FXR1, FXR2, FXYD1, FXYD2, FXYD3, FXYD4, FXYD5, FXYD6, FXYD6-FXYD2, FXYD7, FYB1, FYB2, FYCO1, FYN, FYTTD1, FZD1, FZD10, FZD2, FZD3, FZD4, FZD5, FZD6, FZD7, FZD8, FZD9, FZR1, GOS2, G2E3, G3BP1, G3BP2, G6PC, G6PC2, G6PC3, G6PD, GAA, GAB1, GAB2, GAB3, GAB4, GABARAP, GABARAPL1, GABARAPL2, GABBR1, GABBR₂, GABPA, GABPB1, GABPB2, GABRA1, GABRA2, GABRA3, GABRA4, GABRA5, GABRA6, GABRB1, GABRB2, GABRB3, GABRD, GABRE, GABRG1, GABRG2, GABRG3, GABRP, GABRQ, GABRR1, GABRR2, GABRR3, GAD1, GAD2, GADD45A, GADD45B, GADD45G, GADD45GIP1, GADL1, GAGE1, GAGE10, GAGE12B, GAGE12C, GAGE12D, GAGE12E, GAGE12F, GAGE12G, GAGE12H, GAGE12J, GAGE13, GAGE2A, GAGE2E, GAK, GAL, GAL3ST1, GAL3ST2, GAL3ST3, GAL3ST4, GALC, GALE, GALK1, GALK2, GALM, GALNS, GALNT1, GALNT10, GALNT11, GALNT12, GALNT13, GALNT14, GALNT15, GALNT16, GALNT17, GALNT18, GALNT2, GALNT3, GALNT4, GALNT5, GALNT6, GALNT7, GALNT8, GALNT9, GALNTL5, GALNTL6, GALP, GALR1, GALR2, GALR3, GALT, GAMT, GAN, GANAB, GANC, GAP43, GAPDH, GAPDHS, GAPT, GAPVD1, GAR1, GAREM1, GAREM2, GARNL3, GARS, GART, GAS1, GAS2, GAS2L1, GAS2L2, GAS2L3, GAS6, GAS7, GAS8, GAST, GATA1, GATA2, GATA3, GATA4, GATA5, GATA6, GATAD1, GATAD2A, GATAD2B, GATB, GATC, GATD1, GATM, GATS, GBA, GBA2, GBA3, GBE1, GBF1, GBGT1, GBP1, GBP2, GBP3, GBP4, GBP5, GBP6, GBP7, GBX1, GBX2, GC, GCA, GCAT, GCC1, GCC2, GCDH, GCFC2, GCG, GCGR, GCH1, GCHFR, GCK, GCKR, GCLC, GCLM, GCM1, GCM2, GCN1, GCNA, GCNT1, GCNT2, GCNT3, GCNT4, GCNT7, GCOM1, GCSAM, GCSAML, GCSH, GDA, GDAP1, GDAP1L1, GDAP2, GDE1, GDF1, GDF10, GDF11, GDF15, GDF2, GDF3, GDF5, GDF5OS, GDF6, GDF7, GDF9, GDI1, GDI2, GDNF, GDPD1, GDPD2, GDPD3, GDPD4, GDPD5, GDPGP1, GEM, GEMIN2, GEMIN4, GEMIN5, GEMIN6, GEMIN7, GEMIN8, GEN1, GET4, GFAP, GFER, GFI1, GFI1B, GFM1, GFM2, GFOD1, GFOD2, GFPT1, GFPT2, GFRA1, GFRA2, GFRA3, GFRA4, GFRAL, GFY, GGA1, GGA2, GGA3, GGACT, GGCT, GGCX, GGH, GGN, GGNBP2, GGPS1, GGT1, GGT2, GGT5, GGT6, GGT7, GGTLC1, GGTLC2, GGTLC3, GH1, GH2, GHDC, GHITM, GHR, GHRH, GHRHR, GHRL, GHSR, GID4, GID8, GIF, GIGYF1, GIGYF2, GIMAP1, GIMAP1-GIMAP5, GIMAP2, GIMAP4, GIMAP5, GIMAP6, GIMAP7, GIMAP8, GIMD1, GIN1, GINM1, GINS1, GINS2, GINS3, GINS4, GIP, GIPC1, GIPC2, GIPC3, GIPR, GIT1, GIT2, GJA1, GJA10, GJA3, GJA4, GJA5, GJA8, GJA9, GJB1, GJB2, GJB3, GJB4, GJB5, GJB6, GJB7, GJC1, GJC2, GJC3, GJD2, GJD3, GJD4, GJE1, GK, GK2, GK3P, GK5, GKAP1, GKN1, GKN2, GLA, GLB1, GLB1L, GLB1L2, GLB1L3, GLCCIl, GLCE, GLDC, GLDN, GLE1, GLG1, GLI1, GLI2, GLI3, GLI4, GLIPR1, GLIPR1L1, GLIPR1L2, GLIPR2, GLIS1, GLIS2, GLIS3, GLMN, GLMP, GLO1, GLOD4, GLOD5, GLP1R, GLP2R, GLRA1, GLRA2, GLRA3, GLRA4, GLRB, GLRX, GLRX2, GLRX3, GLRX5, GLS, GLS2, GLT1D1, GLT6D1, GLT8D1, GLT8D2, GLTP, GLTPD2, GLUD1, GLUD2, GLUL, GLYAT, GLYATL1, GLYATL1P3, GLYATL2, GLYATL3, GLYCTK, GLYR1, GM2A, GMCL1, GMDS, GMEB1, GMEB2, GMFB, GMFG, GMIP, GML, GMNC, GMNN, GMPPA, GMPPB, GMPR, GMPR2, GMPS, GNA11, GNA12, GNA13, GNA14, GNA15, GNAI1, GNAI2, GNAI3, GNAL, GNAO1, GNAQ, GNAS, GNAT1, GNAT2, GNAT3, GNAZ, GNB1, GNB1L, GNB2, GNB3, GNB4, GNB5, GNE, GNG10, GNG11, GNG12, GNG13, GNG14, GNG2, GNG3, GNG4, GNG5, GNG7, GNG8, GNGT1, GNGT2, GNL1, GNL2, GNL3, GNL3L, GNLY, GNMT, GNPAT, GNPDA1, GNPDA2, GNPNAT1, GNPTAB, GNPTG, GNRH1, GNRH2, GNRHR, GNS, GOLGA1, GOLGA2, GOLGA3, GOLGA4, GOLGA5, GOLGA6A, GOLGA6B, GOLGA6C, GOLGA6D, GOLGA6L1, GOLGA6L10, GOLGA6L2, GOLGA6L22, GOLGA6L4, GOLGA6L6, GOLGA6L7P, GOLGA6L9, GOLGA7, GOLGA7B, GOLGA8A, GOLGA8B, GOLGA8F, GOLGA8G, GOLGA8H, GOLGA8J, GOLGA8K, GOLGA8M, GOLGA8N, GOLGA80, GOLGA8Q, GOLGA8R, GOLGA8S, GOLGA8T, GOLGB1, GOLIM4, GOLM1, GOLPH3, GOLPH3L, GOLTIA, GOLTIB, GON4L, GON7, GOPC, GORAB, GORASP1, GORASP2, GOSR1, GOSR2, GOT1, GOT1L1, GOT2, GP1BA, GP1BB, GP2, GP5, GP6, GP9, GPA33, GPAA1, GPALPP1, GPAM, GPANK1, GPAT2, GPAT3, GPAT4, GPATCH1, GPATCH11, GPATCH2, GPATCH2L, GPATCH3, GPATCH4, GPATCH8, GPBAR1, GPBP1, GPBP1L1, GPC1, GPC2, GPC3, GPC4, GPC5, GPC6, GPCPD1, GPD1, GPD1L, GPD2, GPER1, GPHA2, GPHB5, GPHN, GPI, GPIHBP1, GPKOW, GPLD1, GPM6A, GPM6B, GPN1, GPN2, GPN3, GPNMB, GPR1, GPR101, GPR107, GPR108, GPR119, GPR12, GPR132, GPR135, GPR137, GPR137B, GPR137C, GPR139, GPR141, GPR142, GPR143, GPR146, GPR148, GPR149, GPR15, GPR150, GPR151, GPR152, GPR153, GPR155, GPR156, GPR157, GPR158, GPR160, GPR161, GPR162, GPR17, GPR171, GPR173, GPR174, GPR176, GPR179, GPR18, GPR180, GPR182, GPR183, GPR19, GPR20, GPR21, GPR22, GPR25, GPR26, GPR27, GPR3, GPR31, GPR32, GPR33, GPR34, GPR35, GPR37, GPR37L1, GPR39, GPR4, GPR42, GPR45, GPR50, GPR52, GPR55, GPR6, GPR61, GPR62, GPR63, GPR65, GPR68, GPR75, GPR75-ASB3, GPR78, GPR82, GPR83, GPR84, GPR85, GPR87, GPR88, GPR89A, GPR89B, GPRASP1, GPRASP2, GPRC5A, GPRC5B, GPRC5C, GPRC5D, GPRC6A, GPRIN1, GPRIN2, GPRIN3, GPS1, GPS2, GPSM1, GPSM2, GPSM3, GPT, GPT2, GPX1, GPX2, GPX3, GPX4, GPX5, GPX6, GPX7, GPX8, GRAMD1A, GRAMD1B, GRAMD1C, GRAMD2A, GRAMD2B, GRAMD4, GRAP, GRAP2, GRAPL, GRASP, GRB10, GRB14, GRB2, GRB7, GREB1, GREB1L, GREM1, GREM2, GRHL1, GRHL2, GRHL3, GRHPR, GRIA1, GRIA2, GRIA3, GRIA4, GRID1, GRID2, GRID2IP, GRIFIN, GRIK1, GRIK2, GRIK3, GRIK4, GRIK5, GRIN1, GRIN2A, GRIN2B, GRIN2C, GRIN2D, GRIN3A, GRIN3B, GRINA, GRIP1, GRIP2, GRIPAP1, GRK1, GRK2, GRK3, GRK4, GRK5, GRK6, GRK7, GRM1, GRM2, GRM3, GRM4, GRM5, GRM6, GRM7, GRM8, GRN, GRP, GRPEL1, GRPEL2, GRPR, GRSF1, GRTP1, GRWD1, GRXCR1, GRXCR2, GSAP, GSC, GSC2, GSDMA, GSDMB, GSDMC, GSDMD, GSE1, GSG1, GSG1L, GSG1L2, GSK3A, GSK3B, GSKIP, GSN, GSPT1, GSPT2, GSR, GSS, GSTA1, GSTA2, GSTA3, GSTA4, GSTA5, GSTCD, GSTK1, GSTM1, GSTM2, GSTM3, GSTM4, GSTM5, GSTO1, GSTO2, GSTP1, GSTT1, GSTT2, GSTT2B, GSTTP1, GSTZ1, GSX1, GSX2, GTDC1, GTF2A1, GTF2A1L, GTF2A2, GTF2B, GTF2E1, GTF2E2, GTF2F1, GTF2F2, GTF2H1, GTF2H2, GTF2H2C, GTF2H2C_2, GTF2H3, GTF2H4, GTF2H5, GTF2I, GTF2IRD1, GTF2IRD2, GTF2IRD2B, GTF3A, GTF3C1, GTF3C2, GTF3C3, GTF3C4, GTF3C5, GTF3C6, GTPBP1, GTPBP10, GTPBP2, GTPBP3, GTPBP4, GTPBP6, GTPBP8, GTSE1, GTSF1, GTSF1L, GU182339.1, GU182339.3, GU182343.1, GU182343.2, GU182345.1, GU182345.2, GU182347.1, GU182351.2, GU182352.2, GU182353.1, GU182355.1, GU182355.2, GU182355.3, GU182357.1, GU182357.3, GU182359.1, GU182359.2, GUCA1A, GUCA1B, GUCA1C, GUCA2A, GUCA2B, GUCD1, GUCY1A2, GUCY1A3, GUCY1B3, GUCY2C, GUCY2D, GUCY2F, GUF1, GUK1, GULP1, GUSB, GVQW2, GXYLT1, GXYLT2, GYG1, GYG2, GYPA, GYPB, GYPC, GYPE, GYS1, GYS2, GZF1, GZMA, GZMB, GZMH, GZMK, GZMM, H1F0, H1FNT, H1FOO, H1FX, H2AFB1, H2AFB2, H2AFB3, H2AFJ, H2AFV, H2AFX, H2AFY, H2AFY2, H2AFZ, H2BFM, H2BFS, H2BFWT, H3F3A, H3F3B, H3F3C, H6PD, HAAO, HABP2, HABP4, HACD1, HACD2, HACD3, HACD4, HACE1, HACL1, HADH, HADHA, HADHB, HAGH, HAGHL, HAL, HAMP, HAND1, HAND2, HAO1, HAG2, HAP1, HAPLN1, HAPLN2, HAPLN3, HAPLN4, HARBII, HARS, HARS2, HAS1, HAS2, HAS3, HASPIN, HAT1, HAUS1, HAUS2, HAUS3, HAUS4, HAUS5, HAUS6, HAUS7, HAUS8, HAVCR1, HAVCR₂, HAX1, HBA1, HBA2, HBB, HBD, HBE1, HBEGF, HBG1, HBG2, HBM, HBP1, HBQ1, HBS1L, HBZ, HCAR1, HCAR2, HCAR3, HCCS, HCFC1, HCFC1R1, HCFC2, HCK, HCLS1, HCN1, HCN2, HCN3, HCN4, HCRT, HCRTR1, HCRTR2, HCST, HDAC1, HDAC10, HDAC11, HDAC2, HDAC3, HDAC4, HDAC5, HDAC6, HDAC7, HDAC8, HDAC9, HDC, HDDC2, HDDC3, HDGF, HDGFL1, HDGFL2, HDGFL3, HDHD2, HDHD3, HDHD5, HDLBP, HDX, HEATR1, HEATR3, HEATR4, HEATR5A, HEATR5B, HEATR6, HEATR9, HEBP1, HEBP2, HECA, HECTD1, HECTD2, HECTD3, HECTD4, HECW1, HECW2, HEG1, HELB, HELLS, HELQ, HELT, HELZ, HELZ2, HEMGN, HEMK1, HENMT1, HEPACAM, HEPACAM2, HEPH, HEPHL1, HEPN1, HERC1, HERC2, HERC3, HERC4, HERC5, HERC6, HERPUD1, HERPUD2, HES1, HES2, HES3, HES4, HES5, HES6, HES7, HESX1, HEXA, HEXB, HEXDC, HEXIM1, HEXIM2, HEY1, HEY2, HEYL, HFE, HFE2, HFM1, HGD, HGF, HGFAC, HGH1, HGNC:18790, HGNC:24955, HGS, HGSNAT, HHAT, HHATL, HHEX, HHIP, HHIPL1, HHIPL2, HHLA1, HHLA2, HHLA3, HIBADH, HIBCH, HIC1, HIC2, HID1, HIF1A, HIF1AN, HIF3A, HIGD1A, HIGD1B, HIGD1C, HIGD2A, HIGD2B, HIKESHI, HILPDA, HINFP, HINT1, HINT2, HINT3, HIP1, HIP1R, HIPK1, HIPK2, HIPK3, HIPK4, HIRA, HIRIP3, HIST1H1A, HIST1H1B, HIST1H1C, HIST1H1D, HIST1H1E, HIST1H1T, HIST1H2AA, HIST1H2AB, HIST1H2AC, HIST1H2AD, HIST1H2AE, HIST1H2AG, HIST1H2AH, HIST1H2AI, HIST1H2AJ, HIST1H2AK, HIST1H2AL, HIST1H2AM, HIST1H2BA, HIST1H2BB, HIST1H2BC, HIST1H2BD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, HIST1H3A, HIST1H3B, HIST1H3C, HIST1H3D, HIST1H3E, HIST1H3F, HIST1H3G, HIST1H3H, HIST1H3I, HIST1H3J, HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, HIST2H2AA3, HIST2H2AA4, HIST2H2AB, HIST2H2AC, HIST2H2BE, HIST2H2BF, HIST2H3A, HIST2H3C, HIST2H3D, HIST2H3PS2, HIST2H4A, HIST2H4B, HIST3H2A, HIST3H2BB, HIST3H3, HIST4H4, HIVEP1, HIVEP2, HIVEP3, HJURP, HK1, HK2, HK3, HKDC1, HKR1, HLA-A, HLA-B, HLA-C, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, HLA-E, HLA-F, HLA-G, HLCS, HLF, HLTF, HLX, HM13, HM190170.1, HMBOXI, HMBS, HMCES, HMCN1, HMCN2, HMG20A, HMG20B, HMGA1, HMGA2, HMGB1, HMGB2, HMGB3, HMGB4, HMGCL, HMGCLL1, HMGCR, HMGCS1, HMGCS2, HMGN1, HMGN2, HMGN3, HMGN4, HMGN5, HMGXB3, HMGXB4, HWMB1, HMMR, HMOX1, HMOX2, HMSD, HMX1, HMX2, HMX3, HNF1A, HNF1B, HNF4A, HNF4G, HNMT, HNRNPA0, HNRNPA1, HNRNPA1L2, HNRNPA2B1, HNRNPA3, HNRNPAB, HNRNPC, HNRNPCL1, HNRNPCL2, HNRNPCL3, HNRNPCL4, HNRNPD, HNRNPDL, HNRNPF, HNRNPH1, HNRNPH2, HNRNPH3, HNRNPK, HNRNPL, HNRNPLL, HNRNPM, HNRNPR, HNRNPU, HNRNPUL1, HNRNPUL2, HNRNPUL2-BSCL2, HOGA1, HOMER1, HOMER2, HOMER3, HOMEZ, HOOK1, HOOK2, HOOK3, HOPX, HORMAD1, HORMAD2, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HP, HP1BP3, HPCA, HPCAL1, HPCAL4, HPD, HPDL, HPF1, HPGD, HPGDS, HPN, HPR, HPRT1, HPS1, HPS3, HPS4, HPS5, HPS6, HPSE, HPSE2, HPX, HR, HRAS, HRASLS, HRASLS2, HRASLS5, HRC, HRCT1, HRG, HRH1, HRH2, HRH3, HRH4, HRK, HRNR, HS1BP3, HS2ST1, HS3ST1, HS3ST2, HS3ST3A1, HS3ST3B1, HS3ST4, HS3ST5, HS3ST6, HS6ST1, HS6ST2, HS6ST3, HSBP1, HSBP1L1, HSCB, HSD11B1, HSD11B1L, HSD11B2, HSD17B1, HSD17B10, HSD17B11, HSD17B12, HSD17B13, HSD17B14, HSD17B2, HSD17B3, HSD17B4, HSD17B6, HSD17B7, HSD17B8, HSD3B1, HSD3B2, HSD3B7, HSDL1, HSDL2, HSF1, HSF2, HSF2BP, HSF4, HSF5, HSFX1, HSFX2, HSFX3, HSFX4, HSFY1, HSFY2, HSH2D, HSP90AA1, HSP90AB1, HSP90B1, HSPA12A, HSPA12B, HSPA13, HSPA14, HSPA1A, HSPA1B, HSPA1L, HSPA2, HSPA4, HSPA4L, HSPA5, HSPA6, HSPA8, HSPA9, HSPB1, HSPB11, HSPB2, HSPB2-C11orf52, HSPB3, HSPB6, HSPB7, HSPB8, HSPB9, HSPBAP1, HSPBP1, HSPD1, HSPE1, HSPE1-MOB4, HSPG2, HSPH1, HTATIP2, HTATSF1, HTD2, HTN1, HTN3, HTR1A, HTR1B, HTR1D, HTR1E, HTR1F, HTR2A, HTR2B, HTR2C, HTR3A, HTR3B, HTR3C, HTR3D, HTR3E, HTR4, HTR5A, HTR6, HTR7, HTRA1, HTRA2, HTRA3, HTRA4, HTT, HUNK, HUS1, HUS1B, HUWEl, HVCN1, HYAL1, HYAL2, HYAL3, HYAL4, HYDIN, HYI, HYKK, HYLS1, HYOU1, HYPK, HYPM, IAH1, IAPP, IARS, IARS2, IBA57, IBSP, IBTK, ICA1, ICA1L, ICAM1, ICAM2, ICAM3, ICAM4, ICAM5, ICEl, ICE2, ICK, ICMT, ICOS, ICOSLG, ID1, ID2, ID3, ID4, IDE, IDH1, IDH2, IDH3A, IDH3B, IDH3G, IDIl, IDI2, IDNK, IDO1, IDO2, IDS, IDUA, IER2, IER3, IER3IP1, IER5, IER5L, IFFO1, IFF02, IFI16, IFI27, IFI27L1, IFI27L2, IFI30, IFI35, IFI44, IFI44L, IFI6, IFIH1, IFIT1, IFITIB, IFIT2, IFIT3, IFIT5, IFITM1, IFITM10, IFITM2, IFITM3, IFITM5, IFNA1, IFNA10, IFNA13, IFNA14, IFNA16, IFNA17, IFNA2, IFNA21, IFNA4, IFNA5, IFNA6, IFNA7, IFNA8, IFNAR1, IFNAR2, IFNB1, IFNE, IFNG, IFNGR1, IFNGR2, IFNK, IFNL1, IFNL2, IFNL3, IFNL4, IFNLR1, IFNW1, IFRD1, IFRD2, IFT122, IFT140, IFT172, IFT20, IFT22, IFT27, IFT43, IFT46, IFT52, IFT57, IFT74, IFT80, IFT81, IFT88, IGBP1, IGDCC3, IGDCC4, IGF1, IGF1R, IGF2, IGF2BP1, IGF2BP2, IGF2BP3, IGF2R, IGFALS, IGFBP1, IGFBP2, IGFBP3, IGFBP4, IGFBP5, IGFBP6, IGFBP7, IGFBPL1, IGFL1, IGFL2, IGFL3, IGFL4, IGFLR1, IGFN1, IGHA1, IGHA2, IGHD, IGHD1-1, IGHD1-14, IGHD1-20, IGHD1-26, IGHD1-7, IGHD1OR15-1A, IGHD1OR15-1B, IGHD2-15, IGHD2-2, IGHD2-21, IGHD2-8, IGHD2OR15-2A, IGHD2OR15-2B, IGHD3-10, IGHD3-16, IGHD3-22, IGHD3-3, IGHD3-9, IGHD30R15-3A, IGHD30R15-3B, IGHD4-11, IGHD4-17, IGHD4-23, IGHD4-4, IGHD40R15-4A, IGHD40R15-4B, IGHD5-12, IGHD5-18, IGHD5-24, IGHD5-5, IGHD50R15-5A, IGHD50R15-5B, IGHD6-13, IGHD6-19, IGHD6-25, IGHD6-6, IGHD7-27, IGHE, IGHG1, IGHG2, IGHG3, IGHG4, IGHJ1, IGHJ2, IGHJ3, IGHJ4, IGHJ5, IGHJ6, IGHM, IGHMBP2, IGHV1-18, IGHV1-2, IGHV1-24, IGHV1-3, IGHV1-45, IGHV1-46, IGHV1-58, IGHV1-69, IGHV1OR15-1, IGHV1OR15-9, IGHV1OR21-1, IGHV2-26, IGHV2-5, IGHV2-70, IGHV2OR16-5, IGHV3-11, IGHV3-13, IGHV3-15, IGHV3-16, IGHV3-20, IGHV3-21, IGHV3-23, IGHV3-30, IGHV3-33, IGHV3-35, IGHV3-38, IGHV3-43, IGHV3-48, IGHV3-49, IGHV3-53, IGHV3-64, IGHV3-66, IGHV3-7, IGHV3-72, IGHV3-73, IGHV3-74, IGHV30R15-7, IGHV30R16-10, IGHV30R16-12, IGHV30R16-13, IGHV30R16-8, IGHV30R16-9, IGHV4-28, IGHV4-31, IGHV4-34, IGHV4-39, IGHV4-4, IGHV4-59, IGHV4-61, IGHV4OR15-8, IGHV5-51, IGHV6-1, IGHV7-81, IGIP, IGKC, IGKJ1, IGKJ2, IGKJ3, IGKJ4, IGKJ5, IGKV1-12, IGKV1-16, IGKV1-17, IGKV1-27, IGKV1-33, IGKV1-37, IGKV1-39, IGKV1-5, IGKV1-6, IGKV1-8, IGKV1-9, IGKV1D-12, IGKV1D-13, IGKV1D-16, IGKV1D-17, IGKV1D-33, IGKV1D-37, IGKV1D-39, IGKV1D-42, IGKV1D-43, IGKV1D-8, IGKV1OR₂-108, IGKV2-24, IGKV2-28, IGKV2-30, IGKV2-40, IGKV2D-24, IGKV2D-26, IGKV2D-28, IGKV2D-29, IGKV2D-30, IGKV2D-40, IGKV3-11, IGKV3-15, IGKV3-20, IGKV3-7, IGKV3D-11, IGKV3D-15, IGKV3D-20, IGKV3D-7, IGKV3OR₂-268, IGKV4-1, IGKV5-2, IGKV6-21, IGKV6D-21, IGKV6D-41, IGLC1, IGLC2, IGLC3, IGLC7, IGLJ1, IGLJ2, IGLJ3, IGLJ4, IGLJ5, IGLJ6, IGLJ7, IGLL1, IGLL5, IGLON5, IGLV10-54, IGLV11-55, IGLV1-36, IGLV1-40, IGLV1-44, IGLV1-47, IGLV1-50, IGLV1-51, IGLV2-11, IGLV2-14, IGLV2-18, IGLV2-23, IGLV2-33, IGLV2-8, IGLV3-1, IGLV3-10, IGLV3-12, IGLV3-16, IGLV3-19, IGLV3-21, IGLV3-22, IGLV3-25, IGLV3-27, IGLV3-32, IGLV3-9, IGLV4-3, IGLV4-60, IGLV4-69, IGLV5-37, IGLV5-45, IGLV5-48, IGLV5-52, IGLV6-57, IGLV7-43, IGLV7-46, IGLV8-61, IGLV9-49, IGSF1, IGSF10, IGSF11, IGSF21, IGSF22, IGSF23, IGSF3, IGSF5, IGSF6, IGSF8, IGSF9, IGSF9B, IHH, IK, IKBIP, IKBKB, IKBKE, IKBKG, IKZF1, IKZF2, IKZF3, IKZF4, IKZF5, IL10, IL10RA, IL10RB, IL11, IL11RA, IL12A, IL12B, IL12RB1, IL12RB2, IL13, IL13RA1, IL13RA2, IL15, IL15RA, IL16, IL17A, IL17B, IL17C, IL17D, IL17F, IL17RA, IL17RB, IL17RC, IL17RD, IL17RE, IL17REL, IL18, IL18BP, IL18R1, IL18RAP, IL19, IL1A, IL1B, IL1F10, IL1R1, IL1R2, IL1RAP, IL1RAPL1, IL1RAPL2, IL1RL1, IL1RL2, IL1RN, IL2, IL20, IL20RA, IL20RB, IL21, IL21R, IL22, IL22RA1, IL22RA2, IL23A, IL23R, IL24, IL25, IL26, IL27, IL27RA, IL2RA, IL2RB, IL2RG, TL3, IL31, IL31RA, IL32, IL33, IL34, IL36A, IL36B, IL36G, IL36RN, IL37, IL3RA, IL4, IL4I1, IL4R, IL5, IL5RA, IL6, IL6R, IL6ST, IL7, IL7R, IL9, IL9R, ILDR1, ILDR2, ILF2, ILF3, ILK, ILKAP, ILVBL, IMMP1L, IMMP2L, IMMT, IMP3, IMP4, IMPA1, IMPA2, IMPACT, IMPAD1, IMPDH1, IMPDH2, IMPG1, IMPG2, INA, INAFM1, INAFM2, INAVA, INCA1, INCENP, INF2, ING1, ING2, ING3, ING4, ING5, INHA, INHBA, INHBB, INHBC, INHBE, INIP, INMT, INMT-MINDY4, INO80, INO80B, INO80B-WBP1, INO80C, INO80D, INO80E, INPP1, INPP4A, INPP4B, INPP5A, INPP5B, INPP5D, INPP5E, INPP5F, INPP5J, INPP5K, INPPL1, INS, INSC, INSIGI, INSIG2, INS-IGF2, INSL3, INSL4, INSL5, INSL6, INSM1, INSM2, INSR, INSRR, INTS1, INTS10, INTS11, INTS12, INTS13, INTS14, INTS2, INTS3, INTS4, INTS5, INTS6, INTS6L, INTS7, INTS8, INTS9, INTU, INVS, IP6K1, IP6K2, IP6K3, IPCEF1, IPMK, IPO11, IP013, IP04, IPO5, IP07, IP08, IP09, IPP, IPPK, IQANKI, IQCA1, IQCA1L, IQCB1, IQCC, IQCD, IQCE, IQCF1, IQCF2, IQCF3, IQCF5, IQCF6, IQCG, IQCH, IQCJ, IQCJ-SCHIP1, IQCK, IQCM, IQGAP1, IQGAP2, IQGAP3, IQSEC1, IQSEC2, IQSEC3, IQUB, IREB2, IRF1, IRF2, IRF2BP1, IRF2BP2, IRF2BPL, IRF3, IRF4, IRF5, IRF6, IRF7, IRF8, IRF9, IRGC, IRGM, IRGQ, IRS1, IRS2, IRS4, IRX1, IRX2, IRX3, IRX4, IRX5, IRX6, ISCA1, ISCA2, ISCU, ISG15, ISG20, ISG20L2, ISL1, ISL2, ISLR, ISLR2, ISM1, ISM2, ISOC1, ISOC2, ISPD, IST1, ISX, ISYl, ISY1-RAB43, ISYNA1, ITCH, ITFG1, ITFG2, ITGA1, ITGA10, ITGA11, ITGA2, ITGA2B, ITGA3, ITGA4, ITGA5, ITGA6, ITGA7, ITGA8, ITGA9, ITGAD, ITGAE, ITGAL, ITGAM, ITGAV, ITGAX, ITGB1, ITGB1BP1, ITGB1BP2, ITGB2, ITGB3, ITGB3BP, ITGB4, ITGB5, ITGB6, ITGB7, ITGB8, ITGBL1, ITIH1, ITIH2, ITIH3, ITIH4, ITIH5, ITIH6, ITK, ITLN1, ITLN2, ITM2A, ITM2B, ITM2C, ITPA, ITPK1, ITPKA, ITPKB, ITPKC, ITPR1, ITPR2, ITPR3, ITPRIP, ITPRIPL1, ITPRIPL2, ITSN1, ITSN2, IVD, IVL, IVNS1ABP, IWS1, IYD, IZUMO1, IZUMOIR, IZUMO2, IZUMO3, IZUMO4, JADE1, JADE2, JADE3, JAG1, JAG2, JAGN1, JAK1, JAK2, JAK3, JAKMIP1, JAKMIP2, JAKMIP3, JAM2, JAM3, JAML, JARID2, JAZF1, JCAD, JCHAIN, JDP2, JKAMP, JMJDIC, JMJD4, JMJD6, JMJD7, JMJD7-PLA2G4B, JMJD8, JMY, JOSD1, JOSD2, JPH1, JPH2, JPH3, JPH4, JPT1, JPT2, JRK, JRKL, JSRP1, JTB, JUN, JUNB, JUND, JUP, KAAG1, KALRN, KANK1, KANK2, KANK3, KANK4, KANSLI, KANSLIL, KANSL2, KANSL3, KANTR, KARS, KAT14, KAT2A, KAT2B, KAT5, KAT6A, KAT6B, KAT7, KAT8, KATNA1, KATNAL1, KATNAL2, KATNB1, KATNBL1, KAZALD1, KAZN, KBTBD11, KBTBD11-OT1, KBTBD12, KBTBD13, KBTBD2, KBTBD3, KBTBD4, KBTBD6, KBTBD7, KBTBD8, KCMF1, KCNA1, KCNA10, KCNA2, KCNA3, KCNA4, KCNA5, KCNA7, KCNAB1, KCNAB2, KCNAB3, KCNB1, KCNB2, KCNC1, KCNC2, KCNC3, KCNC4, KCND1, KCND2, KCND3, KCNE1, KCNElB, KCNE2, KCNE3, KCNE4, KCNE5, KCNF1, KCNG1, KCNG2, KCNG3, KCNG4, KCNH1, KCNH2, KCNH3, KCNH4, KCNH5, KCNH6, KCNH7, KCNH8, KCNIP1, KCNIP2, KCNIP3, KCNIP4, KCNJ1, KCNJ10, KCNJ11, KCNJ12, KCNJ13, KCNJ14, KCNJ15, KCNJ16, KCNJ18, KCNJ2, KCNJ3, KCNJ4, KCNJ5, KCNJ6, KCNJ8, KCNJ9, KCNK1, KCNK10, KCNK12, KCNK13, KCNK15, KCNK16, KCNK17, KCNK18, KCNK2, KCNK3, KCNK4, KCNK5, KCNK6, KCNK7, KCNK9, KCNMA1, KCNMB1, KCNMB2, KCNMB3, KCNMB4, KCNN1, KCNN2, KCNN3, KCNN4, KCNQ1, KCNQ2, KCNQ3, KCNQ4, KCNQ5, KCNRG, KCNS1, KCNS2, KCNS3, KCNT1, KCNT2, KCNU1, KCNV1, KCNV2, KCP, KCTD1, KCTD10, KCTD11, KCTD12, KCTD13, KCTD14, KCTD15, KCTD16, KCTD17, KCTD18, KCTD19, KCTD2, KCTD20, KCTD21, KCTD3, KCTD4, KCTD5, KCTD6, KCTD7, KCTD8, KCTD9, KDELC1, KDELC2, KDELR1, KDELR2, KDELR3, KDF1, KDM1A, KDM1B, KDM2A, KDM2B, KDM3A, KDM3B, KDM4A, KDM4B, KDM4C, KDM4D, KDM4E, KDM4F, KDM5A, KDM5B, KDM5C, KDM5D, KDM6A, KDM6B, KDM7A, KDM8, KDR, KDSR, KEAP1, KEL, KERA, KF459570.1, KHDC1, KHDC1L, KHDC3L, KHDRBS1, KHDRBS2, KHDRBS3, KHK, KHNYN, KHSRP, KIAA0040, KIAA0100, KIAA0141, KIAA0232, KIAA0319, KIAA0319L, KIAA0355, KIAA0368, KIAA0391, KIAA0408, KIAA0513, KIAA0556, KIAA0586, KIAA0753, KIAA0825, KIAA0895, KIAA0895L, KIAA0907, KIAA0930, KIAA1024, KIAA1024L, KIAA1107, KIAA1109, KIAA1143, KIAA1147, KIAA1161, KIAA1191, KIAA1210, KIAA1211, KIAA1211L, KIAA1217, KIAA1257, KIAA1324, KIAA1324L, KIAA1328, KIAA1456, KIAA1468, KIAA1522, KIAA1524, KIAA1549, KIAA1549L, KIAA1551, KIAA1586, KIAA1614, KIAA1644, KIAA1671, KIAA1683, KIAA1755, KIAA1841, KIAA1958, KIAA2012, KIAA2013, KIAA2026, KIDINS220, KIF11, KIF12, KIF13A, KIF13B, KIF14, KIF15, KIF16B, KIF17, KIF18A, KIF18B, KIF19, KIF1A, KIF1B, KIF1BP, KIF1C, KIF20A, KIF20B, KIF21A, KIF21B, KIF22, KIF23, KIF24, KIF25, KIF26A, KIF26B, KIF27, KIF2A, KIF2B, KIF2C, KIF3A, KIF3B, KIF3C, KIF4A, KIF4B, KIF5A, KIF5B, KIF5C, KIF6, KIF7, KIF9, KIFAP3, KIFC1, KIFC2, KIFC3, KIN, KIR2DL1, KIR2DL2, KIR2DL3, KIR2DL4, KIR2DL5A, KIR2DL5B, KIR2DP1, KIR2DS1, KIR2DS2, KIR2DS3, KIR2DS4, KIR2DS5, KIR3DL1, KIR3DL2, KIR3DL3, KIR3DP1, KIR3DS1, KIR3DX1, KIRREL1, KIRREL2, KIRREL3, KISS1, KISS1R, KIT, KITLG, KIZ, KL, KLB, KLC1, KLC2, KLC3, KLC4, KLF1, KLF10, KLF11, KLF12, KLF13, KLF14, KLF15, KLF16, KLF17, KLF18, KLF2, KLF3, KLF4, KLF5, KLF6, KLF7, KLF8, KLF9, KLHDC1, KLHDC10, KLHDC2, KLHDC3, KLHDC4, KLHDC7A, KLHDC7B, KLHDC8A, KLHDC8B, KLHDC9, KLHL1, KLHL10, KLHL11, KLHL12, KLHL13, KLHL14, KLHL15, KLHL17, KLHL18, KLHL2, KLHL20, KLHL21, KLHL22, KLHL23, KLHL24, KLHL25, KLHL26, KLHL28, KLHL29, KLHL3, KLHL30, KLHL31, KLHL32, KLHL33, KLHL34, KLHL35, KLHL36, KLHL38, KLHL4, KLHL40, KLHL41, KLHL42, KLHL5, KLHL6, KLHL7, KLHL8, KLHL9, KLK1, KLK10, KLK11, KLK12, KLK13, KLK14, KLK15, KLK2, KLK3, KLK4, KLK5, KLK6, KLK7, KLK8, KLK9, KLKB1, KLLN, KLRB1, KLRC1, KLRC2, KLRC3, KLRC4, KLRC4-KLRK1, KLRD1, KLRF1, KLRF2, KLRG1, KLRG2, KLRK1, KMO, KMT2A, KMT2B, KMT2C, KMT2D, KMT2E, KMT5A, KMT5B, KMT5C, KNCN, KNDC1, KNG1, KNL1, KNOP1, KNSTRN, KNTC1, KP420437.1, KP420437.2, KP420437.3, KP420439.1, KP420440.1, KP420440.2, KP420440.3, KP420440.4, KP420440.5, KP420440.6, KP420440.7, KP420440.8, KP420440.9, KP420441.1, KP420441.2, KP420441.3, KP420441.4, KP420441.5, KP420442.2, KP420442.3, KP420443.1, KP420444.1, KP420444.2, KP420444.3, KP420444.4, KP420444.5, KP420444.6, KP420444.7, KP420446.1, KP420446.2, KPNA1, KPNA2, KPNA3, KPNA4, KPNA5, KPNA6, KPNA7, KPNB1, KPRP, KPTN, KRBA1, KRBA2, KRBOX1, KRBOX4, KRCC1, KREMEN1, KREMEN2, KRI1, KRIT1, KRR1, KRT1, KRT10, KRT12, KRT13, KRT14, KRT15, KRT16, KRT17, KRT18, KRT19, KRT2, KRT20, KRT222, KRT23, KRT24, KRT25, KRT26, KRT27, KRT28, KRT3, KRT31, KRT32, KRT33A, KRT33B, KRT34, KRT35, KRT36, KRT37, KRT38, KRT39, KRT4, KRT40, KRT5, KRT6A, KRT6B, KRT6C, KRT7, KRT71, KRT72, KRT73, KRT74, KRT75, KRT76, KRT77, KRT78, KRT79, KRT8, KRT80, KRT81, KRT82, KRT83, KRT84, KRT85, KRT86, KRT9, KRTAP10-1, KRTAP10-10, KRTAP10-11, KRTAP10-12, KRTAP10-2, KRTAP10-3, KRTAP10-4, KRTAP10-5, KRTAP10-6, KRTAP10-7, KRTAP10-8, KRTAP10-9, KRTAP1-1, KRTAP11-1, KRTAP12-1, KRTAP12-2, KRTAP12-3, KRTAP12-4, KRTAP1-3, KRTAP13-1, KRTAP13-2, KRTAP13-3, KRTAP13-4, KRTAP1-4, KRTAP1-5, KRTAP15-1, KRTAP16-1, KRTAP17-1, KRTAP19-1, KRTAP19-2, KRTAP19-3, KRTAP19-4, KRTAP19-5, KRTAP19-6, KRTAP19-7, KRTAP19-8, KRTAP20-1, KRTAP20-2, KRTAP20-3, KRTAP20-4, KRTAP2-1, KRTAP21-1, KRTAP21-2, KRTAP21-3, KRTAP2-2, KRTAP22-1, KRTAP22-2, KRTAP2-3, KRTAP23-1, KRTAP2-4, KRTAP24-1, KRTAP25-1, KRTAP26-1, KRTAP27-1, KRTAP29-1, KRTAP3-1, KRTAP3-2, KRTAP3-3, KRTAP4-1, KRTAP4-11, KRTAP4-12, KRTAP4-16, KRTAP4-2, KRTAP4-3, KRTAP4-4, KRTAP4-5, KRTAP4-6, KRTAP4-7, KRTAP4-8, KRTAP4-9, KRTAP5-1, KRTAP5-10, KRTAP5-11, KRTAP5-2, KRTAP5-3, KRTAP5-4, KRTAP5-5, KRTAP5-6, KRTAP5-7, KRTAP5-8, KRTAP5-9, KRTAP6-1, KRTAP6-2, KRTAP6-3, KRTAP7-1, KRTAP8-1, KRTAP9-1, KRTAP9-2, KRTAP9-3, KRTAP9-4, KRTAP9-6, KRTAP9-7, KRTAP9-8, KRTAP9-9, KRTCAP2, KRTCAP3, KRTDAP, KSR1, KSR2, KTI12, KTN1, KU645196.1, KU645196.2, KU645196.3, KU645196.4, KU645196.5, KU645196.6, KU645196.7, KU645196.8, KU645196.9, KU645197.1, KU645197.2, KU645197.3, KU645197.4, KU645197.5, KU645198.1, KXD1, KY, KYATI, KYAT3, KYNU, LICAM, L1TD1, L2HGDH, L34079.1, L3HYPDH, L3MBTL1, L3MBTL2, L3MBTL3, L3MBTL4, LACCI, LACRT, LACTB, LACTB2, LACTBL1, LADI, LAG3, LAGE3, LAIRI, LAIR2, LALBA, LAMA1, LAMA2, LAMA3, LAMA4, LAMA5, LAMBI, LAMB2, LAMB3, LAMB4, LAMC1, LAMC2, LAMC3, LAMPI, LAMP2, LAMP3, LAMP5, LAMTOR1, LAMTOR₂, LAMTOR3, LAMTOR4, LAMTOR5, LANCLI, LANCL2, LANCL3, LAP3, LAPTM4A, LAPTM4B, LAPTM5, LARGE1, LARGE2, LARPI, LARPIB, LARP4, LARP4B, LARP6, LARP7, LARS, LARS2, LASIL, LASPI, LAT, LAT2, LATS1, LATS2, LAXI, LAYN, LBH, LBHD1, LBP, LBR, LBX1, LBX2, LCA5, LCA5L, LCAT, LCE1A, LCElB, LCE1C, LCE1D, LCE1E, LCE1F, LCE2A, LCE2B, LCE2C, LCE2D, LCE3A, LCE3B, LCE3C, LCE3D, LCE3E, LCE4A, LCE5A, LCE6A, LCK, LCLAT1, LCMT1, LCMT2, LCN1, LCN10, LCN12, LCN15, LCN2, LCN6, LCN8, LCN9, LCNL1, LCOR, LCORL, LCP1, LCP2, LCT, LCTL, LDAH, LDB1, LDB2, LDB3, LDHA, LDHAL6A, LDHAL6B, LDHB, LDHC, LDHD, LDLR, LDLRAD1, LDLRAD2, LDLRAD3, LDLRAD4, LDLRAP1, LDOC1, LEAP2, LECT2, LEFI, LEFTY1, LEFTY2, LEKR1, LELPI, LEMDI, LEMD2, LEMD3, LENEP, LENGI, LENG8, LENG9, LEO1, LEP, LEPR, LEPROT, LEPROTLI, LETMI, LETM2, LETMDI, LEUTX, LEXM, LFNG, LGALS1, LGALS12, LGALS13, LGALS14, LGALS16, LGALS2, LGALS3, LGALS3BP, LGALS4, LGALS7, LGALS7B, LGALS8, LGALS9, LGALS9B, LGALS9C, LGALSL, LGI1, LGI2, LGI3, LGI4, LGMN, LGR4, LGR5, LGR6, LGSN, LHB, LHCGR, LHFPL1, LHFPL2, LHFPL3, LHFPL4, LHFPL5, LHFPL6, LHPP, LHX1, LHX2, LHX3, LHX4, LHX5, LHX6, LHX8, LHX9, LIAS, LIF, LIFR, LIGI, LIG3, LIG4, LILRA1, LILRA2, LILRA3, LILRA4, LILRA5, LILRA6, LILRB1, LILRB2, LILRB3, LILRB4, LILRB5, LIM2, LIMA1, LIMCH1, LIMDI, LIMD2, LIME1, LIMK1, LIMK2, LIMS1, LIMS2, LIMS3, LIMS4, LIN28A, LIN28B, LIN37, LIN52, LIN54, LIN7A, LIN7B, LIN7C, LIN9, LINC00094, LINC00116, LINC00282, LINC00672, LINC00675, LINC00694, LINC00854, LINC00890, LINC00959, LINC01125, LINC01556, LINC02210-CRHR1, LINGO1, LINGO2, LINGO3, LINGO4, LINS1, LIPA, LIPC, LIPE, LIPF, LIPG, LIPH, LIPI, LIPJ, LIPK, LIPM, LIPN, LIPT1, LIPT2, LITAF, LIX1, LIX1L, LKAAEAR1, LLGL1, LLGL2, LLPH, LMAN1, LMAN1L, LMAN2, LMAN2L, LMBR1, LMBR1L, LMBRD1, LMBRD2, LMCD1, LMF1, LMF2, LMLN, LMNA, LMNB1, LMNB2, LMNTD1, LMNTD2, LMO1, LMO2, LMO3, LMO4, LMO7, LMO7DN, LMOD1, LMOD2, LMOD3, LMTK2, LMTK3, LMX1A, LMX1B, LNP1, LNPEP, LNPK, LNX1, LNX2, LO000005.1, LONP1, LONP2, LONRF1, LONRF2, LONRF3, LOR, LOX, LOXHD1, LOXL1, LOXL2, LOXL3, LOXL4, LPA, LPAR1, LPAR2, LPAR3, LPAR4, LPAR5, LPAR6, LPCAT1, LPCAT2, LPCAT3, LPCAT4, LPGAT1, LPIN1, LPIN2, LPIN3, LPL, LPO, LPP, LPXN, LRAT, LRBA, LRCH1, LRCH2, LRCH3, LRCH4, LRCOL1, LRFN1, LRFN2, LRFN3, LRFN4, LRFN5, LRG1, LRGUK, LRIF1, LRIG1, LRIG2, LRIG3, LRIT1, LRIT2, LRIT3, LRMDA, LRMP, LRP1, LRP10, LRP11, LRP12, LRP1B, LRP2, LRP2BP, LRP3, LRP4, LRP5, LRP5L, LRP6, LRP8, LRPAP1, LRPPRC, LRR1, LRRC1, LRRC10, LRRC10B, LRRC14, LRRC14B, LRRC15, LRRC17, LRRC18, LRRC19, LRRC2, LRRC20, LRRC23, LRRC24, LRRC25, LRRC26, LRRC27, LRRC28, LRRC29, LRRC3, LRRC30, LRRC31, LRRC32, LRRC34, LRRC36, LRRC37A, LRRC37A2, LRRC37A3, LRRC37B, LRRC38, LRRC39, LRRC3B, LRRC3C, LRRC4, LRRC40, LRRC41, LRRC42, LRRC43, LRRC45, LRRC46, LRRC47, LRRC49, LRRC4B, LRRC4C, LRRC52, LRRC53, LRRC55, LRRC56, LRRC57, LRRC58, LRRC59, LRRC6, LRRC61, LRRC63, LRRC66, LRRC69, LRRC7, LRRC70, LRRC71, LRRC72, LRRC73, LRRC74A, LRRC74B, LRRC75A, LRRC75B, LRRC8A, LRRC8B, LRRC8C, LRRC8D, LRRC8E, LRRC9, LRRCC1, LRRD1, LRRFIP1, LRRFIP2, LRRIQ1, LRRIQ3, LRRIQ4, LRRK1, LRRK2, LRRN1, LRRN2, LRRN3, LRRN4, LRRN4CL, LRRTM1, LRRTM2, LRRTM3, LRRTM4, LRSAM1, LRTM1, LRTM2, LRTOMT, LRWD1, LSAMP, LSG1, LSM1, LSM10, LSM11, LSM12, LSM14A, LSM14B, LSM2, LSM3, LSM4, LSM5, LSM6, LSM7, LSM8, LSMEM1, LSMEM2, LSP1, LSR, LSS, LST1, LTA, LTA4H, LTB, LTB4R, LTB4R2, LTBP1, LTBP2, LTBP3, LTBP4, LTBR, LTC4S, LTF, LTK, LTN1, LTV1, LUC7L, LUC7L2, LUC7L3, LUM, LURAPI, LURAPIL, LUZP1, LUZP2, LUZP4, LUZP6, LVRN, LXN, LY6D, LY6E, LY6G5B, LY6G5C, LY6G6C, LY6G6D, LY6G6E, LY6G6F, LY6H, LY6K, LY6L, LY75, LY75-CD302, LY86, LY9, LY96, LYAR, LYG1, LYG2, LYL1, LYN, LYNX1, LYPD1, LYPD2, LYPD3, LYPD4, LYPD5, LYPD6, LYPD6B, LYPD8, LYPLA1, LYPLA2, LYPLAL1, LYRM1, LYRM2, LYRM4, LYRM7, LYRM9, LYSMD1, LYSMD2, LYSMD3, LYSMD4, LYST, LYVE1, LYZ, LYZL1, LYZL2, LYZL4, LYZL6, LZIC, LZTFL1, LZTR1, LZTS1, LZTS2, LZTS3, M1AP, M6PR, MAATS1, MAB21L1, MAB21L2, MAB21L3, MACC1, MACF1, MACRODI, MACROD2, MAD1L1, MAD2L1, MAD2L1BP, MAD2L2, MADCAM1, MADD, MAEA, MAEL, MAF, MAF1, MAFA, MAFB, MAFF, MAFG, MAFK, MAG, MAGEA1, MAGEA10, MAGEAI1, MAGEA12, MAGEA2, MAGEA2B, MAGEA3, MAGEA4, MAGEA6, MAGEA8, MAGEA9, MAGEA9B, MAGEB1, MAGEB10, MAGEB16, MAGEB17, MAGEB18, MAGEB2, MAGEB3, MAGEB4, MAGEB5, MAGEB6, MAGEB6P1, MAGECI, MAGEC2, MAGEC3, MAGEDI, MAGED2, MAGED4, MAGED4B, MAGEE1, MAGEE2, MAGEFI, MAGEHI, MAGEL2, MAGI1, MAGI2, MAGI3, MAGIX, MAGOH, MAGOHB, MAGT1, MAIP1, MAJIN, MAK, MAK16, MAL, MAL2, MALL, MALRD1, MALSU1, MALT1, MAMDC2, MAMDC4, MAML1, MAML2, MAML3, MAMLD1, MAMSTR, MAN1A1, MAN1A2, MAN1B1, MAN1C1, MAN2A1, MAN2A2, MAN2B1, MAN2B2, MAN2C1, MANBA, MANBAL, MANEA, MANEAL, MANF, MANSC1, MANSC4, MAOA, MAOB, MAP10, MAP1A, MAP1B, MAP1LC3A, MAP1LC3B, MAP1LC3B2, MAP1LC3C, MAPlS, MAP2, MAP2K1, MAP2K2, MAP2K3, MAP2K4, MAP2K5, MAP2K6, MAP2K7, MAP3K1, MAP3K10, MAP3K11, MAP3K12, MAP3K13, MAP3K14, MAP3K15, MAP3K19, MAP3K2, MAP3K20, MAP3K21, MAP3K3, MAP3K4, MAP3K5, MAP3K6, MAP3K7, MAP3K7CL, MAP3K8, MAP3K9, MAP4, MAP4K1, MAP4K2, MAP4K3, MAP4K4, MAP4K5, MAP6, MAP6D1, MAP7, MAP7D1, MAP7D2, MAP7D3, MAP9, MAPK1, MAPK10, MAPK11, MAPK12, MAPK13, MAPK14, MAPK15, MAPK1IP1L, MAPK3, MAPK4, MAPK6, MAPK7, MAPK8, MAPK8IP1, MAPK8IP2, MAPK8IP3, MAPK9, MAPKAP1, MAPKAPK2, MAPKAPK3, MAPKAPK5, MAPKBP1, MAPRE1, MAPRE2, MAPRE3, MAPT, MARC1, MARC2, MARCH1, MARCH10, MARCH11, MARCH2, MARCH3, MARCH4, MARCH5, MARCH6, MARCH7, MARCH8, MARCH9, MARCKS, MARCKSL1, MARCO, MARF1, MARK1, MARK2, MARK3, MARK4, MARS, MARS2, MARVELD1, MARVELD2, MARVELD3, MAS1, MAS1L, MASP1, MASP2, MAST1, MAST2, MAST3, MAST4, MASTL, MAT1A, MAT2A, MAT2B, MATK, MATN1, MATN2, MATN3, MATN4, MATR3, MAU2, MAVS, MAX, MAZ, MB, MB21D1, MB21D2, MBD1, MBD2, MBD3, MBD3L1, MBD3L2, MBD3L2B, MBD3L3, MBD3L4, MBD3L5, MBD4, MBD5, MBD6, MBIP, MBL2, MBLAC1, MBLAC2, MBNL1, MBNL2, MBNL3, MBOAT1, MBOAT2, MBOAT4, MBOAT7, MBP, MBTD1, MBTPS1, MBTPS2, MC1R, MC2R, MC3R, MC4R, MC5R, MCAM, MCAT, MCC, MCCC1, MCCC2, MCCD1, MCEE, MCEMP1, MCF2, MCF2L, MCF2L2, MCFD2, MCHR1, MCHR2, MCIDAS, MCL1, MCM10, MCM2, MCM3, MCM3AP, MCM4, MCM5, MCM6, MCM7, MCM8, MCM9, MCMBP, MCMDC2, MCOLN1, MCOLN2, MCOLN3, MCPH1, MCRIP1, MCRIP2, MCRS1, MCTP1, MCTP2, MCTS1, MCU, MCUB, MCUR1, MDC1, MDFI, MDFIC, MDFIC2, MDGA1, MDGA2, MDH1, MDH1B, MDH2, MDK, MDM1, MDM2, MDM4, MDN1, MDP1, MDS2, ME1, ME2, ME3, MEAl, MEAF6, MECOM, MECP2, MECR, MED1, MED10, MED11, MED12, MED12L, MED13, MED13L, MED14, MED14OS, MED15, MED16, MED17, MED18, MED19, MED20, MED21, MED22, MED23, MED24, MED25, MED26, MED27, MED28, MED29, MED30, MED31, MED4, MED6, MED7, MED8, MED9, MEDAG, MEF2A, MEF2B, MEF2C, MEF2D, MEFV, MEGF10, MEGF11, MEGF6, MEGF8, MEGF9, MEIl, MEI4, MEIG1, MEIKIN, MEIOB, MEIOC, MEIS1, MEIS2, MEIS3, MELK, MELTF, MEMO1, MEN1, MEOX1, MEOX2, MEPlA, MEP1B, MEPCE, MEPE, MESD, MESP1, MESP2, MEST, MET, METAP1, METAPlD, METAP2, METRN, METRNL, METTL1, METTL11B, METTL12, METTL13, METTL14, METTL15, METTL16, METTL17, METTL18, METTL21A, METTL21C, METTL22, METTL23, METTL24, METTL25, METTL26, METTL27, METTL2A, METTL2B, METTL3, METTL4, METTL5, METTL6, METTL7A, METTL7B, METTL8, METTL9, MEX3A, MEX3B, MEX3C, MEX3D, MFAP1, MFAP2, MFAP3, MFAP3L, MFAP4, MFAP5, MFF, MFGE8, MFHAS1, MFN1, MFN2, MFNG, MFRP, MFSD1, MFSD10, MFSD11, MFSD12, MFSD13A, MFSD14A, MFSD14B, MFSD14C, MFSD2A, MFSD2B, MFSD3, MFSD4A, MFSD4B, MFSD5, MFSD6, MFSD6L, MFSD7, MFSD8, MFSD9, MGA, MGAM, MGAM2, MGARP, MGAT1, MGAT2, MGAT3, MGAT4A, MGAT4B, MGAT4C, MGAT4D, MGAT5, MGAT5B, MGEA5, MGLL, MGME1, MGMT, MGP, MGRN1, MGST1, MGST2, MGST3, MIA, MIA3, MIA-RAB4B, MIB1, MIB2, MICA, MICALl, MICAL2, MICAL3, MICALCL, MICALL1, MICALL2, MICB, MICU1, MICU2, MICU3, MIDi, MID1IP1, MID2, MIDN, MIEF1, MIEF2, MIEN1, MIER1, MIER2, MIER3, MIF, MIF4GD, MIGA1, MIGA2, MIIP, MILR1, MINDY1, MINDY2, MINDY3, MINDY4, MINDY4B, MINK1, MINOS1, MINOS1-NBL1, MINPP1, MIOS, MIOX, MIP, MIPEP, MIPOL1, MIS12, MIS18A, MIS18BP1, MISP, MISP3, MITD1, MITF, MIXL1, MKI67, MKKS, MKL1, MKL2, MKLN1, MKNK1, MKNK2, MKRN1, MKRN2, MKRN2OS, MKRN3, MKS1, MKX, MLANA, MLC1, MLEC, MLF1, MLF2, MLH1, MLH3, MLIP, MLKL, MLLT1, MLLT10, MLLT11, MLLT3, MLLT6, MLN, MLNR, MLPH, MLST8, MLX, MLXIP, MLXIPL, MLYCD, MMAA, MMAB, MMACHC, MMADHC, MMD, MMD2, MME, MMEL1, MMGT1, MMP1, MMP10, MMP11, MMP12, MMP13, MMPP14, MMP15, MMP16, MMPP17, MMP19, MMP2, MMP20, MMP21, MMP23B, MMP24, MMP24-AS1, MMP25, MMP26, MMP27, MMP28, MMP3, MMP7, MMP8, MMP9, MMRN1, MMRN2, MMS19, MMS22L, MN1, MNAT1, MND1, MNDA, MNS1, MNT, MNX1, MOAP1, MOB1A, MOB1B, MOB2, MOB3A, MOB3B, MOB3C, MOB4, MOBP, MOCOS, MOCS1, MOCS2, MOCS3, MOG, MOGATI, MOGAT2, MOGAT3, MOGS, MOK, MON1A, MON1B, MON2, MORC1, MORC2, MORC3, MORC4, MORF4L1, MORF4L2, MORN1, MORN2, MORN3, MORN4, MORN5, MOS, MOSPD1, MOSPD2, MOSPD3, MOV10, MOV10L1, MOXD1, MPC1, MPC1L, MPC2, MPDU1, MPDZ, MPEG1, MPG, MPHOSPH10, MPHOSPH6, MPHOSPH8, MPHOSPH9, MPI, MPIG6B, MPL, MPLKIP, MPND, MPO, MPP1, MPP2, MPP3, MPP4, MPP5, MPP6, MPP7, MPPEl, MPPED1, MPPED2, MPRIP, MPST, MPV17, MPV17L, MPV17L2, MPZ, MPZL1, MPZL2, MPZL3, MR1, MRAP, MRAP2, MRAS, MRC1, MRC2, MRE11, MREG, MRFAP1, MRFAP1L1, MRGBP, MRGPRD, MRGPRE, MRGPRF, MRGPRG, MRGPRX1, MRGPRX2, MRGPRX3, MRGPRX4, MRI1, MRLN, MRM1, MRM2, MRM3, MRNIP, MRO, MROH1, MROH2A, MROH2B, MROH5, MROH6, MROH7, MROH7-TTC4, MROH8, MROH9, MRPL1, MRPL10, MRPL11, MRPL12, MRPL13, MRPL14, MRPL15, MRPL16, MRPL17, MRPL18, MRPL19, MRPL2, MRPL20, MRPL21, MRPL22, MRPL23, MRPL24, MRPL27, MRPL28, MRPL3, MRPL30, MRPL32, MRPL33, MRPL34, MRPL35, MRPL36, MRPL37, MRPL38, MRPL39, MRPL4, MRPL40, MRPL41, MRPL42, MRPL43, MRPL44, MRPL45, MRPL46, MRPL47, MRPL48, MRPL49, MRPL50, MRPL51, MRPL52, MRPL53, MRPL54, MRPL55, MRPL57, MRPL58, MRPL9, MRPS10, MRPS11, MRPS12, MRPS14, MRPS15, MRPS16, MRPS17, MRPS18A, MRPS18B, MRPS18C, MRPS2, MRPS21, MRPS22, MRPS23, MRPS24, MRPS25, MRPS26, MRPS27, MRPS28, MRPS30, MRPS31, MRPS33, MRPS34, MRPS35, MRPS36, MRPS5, MRPS6, MRPS7, MRPS9, MRRF, MRS2, MRTO4, MRVI1, MS4A1, MS4A10, MS4A12, MS4A13, MS4A14, MS4A15, MS4A2, MS4A3, MS4A4A, MS4A4E, MS4A5, MS4A6A, MS4A6E, MS4A7, MS4A8, MSANTD1, MSANTD2, MSANTD3, MSANTD3-TMEFF1, MSANTD4, MSC, MSGN1, MSH2, MSH3, MSH4, MSH5, MSH5-SAPCD1, MSH6, MSI1, MSI2, MSL1, MSL2, MSL3, MSLN, MSLNL, MSMB, MSMO1, MSMP, MSN, MSR1, MSRA, MSRB1, MSRB2, MSRB3, MSS51, MST1, MST1R, MSTN, MSTO1, MSX1, MSX2, MT1A, MT1B, MT1E, MT1F, MT1G, MT1H, MT1IL1, MT1M, MT1X, MT2A, MT3, MT4, MTA1, MTA2, MTA3, MTAP, MT-ATP6, MT-ATP8, MTBP, MTCH1, MTCH2, MTCL1, MT-CO1, MT-C02, MT-C03, MTCP1, MT-CYB, MTDH, MTERF1, MTERF2, MTERF3, MTERF4, MTF1, MTF2, MTFMT, MTFP1, MTFR1, MTFR1L, MTFR2, MTG1, MTG2, MTHFD1, MTHFD1L, MTHFD2, MTHFD2L, MTHFR, MTHFS, MTHFSD, MTIF2, MTIF3, MTM1, MTMR1, MTMR10, MTMR11, MTMR12, MTMR14, MTMR2, MTMR3, MTMR4, MTMR6, MTMR7, MTMR8, MTMR9, MT-ND1, MT-ND2, MT-ND3, MT-ND4, MT-ND4L, MT-ND5, MT-ND6, MTNR1A, MTNR1B, MTO1, MTOR, MTPAP, MTPN, MTR, MTRF1, MTRF1L, MTRNR₂L1, MTRNR₂L10, MTRNR2 L11, MTRNR₂L12, MTRNR₂L13, MTRNR₂L3, MTRNR₂L4, MTRNR₂L5, MTRNR₂L6, MTRNR₂L7, MTRNR₂L8, MTRR, MTSS1, MTSSIL, MTTP, MTURN, MTUS1, MTUS2, MTX1, MTX2, MTX3, MUC1, MUC12, MUC13, MUC15, MUC16, MUC17, MUC2, MUC20, MUC21, MUC22, MUC3A, MUC4, MUC5AC, MUC5B, MUC6, MUC7, MUCL1, MUL1, MUM1, MUM1L1, MUS81, MUSK, MUSTN1, MUT, MUTYH, MVB12A, MVB12B, MVD, MVK, MVP, MX1, MX2, MXD1, MXD3, MXD4, MXIl, MXRA5, MXRA7, MXRA8, MYADM, MYADML2, MYB, MYBBPlA, MYBL1, MYBL2, MYBPC1, MYBPC2, MYBPC3, MYBPH, MYBPHL, MYC, MYCBP, MYCBP2, MYCBPAP, MYCL, MYCN, MYCT1, MYD88, MYDGF, MYEF2, MYEOV, MYF5, MYF6, MYH1, MYH10, MYH11, MYH13, MYH14, MYH15, MYH2, MYH3, MYH4, MYH6, MYH7, MYH7B, MYH8, MYH9, MYL1, MYL10, MYL12A, MYL12B, MYL2, MYL3, MYL4, MYL5, MYL6, MYL6B, MYL7, MYL9, MYLIP, MYLK, MYLK2, MYLK3, MYLK4, MYLPF, MYMK, MYMX, MYNN, MYO10, MYO15A, MYO15B, MYO16, MYO18A, MYO18B, MYO19, MYOIA, MYO1B, MYO1C, MYO1D, MYOIE, MYO1F, MYO1G, MYO1H, MYO3A, MYO3B, MYO5A, MYO5B, MYO5C, MYO6, MYO7A, MYO7B, MYO9A, MYO9B, MYOC, MYOCD, MYOCOS, MYOD1, MYOF, MYOG, MYOM1, MYOM2, MYOM3, MYOT, MYOZ1, MYOZ2, MYOZ3, MYPN, MYPOP, MYRF, MYRFL, MYRIP, MYSM1, MYT1, MYT1L, MYZAP, MZB1, MZF1, MZT1, MZT2A, MZT2B, N4BP1, N4BP2, N4BP2L1, N4BP2L2, N4BP3, N6AMT1, NAA10, NAA11, NAA15, NAA16, NAA20, NAA25, NAA30, NAA35, NAA38, NAA40, NAA50, NAA60, NAAA, NAALAD2, NAALADL1, NAALADL2, NAB1, NAB2, NABP1, NABP2, NACA, NACA2, NACAD, NACC1, NACC2, NADK, NADK2, NADSYN1, NAE1, NAF1, NAGA, NAGK, NAGLU, NAGPA, NAGS, NAIF1, NAIP, NALCN, NAMPT, NANOG, NANOGNB, NANOGP8, NANOSI, NANOS2, NANOS3, NANP, NANS, NAP1L1, NAP1L2, NAP1L3, NAP1L4, NAP1L5, NAPA, NAPB, NAPEPLD, NAPG, NAPRT, NAPSA, NARF, NARFL, NARS, NARS2, NASP, NAT1, NAT10, NAT14, NAT16, NAT2, NAT6, NAT8, NAT8B, NAT8L, NAT9, NATD1, NAV1, NAV2, NAV3, NAXD, NAXE, NBAS, NBDY, NBEA, NBEAL1, NBEAL2, NBL1, NBN, NBPF1, NBPF10, NBPF11, NBPF12, NBPF14, NBPF15, NBPF19, NBPF20, NBPF26, NBPF3, NBPF4, NBPF6, NBPF9, NBR1, NCALD, NCAM1, NCAM2, NCAN, NCAPD2, NCAPD3, NCAPG, NCAPG2, NCAPH, NCAPH2, NCBP1, NCBP2, NCBP2-AS2, NCBP2L, NCBP3, NCCRP1, NCDN, NCEH1, NCF1, NCF2, NCF4, NCK1, NCK2, NCKAP1, NCKAP1L, NCKAP5, NCKAP5L, NCKIPSD, NCL, NCLN, NCMAP, NCOA1, NCOA2, NCOA3, NCOA4, NCOA5, NCOA6, NCOA7, NCOR1, NCOR₂, NCR1, NCR2, NCR3, NCR3LG1, NCS1, NCSTN, NDC1, NDC80, NDE1, NDEL1, NDFIP1, NDFIP2, NDN, NDNF, NDOR1, NDP, NDRG1, NDRG2, NDRG3, NDRG4, NDST1, NDST2, NDST3, NDST4, NDUFA1, NDUFA10, NDUFA11, NDUFA12, NDUFA13, NDUFA2, NDUFA3, NDUFA4, NDUFA4L2, NDUFA5, NDUFA6, NDUFA7, NDUFA8, NDUFA9, NDUFAB1, NDUFAF1, NDUFAF2, NDUFAF3, NDUFAF4, NDUFAF5, NDUFAF6, NDUFAF7, NDUFAF8, NDUFB1, NDUFB10, NDUFB11, NDUFB2, NDUFB3, NDUFB4, NDUFB5, NDUFB6, NDUFB7, NDUFB8, NDUFB9, NDUFC1, NDUFC2, NDUFC2-KCTD14, NDUFS1, NDUFS2, NDUFS3, NDUFS4, NDUFS5, NDUFS6, NDUFS7, NDUFS8, NDUFV1, NDUFV2, NDUFV3, NEB, NEBL, NECABI, NECAB2, NECAB3, NECAPI, NECAP2, NECTINI, NECTIN2, NECTIN3, NECTIN4, NEDD1, NEDD4, NEDD4L, NEDD8, NEDD8-MDP1, NEDD9, NEFH, NEFL, NEFM, NEGR1, NEIL1, NEIL2, NEIL3, NEK1, NEK10, NEK11, NEK2, NEK3, NEK4, NEK5, NEK6, NEK7, NEK8, NEK9, NELFA, NELFB, NELFCD, NELFE, NELL1, NELL2, NEMF, NEMP1, NEMP2, NENF, NEO1, NEPRO, NES, NET1, NETO1, NETO2, NEU1, NEU2, NEU3, NEU4, NEURLI, NEURLIB, NEURL2, NEURL3, NEURL4, NEURODI, NEUROD2, NEUROD4, NEUROD6, NEUROGI, NEUROG2, NEUROG3, NEXMIF, NEXN, NF1, NF2, NFAM1, NFASC, NFAT5, NFATC1, NFATC2, NFATC2IP, NFATC3, NFATC4, NFE2, NFE2L1, NFE2L2, NFE2L3, NFE4, NFIA, NFIB, NFIC, NFIL3, NFIX, NFKB1, NFKB2, NFKBIA, NFKBIB, NFKBID, NFKBIE, NFKBIL1, NFKBIZ, NFRKB, NFSl, NFU1, NFX1, NFXL1, NFYA, NFYB, NFYC, NGB, NGDN, NGEF, NGF, NGFR, NGLY1, NGRN, NHEJ1, NHLH1, NHLH2, NHLRC1, NHLRC2, NHLRC3, NHLRC4, NHP2, NHS, NHSL1, NHSL2, NICN1, NID1, NID2, NIF3L1, NIFK, NIM1K, NIN, NINJ1, NINJ2, NINL, NIP7, NIPA1, NIPA2, NIPAL1, NIPAL2, NIPAL3, NIPAL4, NIPBL, NIPSNAP1, NIPSNAP2, NIPSNAP3A, NIPSNAP3B, NISCH, NIT1, NIT2, NKAIN1, NKAIN2, NKAIN3, NKAIN4, NKAP, NKAPL, NKD1, NKD2, NKG7, NKIRAS1, NKIRAS2, NKPD1, NKRF, NKTR, NKX1-1, NKX1-2, NKX2-1, NKX2-2, NKX2-3, NKX2-4, NKX2-5, NKX2-6, NKX2-8, NKX3-1, NKX3-2, NKX6-1, NKX6-2, NKX6-3, NLE1, NLGN1, NLGN2, NLGN3, NLGN4X, NLGN4Y, NLK, NLN, NLRC3, NLRC4, NLRC5, NLRP1, NLRP10, NLRP11, NLRP12, NLRP13, NLRP14, NLRP2, NLRP2B, NLRP3, NLRP4, NLRP5, NLRP6, NLRP7, NLRP8, NLRP9, NLRX1, NMB, NMBR, NMD3, NME1, NME1-NME2, NME2, NME3, NME4, NME5, NME6, NME7, NME8, NME9, NMI, NMNAT1, NMNAT2, NMNAT3, NMRAL1, NMRK1, NMRK2, NMS, NMT1, NMT2, NMU, NMUR1, NMUR2, NNAT, NNMT, NNT, NOA1, NOB1, NOBOX, NOC2L, NOC3L, NOC4L, NOCT, NOD1, NOD2, NODAL, NOG, NOL10, NOL11, NOL12, NOL3, NOL4, NOL4L, NOL6, NOL7, NOL8, NOL9, NOLC1, NOM1, NOMO1, NOMO2, NOMO3, NONO, NOP10, NOP14, NOP16, NOP2, NOP53, NOP56, NOP58, NOP9, NOS1, NOS1AP, NOS2, NOS3, NOSIP, NOSTRIN, NOTCHI, NOTCH2, NOTCH2NL, NOTCH3, NOTCH4, NOTO, NOTUM, NOV, NOVAl, NOVA2, NOX1, NOX3, NOX4, NOX5, NOXA1, NOXO1, NOXRED1, NPAP1, NPAS1, NPAS2, NPAS3, NPAS4, NPAT, NPB, NPBWR1, NPBWR2, NPC1, NPC1L1, NPC2, NPDC1, NPEPL1, NPEPPS, NPFF, NPFFR1, NPFFR2, NPHP1, NPHP3, NPHP3-ACAD11, NPHP4, NPHS1, NPHS2, NPIPA1, NPIPA2, NPIPA3, NPIPA5, NPIPA7, NPIPA8, NPIPB11, NPIPB12, NPIPB13, NPIPB15, NPIPB2, NPIPB3, NPIPB4, NPIPB5, NPIPB6, NPIPB7, NPIPB8, NPIPB9, NPL, NPLOC4, NPM1, NPM2, NPM3, NPNT, NPPA, NPPB, NPPC, NPR1, NPR2, NPR3, NPRL2, NPRL3, NPS, NPSR1, NPTN, NPTX1, NPTX2, NPTXR, NPVF, NPW, NPY, NPY1R, NPY2R, NPY4R, NPY4R2, NPY5R, NQO1, NQO2, NROB1, NROB2, NR1D1, NR1D2, NR1H2, NR1H3, NR1H4, NR1I2, NR1I3, NR₂C1, NR₂C2, NR₂C2AP, NR₂E1, NR2E3, NR₂F1, NR₂F2, NR2F6, NR3C1, NR3C2, NR4A1, NR4A2, NR4A3, NR5A1, NR5A2, NR6A1, NRAP, NRARP, NRAS, NRBF2, NRBP1, NRBP2, NRCAM, NRDC, NRDE2, NREP, NRF1, NRG1, NRG2, NRG3, NRG4, NRGN, NRIP1, NRIP2, NRIP3, NRK, NRL, NRM, NRN1, NRN1L, NRP1, NRP2, NRROS, NRSN1, NRSN2, NRTN, NRXN1, NRXN2, NRXN3, NSA2, NSD1, NSD2, NSD3, NSDHL, NSF, NSFL1C, NSL1, NSMAF, NSMCEl, NSMCE2, NSMCE3, NSMCE4A, NSMF, NSRP1, NSUN2, NSUN3, NSUN4, NSUN5, NSUN6, NSUN7, NT5C, NT5C1A, NT5C1B, NT5C1B-RDH14, NT5C2, NT5C3A, NT5C3B, NT5DC1, NT5DC2, NT5DC3, NT5DC4, NT5E, NT5M, NTAN1, NTF3, NTF4, NTHL1, NTM, NTMT1, NTN1, NTN3, NTN4, NTN5, NTNG1, NTNG2, NTPCR, NTRK1, NTRK2, NTRK3, NTS, NTSR1, NTSR2, NUAK1, NUAK2, NUB1, NUBP1, NUBP2, NUBPL, NUCB1, NUCB2, NUCKS1, NUDC, NUDCD1, NUDCD2, NUDCD3, NUDT1, NUDT10, NUDT11, NUDT12, NUDT13, NUDT14, NUDT15, NUDT16, NUDT16L1, NUDT17, NUDT18, NUDT19, NUDT2, NUDT21, NUDT22, NUDT3, NUDT4, NUDT4P1, NUDT5, NUDT6, NUDT7, NUDT8, NUDT9, NUF2, NUFIPI, NUFIP2, NUGGC, NUMA1, NUMB, NUMBL, NUP107, NUP133, NUP153, NUP155, NUP160, NUP188, NUP205, NUP210, NUP210L, NUP214, NUP35, NUP37, NUP43, NUP50, NUP54, NUP58, NUP62, NUP62CL, NUP85, NUP88, NUP93, NUP98, NUPL2, NUPR1, NUPR2, NUS1, NUSAPI, NUTF2, NUTM1, NUTM2A, NUTM2B, NUTM2D, NUTM2E, NUTM2F, NUTM2G, NVL, NWD1, NWD2, NXF1, NXF2, NXF2B, NXF3, NXF5, NXN, NXNL1, NXNL2, NXPE1, NXPE2, NXPE3, NXPE4, NXPH1, NXPH2, NXPH3, NXPH4, NXT1, NXT2, NYAP1, NYAP2, NYNRIN, NYX, OAF, OARD1, OAS1, OAS2, OAS3, OASL, OAT, OAZ1, OAZ2, OAZ3, OBP2A, OBP2B, OBSCN, OBSCN-AS1, OBSL1, OC90, OCA2, OCEL1, OCIAD1, OCIAD2, OCLM, OCLN, OCM, OCM2, OCRL, OCSTAMP, ODAM, ODC1, ODF1, ODF2, ODF2L, ODF3, ODF3B, ODF3L1, ODF3L2, ODF4, OFCC1, OFD1, OGDH, OGDHL, OGFOD1, OGFOD2, OGFOD3, OGFR, OGFRL1, OGG1, OGN, OGT, OIP5, OIT3, OLA1, OLAH, OLFM1, OLFM2, OLFM3, OLFM4, OLFML1, OLFML2A, OLFML2B, OLFML3, OLIG1, OLIG2, OLIG3, OLR1, OMA1, OMD, OMG, OMP, ONECUT1, ONECUT2, ONECUT3, OOEP, OOSP2, OPAl, OPA3, OPALIN, OPCML, OPHN1, OPLAH, OPN1LW, OPN1MW, OPN1MW2, OPN1MW3, OPN1SW, OPN3, OPN4, OPN5, OPRD1, OPRK1, OPRL1, OPRM1, OPRPN, OPTC, OPTN, OR10A2, OR10A3, OR10A4, OR10A5, OR10A6, OR10A7, OR10AC1, OR10AD1, OR10AG1, OR10C1, OR10D3, OR10G2, OR10G3, OR10G4, OR10G6, OR10G7, OR10G8, OR10G9, OR10H1, OR10H2, OR10H3, OR10H4, OR10H5, OR10J1, OR10J3, OR10J4, OR10J5, OR10K1, OR10K2, OR10P1, OR10Q1, OR10R2, OR10S1, OR10T2, OR10V1, OR10W1, OR10X1, OR10Z1, OR11A1, OR11G2, OR11H1, OR11H12, OR11H2, OR11H4, OR11H6, OR11H7, OR11L1, OR12D1, OR12D2, OR12D3, OR13A1, OR13C2, OR13C3, OR13C4, OR13C5, OR13C7, OR13C8, OR13C9, OR13D1, OR13F1, OR13G1, OR13H1, OR13J1, OR14A16, OR14A2, OR14C36, OR14I1, OR14J1, OR14K1, OR1A1, OR1A2, OR1B1, OR1C1, OR1D2, OR1D5, OR1E1, OR1E2, OR1F1, OR1G1, OR1I1, OR1J1, OR1J2, OR1J4, OR1K1, OR1L1, OR1L3, OR1L4, OR1L6, OR1L8, OR1M1, OR1N1, OR1N2, OR1P1, OR1Q1, OR1S1, OR1S2, OR₂A1, OR₂A12, OR₂A14, OR₂A2, OR₂A25, OR₂A4, OR₂A42, OR₂A5, OR₂A7, OR₂AEI, OR₂AGI, OR₂AG2, OR₂AJ1, OR₂AK2, OR₂API, OR₂AT4, OR₂B11, OR₂B2, OR₂B3, OR₂B6, OR₂C1, OR₂C3, OR₂D2, OR₂D3, OR₂F1, OR₂F2, OR₂G2, OR₂G3, OR₂G6, OR₂H1, OR₂H2, OR₂J1, OR₂J2, OR₂J3, OR₂K2, OR₂L13, OR₂L2, OR₂L3, OR₂L5, OR₂L8, OR₂M2, OR₂M3, OR₂M4, OR₂M5, OR₂M7, OR₂S2, OR₂T1, OR₂T10, OR₂T11, OR₂T12, OR₂T2, OR₂T27, OR₂T29, OR₂T3, OR₂T33, OR₂T34, OR₂T35, OR₂T4, OR2 T5, OR₂T6, OR₂T7, OR₂T8, OR₂VI, OR₂V2, OR₂W1, OR₂W3, OR₂Y1, OR₂Z1, OR3A1, OR3A2, OR3A3, OR4A15, OR4A16, OR4A47, OR4A5, OR4A8, OR4B1, OR4C11, OR4C12, OR4C13, OR4C15, OR4C16, OR4C3, OR4C45, OR4C46, OR4C5, OR4C6, OR4D1, OR4D10, OR4D11, OR4D2, OR4D5, OR4D6, OR4D9, OR4E1, OR4E2, OR4F15, OR4F16, OR4F17, OR4F21, OR4F29, OR4F3, OR4F4, OR4F5, OR4F6, OR4K1, OR4K13, OR4K14, OR4K15, OR4K17, OR4K2, OR4K3, OR4K5, OR4L1, OR4M1, OR4M2, OR4N2, OR4N4, OR4N5, OR4P4, OR4Q2, OR4Q3, OR4S1, OR4S2, OR4X1, OR4X2, OR51A2, OR51A4, OR51A7, OR51B2, OR51B4, OR51B5, OR51B6, OR51D1, OR51E1, OR51E2, OR51F1, OR51F2, OR51G1, OR51G2, OR51H1, OR51I1, OR51I2, OR51J1, OR51L1, OR51M1, OR51Q1, OR51S1, OR51T1, OR51V1, OR52A1, OR52A5, OR52B2, OR52B4, OR52B6, OR52D1, OR52E2, OR52E4, OR52E5, OR52E6, OR52E8, OR52H1, OR52I1, OR52I2, OR52J3, OR52K1, OR52K2, OR52L1, OR52M1, OR52N1, OR52N2, OR52N4, OR52N5, OR52R1, OR52W1, OR52Z1, OR56A1, OR56A3, OR56A4, OR56A5, OR56B1, OR56B4, OR5A1, OR5A2, OR5AC1, OR5AC2, OR5AK2, OR5ANI, OR5AP2, OR5AR1, OR5AS1, OR5AU1, OR5B12, OR5B17, OR5B2, OR5B21, OR5B3, OR5C1, OR5D13, OR5D14, OR5D16, OR5D18, OR5F1, OR5G3, OR5H1, OR5H14, OR5H15, OR5H2, OR5H6, OR5H8, OR5I1, OR5J2, OR5K1, OR5K2, OR5K3, OR5K4, OR5L1, OR5L2, OR5M1, OR5M10, OR5M11, OR5M3, OR5M8, OR5M9, OR5P2, OR5P3, OR5R1, OR5T1, OR5T2, OR5T3, OR5V1, OR5W2, OR6A2, OR6B1, OR6B2, OR6B3, OR6C1, OR6C2, OR6C3, OR6C4, OR6C6, OR6C65, OR6C68, OR6C70, OR6C74, OR6C75, OR6C76, OR6F1, OR6J1, OR6K2, OR6K3, OR6K6, OR6M1, OR6N1, OR6N2, OR6P1, OR6Q1, OR6S1, OR6T1, OR6V1, OR6X1, OR6Y1, OR7A10, OR7A17, OR7A5, OR7C1, OR7C2, OR7D2, OR7D4, OR7E24, OR7G1, OR7G2, OR7G3, OR8A1, OR8B12, OR8B2, OR8B3, OR8B4, OR8B8, OR8D1, OR8D2, OR8D4, OR8G1, OR8G5, OR8H1, OR8H2, OR8H3, OR8I2, OR8J1, OR8J2, OR8J3, OR8K1, OR8K3, OR8K5, OR8S1, OR8U1, OR8U8, OR9A2, OR9A4, OR9G1, OR9G4, OR9G9, OR9H1P, OR9I1, OR9K2, OR9Q1, OR9Q2, ORAIl, ORAI2, ORAI3, ORAOV1, ORC1, ORC2, ORC3, ORC4, ORC5, ORC6, ORM1, ORM2, ORMDL1, ORMDL2, ORMDL3, OS9, OSBP, OSBP2, OSBPL10, OSBPL11, OSBPL1A, OSBPL2, OSBPL3, OSBPL5, OSBPL6, OSBPL7, OSBPL8, OSBPL9, OSCAR, OSCP1, OSER1, OSGEP, OSGEPLI, OSGINI, OSGIN2, OSM, OSMR, OSR1, OSR2, OST4, OSTC, OSTF1, OSTM1, OSTN, OTC, OTOA, OTOF, OTOG, OTOGL, OTOL1, OTOP1, OTOP2, OTOP3, OTOR, OTOS, OTP, OTUB1, OTUB2, OTUD1, OTUD3, OTUD4, OTUD5, OTUD6A, OTUD6B, OTUD7A, OTUD7B, OTULIN, OTX1, OTX2, OVCA2, OVCH1, OVCH₂, OVGP1, OVOL1, OVOL2, OVOL3, OXA1L, OXCT1, OXCT2, OXER1, OXGR1, OXLD1, OXNAD1, OXR1, OXSM, OXSR1, OXT, OXTR, P2RX1, P2RX2, P2RX3, P2RX4, P2RX5, P2RX5-TAX1BP3, P2RX6, P2RX7, P2RY1, P2RY10, P2RY11, P2RY12, P2RY13, P2RY14, P2RY2, P2RY4, P2RY6, P2RY8, P3H1, P3H2, P3H3, P3H4, P4HA1, P4HA2, P4HA3, P4HB, P4HTM, PA2G4, PAAF1, PABPC1, PABPC1L, PABPC1L2A, PABPC1L2B, PABPC3, PABPC4, PABPC4L, PABPC5, PABPN1, PABPN1L, PACRG, PACRGL, PACS1, PACS2, PACSIN1, PACSIN2, PACSIN3, PADI1, PADI2, PADI3, PADI4, PADI6, PAEP, PAF1, PAFAHIBI, PAFAH1B2, PAFAH1B3, PAFAH2, PAG1, PAGEl, PAGE2, PAGE2B, PAGE3, PAGE4, PAGE5, PAGR1, PAH, PAICS, PAIP1, PAIP2, PAIP2B, PAK1, PAK1IP1, PAK2, PAK3, PAK4, PAK5, PAK6, PALB2, PALD1, PALLD, PALM, PALM2, PALM2-AKAP2, PALM3, PALMD, PAM, PAM16, PAMR1, PAN2, PAN3, PANK1, PANK2, PANK3, PANK4, PANO1, PANX1, PANX2, PANX3, PAOX, PAPD4, PAPD5, PAPD7, PAPLN, PAPOLA, PAPOLB, PAPOLG, PAPPA, PAPPA2, PAPSS1, PAPSS2, PAQR3, PAQR4, PAQR5, PAQR6, PAQR7, PAQR8, PAQR9, PARD3, PARD3B, PARD6A, PARD6B, PARD6G, PARG, PARK7, PARL, PARM1, PARN, PARP1, PARP10, PARP11, PARP12, PARP14, PARP15, PARP16, PARP2, PARP3, PARP4, PARP6, PARP8, PARP9, PARPBP, PARS2, PARVA, PARVB, PARVG, PASD1, PASK, PATE1, PATE2, PATE3, PATE4, PATJ, PATL1, PATL2, PATZ1, PAWR, PAX1, PAX2, PAX3, PAX4, PAX5, PAX6, PAX7, PAX8, PAX9, PAXBP1, PAXIP1, PAXX, PBDC1, PBK, PBLD, PBOV1, PBRM1, PBX1, PBX2, PBX3, PBX4, PBXIP1, PC, PCBD1, PCBD2, PCBP1, PCBP2, PCBP3, PCBP4, PCCA, PCCB, PCDH1, PCDH10, PCDH11X, PCDH11Y, PCDH12, PCDH15, PCDH17, PCDH18, PCDH19, PCDH20, PCDH7, PCDH8, PCDH9, PCDHA1, PCDHA10, PCDHA11, PCDHA12, PCDHA13, PCDHA2, PCDHA3, PCDHA4, PCDHA5, PCDHA6, PCDHA7, PCDHA8, PCDHA9, PCDHAC1, PCDHAC2, PCDHB1, PCDHB10, PCDHB11, PCDHB12, PCDHB13, PCDHB14, PCDHB15, PCDHB16, PCDHB2, PCDHB3, PCDHB4, PCDHB5, PCDHB6, PCDHB7, PCDHB8, PCDHB9, PCDHGA1, PCDHGA10, PCDHGA11, PCDHGA12, PCDHGA2, PCDHGA3, PCDHGA4, PCDHGA5, PCDHGA6, PCDHGA7, PCDHGA8, PCDHGA9, PCDHGB1, PCDHGB2, PCDHGB3, PCDHGB4, PCDHGB5, PCDHGB6, PCDHGB7, PCDHGC3, PCDHGC4, PCDHGC5, PCED1A, PCED1B, PCF11, PCGF1, PCGF2, PCGF3, PCGF5, PCGF6, PCID2, PCIF1, PCK1, PCK2, PCLAF, PCLO, PCM1, PCMT1, PCMTD1, PCMTD2, PCNA, PCNP, PCNT, PCNX1, PCNX2, PCNX3, PCNX4, PCOLCE, PCOLCE2, PCOTH, PCP2, PCP4, PCP4L1, PCSK1, PCSKlN, PCSK2, PCSK4, PCSK5, PCSK6, PCSK7, PCSK9, PCTP, PCYOX1, PCYOX1L, PCYT1A, PCYTIB, PCYT2, PDAP1, PDC, PDCD1, PDCD10, PDCD11, PDCD1LG2, PDCD2, PDCD2L, PDCD4, PDCD5, PDCD6, PDCD6IP, PDCD7, PDCL, PDCL2, PDCL3, PDE10A, PDE11A, PDE12, PDE1A, PDE1B, PDE1C, PDE2A, PDE3A, PDE3B, PDE4A, PDE4B, PDE4C, PDE4D, PDE4DIP, PDE5A, PDE6A, PDE6B, PDE6C, PDE6D, PDE6G, PDE6H, PDE7A, PDE7B, PDE8A, PDE8B, PDE9A, PDF, PDGFA, PDGFB, PDGFC, PDGFD, PDGFRA, PDGFRB, PDGFRL, PDHA1, PDHA2, PDHB, PDHX, PDIA2, PDIA3, PDIA4, PDIA5, PDIA6, PDIK1L, PDILT, PDK1, PDK2, PDK3, PDK4, PDLIM1, PDLIM2, PDLIM3, PDLIM4, PDLIM5, PDLIM7, PDP1, PDP2, PDPK1, PDPN, PDPR, PDRG1, PDS5A, PDS5B, PDSS1, PDSS2, PDX1, PDXDC1, PDXK, PDXP, PDYN, PDZD11, PDZD2, PDZD3, PDZD4, PDZD7, PDZD8, PDZD9, PDZK1, PDZK1IP1, PDZRN3, PDZRN4, PEA15, PEAK1, PEAR1, PEBP1, PEBP4, PECAM1, PECR, PEF1, PEG10, PEG3, PELI1, PELI2, PELI3, PELO, PELP1, PEMT, PENK, PEPD, PER1, PER2, PER3, PERM1, PERP, PES1, PET100, PET117, PEX1, PEX10, PEX11A, PEX11B, PEX11G, PEX12, PEX13, PEX14, PEX16, PEX19, PEX2, PEX26, PEX3, PEX5, PEX5L, PEX6, PEX7, PF4, PF4V1, PFAS, PFDN1, PFDN2, PFDN4, PFDN5, PFDN6, PFKFB1, PFKFB2, PFKFB3, PFKFB4, PFKL, PFKM, PFKP, PFN1, PFN2, PFN3, PFN4, PGA3, PGA4, PGA5, PGAM1, PGAM2, PGAM4, PGAM5, PGAP1, PGAP2, PGAP3, PGBD1, PGBD2, PGBD4, PGBD5, PGC, PGD, PGF, PGGHG, PGGT1B, PGK1, PGK2, PGLS, PGLYRP1, PGLYRP2, PGLYRP3, PGLYRP4, PGM1, PGM2, PGM2L1, PGM3, PGM5, PGP, PGPEP1, PGPEP1L, PGR, PGRMC1, PGRMC2, PGS1, PHACTRI, PHACTR2, PHACTR3, PHACTR4, PHAX, PHB, PHB2, PHC1, PHC2, PHC3, PHEX, PHF1, PHF10, PHF11, PHF12, PHF13, PHF14, PHF19, PHF2, PHF20, PHF20L1, PHF21A, PHF21B, PHF23, PHF24, PHF3, PHF5A, PHF6, PHF7, PHF8, PHGDH, PHGR1, PHIP, PHKA1, PHKA2, PHKB, PHKG1, PHKG2, PHLDAl, PHLDA2, PHLDA3, PHLDB1, PHLDB2, PHLDB3, PHLPP1, PHLPP2, PHOSPHO1, PHOSPHO2, PHOX2A, PHOX2B, PHPT1, PHRF1, PHTF1, PHTF2, PHYH, PHYHD1, PHYHIP, PHYHIPL, PHYKPL, PI15, PI16, PI3, PI4K2A, PI4K2B, PI4KA, PI4 KB, PIANP, PIAS1, PIAS2, PIAS3, PIAS4, PIBF1, PICALM, PICK1, PID1, PIDD1, PIEZO1, PIEZO2, PIF1, PIFO, PIGA, PIGB, PIGBOS1, PIGC, PIGF, PIGG, PIGH, PIGK, PIGL, PIGM, PIGN, PIGO, PIGP, PIGQ, PIGR, PIGS, PIGT, PIGU, PIGV, PIGW, PIGX, PIGY, PIGZ, PIH1D1, PIH1D2, PIH1D3, PIK3AP1, PIK3C2A, PIK3C2B, PIK3C2G, PIK3C3, PIK3CA, PIK3CB, PIK3CD, PIK3CG, PIK3IP1, PIK3R1, PIK3R2, PIK3R3, PIK3R4, PIK3R5, PIK3R6, PIKFYVE, PILRA, PILRB, PIM1, PIM2, PIM3, PIMREG, PIN1, PIN4, PINK1, PINLYP, PINX1, PIP, PIP4K2A, PIP4K2B, PIP4K2C, PIP5K1A, PIP5K1B, PIP5K1C, PIP5KL1, PIPOX, PIR, PIRT, PISD, PITHD1, PITPNA, PITPNB, PITPNC1, PITPNM1, PITPNM2, PITPNM3, PITRM1, PITX1, PITX2, PITX3, PIWIL1, PIWIL2, PIWIL3, PIWIL4, PJA1, PJA2, PKD1, PKD1L1, PKD1L2, PKD1L3, PKD2, PKD2L1, PKD2L2, PKDCC, PKDREJ, PKHD1, PKHD1L1, PKIA, PKIB, PKIG, PKLR, PKM, PKMYT1, PKN1, PKN2, PKN3, PKNOX1, PKNOX2, PKP1, PKP2, PKP3, PKP4, PLAlA, PLA2G10, PLA2G12A, PLA2G12B, PLA2G15, PLA2G16, PLA2G1B, PLA2G2A, PLA2G2C, PLA2G2D, PLA2G2E, PLA2G2F, PLA2G3, PLA2G4A, PLA2G4B, PLA2G4C, PLA2G4D, PLA2G4E, PLA2G4F, PLA2G5, PLA2G6, PLA2G7, PLA2R1, PLAA, PLAC1, PLAC4, PLAC8, PLAC8L1, PLAC9, PLAG1, PLAGL1, PLAGL2, PLAT, PLAU, PLAUR, PLB1, PLBD1, PLBD2, PLCB1, PLCB2, PLCB3, PLCB4, PLCD1, PLCD3, PLCD4, PLCE1, PLCG1, PLCG2, PLCH1, PLCH2, PLCL1, PLCL2, PLCXD1, PLCXD2, PLCXD3, PLCZ1, PLD1, PLD2, PLD3, PLD4, PLD5, PLD6, PLEC, PLEK, PLEK2, PLEKHA1, PLEKHA2, PLEKHA3, PLEKHA4, PLEKHA5, PLEKHA6, PLEKHA7, PLEKHA8, PLEKHB1, PLEKHB2, PLEKHD1, PLEKHF1, PLEKHF2, PLEKHG1, PLEKHG2, PLEKHG3, PLEKHG4, PLEKHG4B, PLEKHG5, PLEKHG6, PLEKHG7, PLEKHHI, PLEKHH2, PLEKHH3, PLEKHJ1, PLEKHMI, PLEKHM2, PLEKHM3, PLEKHNI, PLEKHO1, PLEKHO2, PLEKHS1, PLET1, PLG, PLGLB1, PLGLB2, PLGRKT, PLIN1, PLIN2, PLIN3, PLIN4, PLIN5, PLK1, PLK2, PLK3, PLK4, PLK5, PLLP, PLN, PLOD1, PLOD2, PLOD3, PLP1, PLP2, PLPBP, PLPP1, PLPP2, PLPP3, PLPP4, PLPP5, PLPP6, PLPP7, PLPPR1, PLPPR2, PLPPR3, PLPPR4, PLPPR5, PLRG1, PLS1, PLS3, PLSCR1, PLSCR2, PLSCR3, PLSCR4, PLSCR5, PLTP, PLVAP, PLXDC1, PLXDC2, PLXNA1, PLXNA2, PLXNA3, PLXNA4, PLXNB1, PLXNB2, PLXNB3, PLXNC1, PLXND1, PM20D1, PM20D2, PMAIP1, PMCH, PMEL, PMEPA1, PMF1, PMF1-BGLAP, PMFBP1, PML, PMM1, PMM2, PMP2, PMP22, PMPCA, PMPCB, PMS1, PMS2, PMVK, PNCK, PNISR, PNKD, PNKP, PNLDC1, PNLIP, PNLIPRP1, PNLIPRP2, PNLIPRP3, PNMA1, PNMA2, PNMA3, PNMA5, PNMA6A, PNMA6E, PNMA6F, PNMA8A, PNMA8B, PNMA8C, PNMT, PNN, PNO1, PNOC, PNP, PNPLA1, PNPLA2, PNPLA3, PNPLA4, PNPLA5, PNPLA6, PNPLA7, PNPLA8, PNPO, PNPT1, PNRC1, PNRC2, POC1A, POC1B, POC1B-GALNT4, POC5, PODN, PODNL1, PODXL, PODXL2, POF1B, POFUT1, POFUT2, POGK, POGLUT1, POGZ, POLA1, POLA2, POLB, POLD1, POLD2, POLD3, POLD4, POLDIP2, POLDIP3, POLE, POLE2, POLE3, POLE4, POLG, POLG2, POLH, POLI, POLK, POLL, POLM, POLN, POLQ, POLR1A, POLR1B, POLR1C, POLR1D, POLR1E, POLR2A, POLR2B, POLR2C, POLR2D, POLR2E, POLR2F, POLR2G, POLR2H, POLR2I, POLR₂J, POLR₂J2, POLR2 J3, POLR2K, POLR2L, POLR2M, POLR3A, POLR3B, POLR3C, POLR3D, POLR3E, POLR3F, POLR3G, POLR3GL, POLR3H, POLR3K, POLRMT, POM121, POM121C, POM121L12, POM121L2, POMC, POMGNT1, POMGNT2, POMK, POMP, POMT1, POMT2, POMZP3, PON1, PON2, PON3, POP1, POP4, POP5, POP7, POPDC2, POPDC3, POR, PORCN, POSTN, POT1, POTEA, POTEB, POTEB2, POTEB3, POTEC, POTED, POTEE, POTEF, POTEG, POTEH, POTEI, POTEJ, POTEM, POU1F1, POU2AF1, POU2F1, POU2F2, POU2F3, POU3F1, POU3F2, POU3F3, POU3F4, POU4F1, POU4F2, POU4F3, POUSF1, POUSF1B, POU5F2, POU6F1, POU6F2, PP2D1, PPA1, PPA2, PPAN, PPAN-P2RY11, PPARA, PPARD, PPARG, PPARGC1A, PPARGCIB, PPAT, PPBP, PPCDC, PPCS, PPDPF, PPEF1, PPEF2, PPFIA1, PPFIA2, PPFIA3, PPFIA4, PPFIBP1, PPFIBP2, PPHLN1, PPIA, PPIAL4A, PPIAL4C, PPIAL4D, PPIAL4E, PPIAL4F, PPIAL4G, PPIB, PPIC, PPID, PPIE, PPIF, PPIG, PPIH, PPIL1, PPIL2, PPIL3, PPIL4, PPIL6, PPIP5K1, PPIP5K2, PPL, PPM1A, PPM1B, PPM1D, PPM1E, PPM1F, PPM1G, PPM1H, PPM1J, PPM1K, PPM1L, PPM1M, PPM1N, PPME1, PPOX, PPP1CA, PPP1CB, PPP1CC, PPP1R10, PPP1R11, PPP1R12A, PPP1R12B, PPP1R12C, PPP1R13B, PPP1R13L, PPP1R14A, PPP1R14B, PPP1R14C, PPP1R14D, PPP1R15A, PPP1R15B, PPP1R16A, PPP1R16B, PPP1R17, PPP1R18, PPP1R1A, PPP1R1B, PPP1R1C, PPP1R₂, PPP1R₂1, PPP1R₂6, PPP1R₂7, PPP1R₂P3, PPP1R₂P9, PPP1R32, PPP1R35, PPP1R36, PPP1R37, PPP1R3A, PPP1R3B, PPP1R3C, PPP1R3D, PPP1R3E, PPP1R3F, PPP1R3G, PPP1R42, PPP1R7, PPP1R8, PPP1R9A, PPP1R9B, PPP2CA, PPP2CB, PPP2R1A, PPP2R1B, PPP2R₂A, PPP2R₂B, PPP2R₂C, PPP2R₂D, PPP2R3A, PPP2R3B, PPP2R3C, PPP2R5A, PPP2R5B, PPP2R5C, PPP2R5D, PPP2R5E, PPP3CA, PPP3CB, PPP3CC, PPP3R1, PPP3R₂, PPP4C, PPP4R1, PPP4R₂, PPP4R3A, PPP4R3B, PPP4R3CP, PPP4R4, PPP5C, PPP5D1, PPP6C, PPP6R1, PPP6R2, PPP6R3, PPRC1, PPT1, PPT2, PPT2-EGFL8, PPTC7, PPWD1, PPY, PQBP1, PQLC1, PQLC2, PQLC2L, PQLC3, PRAC1, PRAC2, PRADC1, PRAF2, PRAG1, PRAM1, PRAME, PRAMEF1, PRAMEF10, PRAMEF11, PRAMEF12, PRAMEF13, PRAMEF14, PRAMEF15, PRAMEF17, PRAMEF18, PRAMEF19, PRAMEF2, PRAMEF20, PRAMEF25, PRAMEF26, PRAMEF27, PRAMEF33, PRAMEF4, PRAMEF5, PRAMEF6, PRAMEF7, PRAMEF8, PRAMEF9, PRAP1, PRB1, PRB2, PRB3, PRB4, PRC1, PRCC, PRCD, PRCP, PRDM1, PRDM10, PRDM11, PRDM12, PRDM13, PRDM14, PRDM15, PRDM16, PRDM2, PRDM4, PRDM5, PRDM6, PRDM7, PRDM8, PRDM9, PRDX1, PRDX2, PRDX3, PRDX4, PRDX5, PRDX6, PREB, PRELIDI, PRELID2, PRELID3A, PRELID3B, PRELP, PREP, PREPL, PREX1, PREX2, PRF1, PRG2, PRG3, PRG4, PRH1, PRH2, PRICKLE1, PRICKLE2, PRICKLE3, PRICKLE4, PRIM1, PRIM2, PRIMAl, PRIMPOL, PRKAA1, PRKAA2, PRKAB1, PRKAB2, PRKACA, PRKACB, PRKACG, PRKAG1, PRKAG2, PRKAG3, PRKAR1A, PRKAR1B, PRKAR2A, PRKAR2B, PRKCA, PRKCB, PRKCD, PRKCE, PRKCG, PRKCH, PRKCI, PRKCQ, PRKCSH, PRKCZ, PRKD1, PRKD2, PRKD3, PRKDC, PRKG1, PRKG2, PRKN, PRKRA, PRKRIP1, PRKX, PRL, PRLH, PRLHR, PRLR, PRM1, PRM2, PRM3, PRMT1, PRMT2, PRMT3, PRMT5, PRMT6, PRMT7, PRMT8, PRMT9, PRND, PRNP, PRNT, PROB1, PROC, PROCA1, PROCR, PRODH, PRODH2, PROK1, PROK2, PROKR1, PROKR2, PROM1, PROM2, PROP1, PRORY, PROS1, PROSERI, PROSER2, PROSER3, PROX1, PROX2, PROZ, PRPF18, PRPF19, PRPF3, PRPF31, PRPF38A, PRPF38B, PRPF39, PRPF4, PRPF40A, PRPF40B, PRPF4B, PRPF6, PRPF8, PRPH, PRPH2, PRPS1, PRPS1L1, PRPS2, PRPSAP1, PRPSAP2, PRR11, PRR12, PRR13, PRR14, PRR14L, PRR15, PRR15L, PRR16, PRR18, PRR19, PRR₂0A, PRR₂0B, PRR₂0C, PRR₂0D, PRR₂0E, PRR₂1, PRR₂2, PRR₂3A, PRR₂3B, PRR₂3C, PRR₂3D1, PRR₂3D2, PRR₂5, PRR₂6, PRR₂7, PRR₂9, PRR3, PRR30, PRR32, PRR34, PRR35, PRR36, PRR4, PRR5, PRR5-ARHGAP8, PRR5L, PRR7, PRR9, PRRC1, PRRC2A, PRRC2B, PRRC2C, PRRG1, PRRG2, PRRG3, PRRG4, PRRT1, PRRT2, PRRT3, PRRT4, PRRX1, PRRX2, PRSS1, PRSS12, PRSS16, PRSS2, PRSS21, PRSS22, PRSS23, PRSS27, PRSS3, PRSS33, PRSS35, PRSS36, PRSS37, PRSS38, PRSS41, PRSS42, PRSS45, PRSS46, PRSS48, PRSS50, PRSS51, PRSS53, PRSS54, PRSS55, PRSS56, PRSS57, PRSS58, PRSS8, PRTFDC1, PRTG, PRTN3, PRUNE1, PRUNE2, PRX, PRY, PRY2, PSAP, PSAPLI, PSAT1, PSCA, PSD, PSD2, PSD3, PSD4, PSEN1, PSEN2, PSENEN, PSG1, PSG11, PSG2, PSG3, PSG4, PSG5, PSG6, PSG7, PSG8, PSG9, PSIP1, PSKH1, PSKH2, PSMA1, PSMA2, PSMA3, PSMA4, PSMA5, PSMA6, PSMA7, PSMA8, PSMB1, PSMB10, PSMB11, PSMB2, PSMB3, PSMB4, PSMB5, PSMB6, PSMB7, PSMB8, PSMB9, PSMC1, PSMC2, PSMC3, PSMC3IP, PSMC4, PSMC5, PSMC6, PSMD1, PSMD10, PSMD11, PSMD12, PSMD13, PSMD14, PSMD2, PSMD3, PSMD4, PSMD5, PSMD6, PSMD7, PSMD8, PSMD9, PSME1, PSME2, PSME3, PSME4, PSMF1, PSMG1, PSMG2, PSMG3, PSMG4, PSORSICI, PSORS1C2, PSPC1, PSPH, PSPN, PSRC1, PSTK, PSTPIP1, PSTPIP2, PTAFR, PTAR1, PTBP1, PTBP2, PTBP3, PTCD1, PTCD2, PTCD3, PTCH1, PTCH2, PTCHD1, PTCHD3, PTCHD4, PTCRA, PTDSS1, PTDSS2, PTEN, PTER, PTF1A, PTGDR, PTGDR₂, PTGDS, PTGER1, PTGER₂, PTGER3, PTGER4, PTGES, PTGES2, PTGES3, PTGES3L, PTGES3L-AARSD1, PTGFR, PTGFRN, PTGIR, PTGIS, PTGR1, PTGR2, PTGS1, PTGS2, PTH, PTH1R, PTH2, PTH2R, PTHLH, PTK2, PTK2B, PTK6, PTK7, PTMA, PTMS, PTN, PTOV1, PTP4A1, PTP4A2, PTP4A3, PTPA, PTPDC1, PTPMT1, PTPN1, PTPN11, PTPN12, PTPN13, PTPN14, PTPN18, PTPN2, PTPN20, PTPN21, PTPN22, PTPN23, PTPN3, PTPN4, PTPN5, PTPN6, PTPN7, PTPN9, PTPRA, PTPRB, PTPRC, PTPRCAP, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRZl, PTRH1, PTRH2, PTRHD1, PTS, PTTG1, PTTG1IP, PTTG2, PTX3, PTX4, PUDP, PUF60, PUM1, PUM2, PUM3, PURA, PURB, PURG, PUS1, PUS10, PUS3, PUS7, PUS7L, PUSL1, PVALB, PVR, PVRIG, PWP1, PWP2, PWWP2A, PWWP2B, PXDC1, PXDN, PXDNL, PXK, PXMP2, PXMP4, PXN, PXT1, PXYLP1, PYCARD, PYCR1, PYCR2, PYCR3, PYDC1, PYDC2, PYGB, PYGL, PYGM, PYGO1, PYGO2, PYHIN 1, PYM1, PYROXD1, PYROXD2, PYURF, PYY, PZP, QARS, QDPR, QKI, QPCT, QPCTL, QPRT, QRFP, QRFPR, QRICH1, QRICH2, QRSL1, QSER1, QSOX1, QSOX2, QTRT1, QTRT2, R3HCC1, R3HCC1L, R3HDM1, R3HDM2, R3HDM4, R3HDML, RAB10, RAB11A, RAB11B, RAB11FIP1, RABi11FIP2, RABi11FIP3, RABi11FIP4, RABi11FIP5, RAB12, RAB13, RAB14, RAB15, RAB17, RAB18, RAB19, RAB1A, RAB1B, RAB20, RAB21, RAB22A, RAB23, RAB24, RAB25, RAB26, RAB27A, RAB27B, RAB28, RAB29, RAB2A, RAB2B, RAB30, RAB31, RAB32, RAB33A, RAB33B, RAB34, RAB35, RAB36, RAB37, RAB38, RAB39A, RAB39B, RAB3A, RAB3B, RAB3C, RAB3D, RAB3GAP1, RAB3GAP2, RAB3IL1, RAB3IP, RAB40A, RAB40AL, RAB40B, RAB40C, RAB41, RAB42, RAB43, RAB44, RAB4A, RAB4B, RAB4B-EGLN2, RAB5A, RAB5B, RAB5C, RAB6A, RAB6B, RAB6C, RAB7A, RAB7B, RAB8A, RAB8B, RAB9A, RAB9B, RABAC1, RABEP1, RABEP2, RABEPK, RABGAP1, RABGAP1L, RABGEF1, RABGGTA, RABGGTB, RABIF, RABL2A, RABL2B, RABL3, RABL6, RAC1, RAC2, RAC3, RACGAP1, RACK1, RAD1, RAD17, RAD18, RAD21, RAD21L1, RAD23A, RAD23B, RAD50, RAD51, RAD51AP1, RAD51AP2, RAD51B, RAD51C, RAD51D, RAD52, RAD54B, RAD54L, RAD54L2, RAD9A, RAD9B, RADIL, RAE1, RAET1E, RAET1G, RAET1L, RAF1, RAG1, RAG2, RAI1, RAI14, RAI2, RALA, RALB, RALBP1, RALGAPA1, RALGAPA2, RALGAPB, RALGDS, RALGPS1, RALGPS2, RALY, RALYL, RAMP1, RAMP2, RAMP3, RAN, RANBP1, RANBP10, RANBP17, RANBP2, RANBP3, RANBP3L, RANBP6, RANBP9, RANGAP1, RANGRF, RAPlA, RAP1B, RAP1GAP, RAP1GAP2, RAP1GDS1, RAP2A, RAP2B, RAP2C, RAPGEF1, RAPGEF2, RAPGEF3, RAPGEF4, RAPGEF5, RAPGEF6, RAPGEFL1, RAPH1, RAPSN, RARA, RARB, RARG, RARRESI, RARRES2, RARRES3, RARS, RARS2, RASA1, RASA2, RASA3, RASA4, RASA4B, RASAL1, RASAL2, RASAL3, RASD1, RASD2, RASEF, RASGEFIA, RASGEFlB, RASGEFlC, RASGRF1, RASGRF2, RASGRP1, RASGRP2, RASGRP3, RASGRP4, RASIP1, RASL10A, RASL10B, RASL11A, RASL11B, RASL12, RASSF1, RASSF10, RASSF2, RASSF3, RASSF4, RASSF5, RASSF6, RASSF7, RASSF8, RASSF9, RAVER1, RAVER₂, RAX, RAX2, RB1, RB1CC1, RBAK, RBAK-RBAKDN, RBBP4, RBBP5, RBBP6, RBBP7, RBBP8, RBBP8NL, RBBP9, RBCK1, RBFA, RBFOX1, RBFOX2, RBFOX3, RBKS, RBL1, RBL2, RBM10, RBM11, RBM12, RBM12B, RBM14, RBM14-RBM4, RBM15, RBM15B, RBM17, RBM18, RBM19, RBM20, RBM22, RBM23, RBM24, RBM25, RBM26, RBM27, RBM28, RBM3, RBM33, RBM34, RBM38, RBM39, RBM4, RBM41, RBM42, RBM43, RBM44, RBM45, RBM46, RBM47, RBM48, RBM4B, RBM5, RBM6, RBM7, RBM8A, RBMS1, RBMS2, RBMS3, RBMX, RBMX2, RBMXL1, RBMXL2, RBMXL3, RBMYlA1, RBMYlB, RBMYlD, RBMY1E, RBMYlF, RBMYlJ, RBP1, RBP2, RBP3, RBP4, RBP5, RBP7, RBPJ, RBPJL, RBPMS, RBPMS2, RBSN, RBX1, RC3H1, RC3H2, RCAN1, RCAN2, RCAN3, RCBTB1, RCBTB2, RCC1, RCC1L, RCC2, RCCD1, RCE1, RCHY1, RCL1, RCN1, RCN2, RCN3, RCOR1, RCOR₂, RCOR3, RCSD1, RCVRN, RD3, RD3L, RDH10, RDH11, RDH12, RDH13, RDH14, RDH16, RDH5, RDH8, RDM1, RDX, REC114, REC8, RECK, RECQL, RECQL4, RECQL5, REEP1, REEP2, REEP3, REEP4, REEP5, REEP6, REGlA, REG1B, REG3A, REG3G, REG4, REL, RELA, RELB, RELL1, RELL2, RELN, RELT, REM1, REM2, REN, RENBP, REP15, REPINI, REPS1, REPS2, RER1, RERE, RERG, RERGL, RESP18, REST, RET, RETN, RETNLB, RETREGI, RETREG2, RETREG3, RETSAT, REV1, REV3L, REXO1, REXO2, REXO4, REXO5, RFC1, RFC2, RFC3, RFC4, RFC5, RFESD, RFFL, RFK, RFLNA, RFLNB, RFNG, RFPL1, RFPL2, RFPL3, RFPL3S, RFPL4A, RFPL4AL1, RFPL4B, RFT1, RFTN1, RFTN2, RFWD2, RFWD3, RFX1, RFX2, RFX3, RFX4, RFX5, RFX6, RFX7, RFX8, RFXANK, RFXAP, RGCC, RGL1, RGL2, RGL3, RGL4, RGMA, RGMB, RGN, RGP1, RGPD1, RGPD2, RGPD3, RGPD4, RGPD5, RGPD6, RGPD8, RGR, RGS1, RGS10, RGS11, RGS12, RGS13, RGS14, RGS16, RGS17, RGS18, RGS19, RGS2, RGS20, RGS21, RGS22, RGS3, RGS4, RGS5, RGS6, RGS7, RGS7BP, RGS8, RGS9, RGS9BP, RGSL1, RHAG, RHBDD1, RHBDD2, RHBDD3, RHBDF1, RHBDF2, RHBDL1, RHBDL2, RHBDL3, RHBG, RHCE, RHCG, RHD, RHEB, RHEBL1, RHNO1, RHO, RHOA, RHOB, RHOBTB1, RHOBTB2, RHOBTB3, RHOC, RHOD, RHOF, RHOG, RHOH, RHOJ, RHOQ, RHOT1, RHOT2, RHOU, RHOV, RHOXF1, RHOXF2, RHOXF2B, RHPN1, RHPN2, RIBC1, RIBC2, RIC1, RIC3, RIC8A, RIC8B, RICTOR, RIDA, RIF1, RIIAD1, RILP, RILPL1, RILPL2, RIMBP2, RIMBP3, RIMBP3B, RIMBP3C, RIMKLA, RIMKLB, RIMS1, RIMS2, RIMS3, RIMS4, RIN1, RIN2, RIN3, RING1, RINL, RINT1, RIOK1, RIOK2, RIOK3, RIOX1, RIOX2, RIPK1, RIPK2, RIPK3, RIPK4, RIPOR1, RIPOR₂, RIPOR3, RIPPLYl, RIPPLY2, RIPPLY3, RIT1, RIT2, RITA1, RLBP1, RLF, RLIM, RLN1, RLN2, RLN3, RMDN1, RMDN2, RMDN3, RMIl, RMI2, RMND1, RMND5A, RMND5B, RNASE1, RNASE10, RNASE11, RNASE12, RNASE13, RNASE2, RNASE3, RNASE4, RNASE6, RNASE7, RNASE8, RNASE9, RNASEH1, RNASEH2A, RNASEH2B, RNASEH2C, RNASEK, RNASEK-C17orf49, RNASEL, RNASET2, RND1, RND2, RND3, RNF10, RNF103, RNF103-CHMP3, RNF11, RNF111, RNF112, RNF113A, RNF113B, RNF114, RNF115, RNF121, RNF122, RNF123, RNF125, RNF126, RNF128, RNF13, RNF130, RNF133, RNF135, RNF138, RNF139, RNF14, RNF141, RNF144A, RNF144B, RNF145, RNF146, RNF148, RNF149, RNF150, RNF151, RNF152, RNF157, RNF165, RNF166, RNF167, RNF168, RNF169, RNF17, RNF170, RNF175, RNF180, RNF181, RNF182, RNF183, RNF185, RNF186, RNF187, RNF19A, RNF19B, RNF2, RNF20, RNF207, RNF208, RNF212, RNF212B, RNF213, RNF214, RNF215, RNF216, RNF217, RNF219, RNF220, RNF222, RNF223, RNF224, RNF225, RNF24, RNF25, RNF26, RNF31, RNF32, RNF34, RNF38, RNF39, RNF4, RNF40, RNF41, RNF43, RNF44, RNF5, RNF6, RNF7, RNF8, RNFT1, RNFT2, RNGTT, RNH1, RNLS, RNMT, RNPC3, RNPEP, RNPEPL1, RNPS1, ROBO1, ROBO2, ROBO3, ROBO4, ROCK1, ROCK2, ROGDI, ROM1, ROMO1, ROPN1, ROPN1B, ROPN1L, ROR1, ROR₂, RORA, RORB, RORC, ROS1, RP1, RP1L1, RP2, RP9, RPA1, RPA2, RPA3, RPA4, RPAIN, RPAP1, RPAP2, RPAP3, RPE, RPE65, RPEL1, RPF1, RPF2, RPGR, RPGRIP1, RPGRIP1L, RPH3A, RPH3AL, RPIA, RPL10, RPL10A, RPL10L, RPL11, RPL12, RPL13, RPL13A, RPL14, RPL15, RPL17, RPL17-C18orf32, RPL18, RPL18A, RPL19, RPL21, RPL22, RPL22L1, RPL23, RPL23A, RPL24, RPL26, RPL26L1, RPL27, RPL27A, RPL28, RPL29, RPL3, RPL30, RPL31, RPL32, RPL34, RPL35, RPL35A, RPL36, RPL36A, RPL36A-HNRNPH2, RPL36AL, RPL37, RPL37A, RPL38, RPL39, RPL39L, RPL3L, RPL4, RPL41, RPL5, RPL6, RPL7, RPL7A, RPL7L1, RPL8, RPL9, RPLPO, RPLP1, RPLP2, RPN1, RPN2, RPP14, RPP21, RPP25, RPP25L, RPP30, RPP38, RPP40, RPRD1A, RPRD1B, RPRD2, RPRM, RPRML, RPS10, RPS10-NUDT3, RPS11, RPS12, RPS13, RPS14, RPS15, RPS15A, RPS16, RPS17, RPS18, RPS19, RPS19BP1, RPS2, RPS20, RPS21, RPS23, RPS24, RPS25, RPS26, RPS27, RPS27A, RPS27L, RPS28, RPS29, RPS3, RPS3A, RPS4X, RPS4Y1, RPS4Y2, RPS5, RPS6, RPS6KA1, RPS6KA2, RPS6KA3, RPS6KA4, RPS6KA5, RPS6KA6, RPS6KB1, RPS6KB2, RPS6KC1, RPS6KL1, RPS7, RPS8, RPS9, RPSA, RPTN, RPTOR, RPUSD1, RPUSD2, RPUSD3, RPUSD4, RRAD, RRAGA, RRAGB, RRAGC, RRAGD, RRAS, RRAS2, RRBP1, RREB1, RRH, RRM1, RRM2, RRM2B, RRN3, RRNAD1, RRP1, RRP12, RRP15, RRP1B, RRP36, RRP7A, RRP8, RRP9, RRS1, RS1, RSAD1, RSAD2, RSBN1, RSBN1L, RSC1A1, RSF1, RSG1, RSL1D1, RSL24D1, RSPH1, RSPH10B, RSPH10B2, RSPH14, RSPH3, RSPH4A, RSPH6A, RSPH9, RSPO1, RSPO2, RSPO3, RSPO4, RSPRYl, RSRC1, RSRC2, RSRP1, RSU1, RTBDN, RTCA, RTCB, RTEL1, RTEL1-TNFRSF6B, RTF1, RTFDC1, RTKN, RTKN2, RTL1, RTL10, RTL3, RTL4, RTL5, RTL6, RTL8A, RTL8B, RTL8C, RTL9, RTN1, RTN2, RTN3, RTN4, RTN4IP1, RTN4R, RTN4RL1, RTN4RL2, RTP1, RTP2, RTP3, RTP4, RTP5, RTTN, RUBCN, RUBCNL, RUFY1, RUFY2, RUFY3, RUFY4, RUNDC1, RUNDC3A, RUNDC3B, RUNX1, RUNX1T1, RUNX2, RUNX3, RUSC1, RUSC2, RUVBL1, RUVBL2, RWDD1, RWDD2A, RWDD2B, RWDD3, RWDD4, RXFP1, RXFP2, RXFP3, RXFP4, RXRA, RXRB, RXRG, RYBP, RYK, RYR1, RYR2, RYR3, S100A1, S100A10, S100A11, S100A12, S100A13, S100A14, S100A16, S100A2, S100A3, S100A4, S100A5, S100A6, S100A7, S100A7A, S100A7L2, S100A8, S100A9, S100B, S100G, S100P, S100PBP, S100Z, SlPRl, S1PR2, S1PR3, S1PR4, S1PR5, SAA1, SAA2, SAA2-SAA4, SAA4, SAAL1, SAC3D1, SACM1L, SACS, SAE1, SAFB, SAFB2, SAG, SAGE1, SALL1, SALL2, SALL3, SALL4, SAMD1, SAMD10, SAMDl11, SAMD12, SAMD13, SAMD14, SAMD15, SAMD3, SAMD4A, SAMD4B, SAMD5, SAMD7, SAMD8, SAMD9, SAMD9L, SAMIHDI, SAMM50, SAMSN1, SAP130, SAP18, SAP25, SAP30, SAP30BP, SAP30L, SAPCD1, SAPCD2, SAR1A, SAR1B, SARAF, SARDH, SARM1, SARNP, SARS, SARS2, SART1, SART3, SASH1, SASH3, SASS6, SAT1, SAT2, SATB1, SATB2, SATL1, SAV1, SAXO1, SAXO2, SAYSD1, SBDS, SBF1, SBF2, SBK1, SBK2, SBK3, SBNO1, SBNO2, SBSN, SBSPON, SC5D, SCAF1, SCAF11, SCAF4, SCAF8, SCAI, SCAMPI, SCAMP2, SCAMP3, SCAMP4, SCAMP5, SCANDI, SCAP, SCAPER, SCARA3, SCARA5, SCARBI, SCARB2, SCARFI, SCARF2, SCARTI, SCCPDH, SCD, SCD5, SCEL, SCFD1, SCFD2, SCG2, SCG3, SCG5, SCGBlA1, SCGB1C1, SCGB1C2, SCGB1D1, SCGB1D2, SCGB1D4, SCGB2A1, SCGB2A2, SCGB2B2, SCGB3A1, SCGB3A2, SCGN, SCHIPI, SCIMP, SCIN, SCLT1, SCLY, SCMH1, SCML1, SCML2, SCML4, SCN10A, SCN11A, SCN1A, SCN1B, SCN2A, SCN2B, SCN3A, SCN3B, SCN4A, SCN4B, SCN5A, SCN7A, SCN8A, SCN9A, SCNM1, SCNN1A, SCNN1B, SCNN1D, SCNN1G, SCO1, SC02, SCOC, SCP2, SCP2D1, SCPEP1, SCRG1, SCRIB, SCRN1, SCRN2, SCRN3, SCRT1, SCRT2, SCT, SCTR, SCUBE1, SCUBE2, SCUBE3, SCX, SCYL1, SCYL2, SCYL3, SDAD1, SDC1, SDC2, SDC3, SDC4, SDCBP, SDCBP2, SDCCAG3, SDCCAG8, SDE2, SDF2, SDF2L1, SDF4, SDHA, SDHAF1, SDHAF2, SDHAF3, SDHAF4, SDHB, SDHC, SDHD, SDK1, SDK2, SDR16C5, SDR39U1, SDR42E1, SDR42E2, SDR9C7, SDS, SDSL, SEBOX, SEC11A, SEC11C, SEC13, SEC14L1, SEC14L2, SEC14L3, SEC14L4, SEC14L5, SEC14L6, SEC16A, SEC16B, SEC22A, SEC22B, SEC22C, SEC23A, SEC23B, SEC23IP, SEC24A, SEC24B, SEC24C, SEC24D, SEC31A, SEC31B, SEC61A1, SEC61A2, SEC61B, SEC61G, SEC62, SEC63, SECISBP2, SECISBP2L, SECTM1, SEH1L, SEL1L, SEL1L2, SEL1L3, SELE, SELENBP1, SELENOF, SELENOH, SELENOI, SELENOK, SELENOM, SELENON, SELENOO, SELENOP, SELENOS, SELENOT, SELENOV, SELENOW, SELL, SELP, SELPLG, SEMi, SEMA3A, SEMA3B, SEMA3C, SEMA3D, SEMA3E, SEMA3F, SEMA3G, SEMA4A, SEMA4B, SEMA4C, SEMA4D, SEMA4F, SEMA4G, SEMA5A, SEMA5B, SEMA6A, SEMA6B, SEMA6C, SEMA6D, SEMA7A, SEMG1, SEMG2, SENP1, SENP2, SENP3, SENP3-EIF4A1, SENP5, SENP6, SENP7, SENP8, SEPHS1, SEPHS2, SEPSECS, SEPT1, SEPT10, SEPT11, SEPT12, SEPT14, SEPT2, SEPT3, SEPT4, SEPT5, SEPT6, SEPT7, SEPT8, SEPT9, SERACI, SERBP1, SERF1A, SERFIB, SERF2, SERGEF, SERHL2, SERINC1, SERINC2, SERINC3, SERINC4, SERINC5, SERP1, SERP2, SERPINA1, SERPINA10, SERPINA11, SERPINA12, SERPINA2, SERPINA3, SERPINA4, SERPINA5, SERPINA6, SERPINA7, SERPINA9, SERPINB1, SERPINB10, SERPINB11, SERPINB12, SERPINB13, SERPINB2, SERPINB3, SERPINB4, SERPINB5, SERPINB6, SERPINB7, SERPINB8, SERPINB9, SERPINCI, SERPINDI, SERPINEl, SERPINE2, SERPINE3, SERPINFI, SERPINF2, SERPINGI, SERPINHI, SERPINIl, SERPINI2, SERTADI, SERTAD2, SERTAD3, SERTAD4, SERTM1, SESN1, SESN2, SESN3, SESTD1, SET, SETBP1, SETD1A, SETD1B, SETD2, SETD3, SETD4, SETD5, SETD6, SETD7, SETD9, SETDB1, SETDB2, SETMAR, SETSIP, SETX, SEZ6, SEZ6L, SEZ6L2, SF1, SF3A1, SF3A2, SF3A3, SF3B1, SF3B2, SF3B3, SF3B4, SF3B5, SF3B6, SFI1, SFMBT1, SFMBT2, SFN, SFPQ, SFR1, SFRP1, SFRP2, SFRP4, SFRP5, SFSWAP, SFT2D1, SFT2D2, SFT2D3, SFTA2, SFTA3, SFTPA1, SFTPA2, SFTPB, SFTPC, SFTPD, SFXN1, SFXN2, SFXN3, SFXN4, SFXN5, SGCA, SGCB, SGCD, SGCE, SGCG, SGCZ, SGF29, SGIP1, SGK1, SGK2, SGK3, SGK494, SGMS1, SGMS2, SGO1, SG02, SGPL1, SGPP1, SGPP2, SGSH, SGSM1, SGSM2, SGSM3, SGTA, SGTB, SH2B1, SH2B2, SH2B3, SH2D1A, SH2D1B, SH2D2A, SH2D3A, SH2D3C, SH2D4A, SH2D4B, SH2D5, SH2D6, SH2D7, SH3BGR, SH3BGRL, SH3BGRL2, SH3BGRL3, SH3BP1, SH3BP2, SH3BP4, SH3BP5, SH3BP5L, SH3D19, SH3D21, SH3GL1, SH3GL2, SH3GL3, SH3GLB1, SH3GLB2, SH3KBP1, SH3PXD2A, SH3PXD2B, SH3RF1, SH3RF2, SH3RF3, SH3TC1, SH3TC2, SH3YL1, SHANK1, SHANK2, SHANK3, SHARPIN, SHB, SHBG, SHC1, SHC2, SHC3, SHC4, SHCBP1, SHCBP1L, SHD, SHE, SHF, SHH, SHISA2, SHISA3, SHISA4, SHISA5, SHISA6, SHISA7, SHISA8, SHISA9, SHKBP1, SHMT1, SHMT2, SHOC2, SHOX, SHOX2, SHPK, SHPRH, SHQ1, SHROOM1, SHROOM2, SHROOM3, SHROOM4, SHTN1, SI, SIAE, SIAH1, SIAH2, SIAH3, SIDT1, SIDT2, SIGIRR, SIGLECI, SIGLEC10, SIGLEC11, SIGLEC12, SIGLEC14, SIGLEC15, SIGLEC5, SIGLEC6, SIGLEC7, SIGLEC8, SIGLEC9, SIGLECLI, SIGMARI, SIK1, SIK2, SIK3, SIKE1, SIL1, SIMI, SIM2, SIMC1, SIN3A, SIN3B, SIPA1, SIPA1LI, SIPA1L2, SIPA1L3, SIRPA, SIRPB1, SIRPB2, SIRPD, SIRPG, SIRT1, SIRT2, SIRT3, SIRT4, SIRT5, SIRT6, SIRT7, SIT1, SIVA1, SIX1, SIX2, SIX3, SIX4, SIX5, SIX6, SKAl, SKA2, SKA3, SKAP1, SKAP2, SKI, SKIDA1, SKIL, SKIV2L, SKIV2L2, SKOR1, SKOR₂, SKP1, SKP2, SLA, SLA2, SLAINI, SLAIN2, SLAMFI, SLAMF6, SLAMF7, SLAMF8, SLAMF9, SLBP, SLC10A1, SLC10A2, SLC10A3, SLC10A4, SLC10A5, SLC10A6, SLC10A7, SLC11A1, SLC11A2, SLC12A1, SLC12A2, SLC12A3, SLC12A4, SLC12A5, SLC12A6, SLC12A7, SLC12A8, SLC12A9, SLC13A1, SLC13A2, SLC13A3, SLC13A4, SLC13A5, SLC14A1, SLC14A2, SLC15A1, SLC15A2, SLC15A3, SLC15A4, SLC15A5, SLC16A1, SLC16A10, SLC16A11, SLC16A12, SLC16A13, SLC16A14, SLC16A2, SLC16A3, SLC16A4, SLC16A5, SLC16A6, SLC16A7, SLC16A8, SLC16A9, SLC17A1, SLC17A2, SLC17A3, SLC17A4, SLC17A5, SLC17A6, SLC17A7, SLC17A8, SLC17A9, SLC18A1, SLC18A2, SLC18A3, SLC18B1, SLC19A1, SLC19A2, SLC19A3, SLC1A1, SLC1A2, SLC1A3, SLC1A4, SLC1A5, SLC1A6, SLC1A7, SLC20A1, SLC20A2, SLC22A1, SLC22A10, SLC22A11, SLC22A12, SLC22A13, SLC22A14, SLC22A15, SLC22A16, SLC22A17, SLC22A18, SLC22A18AS, SLC22A2, SLC22A23, SLC22A24, SLC22A25, SLC22A3, SLC22A31, SLC22A4, SLC22A5, SLC22A6, SLC22A7, SLC22A8, SLC22A9, SLC23A1, SLC23A2, SLC23A3, SLC24A1, SLC24A2, SLC24A3, SLC24A4, SLC24A5, SLC25A1, SLC25A10, SLC25A11, SLC25A12, SLC25A13, SLC25A14, SLC25A15, SLC25A16, SLC25A17, SLC25A18, SLC25A19, SLC25A2, SLC25A20, SLC25A21, SLC25A22, SLC25A23, SLC25A24, SLC25A25, SLC25A26, SLC25A27, SLC25A28, SLC25A29, SLC25A3, SLC25A30, SLC25A31, SLC25A32, SLC25A33, SLC25A34, SLC25A35, SLC25A36, SLC25A37, SLC25A38, SLC25A39, SLC25A4, SLC25A40, SLC25A41, SLC25A42, SLC25A43, SLC25A44, SLC25A45, SLC25A46, SLC25A47, SLC25A48, SLC25A5, SLC25A51, SLC25A52, SLC25A53, SLC25A6, SLC26A1, SLC26A10, SLC26A11, SLC26A2, SLC26A3, SLC26A4, SLC26A5, SLC26A6, SLC26A7, SLC26A8, SLC26A9, SLC27A1, SLC27A2, SLC27A3, SLC27A4, SLC27A5, SLC27A6, SLC28A1, SLC28A2, SLC28A3, SLC29A1, SLC29A2, SLC29A3, SLC29A4, SLC2A1, SLC2A10, SLC2A11, SLC2A12, SLC2A13, SLC2A14, SLC2A2, SLC2A3, SLC2A4, SLC2A4RG, SLC2A5, SLC2A6, SLC2A7, SLC2A8, SLC2A9, SLC30A1, SLC30A10, SLC30A2, SLC30A3, SLC30A4, SLC30A5, SLC30A6, SLC30A7, SLC30A8, SLC30A9, SLC31A1, SLC31A2, SLC32A1, SLC33A1, SLC34A1, SLC34A2, SLC34A3, SLC35A1, SLC35A2, SLC35A3, SLC35A4, SLC35A5, SLC35B1, SLC35B2, SLC35B3, SLC35B4, SLC35C1, SLC35C2, SLC35D1, SLC35D2, SLC35D3, SLC35E1, SLC35E2, SLC35E2B, SLC35E3, SLC35E4, SLC35F1, SLC35F2, SLC35F3, SLC35F4, SLC35F5, SLC35F6, SLC35G1, SLC35G2, SLC35G3, SLC35G4, SLC35G5, SLC35G6, SLC36A1, SLC36A2, SLC36A3, SLC36A4, SLC37A1, SLC37A2, SLC37A3, SLC37A4, SLC38A1, SLC38A10, SLC38A11, SLC38A2, SLC38A3, SLC38A4, SLC38A5, SLC38A6, SLC38A7, SLC38A8, SLC38A9, SLC39A1, SLC39A10, SLC39A11, SLC39A12, SLC39A13, SLC39A14, SLC39A2, SLC39A3, SLC39A4, SLC39A5, SLC39A6, SLC39A7, SLC39A8, SLC39A9, SLC3A1, SLC3A2, SLC40A1, SLC41A1, SLC41A2, SLC41A3, SLC43A1, SLC43A2, SLC43A3, SLC44A1, SLC44A2, SLC44A3, SLC44A4, SLC44A5, SLC45A1, SLC45A2, SLC45A3, SLC45A4, SLC46A1, SLC46A2, SLC46A3, SLC47A1, SLC47A2, SLC48A1, SLC4A1, SLC4A10, SLC4A11, SLC4A1AP, SLC4A2, SLC4A3, SLC4A4, SLC4A5, SLC4A7, SLC4A8, SLC4A9, SLC50A1, SLC51A, SLC51B, SLC52A1, SLC52A2, SLC52A3, SLC5A1, SLC5A10, SLC5A11, SLC5A12, SLC5A2, SLC5A3, SLC5A4, SLC5A5, SLC5A6, SLC5A7, SLC5A8, SLC5A9, SLC6A1, SLC6A11, SLC6A12, SLC6A13, SLC6A14, SLC6A15, SLC6A16, SLC6A17, SLC6A18, SLC6A19, SLC6A2, SLC6A20, SLC6A3, SLC6A4, SLC6A5, SLC6A6, SLC6A7, SLC6A8, SLC6A9, SLC7A1, SLC7A10, SLC7A11, SLC7A13, SLC7A14, SLC7A2, SLC7A3, SLC7A4, SLC7A5, SLC7A6, SLC7A6OS, SLC7A7, SLC7A8, SLC7A9, SLC8A1, SLC8A2, SLC8A3, SLC8B1, SLC9A1, SLC9A2, SLC9A3, SLC9A3R1, SLC9A3R2, SLC9A4, SLC9A5, SLC9A6, SLC9A7, SLC9A8, SLC9A9, SLC9B1, SLC9B2, SLC9C1, SLC9C2, SLCO1A2, SLCO1B1, SLCO1B3, SLCO1B7, SLCO1C1, SLCO2A1, SLCO2B1, SLCO3A1, SLCO4A1, SLCO4C1, SLCO5A1, SLCO6A1, SLF1, SLF2, SLFN11, SLFN12, SLFN12L, SLFN13, SLFN14, SLFN5, SLFNL1, SLIRP, SLIT1, SLIT2, SLIT3, SLITRK1, SLITRK2, SLITRK3, SLITRK4, SLITRK5, SLITRK6, SLK, SLMAP, SLN, SLPI, SLTM, SLU7, SLURPI, SLURP2, SLX1A, SLX1B, SLX4, SLX4IP, SMAD1, SMAD2, SMAD3, SMAD4, SMAD5, SMAD6, SMAD7, SMAD9, SMAGP, SMAP1, SMAP2, SMARCB1, SMARCC1, SMARCC2, SMARCD1, SMARCD2, SMARCD3, SMARCEl, SMC1A, SMC1B, SMC2, SMC3, SMC4, SMC5, SMC6, SMCHD1, SMCO1, SMCO2, SMCO3, SMCO4, SMCP, SMCR8, SMDT1, SMG1, SMG5, SMG6, SMG7, SMG8, SMG9, SMIM1, SMIM10, SMIM10L1, SMIM10L2A, SMIM10L2B, SMIM11A, SMIM11B, SMIM12, SMIM13, SMIM14, SMIM15, SMIM17, SMIM18, SMIM19, SMIM2, SMIM20, SMIM21, SMIM22, SMIM23, SMIM24, SMIM26, SMIM27, SMIM28, SMIM29, SMIM3, SMIM30, SMIM31, SMIM4, SMIM5, SMIM6, SMIM7, SMIM8, SMIM9, SMKR1, SMLR1, SMN1, SMN2, SMNDC1, SMO, SMOC1, SMOC2, SMOX, SMPD1, SMPD2, SMPD3, SMPD4, SMPDL3A, SMPDL3B, SMPX, SMR3A, SMR3B, SMS, SMTN, SMTNL1, SMTNL2, SMU1, SMUG1, SMURF1, SMURF2, SMYD1, SMYD2, SMYD3, SMYD4, SMYD5, SNAIl, SNAI2, SNAI3, SNAP23, SNAP25, SNAP29, SNAP47, SNAP91, SNAPC1, SNAPC2, SNAPC3, SNAPC4, SNAPC5, SNAPIN, SNCA, SNCAIP, SNCB, SNCG, SND1, SNED1, SNF8, SNHG28, SNIP1, SNN, SNPH, SNRK, SNRNP200, SNRNP25, SNRNP27, SNRNP35, SNRNP40, SNRNP48, SNRNP70, SNRPA, SNRPA1, SNRPB, SNRPB2, SNRPC, SNRPD1, SNRPD2, SNRPD3, SNRPE, SNRPF, SNRPG, SNRPN, SNTA1, SNTB1, SNTB2, SNTG1, SNTG2, SNTN, SNU13, SNUPN, SNURF, SNW1, SNX1, SNX10, SNX11, SNX12, SNX13, SNX14, SNX15, SNX16, SNX17, SNX18, SNX19, SNX2, SNX20, SNX21, SNX22, SNX24, SNX25, SNX27, SNX29, SNX3, SNX30, SNX31, SNX32, SNX33, SNX4, SNX5, SNX6, SNX7, SNX8, SNX9, SOAT1, SOAT2, SOBP, SOCS1, SOCS2, SOCS3, SOCS4, SOCS5, SOCS6, SOCS7, SOD1, SOD2, SOD3, SOGA1, SOGA3, SOHLH1, SOHLH2, SON, SORBS1, SORBS2, SORBS3, SORCS1, SORCS2, SORCS3, SORD, SORL1, SORT1, SOS1, SOS2, SOST, SOSTDC1, SOWAHA, SOWAHB, SOWAHC, SOWAHD, SOX1, SOX10, SOX11, SOX12, SOX13, SOX14, SOX15, SOX17, SOX18, SOX2, SOX21, SOX3, SOX30, SOX4, SOX5, SOX6, SOX7, SOX8, SOX9, SP1, SP100, SP110, SP140, SP140L, SP2, SP3, SP4, SP5, SP6, SP7, SP8, SP9, SPA17, SPAAR, SPACA1, SPACA3, SPACA4, SPACA5, SPACA5B, SPACA6, SPACA7, SPACA9, SPAG1, SPAGI1A, SPAGI1B, SPAG16, SPAG17, SPAG4, SPAG5, SPAG6, SPAG7, SPAG8, SPAG9, SPAM1, SPANXA1, SPANXA2, SPANXB1, SPANXC, SPANXD, SPANXN1, SPANXN2, SPANXN3, SPANXN4, SPANXN5, SPARC, SPARCL1, SPART, SPAST, SPATA1, SPATA12, SPATA13, SPATA16, SPATA17, SPATA18, SPATA19, SPATA2, SPATA20, SPATA21, SPATA22, SPATA24, SPATA25, SPATA2L, SPATA3, SPATA31A1, SPATA31A3, SPATA31A5, SPATA31A6, SPATA31A7, SPATA31D1, SPATA31D3, SPATA31D4, SPATA31E1, SPATA32, SPATA33, SPATA4, SPATA45, SPATA46, SPATA5, SPATA5L1, SPATA6, SPATA6L, SPATA7, SPATA8, SPATA9, SPATC1, SPATCIL, SPATS1, SPATS2, SPATS2L, SPC24, SPC25, SPCS1, SPCS2, SPCS3, SPDEF, SPDL1, SPDYA, SPDYC, SPDYEl, SPDYE16, SPDYE2, SPDYE2B, SPDYE3, SPDYE4, SPDYE5, SPDYE6, SPECCI, SPECCIL, SPECC1L-ADORA2A, SPEF1, SPEF2, SPEG, SPEM1, SPEN, SPERT, SPESPI, SPG11, SPG21, SPG7, SPHAR, SPHK1, SPHK2, SPHKAP, SPIl, SPIB, SPIC, SPICE1, SPIDR, SPIN1, SPIN2A, SPIN2B, SPIN3, SPIN4, SPINK1, SPINK13, SPINK14, SPINK2, SPINK4, SPINK5, SPINK6, SPINK7, SPINK8, SPINK9, SPINTI, SPINT2, SPINT3, SPINT4, SPIRE1, SPIRE2, SPN, SPNS1, SPNS2, SPNS3, SPO11, SPOCD1, SPOCK1, SPOCK2, SPOCK3, SPON1, SPON2, SPOP, SPOPL, SPOUT1, SPP1, SPP2, SPPL2A, SPPL2B, SPPL2C, SPPL3, SPR, SPRED1, SPRED2, SPRED3, SPRN, SPRR1A, SPRR1B, SPRR₂A, SPRR₂B, SPRR₂D, SPRR₂E, SPRR₂F, SPRR₂G, SPRR3, SPRR4, SPRR5, SPRTN, SPRY1, SPRY2, SPRY3, SPRY4, SPRYD3, SPRYD4, SPRYD7, SPSB1, SPSB2, SPSB3, SPSB4, SPTA1, SPTAN1, SPTB, SPTBN1, SPTBN2, SPTBN4, SPTBN5, SPTLC1, SPTLC2, SPTLC3, SPTSSA, SPTSSB, SPTY2D1, SPTY2D1-AS1, SPX, SPZ1, SQLE, SQOR, SQSTM1, SRA1, SRBD1, SRC, SRCAP, SRCIN1, SRD5A1, SRD5A2, SRD5A3, SREBF1, SREBF2, SREK1, SREK1IP1, SRF, SRFBP1, SRGAP1, SRGAP2, SRGAP2B, SRGAP2C, SRGAP3, SRGN, SRI, SRL, SRM, SRMS, SRP14, SRP19, SRP54, SRP68, SRP72, SRP9, SRPK1, SRPK2, SRPK3, SRPRA, SRPRB, SRPX, SRPX2, SRR, SRRD, SRRM1, SRRM2, SRRM3, SRRM4, SRRM5, SRRT, SRSF1, SRSF10, SRSF11, SRSF12, SRSF2, SRSF3, SRSF4, SRSF5, SRSF6, SRSF7, SRSF8, SRSF9, SRXN1, SRY, SS18, SS18L1, SS18L2, SSB, SSBP1, SSBP2, SSBP3, SSBP4, SSC4D, SSC5D, SSFA2, SSH1, SSH2, SSH3, SSMEM1, SSNA1, SSPN, SSPO, SSR1, SSR2, SSR3, SSR4, SSRP1, SSSCA1, SST, SSTR1, SSTR2, SSTR3, SSTR4, SSTR5, SSU72, SSU72P8, SSUH2, SSX1, SSX2, SSX2B, SSX2IP, SSX3, SSX4, SSX4B, SSX5, SSX7, ST13, ST14, ST18, ST20, ST20-MTHFS, ST3GAL1, ST3GAL2, ST3GAL3, ST3GAL4, ST3GAL5, ST3GAL6, ST5, ST6GAL1, ST6GAL2, ST6GALNAC1, ST6GALNAC2, ST6GALNAC3, ST6GALNAC4, ST6GALNAC5, ST6GALNAC6, ST7, ST7L, ST8SIA1, ST8SIA2, ST8SIA3, ST8SIA4, ST8SIA5, ST8SIA6, STAB1, STAB2, STAC, STAC2, STAC3, STAG1, STAG2, STAG3, STAM, STAM2, STAMBP, STAMBPL1, STAP1, STAP2, STAR, STARD10, STARD13, STARD3, STARD3NL, STARD4, STARD5, STARD6, STARD7, STARD8, STARD9, STAU1, STAU2, STBD1, STC1, STC2, STEAPI, STEAPIB, STEAP2, STEAP3, STEAP4, STH, STIL, STIM1, STIM2, STIP1, STK10, STK11, STK11IP, STK16, STK17A, STK17B, STK19, STK24, STK25, STK26, STK3, STK31, STK32A, STK32B, STK32C, STK33, STK35, STK36, STK38, STK38L, STK39, STK4, STK40, STKLD1, STMN1, STMN2, STMN3, STMN4, STMND1, STN1, STOM, STOMLI, STOML2, STOML3, STON1, STON1-GTF2A1L, STON2, STOX1, STOX2, STPG1, STPG2, STPG3, STPG4, STRA6, STRA8, STRADA, STRADB, STRAP, STRBP, STRC, STRIP1, STRIP2, STRN, STRN3, STRN4, STS, STT3A, STT3B, STUB1, STUM, STX10, STX11, STX12, STX16, STX16-NPEPL1, STX17, STX18, STX19, STX1A, STX1B, STX2, STX3, STX4, STX5, STX6, STX7, STX8, STXBP1, STXBP2, STXBP3, STXBP4, STXBP5, STXBP5L, STXBP6, STYK1, STYX, STYXL1, SUB1, SUCLA2, SUCLG1, SUCLG2, SUCNR1, SUCO, SUDS3, SUFU, SUGCT, SUGP1, SUGP2, SUGT1, SULF1, SULF2, SULTIA1, SULT1A2, SULT1A3, SULT1A4, SULT1B1, SULT1C2, SULT1C3, SULT1C4, SULTIEl, SULT2A1, SULT2B1, SULT4A1, SULT6B1, SUMF1, SUMF2, SUMO1, SUMO2, SUMO3, SUMO4, SUN1, SUN2, SUN3, SUN5, SUOX, SUPT16H, SUPT20H, SUPT3H, SUPT4H1, SUPT5H, SUPT6H, SUPT7L, SUPV3L1, SURF1, SURF2, SURF4, SURF6, SUSD1, SUSD2, SUSD3, SUSD4, SUSD5, SUSD6, SUV39H1, SUV39H2, SUZ12, SV2A, SV2B, SV2C, SVBP, SVEP1, SVIL, SVIP, SVOP, SVOPL, SWAP70, SWI5, SWSAP1, SWT1, SYAP1, SYBU, SYCEl, SYCE1L, SYCE2, SYCE3, SYCN, SYCP1, SYCP2, SYCP2L, SYCP3, SYDE1, SYDE2, SYF2, SYK, SYMPK, SYN1, SYN2, SYN3, SYNC, SYNCRIP, SYNDIGI, SYNDIGIL, SYNE1, SYNE2, SYNE3, SYNE4, SYNGAPI, SYNGRI, SYNGR2, SYNGR3, SYNGR4, SYNJ1, SYNJ2, SYNJ2BP, SYNJ2BP-COX16, SYNM, SYNPO, SYNPO2, SYNPO2L, SYNPR, SYNRG, SYP, SYPL1, SYPL2, SYS1, SYS1-DBNDD2, SYT1, SYT10, SYT11, SYT12, SYT13, SYT14, SYT15, SYT16, SYT17, SYT2, SYT3, SYT4, SYT5, SYT6, SYT7, SYT8, SYT9, SYTL1, SYTL2, SYTL3, SYTL4, SYTL5, SYVN1, SZRD1, SZT2, T, TAAR1, TAAR2, TAAR5, TAAR6, TAAR8, TAAR9, TAB1, TAB2, TAB3, TACl, TAC3, TAC4, TACC1, TACC2, TACC3, TACO1, TACR1, TACR2, TACR3, TACSTD2, TADA1, TADA2A, TADA2B, TADA3, TAF1, TAF10, TAF11, TAF12, TAF13, TAF15, TAFlA, TAF1B, TAF1C, TAF1D, TAF1L, TAF2, TAF3, TAF4, TAF4B, TAF5, TAF5L, TAF6, TAF6L, TAF7, TAF7L, TAF8, TAF9, TAF9B, TAGAP, TAGLN, TAGLN2, TAGLN3, TALl, TAL2, TALDO1, TAMM41, TANC1, TANC2, TANGO2, TANGO6, TANK, TAOK1, TAOK2, TAOK3, TAP1, TAP2, TAPBP, TAPBPL, TAPT1, TARBP1, TARBP2, TARDBP, TARM1, TARS, TARS2, TARSL2, TAS1R1, TAS1R2, TAS1R3, TAS2R1, TAS2R10, TAS2R13, TAS2R14, TAS2R16, TAS2R19, TAS2R20, TAS2R3, TAS2R30, TAS2R31, TAS2R38, TAS2R39, TAS2R4, TAS2R40, TAS2R41, TAS2R42, TAS2R43, TAS2R46, TAS2R5, TAS2R50, TAS2R60, TAS2R7, TAS2R8, TAS2R9, TASP1, TAT, TATDN1, TATDN2, TATDN3, TAXIBPI, TAX1BP3, TAZ, TBATA, TBC1D1, TBC1D10A, TBC1D10B, TBC1D10C, TBC1D12, TBC1D13, TBC1D14, TBC1D15, TBC1D16, TBC1D17, TBC1D19, TBC1D2, TBC1D20, TBC1D21, TBC1D22A, TBC1D22B, TBC1D23, TBC1D24, TBC1D25, TBC1D26, TBC1D28, TBC1D29, TBC1D2B, TBC1D3, TBC1D30, TBC1D31, TBC1D32, TBC1D3B, TBC1D3C, TBC1D3D, TBC1D3E, TBC1D3F, TBC1D3G, TBC1D3H, TBC1D3I, TBC1D3K, TBC1D3L, TBC1D4, TBC1D5, TBC1D7, TBC1D8, TBC1D8B, TBC1D9, TBC1D9B, TBCA, TBCB, TBCC, TBCCD1, TBCD, TBCE, TBCEL, TBCK, TBK1, TBKBP1, TBL1X, TBL1XR1, TBL1Y, TBL2, TBL3, TBP, TBPL1, TBPL2, TBR1, TBRG1, TBRG4, TBX1, TBX10, TBX15, TBX18, TBX19, TBX2, TBX20, TBX21, TBX22, TBX3, TBX4, TBX5, TBX6, TBXA2R, TBXAS1, TC2N, TCAF1, TCAF2, TCAIM, TCAP, TCEA1, TCEA2, TCEA3, TCEAL1, TCEAL2, TCEAL3, TCEAL4, TCEAL5, TCEAL6, TCEAL7, TCEAL8, TCEAL9, TCEANC, TCEANC2, TCERG1, TCERG1L, TCF12, TCF15, TCF19, TCF20, TCF21, TCF23, TCF24, TCF25, TCF3, TCF4, TCF7, TCF7L1, TCF7L2, TCFL5, TCHH, TCHHL1, TCHP, TCIRG1, TCL1A, TCL1B, TCN1, TCN2, TCOF1, TCP1, TCP10, TCP10L, TCP10L2, TCP11, TCP11L1, TCP11L2, TCP11X2, TCTA, TCTE1, TCTE3, TCTEX1D1, TCTEX1D2, TCTEX1D4, TCTN1, TCTN2, TCTN3, TDG, TDGF1, TDO2, TDP1, TDP2, TDRD1, TDRD10, TDRD12, TDRD15, TDRD3, TDRD5, TDRD6, TDRD7, TDRD9, TDRKH, TDRP, TEAD1, TEAD2, TEAD3, TEAD4, TEC, TECPR1, TECPR2, TECR, TECRL, TECTA, TECTB, TEDDM1, TEF, TEFM, TEK, TEKT1, TEKT2, TEKT3, TEKT4, TEKT5, TELO2, TEN1, TEN1-CDK3, TENM1, TENM2, TENM3, TENM4, TEP1, TEPP, TEPSIN, TERB1, TERB2, TERF1, TERF2, TERF2IP, TERT, TES, TESC, TESK1, TESK2, TESMIN, TESPA1, TET1, TET2, TET3, TEX10, TEX101, TEX11, TEX12, TEX13A, TEX13B, TEX13C, TEX13D, TEX14, TEX15, TEX19, TEX2, TEX22, TEX26, TEX261, TEX264, TEX28, TEX29, TEX30, TEX33, TEX35, TEX36, TEX37, TEX38, TEX43, TEX44, TEX45, TEX46, TEX47, TEX48, TEX49, TEX50, TEX51, TEX9, TF, TFAM, TFAP2A, TFAP2B, TFAP2C, TFAP2D, TFAP2E, TFAP4, TFB1M, TFB2M, TFCP2, TFCP2L1, TFDP1, TFDP2, TFDP3, TFE3, TFEB, TFEC, TFF1, TFF2, TFF3, TFG, TFIP11, TFPI, TFPI2, TFPT, TFR2, TFRC, TG, TGDS, TGFA, TGFB1, TGFB1I, TGFB2, TGFB3, TGFBI, TGFBR1, TGFBR2, TGFBR3, TGFBR3L, TGFBRAP1, TGIF1, TGIF2, TGIF2-C20orf24, TGIF2LX, TGIF2LY, TGM1, TGM2, TGM3, TGM4, TGM5, TGM6, TGM7, TGOLN2, TGS1, TH, THADA, THAP1, THAP10, THAP11, THAP12, THAP2, THAP3, THAP4, THAP5, THAP6, THAP7, THAP8, THAP9, THBD, THBS1, THBS2, THBS3, THBS4, THEG, THEGL, THEM4, THEM5, THEM6, THEMIS, THEMIS2, THG1L, THNSL1, THNSL2, THOC1, THOC2, THOC3, THOC5, THOC6, THOC7, THOP1, THPO, THRA, THRAP3, THRB, THRSP, THSD1, THSD4, THSD7A, THSD7B, THTPA, THUMPD1, THUMPD2, THUMPD3, THY1, THYN1, TIAl, TIAF1, TIAL1, TIAM1, TIAM2, TICAMI, TICAM2, TICRR, TIE1, TIFA, TIFAB, TIGAR, TIGD1, TIGD2, TIGD3, TIGD4, TIGD5, TIGD6, TIGD7, TIGIT, TIMD4, TIMELESS, TIMM10, TIMMIOB, TIMM13, TIMM17A, TIMM17B, TIMM21, TIMM22, TIMM23, TIMM23B, TIMM29, TIMM44, TIMM50, TIMM8A, TIMM8B, TIMM9, TIMMDC1, TIMP1, TIMP2, TIMP3, TIMP4, TINAG, TINAGL1, TINCR, TINF2, TIPARP, TIPIN, TIPRL, TIRAP, TISP43, TJAP1, TJP1, TJP2, TJP3, TK1, TK2, TKFC, TKT, TKTL1, TKTL2, TLCD1, TLCD2, TLDC1, TLDC2, TLE1, TLE2, TLE3, TLE4, TLE6, TLK1, TLK2, TLL1, TLL2, TLN1, TLN2, TLNRD1, TLR1, TLR10, TLR2, TLR3, TLR4, TLR5, TLR6, TLR7, TLR8, TLR9, TLX1, TLX2, TLX3, TM2D1, TM2D2, TM2D3, TM4SF1, TM4SF18, TM4SF19, TM4SF19-TCTEX1D2, TM4SF20, TM4SF4, TM4SF5, TM6SF1, TM6SF2, TM7SF2, TM7SF3, TM9SF1, TM9SF2, TM9SF3, TM9SF4, TMA16, TMA7, TMBIM1, TMBIM4, TMBIM6, TMC1, TMC2, TMC3, TMC4, TMC5, TMC6, TMC7, TMC8, TMCC1, TMCC2, TMCC3, TMCO1, TMCO2, TMCO3, TMCO4, TMCO5A, TMCO6, TMED1, TMED10, TMED2, TMED3, TMED4, TMED5, TMED6, TMED7, TMED7-TICAM2, TMED8, TMED9, TMEFF1, TMEFF2, TMEM100, TMEM101, TMEM102, TMEM104, TMEM105, TMEM106A, TMEM106B, TMEM106C, TMEM107, TMEM108, TMEM109, TMEM11, TMEM110, TMEM110-MUSTN1, TMEM114, TMEM115, TMEM116, TMEM117, TMEM119, TMEM120A, TMEM120B, TMEM121, TMEM121B, TMEM123, TMEM125, TMEM126A, TMEM126B, TMEM127, TMEM128, TMEM129, TMEM130, TMEM131, TMEM131L, TMEM132A, TMEM132B, TMEM132C, TMEM132D, TMEM132E, TMEM133, TMEM134, TMEM135, TMEM136, TMEM138, TMEM139, TMEM140, TMEM141, TMEM143, TMEM144, TMEM145, TMEM147, TMEM14A, TMEM14B, TMEM14C, TMEM150A, TMEM150B, TMEM150C, TMEM151A, TMEM151B, TMEM154, TMEM155, TMEM156, TMEM158, TMEM159, TMEM160, TMEM161A, TMEM161B, TMEM163, TMEM164, TMEM165, TMEM167A, TMEM167B, TMEM168, TMEM169, TMEM17, TMEM170A, TMEM170B, TMEM171, TMEM173, TMEM174, TMEM175, TMEM176A, TMEM176B, TMEM177, TMEM178A, TMEM178B, TMEM179, TMEM179B, TMEM18, TMEM181, TMEM182, TMEM183A, TMEM184A, TMEM184B, TMEM184C, TMEM185A, TMEM185B, TMEM186, TMEM187, TMEM189, TMEM189-UBE2V1, TMEM19, TMEM190, TMEM191B, TMEM191C, TMEM192, TMEM196, TMEM198, TMEM199, TMEM2, TMEM200A, TMEM200B, TMEM200C, TMEM201, TMEM202, TMEM203, TMEM204, TMEM205, TMEM206, TMEM207, TMEM208, TMEM209, TMEM210, TMEM211, TMEM212, TMEM213, TMEM214, TMEM215, TMEM216, TMEM217, TMEM218, TMEM219, TMEM220, TMEM221, TMEM222, TMEM223, TMEM225, TMEM225B, TMEM229A, TMEM229B, TMEM230, TMEM231, TMEM232, TMEM233, TMEM234, TMEM235, TMEM236, TMEM237, TMEM238, TMEM239, TMEM240, TMEM241, TMEM242, TMEM243, TMEM244, TMEM245, TMEM246, TMEM247, TMEM248, TMEM249, TMEM25, TMEM250, TMEM251, TMEM252, TMEM253, TMEM254, TMEM255A, TMEM255B, TMEM256, TMEM256-PLSCR3, TMEM257, TMEM258, TMEM259, TMEM26, TMEM260, TMEM262, TMEM263, TMEM265, TMEM266, TMEM267, TMEM268, TMEM269, TMEM27, TMEM270, TMEM30A, TMEM30B, TMEM31, TMEM33, TMEM35A, TMEM35B, TMEM37, TMEM38A, TMEM38B, TMEM39A, TMEM39B, TMEM40, TMEM41A, TMEM41B, TMEM42, TMEM43, TMEM44, TMEM45A, TMEM45B, TMEM47, TMEM5, TMEM50A, TMEM50B, TMEM51, TMEM52, TMEM52B, TMEM53, TMEM54, TMEM55A, TMEM55B, TMEM56, TMEM56-RWDD3, TMEM57, TMEM59, TMEM59L, TMEM60, TMEM61, TMEM62, TMEM63A, TMEM63B, TMEM63C, TMEM64, TMEM65, TMEM67, TMEM68, TMEM69, TMEM70, TMEM71, TMEM72, TMEM74, TMEM74B, TMEM78, TMEM79, TMEM80, TMEM81, TMEM82, TMEM86A, TMEM86B, TMEM87A, TMEM87B, TMEM88, TMEM88B, TMEM89, TMEM8A, TMEM8B, TMEM9, TMEM91, TMEM92, TMEM94, TMEM95, TMEM97, TMEM98, TMEM99, TMEM9B, TMF1, TMIE, TMIGD1, TMIGD2, TMIGD3, TMLHE, TMOD1, TMOD2, TMOD3, TMOD4, TMPO, TMPPE, TMPRSS11 A, TMPRSS11B, TMPRSS11D, TMPRSS11E, TMPRSS11F, TMPRSS12, TMPRSS13, TMPRSS15, TMPRSS2, TMPRSS3, TMPRSS4, TMPRSS4-AS1, TMPRSS5, TMPRSS6, TMPRSS7, TMPRSS9, TMSB10, TMSB15A, TMSB15B, TMSB4X, TMSB4Y, TMTC1, TMTC2, TMTC3, TMTC4, TMUB1, TMUB2, TMX1, TMX2, TMX2-CTNND1, TMX3, TMX4, TNC, TNF, TNFAIP1, TNFAIP2, TNFAIP3, TNFAIP6, TNFAIP8, TNFAIP8L1, TNFAIP8L2, TNFAIP8L3, TNFRSF10A, TNFRSF10B, TNFRSF10C, TNFRSF10D, TNFRSF11A, TNFRSF11B, TNFRSF12A, TNFRSF13B, TNFRSF13C, TNFRSF14, TNFRSF17, TNFRSF18, TNFRSF19, TNFRSF1A, TNFRSF1B, TNFRSF21, TNFRSF25, TNFRSF4, TNFRSF6B, TNFRSF8, TNFRSF9, TNFSF10, TNFSF11, TNFSF12, TNFSF12-TNFSF13, TNFSF13, TNFSF13B, TNFSF14, TNFSF15, TNFSF18, TNFSF4, TNFSF8, TNFSF9, TNIK, TNIP1, TNIP2, TNIP3, TNK1, TNK2, TNKS, TNKS1BP1, TNKS2, TNMD, TNN, TNNC1, TNNC2, TNNI1, TNNI2, TNNI3, TNNI3K, TNNT1, TNNT2, TNNT3, TNP1, TNP2, TNPO1, TNPO2, TNPO3, TNR, TNRC18, TNRC6A, TNRC6B, TNRC6C, TNS1, TNS2, TNS3, TNS4, TNXB, TOB1, TOB2, TOE1, TOGARAMI, TOGARAM2, TOLLIP, TOM1, TOM1L1, TOM1L2, TOMM20, TOMM20L, TOMM22, TOMM34, TOMM40, TOMM40L, TOMM5, TOMM6, TOMM7, TOMM70, TONSL, TOP1, TOP1MT, TOP2A, TOP2B, TOP3A, TOP3B, TOPAZI, TOPBP1, TOPORS, TOR1A, TOR1AIPI, TOR1AIP2, TOR1B, TOR₂A, TOR3A, TOR4A, TOX, TOX2, TOX3, TOX4, TP53, TP53AIP1, TP53BP1, TP53BP2, TP5311l, TP53I13, TP53I3, TP53INP1, TP53INP2, TP53RK, TP53TG3, TP53TG3B, TP53TG3C, TP53TG3D, TP53TG3E, TP53TG3F, TP53TG5, TP63, TP73, TPBG, TPBGL, TPCN1, TPCN2, TPD52, TPD52L1, TPD52L2, TPD52L3, TPGS1, TPGS2, TPH1, TPH2, TPI1, TPK1, TPM1, TPM2, TPM3, TPM4, TPMT, TPO, TPP1, TPP2, TPPP, TPPP2, TPPP3, TPR, TPRA1, TPRG1, TPRG1L, TPRKB, TPRN, TPRX1, TPSAB1, TPSB2, TPSD1, TPSG1, TPST1, TPST2, TPT1, TPTE, TPTE2, TPX2, TRA2A, TRA2B, TRABD, TRABD2A, TRABD2B, TRAC, TRADD, TRAF1, TRAF2, TRAF3, TRAF3IP1, TRAF3IP2, TRAF3IP3, TRAF4, TRAF5, TRAF6, TRAF7, TRAFD1, TRAIP, TRAJ1, TRAJ10, TRAJ11, TRAJ12, TRAJ13, TRAJ14, TRAJ16, TRAJ17, TRAJ18, TRAJ19, TRAJ2, TRAJ20, TRAJ21, TRAJ22, TRAJ23, TRAJ24, TRAJ25, TRAJ26, TRAJ27, TRAJ28, TRAJ29, TRAJ3, TRAJ30, TRAJ31, TRAJ32, TRAJ33, TRAJ34, TRAJ35, TRAJ36, TRAJ37, TRAJ38, TRAJ39, TRAJ4, TRAJ40, TRAJ41, TRAJ42, TRAJ43, TRAJ44, TRAJ45, TRAJ46, TRAJ47, TRAJ48, TRAJ49, TRAJ5, TRAJ50, TRAJ52, TRAJ53, TRAJ54, TRAJ56, TRAJ57, TRAJ58, TRAJ59, TRAJ6, TRAJ61, TRAJ7, TRAJ9, TRAK1, TRAK2, TRAM1, TRAM1L1, TRAM2, TRANK1, TRAP1, TRAPPC1, TRAPPC10, TRAPPC11, TRAPPC12, TRAPPC13, TRAPPC2, TRAPPC2L, TRAPPC3, TRAPPC3L, TRAPPC4, TRAPPC5, TRAPPC6A, TRAPPC6B, TRAPPC8, TRAPPC9, TRAT1, TRAV10, TRAV1-1, TRAV1-2, TRAV12-1, TRAV12-2, TRAV12-3, TRAV13-1, TRAV13-2, TRAV14DV4, TRAV16, TRAV17, TRAV18, TRAV19, TRAV2, TRAV20, TRAV21, TRAV22, TRAV23DV6, TRAV24, TRAV25, TRAV26-1, TRAV26-2, TRAV27, TRAV29DV5, TRAV3, TRAV30, TRAV34, TRAV36DV7, TRAV38-1, TRAV38-2DV8, TRAV39, TRAV4, TRAV40, TRAV41, TRAV5, TRAV6, TRAV7, TRAV8-1, TRAV8-2, TRAV8-3, TRAV8-4, TRAV8-6, TRAV8-7, TRAV9-1, TRAV9-2, TRBC2, TRBJ2-1, TRBJ2-2, TRBJ2-2P, TRBJ2-3, TRBJ2-4, TRBJ2-5, TRBJ2-6, TRBJ2-7, TRBV10-1, TRBV10-2, TRBV10-3, TRBV11-1, TRBV19, TRBV2, TRBV20-1, TRBV200R9-2, TRBV21OR9-2, TRBV23-1, TRBV230R9-2, TRBV24-1, TRBV25-1, TRBV27, TRBV28, TRBV29-1, TRBV30, TRBV3-1, TRBV4-1, TRBV4-2, TRBV5-1, TRBV5-3, TRBV5-4, TRBV5-5, TRBV5-6, TRBV5-7, TRBV6-1, TRBV6-4, TRBV6-5, TRBV6-6, TRBV6-7, TRBV6-8, TRBV7-1, TRBV7-3, TRBV7-4, TRBV7-6, TRBV7-7, TRBV7-9, TRBV9, TRDC, TRDD1, TRDD2, TRDD3, TRDJ1, TRDJ2, TRDJ3, TRDJ4, TRDMT1, TRDN, TRDV1, TRDV2, TRDV3, TREH, TREM1, TREM2, TREMLI, TREML2, TREML4, TRERFI, TREX1, TREX2, TRGC1, TRGC2, TRGJ1, TRGJ2, TRGJP, TRGJP1, TRGJP2, TRGV1, TRGV10, TRGV11, TRGV2, TRGV3, TRGV4, TRGV5, TRGV8, TRGV9, TRH, TRHDE, TRHR, TRIAP1, TRIB1, TRIB2, TRIB3, TRIL, TRIM10, TRIM11, TRIM13, TRIM14, TRIM15, TRIM16, TRIM16L, TRIM17, TRIM2, TRIM21, TRIM22, TRIM23, TRIM24, TRIM25, TRIM26, TRIM27, TRIM28, TRIM29, TRIM3, TRIM31, TRIM32, TRIM33, TRIM34, TRIM35, TRIM36, TRIM37, TRIM38, TRIM39, TRIM39-RPP21, TRIM4, TRIM40, TRIM41, TRIM42, TRIM43, TRIM43B, TRIM44, TRIM45, TRIM46, TRIM47, TRIM48, TRIM49, TRIM49B, TRIM49C, TRIM49D1, TRIM49D2, TRIM5, TRIM50, TRIM51, TRIM52, TRIM54, TRIM55, TRIM56, TRIM58, TRIM59, TRIM6, TRIM60, TRIM61, TRIM62, TRIM63, TRIM64, TRIM64B, TRIM64C, TRIM65, TRIM66, TRIM67, TRIM68, TRIM69, TRIM6-TRIM34, TRIM7, TRIM71, TRIM72, TRIM73, TRIM74, TRIM75P, TRIM77, TRIM8, TRIM9, TRIML1, TRIML2, TRIO, TRIOBP, TRIP10, TRIP11, TRIP12, TRIP13, TRIP4, TRIP6, TRIQK, TRIR, TRIT1, TRMO, TRMT1, TRMT10A, TRMT10B, TRMT10C, TRMT11, TRMT112, TRMT12, TRMT13, TRMT1L, TRMT2A, TRMT2B, TRMT44, TRMT5, TRMT6, TRMT61A, TRMT61B, TRMU, TRNAU1AP, TRNP1, TRNT1, TRO, TROAP, TROVE2, TRPA1, TRPC1, TRPC3, TRPC4, TRPC4AP, TRPC5, TRPC50S, TRPC6, TRPC7, TRPM1, TRPM2, TRPM3, TRPM4, TRPM5, TRPM6, TRPM7, TRPM8, TRPS1, TRPT1, TRPV1, TRPV2, TRPV3, TRPV4, TRPV5, TRPV6, TRRAP, TRUB1, TRUB2, TSACC, TSC1, TSC2, TSC22D1, TSC22D2, TSC22D3, TSC22D4, TSEN15, TSEN2, TSEN34, TSEN54, TSFM, TSG101, TSGA10, TSGA10IP, TSGA13, TSHB, TSHR, TSHZ1, TSHZ2, TSHZ3, TSKS, TSKU, TSLP, TSN, TSNAREl, TSNAX, TSNAX-DISCI, TSNAXIP1, TSPAN1, TSPAN10, TSPAN11, TSPAN12, TSPAN13, TSPAN14, TSPAN15, TSPAN16, TSPAN17, TSPAN18, TSPAN19, TSPAN2, TSPAN3, TSPAN31, TSPAN32, TSPAN33, TSPAN4, TSPAN5, TSPAN6, TSPAN7, TSPAN8, TSPAN9, TSPEAR, TSPO, TSPO2, TSPOAP1, TSPY1, TSPY10, TSPY2, TSPY3, TSPY4, TSPY8, TSPYL1, TSPYL2, TSPYL4, TSPYL5, TSPYL6, TSR1, TSR2, TSR3, TSSC4, TSSKlB, TSSK2, TSSK3, TSSK4, TSSK6, TST, TSTA3, TSTD1, TSTD2, TSTD3, TTBK1, TTBK2, TTC1, TTC12, TTC13, TTC14, TTC16, TTC17, TTC19, TTC21A, TTC21B, TTC22, TTC23, TTC23L, TTC24, TTC25, TTC26, TTC27, TTC28, TTC29, TTC3, TTC30A, TTC30B, TTC31, TTC32, TTC33, TTC34, TTC36, TTC37, TTC38, TTC39A, TTC39B, TTC39C, TTC4, TTC5, TTC6, TTC7A, TTC7B, TTC8, TTC9, TTC9B, TTC9C, TTF1, TTF2, TTI1, TTI2, TTK, TTL, TTLL1, TTLL10, TTLL11, TTLL12, TTLL13P, TTLL2, TTLL3, TTLL4, TTLL5, TTLL6, TTLL7, TTLL8, TTLL9, TTN, TTPA, TTPAL, TTR, TTYH1, TTYH2, TTYH3, TUB, TUBA1A, TUBAIB, TUBAIC, TUBA3C, TUBA3D, TUBA3E, TUBA4A, TUBA4B, TUBA8, TUBAL3, TUBB, TUBB1, TUBB2A, TUBB2B, TUBB3, TUBB4A, TUBB4B, TUBB6, TUBB8, TUBD1, TUBE1, TUBG1, TUBG2, TUBGCP2, TUBGCP3, TUBGCP4, TUBGCP5, TUBGCP6, TUFM, TUFT1, TULP1, TULP2, TULP3, TULP4, TUNAR, TUSC1, TUSC2, TUSC3, TUSC5, TUT1, TVP23A, TVP23B, TVP23C, TVP23C-CDRT4, TWF1, TWF2, TWISTI, TWIST2, TWISTNB, TWNK, TWSG1, TXK, TXLNA, TXLNB, TXLNG, TXN, TXN2, TXNDC11, TXNDC12, TXNDC15, TXNDC16, TXNDC17, TXNDC2, TXNDC5, TXNDC8, TXNDC9, TXNIP, TXNL1, TXNL4A, TXNL4B, TXNRD1, TXNRD2, TXNRD3, TXNRD3NB, TYK2, TYMP, TYMS, TYR, TYRO3, TYROBP, TYRP1, TYSND1, TYW1, TYW1B, TYW3, TYW5, U2AF1, U2AF1L4, U2AF1L5, U2AF2, U2SURP, UACA, UAP1, UAP1L1, UBA1, UBA2, UBA3, UBA5, UBA52, UBA6, UBA7, UBAC1, UBAC2, UBALDI, UBALD2, UBAP1, UBAPIL, UBAP2, UBAP2L, UBASH3A, UBASH3B, UBB, UBC, UBD, UBE2A, UBE2B, UBE2C, UBE2D1, UBE2D2, UBE2D3, UBE2D4, UBE2E1, UBE2E2, UBE2E3, UBE2F, UBE2F-SCLY, UBE2G1, UBE2G2, UBE2H, UBE2I, UBE2J1, UBE2J2, UBE2K, UBE2L3, UBE2L5P, UBE2L6, UBE2M, UBE2N, UBE2NL, UBE20, UBE2Q1, UBE2Q2, UBE2Q2L, UBE2QL1, UBE2R2, UBE2S, UBE2T, UBE2U, UBE2V1, UBE2V2, UBE2W, UBE2Z, UBE3A, UBE3B, UBE3C, UBE3D, UBE4A, UBE4B, UBFD1, UBIADI, UBL3, UBL4A, UBL4B, UBL5, UBL7, UBLCP1, UBN1, UBN2, UBOX5, UBP1, UBQLN1, UBQLN2, UBQLN3, UBQLN4, UBQLNL, UBR1, UBR2, UBR3, UBR4, UBR5, UBR7, UBTD1, UBTD2, UBTF, UBTFL1, UBXN1, UBXN10, UBXN11, UBXN2A, UBXN2B, UBXN4, UBXN6, UBXN7, UBXN8, UCHL1, UCHL3, UCHL5, UCK1, UCK2, UCKL1, UCMA, UCN, UCN2, UCN3, UCP1, UCP2, UCP3, UEVLD, UFC1, UFD1, UFL1, UFM1, UFSP1, UFSP2, UGCG, UGDH, UGGT1, UGGT2, UGP2, UGT1A1, UGT1A10, UGT1A3, UGT1A4, UGT1A5, UGT1A6, UGT1A7, UGT1A8, UGT1A9, UGT2A1, UGT2A2, UGT2A3, UGT2B10, UGT2B11, UGT2B15, UGT2B17, UGT2B28, UGT2B4, UGT2B7, UGT3A1, UGT3A2, UGT8, UHMK1, UHRF1, UHRF1BP1, UHRF1BP1L, UHRF2, UIMC1, ULBP1, ULBP2, ULBP3, ULK1, ULK2, ULK3, ULK4, UMAD1, UMOD, UMODL1, UMPS, UNC119, UNC119B, UNC13A, UNC13B, UNC13C, UNC13D, UNC45A, UNC45B, UNC50, UNC5A, UNC5B, UNC5C, UNC5CL, UNC5D, UNC79, UNC80, UNC93A, UNC93B1, UNCX, UNG, UNK, UNKL, UPB1, UPF1, UPF2, UPF3A, UPF3B, UPK1A, UPK1B, UPK2, UPK3A, UPK3B, UPK3BL1, UPP1, UPP2, UPRT, UQCC1, UQCC2, UQCC3, UQCR10, UQCR11, UQCRB, UQCRC1, UQCRC2, UQCRF S1, UQCRH, UQCRHL, UQCRQ, URAD, URB1, URB2, URGCP, URGCP-MRPS24, URIl, URM1, UROC1, UROD, UROS, USB1, USEl, USF1, USF2, USF3, USH1C, USH1G, USH2A, USHBP1, USMG5, USO1, USP1, USP10, USP11, USP12, USP13, USP14, USP15, USP16, USP17L1, USP17L10, USP17L11, USP17L12, USP17L13, USP17L15, USP17L17, USP17L18, USP17L19, USP17L2, USP17L20, USP17L21, USP17L22, USP17L23, USP17L24, USP17L25, USP17L26, USP17L27, USP17L28, USP17L29, USP17L3, USP17L30, USP17L4, USP17L5, USP17L7, USP17L8, USP18, USP19, USP2, USP20, USP21, USP22, USP24, USP25, USP26, USP27X, USP28, USP29, USP3, USP30, USP31, USP32, USP33, USP34, USP35, USP36, USP37, USP38, USP39, USP4, USP40, USP41, USP42, USP43, USP44, USP45, USP46, USP47, USP48, USP49, USP5, USP50, USP51, USP53, USP54, USP6, USP6NL, USP7, USP8, USP9X, USP9Y, USPL1, UST, UTF1, UTP11, UTP14A, UTP14C, UTP15, UTP18, UTP20, UTP23, UTP3, UTP4, UTP6, UTRN, UTS2, UTS2B, UTS2R, UTY, UVRAG, UVSSA, UXS1, UXT, VAC14, VAMP1, VAMP2, VAMP3, VAMP4, VAMP5, VAMP7, VAMP8, VANGLI, VANGL2, VAPA, VAPB, VARS, VARS2, VASH1, VASH2, VASN, VASP, VAT1, VAT1L, VAV1, VAV2, VAV3, VAX1, VAX2, VBP1, VCAM1, VCAN, VCL, VCP, VCPIP1, VCPKMT, VCX, VCX2, VCX3A, VCX3B, VCY, VCY1B, VDAC1, VDAC2, VDAC3, VDR, VEGFA, VEGFB, VEGFC, VEGFD, VENTX, VEPH1, VEZF1, VEZT, VGF, VGLL1, VGLL2, VGLL3, VGLL4, VHL, VHLL, VIL1, VILL, VIM, VIP, VIPAS39, VIPR1, VIPR2, VIRMA, VIT, VKORC1, VKORCIL1, VLDLR, VMA21, VMAC, VMO1, VMP1, VN1R1, VN1R2, VN1R4, VN1R5, VNN1, VNN2, VNN3, VOPP1, VPREB1, VPREB3, VPS11, VPS13A, VPS13B, VPS13C, VPS13D, VPS16, VPS18, VPS25, VPS26A, VPS26B, VPS28, VPS29, VPS33A, VPS33B, VPS35, VPS36, VPS37A, VPS37B, VPS37C, VPS37D, VPS39, VPS41, VPS45, VPS4A, VPS4B, VPS50, VPS51, VPS52, VPS53, VPS54, VPS72, VPS8, VPS9D1, VRK1, VRK2, VRK3, VRTN, VSIG1, VSIG10, VSIG10L, VSIG10L2, VSIG2, VSIG4, VSIG8, VSIR, VSNL1, VSTM1, VSTM2A, VSTM2B, VSTM2L, VSTM4, VSTM5, VSX1, VSX2, VTA1, VTCN1, VTI1A, VTI1B, VTN, VWA1, VWA2, VWA3A, VWA3B, VWA5A, VWA5B1, VWA5B2, VWA7, VWA8, VWC2, VWC2L, VWCE, VWDE, VWF, WAC, WAPL, WARS, WARS2, WAS, WASF1, WASF2, WASF3, WASHC1, WASHC2A, WASHC2C, WASHC3, WASHC4, WASHC5, WASL, WBP1, WBP11, WBP1L, WBP2, WBP2NL, WBP4, WDCP, WDFY1, WDFY2, WDFY3, WDFY4, WDHD1, WDPCP, WDR1, WDR11, WDR12, WDR13, WDR17, WDR18, WDR19, WDR20, WDR24, WDR25, WDR26, WDR27, WDR3, WDR31, WDR33, WDR34, WDR35, WDR36, WDR37, WDR38, WDR4, WDR41, WDR43, WDR44, WDR45, WDR45B, WDR46, WDR47, WDR48, WDR49, WDR5, WDR53, WDR54, WDR55, WDR59, WDR5B, WDR6, WDR60, WDR61, WDR62, WDR63, WDR64, WDR66, WDR7, WDR70, WDR72, WDR73, WDR74, WDR75, WDR76, WDR77, WDR78, WDR81, WDR82, WDR83, WDR83OS, WDR86, WDR87, WDR88, WDR89, WDR90, WDR91, WDR92, WDR93, WDR97, WDSUB1, WDTC1, WDYHV1, WEE1, WEE2, WFDC1, WFDC10A, WFDC10B, WFDC11, WFDC12, WFDC13, WFDC2, WFDC3, WFDC5, WFDC6, WFDC8, WFDC9, WFIKKN1, WFIKKN2, WFS1, WHAMM, WHRN, WIF1, WIPF1, WIPF2, WIPF3, WIPIl, WIPI2, WISP1, WISP2, WISP3, WIZ, WLS, WNK1, WNK2, WNK3, WNK4, WNT1, WNT10A, WNT10B, WNT11, WNT16, WNT2, WNT2B, WNT3, WNT3A, WNT4, WNT5A, WNT5B, WNT6, WNT7A, WNT7B, WNT8A, WNT8B, WNT9A, WNT9B, WRAP53, WRAP73, WRB, WRN, WRNIP1, WSB1, WSB2, WSCD1, WSCD2, WT1, WTAP, WTH3DI, WTIP, WWC1, WWC2, WWC3, WWOX, WWP1, WWP2, WWTR1, XAB2, XAF1, XAGE1A, XAGElB, XAGE2, XAGE3, XAGE5, XBP1, XCL1, XCL2, XCR1, XDH, XG, XIAP, XIRP1, XIRP2, XK, XKR3, XKR4, XKR5, XKR6, XKR7, XKR8, XKR9, XKRX, XPA, XPC, XPNPEP1, XPNPEP2, XPNPEP3, XPO1, XPO4, XPO5, XPO6, XPO7, XPOT, XPR1, XRCC1, XRCC2, XRCC3, XRCC4, XRCC5, XRCC6, XRN1, XRN2, XRRA1, XXYLT1, XYLB, XYLT1, XYLT2, YAE1D1, YAF2, YAP1, YARS, YARS2, YBEY, YBX1, YBX2, YBX3, YDJC, YEATS2, YEATS4, YES1, YIF1A, YIF1B, YIPF1, YIPF2, YIPF3, YIPF4, YIPF5, YIPF6, YIPF7, YJEFN3, YKT6, YLPM1, YME1L1, YOD1, YPEL1, YPEL2, YPEL3, YPEL4, YPEL5, YRDC, YTHDC1, YTHDC2, YTHDF1, YTHDF2, YTHDF3, YWHAB, YWHAE, YWHAG, YWHAH, YWHAQ, YWHAZ, YY1, YY1AP1, YY2, Z82206.1, Z83844.1, Z84492.1, Z98749.3, Z98752.3, ZACN, ZADH2, ZAN, ZAP70, ZAR1, ZAR1L, ZBBX, ZBED1, ZBED2, ZBED3, ZBED4, ZBED5, ZBED6, ZBED6CL, ZBED8, ZBED9, ZBP1, ZBTB1, ZBTB10, ZBTB11, ZBTB12, ZBTB14, ZBTB16, ZBTB17, ZBTB18, ZBTB2, ZBTB20, ZBTB21, ZBTB22, ZBTB24, ZBTB25, ZBTB26, ZBTB3, ZBTB32, ZBTB33, ZBTB34, ZBTB37, ZBTB38, ZBTB39, ZBTB4, ZBTB40, ZBTB41, ZBTB42, ZBTB43, ZBTB44, ZBTB45, ZBTB46, ZBTB47, ZBTB48, ZBTB49, ZBTB5, ZBTB6, ZBTB7A, ZBTB7B, ZBTB7C, ZBTB8A, ZBTB8B, ZBTB8OS, ZBTB9, ZC2HC1A, ZC2HC1B, ZC2HC1C, ZC3H10, ZC3H11A, ZC3H11B, ZC3H12A, ZC3H12B, ZC3H12C, ZC3H12D, ZC3H13, ZC3H14, ZC3H15, ZC3H18, ZC3H3, ZC3H4, ZC3H6, ZC3H7A, ZC3H7B, ZC3H8, ZC3HAV1, ZC3HAV1L, ZC3HC1, ZC4H2, ZCCHC10, ZCCHC11, ZCCHCl2, ZCCHC13, ZCCHC14, ZCCHC17, ZCCHC18, ZCCHC2, ZCCHC24, ZCCHC3, ZCCHC4, ZCCHC6, ZCCHC7, ZCCHC8, ZCCHC9, ZCRB1, ZCWPW1, ZCWPW2, ZDBF2, ZDHHC1, ZDHHC11, ZDHHC11B, ZDHHC12, ZDHHC13, ZDHHC14, ZDHHC15, ZDHHC16, ZDHHC17, ZDHHC18, ZDHHC19, ZDHHC2, ZDHHC20, ZDHHC21, ZDHHC22, ZDHHC23, ZDHHC24, ZDHHC3, ZDHHC4, ZDHHC5, ZDHHC6, ZDHHC7, ZDHHC8, ZDHHC9, ZEB1, ZEB2, ZER1, ZFAND1, ZFAND2A, ZFAND2B, ZFAND3, ZFAND4, ZFAND5, ZFAND6, ZFAT, ZFC3H1, ZFHX2, ZFHX3, ZFHX4, ZFP1, ZFP14, ZFP2, ZFP28, ZFP3, ZFP30, ZFP36, ZFP36L1, ZFP36L2, ZFP37, ZFP41, ZFP42, ZFP57, ZFP62, ZFP64, ZFP69, ZFP69B, ZFP82, ZFP90, ZFP91, ZFP91-CNTF, ZFP92, ZFPL1, ZFPM1, ZFPM2, ZFR, ZFR2, ZFX, ZFY, ZFYVEl, ZFYVE16, ZFYVE19, ZFYVE21, ZFYVE26, ZFYVE27, ZFYVE28, ZFYVE9, ZG16, ZG16B, ZGLP1, ZGPAT, ZGRF1, ZHX1, ZHX1-C8orf76, ZHX2, ZHX3, ZIC1, ZIC2, ZIC3, ZIC4, ZIC5, ZIK1, ZIM2, ZIM3, ZKSCAN1, ZKSCAN2, ZKSCAN3, ZKSCAN4, ZKSCAN5, ZKSCAN7, ZKSCAN8, ZMAT1, ZMAT2, ZMAT3, ZMAT4, ZMAT5, ZMIZ1, ZMIZ2, ZMPSTE24, ZMYM1, ZMYM2, ZMYM3, ZMYM4, ZMYM5, ZMYM6, ZMYND10, ZMYND11, ZMYND12, ZMYND15, ZMYND19, ZMYND8, ZNF10, ZNF100, ZNF101, ZNF106, ZNF107, ZNF112, ZNF114, ZNF117, ZNF12, ZNF121, ZNF124, ZNF131, ZNF132, ZNF133, ZNF134, ZNF135, ZNF136, ZNF138, ZNF14, ZNF140, ZNF141, ZNF142, ZNF143, ZNF146, ZNF148, ZNF154, ZNF155, ZNF157, ZNF16, ZNF160, ZNF165, ZNF169, ZNF17, ZNF174, ZNF175, ZNF177, ZNF18, ZNF180, ZNF181, ZNF182, ZNF184, ZNF185, ZNF189, ZNF19, ZNF195, ZNF197, ZNF2, ZNF20, ZNF200, ZNF202, ZNF205, ZNF207, ZNF208, ZNF211, ZNF212, ZNF213, ZNF214, ZNF215, ZNF217, ZNF219, ZNF22, ZNF221, ZNF222, ZNF223, ZNF224, ZNF225, ZNF226, ZNF227, ZNF229, ZNF23, ZNF230, ZNF232, ZNF233, ZNF234, ZNF235, ZNF236, ZNF239, ZNF24, ZNF248, ZNF25, ZNF250, ZNF251, ZNF253, ZNF254, ZNF256, ZNF257, ZNF26, ZNF260, ZNF263, ZNF264, ZNF266, ZNF267, ZNF268, ZNF273, ZNF274, ZNF275, ZNF276, ZNF277, ZNF28, ZNF280A, ZNF280B, ZNF280C, ZNF280D, ZNF281, ZNF282, ZNF283, ZNF284, ZNF285, ZNF286A, ZNF286B, ZNF287, ZNF292, ZNF296, ZNF3, ZNF30, ZNF300, ZNF302, ZNF304, ZNF311, ZNF316, ZNF317, ZNF318, ZNF319, ZNF32, ZNF320, ZNF322, ZNF324, ZNF324B, ZNF326, ZNF329, ZNF330, ZNF331, ZNF333, ZNF334, ZNF335, ZNF337, ZNF33A, ZNF33B, ZNF34, ZNF341, ZNF343, ZNF345, ZNF346, ZNF347, ZNF35, ZNF350, ZNF354A, ZNF354B, ZNF354C, ZNF358, ZNF362, ZNF365, ZNF366, ZNF367, ZNF37A, ZNF382, ZNF383, ZNF384, ZNF385A, ZNF385B, ZNF385C, ZNF385D, ZNF391, ZNF394, ZNF395, ZNF396, ZNF397, ZNF398, ZNF404, ZNF407, ZNF408, ZNF41, ZNF410, ZNF414, ZNF415, ZNF416, ZNF417, ZNF418, ZNF419, ZNF420, ZNF423, ZNF425, ZNF426, ZNF428, ZNF429, ZNF43, ZNF430, ZNF431, ZNF432, ZNF433, ZNF436, ZNF438, ZNF439, ZNF44, ZNF440, ZNF441, ZNF442, ZNF443, ZNF444, ZNF445, ZNF446, ZNF449, ZNF45, ZNF451, ZNF454, ZNF460, ZNF461, ZNF462, ZNF467, ZNF468, ZNF469, ZNF470, ZNF471, ZNF473, ZNF474, ZNF479, ZNF48, ZNF480, ZNF483, ZNF484, ZNF485, ZNF486, ZNF487, ZNF488, ZNF490, ZNF491, ZNF492, ZNF493, ZNF496, ZNF497, ZNF500, ZNF501, ZNF502, ZNF503, ZNF506, ZNF507, ZNF510, ZNF511, ZNF512, ZNF512B, ZNF513, ZNF514, ZNF516, ZNF517, ZNF518A, ZNF518B, ZNF519, ZNF521, ZNF524, ZNF525, ZNF526, ZNF527, ZNF528, ZNF529, ZNF530, ZNF532, ZNF534, ZNF536, ZNF540, ZNF541, ZNF543, ZNF544, ZNF546, ZNF547, ZNF548, ZNF549, ZNF550, ZNF551, ZNF552, ZNF554, ZNF555, ZNF556, ZNF557, ZNF558, ZNF559, ZNF559-ZNF177, ZNF560, ZNF561, ZNF562, ZNF563, ZNF564, ZNF565, ZNF566, ZNF567, ZNF568, ZNF569, ZNF57, ZNF570, ZNF571, ZNF572, ZNF573, ZNF574, ZNF575, ZNF576, ZNF577, ZNF578, ZNF579, ZNF580, ZNF581, ZNF582, ZNF583, ZNF584, ZNF585A, ZNF585B, ZNF586, ZNF587, ZNF587B, ZNF589, ZNF592, ZNF593, ZNF594, ZNF595, ZNF596, ZNF597, ZNF598, ZNF599, ZNF600, ZNF605, ZNF606, ZNF607, ZNF608, ZNF609, ZNF610, ZNF611, ZNF613, ZNF614, ZNF615, ZNF616, ZNF618, ZNF619, ZNF620, ZNF621, ZNF622, ZNF623, ZNF624, ZNF625, ZNF625-ZNF20, ZNF626, ZNF627, ZNF628, ZNF629, ZNF630, ZNF638, ZNF639, ZNF641, ZNF644, ZNF645, ZNF646, ZNF648, ZNF649, ZNF652, ZNF653, ZNF654, ZNF655, ZNF658, ZNF66, ZNF660, ZNF662, ZNF664, ZNF665, ZNF667, ZNF668, ZNF669, ZNF670, ZNF670-ZNF695, ZNF671, ZNF672, ZNF674, ZNF675, ZNF676, ZNF677, ZNF678, ZNF679, ZNF680, ZNF681, ZNF682, ZNF683, ZNF684, ZNF687, ZNF688, ZNF689, ZNF69, ZNF691, ZNF692, ZNF695, ZNF696, ZNF697, ZNF699, ZNF7, ZNF70, ZNF700, ZNF701, ZNF703, ZNF704, ZNF705A, ZNF705B, ZNF705D, ZNF705E, ZNF705G, ZNF706, ZNF707, ZNF708, ZNF709, ZNF71, ZNF710, ZNF711, ZNF713, ZNF714, ZNF716, ZNF717, ZNF718, ZNF720, ZNF721, ZNF724, ZNF726, ZNF727, ZNF728, ZNF729, ZNF730, ZNF732, ZNF735, ZNF736, ZNF737, ZNF738, ZNF74, ZNF740, ZNF746, ZNF747, ZNF749, ZNF750, ZNF75A, ZNF75D, ZNF76, ZNF761, ZNF763, ZNF764, ZNF765, ZNF766, ZNF768, ZNF77, ZNF770, ZNF771, ZNF772, ZNF773, ZNF774, ZNF775, ZNF776, ZNF777, ZNF778, ZNF780A, ZNF780B, ZNF781, ZNF782, ZNF783, ZNF784, ZNF785, ZNF786, ZNF787, ZNF788, ZNF789, ZNF79, ZNF790, ZNF791, ZNF792, ZNF793, ZNF799, ZNF8, ZNF80, ZNF800, ZNF804A, ZNF804B, ZNF805, ZNF808, ZNF81, ZNF813, ZNF814, ZNF816, ZNF816-ZNF321P, ZNF821, ZNF823, ZNF827, ZNF829, ZNF83, ZNF830, ZNF831, ZNF835, ZNF836, ZNF837, ZNF839, ZNF84, ZNF841, ZNF843, ZNF844, ZNF845, ZNF846, ZNF85, ZNF850, ZNF852, ZNF853, ZNF860, ZNF862, ZNF865, ZNF878, ZNF879, ZNF880, ZNF883, ZNF888, ZNF891, ZNF90, ZNF91, ZNF92, ZNF93, ZNF98, ZNF99, ZNFX1, ZNHIT1, ZNHIT2, ZNHIT3, ZNHIT6, ZNRD1, ZNRF1, ZNRF2, ZNRF3, ZNRF4, ZP1, ZP2, ZP3, ZP4, ZPBP, ZPBP2, ZPLD1, ZPR1, ZRANB1, ZRANB2, ZRANB3, ZRSR1, ZRSR2, ZSCAN1, ZSCAN10, ZSCAN12, ZSCAN16, ZSCAN18, ZSCAN2, ZSCAN20, ZSCAN21, ZSCAN22, ZSCAN23, ZSCAN25, ZSCAN26, ZSCAN29, ZSCAN30, ZSCAN31, ZSCAN32, ZSCAN4, ZSCAN5A, ZSCAN5B, ZSCAN5C, ZSCAN9, ZSWIM1, ZSWIM2, ZSWIM3, ZSWIM4, ZSWIM5, ZSWIM6, ZSWIM7, ZSWIM8, ZUFSP, ZW10, ZWILCH, ZWINT, ZXDA, ZXDB, ZXDC, ZYG11A, ZYG11B, ZYX, ZZEF1, and ZZZ3.

### Exemplary Methods of Treatment of Diseases Mediated by Target Proteins

The present invention can be used to treat any disorder that is mediated by the Target Protein. Typically, the Protein Recognition Moiety is a targeting ligand or portion of a targeting ligand that binds or is bound by the Protein Recognition Moiety. Nonlimiting examples of disorders that can be treated with a heteroaryl sulfonyl compound of the present invention include abnormal cellular proliferation disorders such as cancer or a tumor. Additional disorders that can be treated with a heteroaryl sulfonyl compound of the present invention include CNS disorders, cardiovascular disorders, bacterial disorders, viral disorders, and gastrointestinal diseases.

Non-limiting examples of cancers that can be treated according to the present invention include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL)—also known as acute lymphoblastic leukemia or acute lymphoid leukemia (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In certain embodiments, the cancer is a hematopoietic cancer. In certain embodiments, the hematopoietic cancer is a lymphoma. In certain embodiments, the hematopoietic cancer is a leukemia. In certain embodiments, the leukemia is acute myelocytic leukemia (AML).

In certain embodiments, the proliferative disorder is a myeloproliferative neoplasm. In certain embodiments, the myeloproliferative neoplasm (MPN) is primary myelofibrosis (PMF).

In certain embodiments, the cancer is a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas, as described above herein.

Additional examples of solid tumors include, but are not limited to, squamous cell carcinoma, colon cancer, breast cancer, prostate cancer, lung cancer, liver cancer, pancreatic cancer, and melanoma.

Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis.

Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other disorders that can include an abnormal cellular proliferative component include Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

Exemplary cancers which may be treated by the present disclosed heteroaryl sulfonyl compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using the disclosed heteroaryl sulfonyl compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+ DLBCL of the elderly; primary mediastinal (thymic) large B-cell lymphoma, primary cutaneous DLBCL, leg type, ALK+ large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

In certain embodiments, the disease mediated, by a target protein described herein, is a neurodegenerative disease.

Non-limiting examples of neurodegenerative diseases that can be treated according to the present invention include, but are not limited to, inclusion body myositis, Amyotrophic lateral sclerosis, Parkinson's disease and any other PD-related disorder, Huntington's disease, Alzheimer's disease and any other form of dementia, Prion disease, Motor neuron diseases (MND), Spinocerebellar ataxia (SCA), Spinal muscular atrophy (SMA), Friedreich's ataxia, Lewy body disease, Spinal muscular atrophy, Pick's disease (PiD), Progressive Supranuclear Palsy (PSP), Corticobasal degeneration (CBD), Argyrophilic Grain Disease (AGD), Multiple System Atrophy (MSA), Ataxia, down syndrome, or Familial British dementia.

In certain embodiments, the disease mediated, by a target protein described herein, is a neurological disorder.

Non-limiting examples of neurodegenerative diseases that can be treated according to the present invention include, but are not limited to, Acute Spinal Cord Injury, Alzheimer's Disease, Amyotrophic Lateral Sclerosis (ALS), Ataxia, Bell's Palsy, Brain Tumors, Cerebral Aneurysm, Epilepsy and Seizures, Guillain-Barre Syndrome, Headache (Cluster Headaches, Tension Headaches, Migraine Headaches), Head Injury, Hydrocephalus, Lumbar Disk Disease (Herniated Disk), Meningitis, Multiple Sclerosis, Muscular Dystrophy, Neurocutaneous Syndromes, Parkinson's Disease, Stroke (Brain Attack), Encephalitis, Septicemia, Neuromuscular Diseases, autism, nerve tumor, dementia, epilepsy, autoimmune disease, neuro-infectious disease, neurogenetic disease, vascular disease, brain disease, nervous system disease, or Myasthenia Gravis.

### Pharmaceutical Compositions

A heteroaryl sulfonyl compound of the present invention or a pharmaceutically acceptable salt, solvate or prodrug thereof as disclosed herein can be administered as a neat chemical, but is more typically administered as a pharmaceutical composition that includes an effective amount for a host, typically a human, in need of such treatment to treat a disorder mediated by the target extracellular protein, as described herein or otherwise well-known for that extracellular protein.

The heteroaryl sulfonyl compounds of the present invention can be administered in any manner that allows the heteroaryl sulfonyl compound to covalently modify the Target Protein. As such, examples of methods to deliver a heteroaryl sulfonyl compound of the present invention include, but are not limited to, oral, systemic, topical, transdermal, parenteral, intravenous, sublingual, subcutaneous, parenteral, buccal, rectal, intra-aortal, intracranial, subdermal, transdermal, controlled drug delivery, intramuscular, or transnasal, or by other means, in dosage unit formulations containing one or more conventional pharmaceutically acceptable carriers, as appropriate. In certain embodiments, a heteroaryl sulfonyl compound of the present invention is provided in a liquid dosage form, a solid dosage form, a gel, particle, etc.

In certain embodiments the heteroaryl sulfonyl compound of the present invention is administered subcutaneously. Typically, the heteroaryl sulfonyl compound will be formulated in a liquid dosage form for subcutaneous injection, such as a buffered solution. Non-limiting examples of solutions for subcutaneous injection include phosphate buffered solution and saline buffered solution. In certain embodiments the solution is buffered with multiple salts.

In certain embodiments the heteroaryl sulfonyl compound of the present invention is administered intravenously. Typically, the heteroaryl sulfonyl compound will be formulated in a liquid dosage form for intravenous injection, such as a buffered solution. Non-limiting examples of solutions for intravenous injection include phosphate buffered solution and saline buffered solution. In certain embodiments the solution is buffered with multiple salts.

Therefore, the disclosure provides pharmaceutical compositions comprising an effective amount of heteroaryl sulfonyl heteroaryl sulfonyl compound or its pharmaceutically acceptable salt together with at least one pharmaceutically acceptable carrier for any appropriate use thereof. The pharmaceutical composition may contain a heteroaryl sulfonyl compound or salt as the only active agent, or, in an alternative embodiment, the heteroaryl sulfonyl compound and at least one additional active agent.

The term “pharmaceutically acceptable salt” as used herein refers to a salt of the described heteroaryl sulfonyl compound which is, within the scope of sound medical judgment, suitable for administration to a host such as a human without undue toxicity, irritation, allergic response, and the like, commensurate with a reasonable benefit/risk ratio, and effective for its intended use. Thus, the term “pharmaceutically acceptable salt” refers to the relatively non-toxic, inorganic and organic acid addition salts of the presently disclosed heteroaryl sulfonyl compounds. These salts can be prepared during the final isolation and purification of the heteroaryl sulfonyl compounds or by separately reacting the purified heteroaryl sulfonyl compound in its free form with a suitable organic or inorganic acid and then isolating the salt thus formed. Basic compounds are capable of forming a wide variety of different salts with various inorganic and organic acids. Acid addition salts of the basic compounds are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents. Pharmaceutically acceptable base addition salts may be formed with a metal or amine, such as alkali and alkaline earth metal hydroxide, or an organic amine. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. The base addition salts of acidic compounds are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

Any dosage form can be used that achieves the desired results. In certain embodiments the pharmaceutical composition is in a dosage form that contains from about 0.1 mg to about 1500 mg, from about 10 mg to about 1000 mg, from about 100 mg to about 800 mg, or from about 200 mg to about 600 mg of the active heteroaryl sulfonyl compound and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 25, 50, 100, 200, 250, 300, 400, 500, 600, 700, or 750 mg of active heteroaryl sulfonyl compound, or its salt.

In certain embodiments the dose ranges from about 0.01-100 mg/kg of patient bodyweight, for example about 0.01 mg/kg, about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

In some embodiments, heteroaryl sulfonyl compounds disclosed herein or used as described are administered once a day (QD), twice a day (BID), or three times a day (TID). In some embodiments, heteroaryl sulfonyl compounds disclosed herein or used as described are administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or longer.

In certain embodiments the heteroaryl sulfonyl compound of the present invention is administered once a day, twice a day, three times a day, or four times a day.

The pharmaceutical composition may be formulated as any pharmaceutically useful form, e.g., a pill, capsule, tablet, an injection or infusion solution, a syrup, an inhalation formulation, a suppository, a buccal or sublingual formulation, a parenteral formulation, or in a medical device. Some dosage forms, such as tablets and capsules, can be subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the heteroaryl sulfonyl compound is sufficient to provide a practical quantity of material for administration per unit dose of the heteroaryl sulfonyl compound. If provided as in a liquid, it can be a solution or a suspension.

Representative carriers include phosphate buffered saline, water, solvent(s), diluents, pH modifying agents, preservatives, antioxidants, suspending agents, wetting agent, viscosity agents, tonicity agents, stabilizing agents, and combinations thereof. In some embodiments, the carrier is an aqueous carrier. Examples of aqueous carries include, but are not limited to, an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, ProVisc®, diluted ProVisc®, Provisc® diluted with PBS, Krebs buffer, Dulbecco's PBS, normal PBS, sodium hyaluronate solution (HA, 5 mg/mL in PBS), citrate buffer, simulated body fluids, plasma platelet concentrate and tissue culture medium or an aqueous solution or suspension comprising an organic solvent. Acceptable solutions include, for example, water, Ringer's solution and isotonic sodium chloride solutions. The formulation may also be a sterile solution, suspension, or emulsion in a non-toxic diluent or solvent such as 1,3-butanediol.

Viscosity agents may be added to the pharmaceutical composition to increase the viscosity of the composition as desired. Examples of useful viscosity agents include, but are not limited to, hyaluronic acid, sodium hyaluronate, carbomers, polyacrylic acid, cellulosic derivatives, polycarbophil, polyvinylpyrrolidone, gelatin, dextin, polysaccharides, polyacrylamide, polyvinyl alcohol (including partially hydrolyzed polyvinyl acetate), polyvinyl acetate, derivatives thereof and mixtures thereof.

Solutions, suspensions, or emulsions for administration may be buffered with an effective amount necessary to maintain a pH suitable for the selected administration. Suitable buffers are well known by those skilled in the art. Some examples of useful buffers are acetate, borate, carbonate, citrate, and phosphate buffers. Solutions, suspensions, or emulsions for topical, for example, ocular administration may also contain one or more tonicity agents to adjust the isotonic range of the formulation. Suitable tonicity agents are well known in the art. Some examples include glycerin, mannitol, sorbitol, sodium chloride, and other electrolytes.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the heteroaryl sulfonyl compound of the present invention.

The pharmaceutical compositions/combinations can be formulated for oral administration. These compositions can contain any amount of active heteroaryl sulfonyl compound that achieves the desired result, for example between 0.1 and 99 weight % (wt. %) of the heteroaryl sulfonyl compound and usually at least about 5 wt. % of the heteroaryl sulfonyl compound. Some embodiments contain from about 25 wt. % to about 50 wt. % or from about 5 wt. % to about 75 wt. % of the heteroaryl sulfonyl compound. Enteric coated oral tablets may also be used to enhance bioavailability of the heteroaryl sulfonyl compounds for an oral route of administration.

Formulations suitable for rectal administration are typically presented as unit dose suppositories. These may be prepared by admixing the active heteroaryl sulfonyl compound with one or more conventional solid carriers, for example, cocoa butter, and then shaping the resulting mixture.

### Processes of Manufacture:

The heteroaryl sulfonyl compounds of the present invention can be manufactured according to routes described in the Working Examples below or as otherwise known in the patent or scientific literature and if appropriate supported by the knowledge of the ordinary worker or common general knowledge.

Some of the carbons in the heteroaryl sulfonyl compounds described herein are drawn with designated stereochemistry. Other carbons are drawn without stereochemical designation. When drawn without designated stereochemistry, that carbon can be in any desired stereochemical configuration that achieves the desired purpose. One skilled in the art will recognize that pure enantiomers, enantiomerically enriched compounds, racemates and diastereomers can be prepared by methods known in the art as guided by the information provided herein. Examples of methods to obtain optically active materials include at least the following:
i) chiral liquid chromatography—a technique whereby diastereomers are separated in a liquid mobile phase by virtue of their differing interactions with a stationary phase (including vial chiral HPLC). The stationary phase can be made of chiral material or the mobile phase can contain an additional chiral material to provoke the differing interactions;
ii) non-chiral chromatography of diastereomers-Often diastereomers can be separated using normal non-chiral column conditions;
iii) chiral gas chromatography—a technique whereby the racemate is volatilized and enantiomers are separated by virtue of their differing interactions in the gaseous mobile phase with a column containing a fixed non-racemic chiral adsorbent phase;
iv) simultaneous crystallization—a technique whereby the individual diastereomers are separately crystallized from a solution;
v) enzymatic resolutions—a technique whereby partial or complete separation of diastereomers are separated by virtue of differing rates of reaction with an enzyme;
vi) chemical asymmetric synthesis—a synthetic technique whereby the desired diastereomer is synthesized from an achiral precursor under conditions that produce asymmetry (i.e. chirality) in the product, which may be achieved by chiral catalysts or chiral auxiliaries;
vii) diastereomer separations—a technique whereby a racemic compound is reaction with an enantiomerically pure reagent (the chiral auxiliary) that converts the individual enantiomers to diastereomers. The resulting diastereomers are then separated by chromatography or crystallization by virtue of their now more distinct structural differences the chiral auxiliary later removed to obtain the desired enantiomer; and
viii) extraction with chiral solvents—a technique whereby diastereomers are separated by virtue of preferential dissolution of one over the others in a particular chiral solvent.

### Example 1. Installation of Heteroaryl Sulfonyl Groups

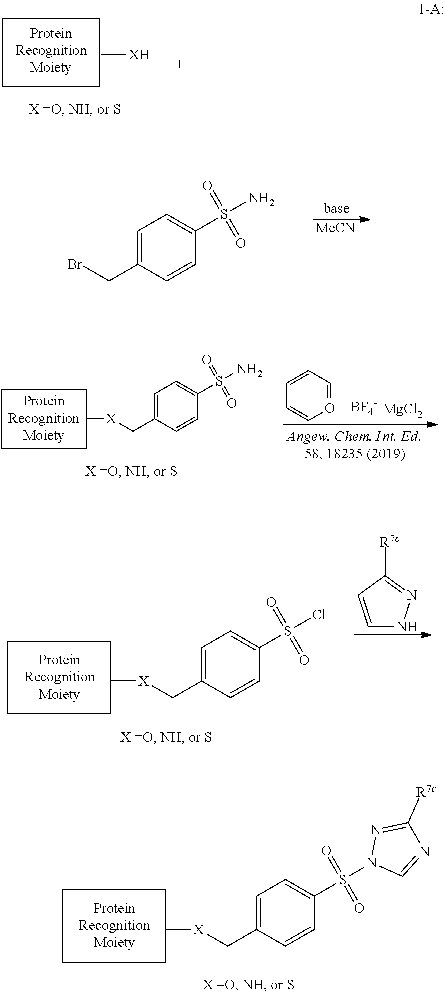

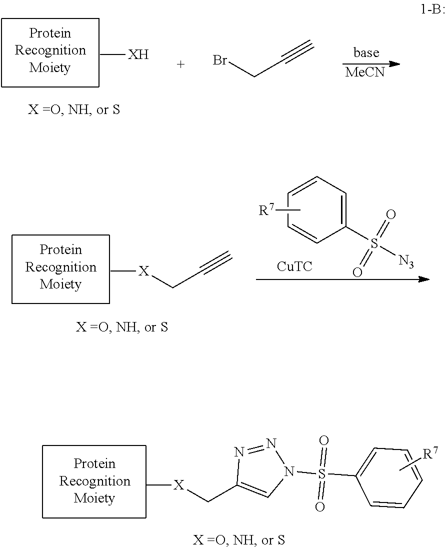

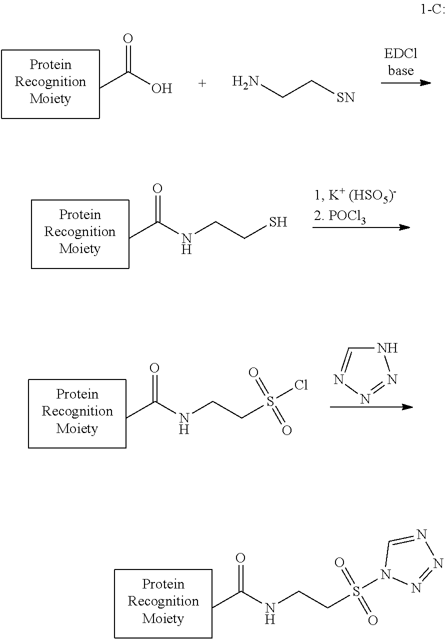

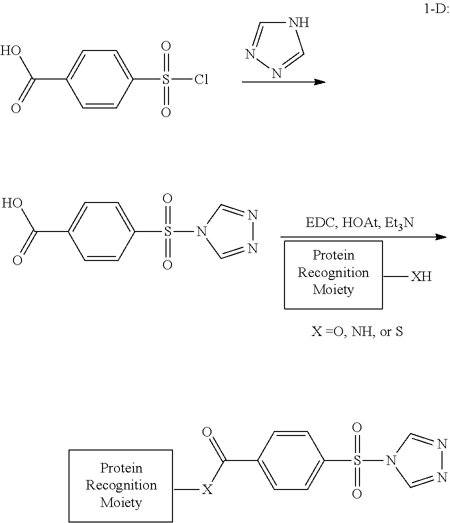

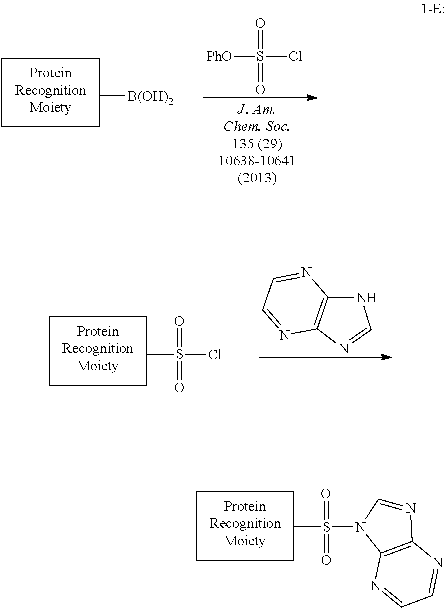

TABLE 1
Non-limiting heteroaryl sulfonyl compounds of the present invention

| Compound # | Structure |
|---|---|
| 1 | 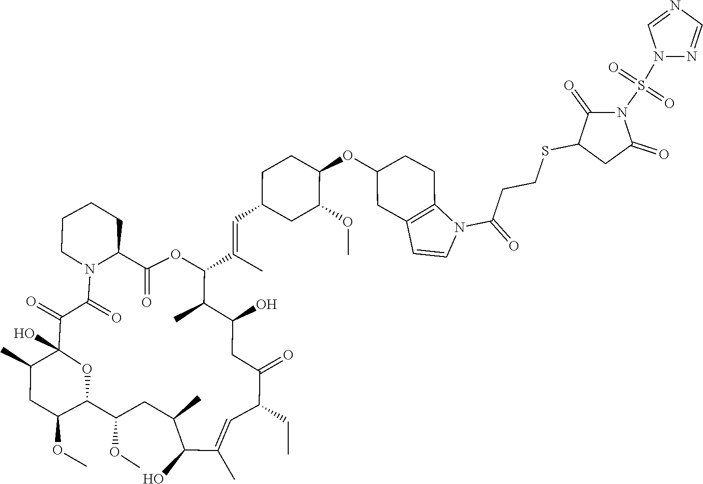 [SMILES: CC[C@@H]1/C=C(\C)[C@@H](O)[C@H](C)C[C@H](OC)[C@H]2O[C@@](O)(C(=O)C(=O)N3CCCC[C@H]3C(=O)O[C@H](/C(C)=C/[C@@H]3CC[C@@H](OC4CCc5c(ccn5C(=O)CCSC5CC(=O)N(S(=O)(=O)n6cncn6)C5=O)C4)[C@H](OC)C3)[C@H](C)[C@@H](O)CC1=O)[C@H](C)C[C@@H]2OC] |
| 2 | 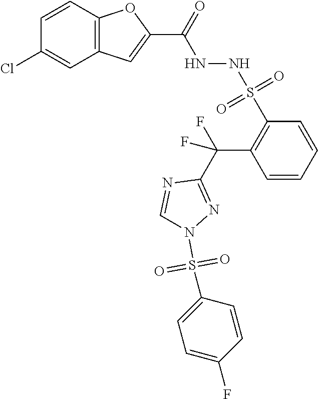 [SMILES: O=C(NNS(=O)(=O)c1ccccc1C(F)(F)c1ncn(S(=O)(=O)c2ccc(F)cc2)n1)c1cc2cc(Cl)ccc2o1] |
| 3 | 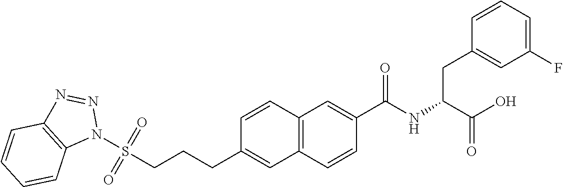 [SMILES: O=C(N[C@H](Cc1cccc(F)c1)C(=O)O)c1ccc2cc(CCCS(=O)(=O)n3nnc4ccccc43)ccc2c1] |
| 4 | 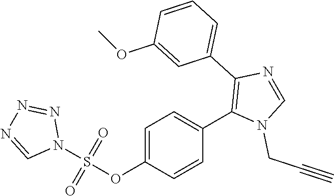 [SMILES: C#CCn1cnc(-c2cccc(OC)c2)c1-c1ccc(OS(=O)(=O)n2cnnn2)cc1] |
| 5 | 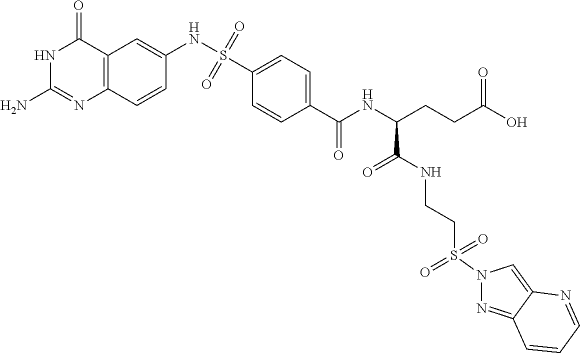 [SMILES: Nc1nc2ccc(NS(=O)(=O)c3ccc(C(=O)N[C@@H](CCC(=O)O)C(=O)NCCS(=O)(=O)n4cc5ncccc5n4)cc3)cc2c(=O)[nH]1] |
| 6 | 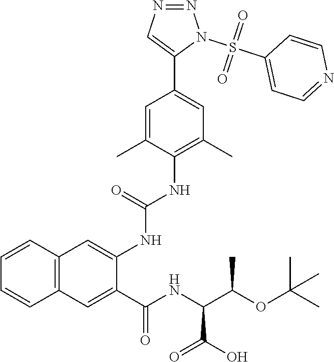 [SMILES: Cc1cc(-c2cnnn2S(=O)(=O)c2ccncc2)cc(C)c1NC(=O)Nc1cc2ccccc2cc1C(=O)N[C@H](C(=O)O)[C@@H](C)OC(C)(C)C] |
| 7 | 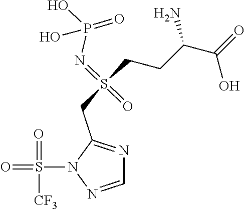 [SMILES: N[C@@H](CC[S@@](=O)(Cc1ncnn1S(=O)(=O)C(F)(F)F)=NP(=O)(O)O)C(=O)O] |
| 8 | 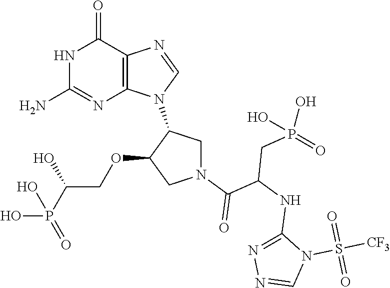 [SMILES: Nc1nc2c(ncn2[C@@H]2CN(C(=O)C(CP(=O)(O)O)Nc3nncn3S(=O)(=O)C(F)(F)F)C[C@H]2OC[C@@H](O)P(=O)(O)O)c(=O)[nH]1] |
| 9 | 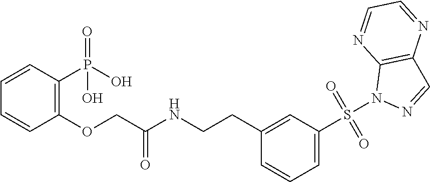 [SMILES: O=C(COc1ccccc1P(=O)(O)O)NCCc1cccc(S(=O)(=O)n2ncc3nccnc32)c1] |
| 10 | 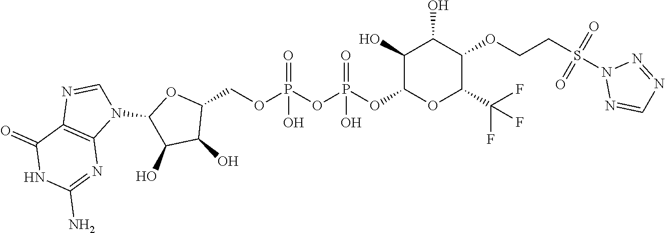 [SMILES: Nc1nc2c(ncn2[C@@H]2O[C@H](COP(=O)(O)OP(=O)(O)O[C@H]3O[C@@H](C(F)(F)F)[C@@H](OCCS(=O)(=O)n4ncnn4)[C@@H](O)[C@@H]3O)[C@@H](O)[C@H]2O)c(=O)[nH]1] |
| 11 | 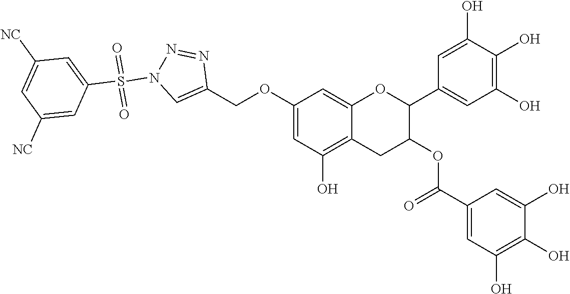 [SMILES: N#Cc1cc(C#N)cc(S(=O)(=O)n2cc(COc3cc(O)c4c(c3)OC(c3cc(O)c(O)c(O)c3)C(OC(=O)c3cc(O)c(O)c(O)c3)C4)nn2)c1] |
| 12 | 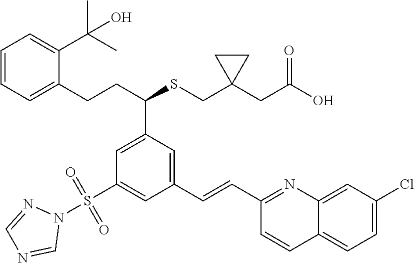 [SMILES: CC(C)(O)c1ccccc1CC[C@@H](SCC1(CC(=O)O)CC1)c1cc(/C=C/c2ccc3ccc(Cl)cc3n2)cc(S(=O)(=O)n2cncn2)c1] |
| 13 | 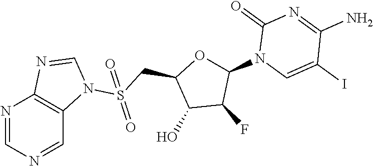 [SMILES: Nc1nc(=O)n([C@@H]2O[C@H](CS(=O)(=O)n3cnc4ncncc43)[C@@H](O)[C@@H]2F)cc1I] |
| 14 | 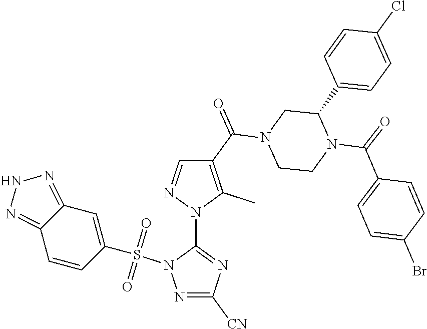 [SMILES: Cc1c(C(=O)N2CCN(C(=O)c3ccc(Br)cc3)[C@@H](c3ccc(Cl)cc3)C2)cnn1-c1nc(C#N)nn1S(=O)(=O)c1ccc2n[nH]nc2c1] |
| 15 | 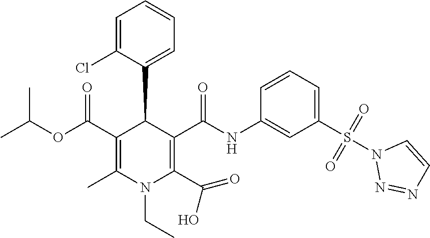 [SMILES: CCN1C(C)=C(C(=O)OC(C)C)[C@@H](c2ccccc2Cl)C(C(=O)Nc2cccc(S(=O)(=O)n3ccnn3)c2)=C1C(=O)O] |
| 16 | 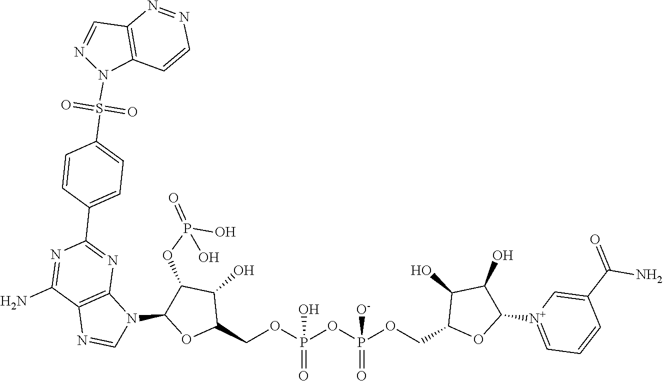 |
| 17 | 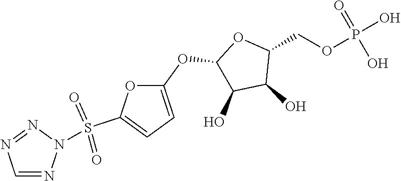 [SMILES: O=P(O)(O)OC[C@H]1O[C@@H](Oc2ccc(S(=O)(=O)n3ncnn3)o2)[C@H](O)[C@@H]1O] |
| 18 | 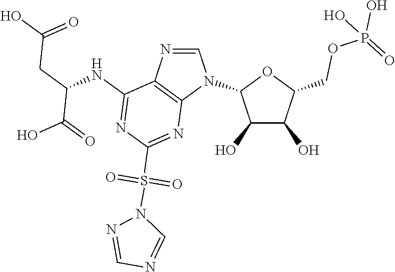 [SMILES: O=C(O)C[C@H](Nc1nc(S(=O)(=O)n2cncn2)nc2c1ncn2[C@@H]1O[C@H](COP(=O)(O)O)[C@@H](O)[C@H]1O)C(=O)O] |
| 19 | 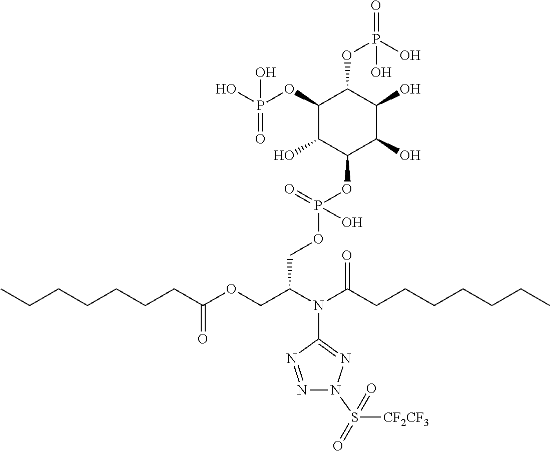 |

All publications and patent applications cited in this specification are herein incorporated by reference as if each individual publication or patent application were specifically and individually indicated to be incorporated by reference.

Although the foregoing invention has been described in some detail by way of illustration and example for the purposes of clarity of understanding, it will be readily apparent to one of ordinary skill in the art in light of the teaching of this invention that certain changes and modification may be made thereto without departing from the spirit or scope of the invention as defined in the claims.

We claim:

1. A pharmaceutically acceptable compound of Formula: 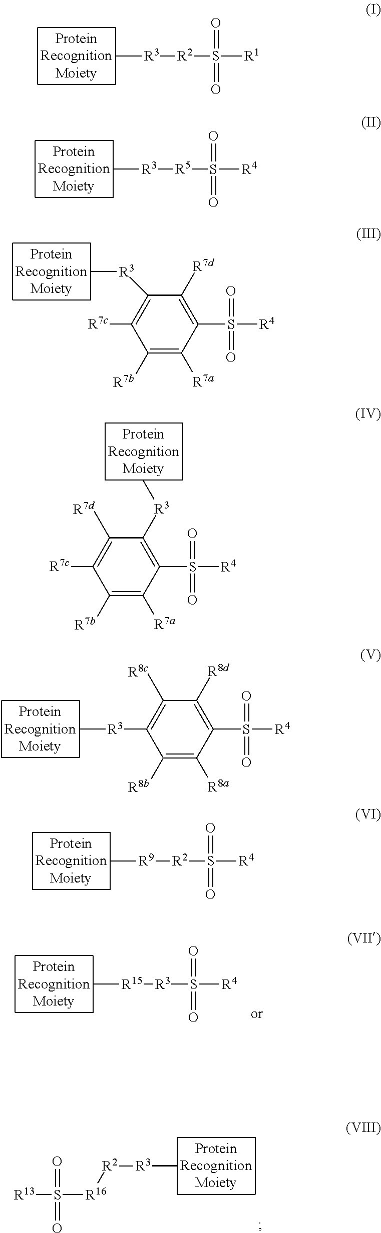
or a pharmaceutically acceptable salt thereof,
wherein:
R¹ is selected from the group consisting of: 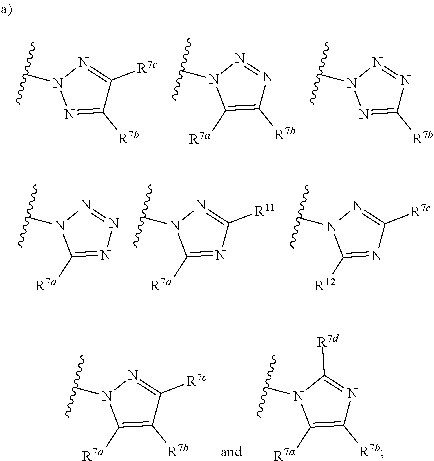
or
b) a bicyclic heteroaryl which is optionally substituted with 1, 2, or 3 substituents selected from R⁷;
R² is independently selected at each instance from the group consisting of bond, alkyl, cycloalkyl, aryl, heterocycle, —S—, —O—, —NR⁶—, —(CH₂)ₚ—C(O)—NR⁶—, —(CH₂CH₂O)ₚ—, —(OCH₂CH₂)ₚ—, —NR⁶C(O)NR⁶—, —C(O)NR⁶—, —OC(O)NR⁶—, aryl-C(O)—NR⁶—, heteroaryl-C(O)—NR⁶—, bicycle, and heteroaryl, each of which is optionally substituted with 1, 2, or 3 substituents selected from R⁷;
R³ is independently selected at each instance from bond, alkyl, cycloalkyl, aryl, heterocycle, —S—, —O—, —NR⁶—, —(CH₂)ₚ—C(O)—, —(CH₂)ₚ—C(O)—NR⁶—, —(CH₂CH₂O)ₚ—, —(OCH₂CH₂)ₚ—, —C(O)—, —NR⁶C(O)—, —NR⁶C(O)NR⁶—, —C(O)NR⁶—, —OC(O)NR⁶—, —NR⁶S(O)₂NR⁶—, —S(O)₂NR⁶—, aryl-C(O)—NR⁶—, heteroaryl-C(O)—NR⁶—, bicycle, and heteroaryl, each of which is optionally substituted with 1, 2, or 3 substituents selected from R⁷;
p is independently selected from 1, 2, 3, 4, 5, and 6;
R⁴ is a heteroaryl group with 1, 2, 3, or 4 nitrogen atoms, where the bond to the sulfur atom is through one of the nitrogen atoms present in the cycle, and each heteroaryl is optionally substituted with 1, 2, or 3 substituents selected from R⁷;
R⁵ is selected from the group consisting of alkyl, cycloalkyl, naphthyl, heterocycle, —S—, —O—, —NR⁶—, —(CH₂)ₚ—C(O)—NR⁶—, —(CH₂CH₂O)ₚ—, —(OCH₂CH₂)ₚ—, —NR⁶C(O)NR⁶—, —C(O)NR⁶—, —OC(O)NR⁶—, heteroaryl-C(O)—NR⁶—, bicycle, and heteroaryl, each of which is optionally substituted with 1, 2, or 3 substituents selected from R⁷;
R⁶ is independently selected at each instance hydrogen or alkyl;
R⁷, R^{7a}, R^{7b}, RC, and R^{7d} are independently selected at each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, and —SR⁶, wherein each alkyl, haloalkyl, alkenyl, cycloalkyl, heterocycle, aryl, and heteroaryl is optionally substituted with 1, 2, or 3 substituents selected from R¹⁷;
R¹⁷ is independently selected in each instance from the group consisting of hydrogen, halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, —S(O)₂N(R⁶)₂, and —SR⁶;
R^{8a}, R^{8b}, R^{8c}, and R^{8d} are independently selected at each instance from the group consisting of R⁷ and R¹² wherein at least one of R^{8a}, R^{8b}, R^{8c}, and R^{8d} is R¹²;
R⁹ is alkyl, alkenyl, haloalkyl, cycloalkyl, heterocycle, —NR⁶C(O)—, —NR⁶C(O)NR⁶—, —C(O)NR⁶—, —OC(O)NR⁶—, —NR⁶S(O)₂NR⁶—, —S(O)₂NR⁶—, bicycle, or heteroaryl, each of which is optionally substituted with 1, 2, or 3 substituents selected from R⁷;
R¹¹ is hydrogen, halogen, alkyl, haloalkyl, alkenyl, cycloalkyl, heterocycle, naphthyl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, or —SR⁶, wherein each alkyl, haloalkyl, alkenyl, cycloalkyl, heterocycle, aryl, and heteroaryl optionally substituted with 1, 2, or 3 substituents selected from R¹⁷;
R¹² is halogen, alkyl, haloalkyl, alkenyl, cycloalkyl, heterocycle, aryl, heteroaryl, cyano, nitro, —C(O)R⁶, —OC(O)R⁶, —NR⁶C(O)R⁶, —C(O)OR⁶, —OC(O)OR⁶, —NR⁶C(O)OR⁶, —C(O)N(R⁶)₂, —OC(O)N(R⁶)₂, —NR⁶C(O)N(R⁶)₂, —OR⁶, —N(R⁶)₂, —S(O)R⁶, —S(O)₂R⁶, —S(O)OR⁶, —S(O)₂OR⁶, —S(O)N(R⁶)₂, S(O)₂N(R⁶)₂, or —SR⁶, wherein each alkyl, haloalkyl, alkenyl, cycloalkyl, heterocycle, aryl, and heteroaryl is optionally substituted with 1, 2, or 3 substituents selected from R¹⁷;
R¹³ is alkyl, haloalkyl, cycloalkyl, heterocycle, heteroaryl, aryl, —OR⁶, —N(R⁶)₂, —C(O)R⁶, —NR⁶C(O)R⁶, —C(O)N(R⁶)₂, or —NR⁶C(O)N(R⁶)₂, each of which is optionally substituted with 1, 2, or 3 substituents selected from R⁷;
R¹⁵ is alkyl, alkenyl, haloalkyl, cycloalkyl, aryl, heterocycle, —S—, —O—, —NR⁶—, —S-alkyl-, —O-alkyl-, —NR⁶-alkyl-, -alkyl-C(O)—, -alkyl-C(O)-alkyl-, -alkyl-C(O)—NR⁶-alkyl-, —C(O)—NR⁶-alkyl-, -alkyl-C(O)—NR⁶—, -alkyl-C(O)—O-alkyl-, —C(O)—O-alkyl-, -alkyl-C(O)—O—, —C(O)—, —NR⁶C(O)NR⁶—, —C(O)NR⁶—, —OC(O)NR⁶—, —NR⁶S(O)₂NR⁶—, —S(O)₂NR⁶—, bicycle, or heteroaryl, each of which except bond is optionally substituted with 1, 2, or 3 substituents independently selected from R⁷;
R¹⁶ is a heteroaryl group, where the bond to the sulfur atom is through one of the nitrogen atoms present in the cycle, and R¹⁶ is optionally substituted with 1, 2, or 3 substituents selected from R⁷;
Protein Recognition Moiety is a molecule which can bind to or otherwise anchors to a Target Protein; and
Target Protein is a mediator of disease.

2. The pharmaceutically acceptable compound of claim 1, wherein R³ is phenyl, alkyl, or heteroaryl optionally substituted with 1, 2, or 3 substituents selected from R⁷.

3. The pharmaceutically acceptable compound of claim 2, wherein the compound is of Formula: 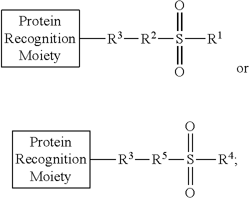
or a pharmaceutically acceptable salt thereof.

4. The pharmaceutically acceptable compound of claim 3, wherein the compound is not substituted.

5. The pharmaceutically acceptable compound of claim 3, wherein R¹ and R⁴ are

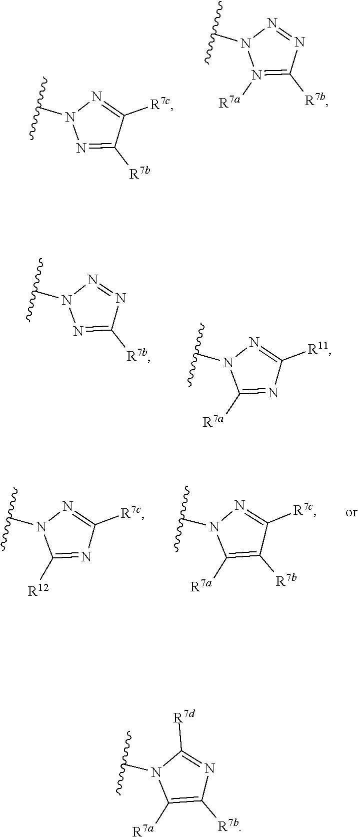

6. The pharmaceutically acceptable compound of claim 5, wherein R² is bond.

7. The pharmaceutically acceptable compound of claim 5, wherein R² is phenyl, alkyl, or heteroaryl optionally substituted with 1, 2, or 3 substituents selected from R⁷.

8. The pharmaceutically acceptable compound of claim 2, wherein the compound is Formula: 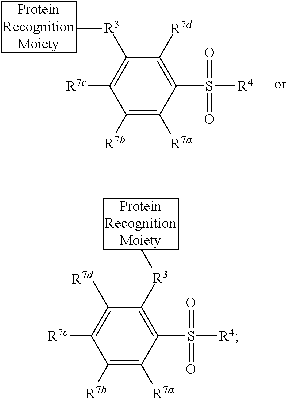
or a pharmaceutically acceptable salt thereof.

9. The pharmaceutically acceptable compound of claim 8, wherein the compound is not substituted.

10. The pharmaceutically acceptable compound of claim 2, wherein the compound is Formula: 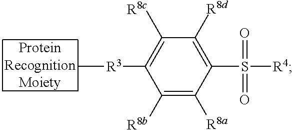
or a pharmaceutically acceptable salt thereof.

11. The pharmaceutically acceptable compound of claim 10, wherein the compound is not substituted.

12. The pharmaceutically acceptable compound of claim 2, wherein the compound is Formula: 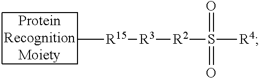
or a pharmaceutically acceptable salt thereof.

13. The pharmaceutically acceptable compound of claim 12, wherein the compound is not substituted.

14. The pharmaceutically acceptable compound of claim 12, wherein R⁴ is

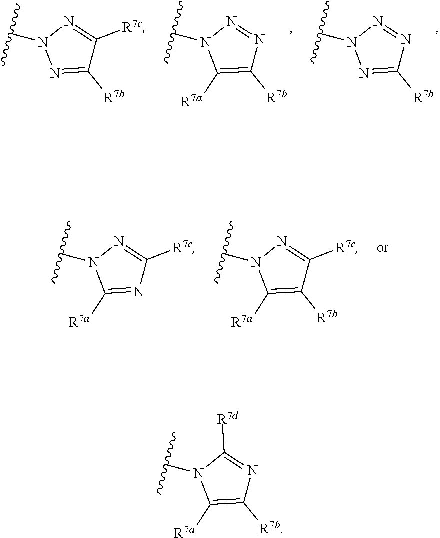

15. The pharmaceutically acceptable compound of claim 14, wherein R² is phenyl, alkyl, or heteroaryl optionally substituted with 1, 2, or 3 substituents selected from R⁷.

16. The pharmaceutically acceptable compound of claim 2, wherein the compound is of Formula: 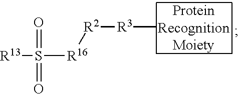
or a pharmaceutically acceptable salt thereof.

17. The pharmaceutically acceptable compound of claim 16, wherein the compound is not substituted.

18. The pharmaceutically acceptable compound of claim 16, wherein R¹³ is phenyl optionally substituted with 1, 2, or 3 substituents selected from R⁷.

19. The pharmaceutically acceptable compound of claim 16, wherein R¹³ is alkyl optionally substituted with 1, 2, or 3 substituents selected from R⁷.

20. The pharmaceutically acceptable compound of claim 16, wherein R¹⁶ is

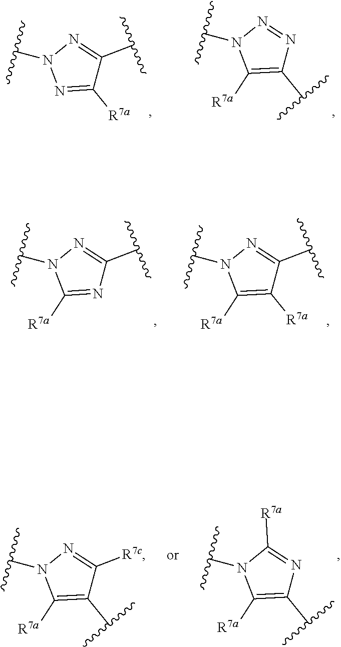

21. The pharmaceutically acceptable compound of claim 2, wherein the compound is of Formula: 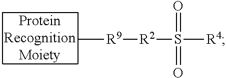
or a pharmaceutically acceptable salt thereof.

22. The pharmaceutically acceptable compound of claim 21, wherein the compound is not substituted.

23. The pharmaceutically acceptable compound of claim 21, wherein R⁴ is

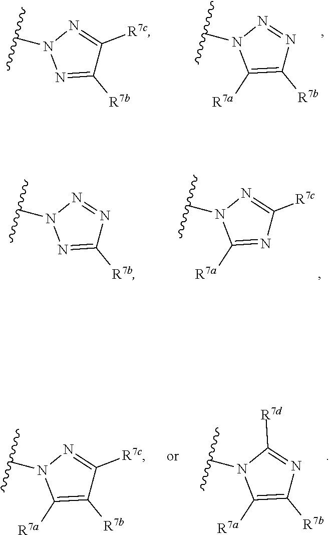

24. The pharmaceutically acceptable compound of claim 23, wherein R⁹ is selected from alkyl, cycloalkyl, and heteroaryl, each of which except bond is optionally substituted with 1, 2, or 3 substituents independently selected from R⁷.

25. A pharmaceutical composition comprising a pharmaceutically acceptable compound of claim 1 or a pharmaceutically acceptable salt thereof and a pharmaceutically acceptable excipient.

26. The pharmaceutical composition of claim 25, wherein the composition is suitable for oral delivery.

27. The pharmaceutical composition of claim 25, wherein the pharmaceutically acceptable compound is of Formula: 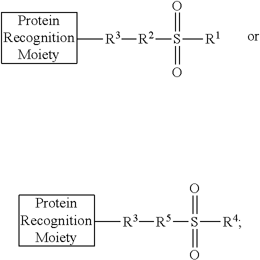
or a pharmaceutically acceptable salt thereof.

28. The pharmaceutical composition of claim 25, wherein the pharmaceutically acceptable compound is Formula: 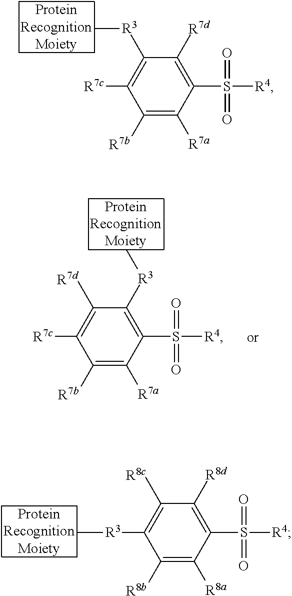
or a pharmaceutically acceptable salt thereof.

29. The pharmaceutical composition of claim 25, wherein the pharmaceutically acceptable compound is of Formula: 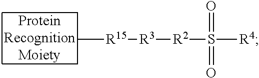
or a pharmaceutically acceptable salt thereof.

30. The pharmaceutical composition of claim 25, wherein the pharmaceutically acceptable compound is of Formula: 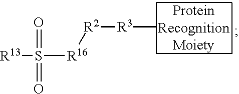
or a pharmaceutically acceptable salt thereof.

31. A method of treating a disorder mediated by a Target Protein comprising administering an effective amount of a pharmaceutically acceptable compound of claim 1 or a pharmaceutically acceptable salt thereof or a pharmaceutical composition thereof to a human in need thereof.

32. The method of claim 31, wherein the disorder is a cancer.

33. The method of claim 32, wherein the cancer is a solid cancer.

34. The method of claim 32, wherein the cancer is a hematological cancer.

35. The method of claim 32, wherein the cancer is metastatic.

36. The method of claim 32, wherein the cancer is relapsed.

37. The method of claim 32, wherein the cancer is refractory.

38. The method of claim 32, wherein the pharmaceutically acceptable compound is of Formula: 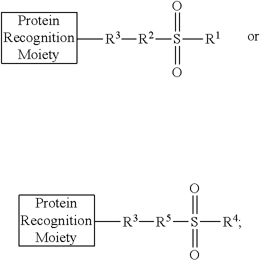
or a pharmaceutically acceptable salt thereof.

39. The method of claim 32, wherein the pharmaceutically acceptable compound is Formula: 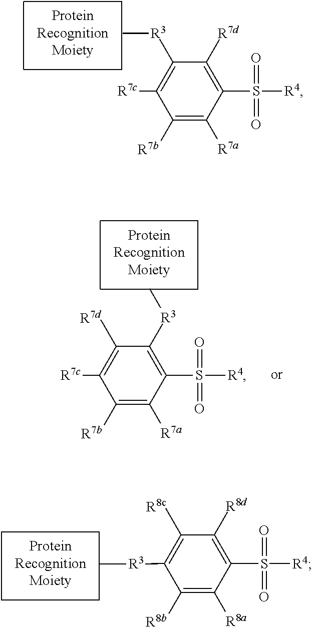
or a pharmaceutically acceptable salt thereof.

40. The method of claim 32, wherein the pharmaceutically acceptable compound is of Formula: 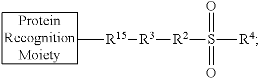
or a pharmaceutically acceptable salt thereof.

41. The method of claim 32, wherein the pharmaceutically acceptable compound is of Formula: 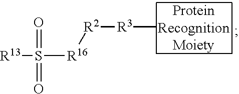
or a pharmaceutically acceptable salt thereof.


SEQUENCE LISTING (11 sequences)

SEQ ID NO: 1 - protein, 14 aa, synthetic construct
MIRSFSEPFGRDLL

SEQ ID NO: 2 - protein, 7 aa, synthetic construct
RRQRSAP

SEQ ID NO: 3 - protein, 4 aa, synthetic construct
SISR

SEQ ID NO: 4 - protein, 8 aa, synthetic construct
RTQSLSAL

SEQ ID NO: 5 - protein, 8 aa, synthetic construct
RSMSETGT

SEQ ID NO: 6 - protein, 10 aa, synthetic construct
TNPESKVFYL

SEQ ID NO: 7 - protein, 6 aa, synthetic construct
RQRSAP

SEQ ID NO: 8 - protein, 27 aa, synthetic construct
GQQDLMINNPLSQDEGSLWNKFFQDKE

SEQ ID NO: 9 - protein, 18 aa, synthetic construct
VEPQKFAEELIHRLEAVQ

SEQ ID NO: 10 - protein, 7 aa, synthetic construct
  VARIANT 1: note = X can be any naturally occurring amino acid
  VARIANT 4: note = X can be any naturally occurring amino acid
  VARIANT 7: note = X can be any naturally occurring amino acid
XFTXAQX

SEQ ID NO: 11 - protein, 14 aa, synthetic construct
YSPTSPSYSPTSPS